(19) United States
(12) Patent Application Publication
(10) Pub. No.: US 2016/0251675 A1
(43) Pub. Date: Sep. 1, 2016

(54) Drought-Resistant Cereal Grasses and Related Materials and Methods

(71) Applicants: Amelia HENRY, Laguna (PH); INTERNATIONAL RICE RESEARCH INSTITUTE, Metro Manila (PH)

(72) Inventors: Amelia Henry, Laguna (PH); Ajay Kohli, Metro Manila (PH); Arvind Kumar, Metro Manila (PH)

(21) Appl. No.: 15/027,896

(22) PCT Filed: Oct. 8, 2014
(86) PCT No.: PCT/US14/59676

§ 371 Date: Apr. 7, 2016

Related U.S. Application Data
(60) Provisional application No. 61/888,397, filed on Oct. 8, 2013.
(60) Provisional application No. 61/994,558, filed on May 16, 2014.

(51) Int. Cl. C12N 15/82; A01H 5/10; C12Q 1/68; A01H 1/02

(52) U.S. Cl. CPC C12N 15/8273; A01H 1/02; A01H 5/10; C12Q 1/6895; C12Q 2600/13; C12Q 2600/156

(57) ABSTRACT

Described herein are methods and materials useful for improving lateral root growth, water uptake, and the yield of grain of cereal grasses grown under drought stress conditions. In particular, the present disclosure provides a quantitative trait locus associated with improved yield under drought stress. The disclosure further provides recombinant DNA for the generation of transgenic plants, transgenic plant cells, and methods of producing the same. The present disclosure further provides methods for generating transgenic seed that can be used to produce a transgenic plant having improved yield under drought stress, and methods for improving yield under drought stress in a cereal grass involving marker assisted selection and backcrossing.

### CROSS REFERENCE TO RELATED APPLICATIONS

This application claims the priority benefit of U.S. Provisional Application No. 61/888,397, filed Oct. 8, 2013, and U.S. Provisional Application No. 61/994,558, filed May 16, 2014, the entire disclosures of which are expressly incorporated herein by reference for all purposes.

### STATEMENT REGARDING GOVERNMENT FUNDING

This invention was not made with United States Government support.

### STATEMENT REGARDING SEQUENCE LISTING

The Sequence Listing, filed electronically and identified as 53-55195-IRRI-13-005_SL.txt, was created on Oct. 8, 2014, is 4,929,609 bytes in size and is hereby incorporated by reference.

### BACKGROUND OF THE INVENTION

Cereal grasses, cultivated for their edible seeds, are grown in greater quantities and provide more food energy worldwide than any other type of crop. Cereal grasses comprise a range of crops, including corn, rice, wheat, barley, sorghum, millet, oats, and rye. Together, maize, wheat and rice account for nearly half of all food calories consumed globally. Drought is one of the most important and damaging abiotic stresses for all cereal grasses. With rice, drought severely hampers rice productivity in rainfed areas. In Asia, more that 23 million ha of rice are rainfed. Eastern India and adjoining areas of Nepal occupy a large drought-affected area with an estimate of around 17 million ha. In 2004, widespread severe drought in much of Asia not only resulted in agricultural production losses of hundreds of millions of dollars, but also pushed millions of people into poverty. In Thailand, drought hit 70 of the country's 76 provinces and affected more than 8 million people. Production loss from major crop failures covering 2 million hectares is estimated at US$326 million, resulting in a 3.9% decline in the 2004 agricultural gross domestic product (GDP). More than half of the rural population of Thailand relies on farm income for their livelihoods. In 2004, the normally lush tropical southern Chinese island of Hainan suffered its worst drought in 50 years, with 12 million hectares of farmland affected. Vietnam's eight central highland provinces suffered their worst drought in 28 years, affecting around 1 million people and causing an estimated $80 million worth of crop losses. In March 2005, Cambodian Prime Minister Hun Sen called for international assistance for a national campaign to help farmers who are short of water. Coping with recurrent drought is part of life for millions of Asia's rural poor.

Drought is an extended period of substantially lower-than-usual rainfall, leading to a shortage of water for domestic use and agriculture. Drought may affect rice by several mechanisms, including: inhibition of leaf production and decline in leaf area, leading to retarded leaf growth and light interception; closure of stomata, leading to reduced transpiration rates and reduced photosynthesis; leaf rolling, leading to reduction in effective leaf area available for light interception; enhanced leaf senescence, or leaf deaths, leading to reduced canopy photosynthesis; reduced plant height and spikelet number, resulting in low yield production; spikelet sterility, resulting in decreased percentage of filled spikelets; delayed flowering, caused by drought during the vegetative development stage; reduced tillering and tiller death, resulting in a reduction in the number of tillers and panicles per hill; and decreased grain weight, if drought occurs during flowering.

With increasing incidence and severity of drought, popular rice varieties grown by Asian farmers are not keeping up with the needs of the farmer or of the global population. Progress has, however, been slow in developing rice varieties that thrive under drought stress. This is mainly due to the complex nature of drought-tolerant mechanisms: large genotype×environment; quantitative trait locus (QTL)×environment and QTL×recipient genetic background interactions; and the absence of QTLs with a large and consistent effect against high-yielding but drought-susceptible varieties. The problem is further complicated by the number of physiological mechanisms and biochemical pathways affected by drought. And while several drought-tolerant rice varieties have been developed, it remains the ultimate aim of plant breeders to identify rice genotypes with a stable performance across a range of environments. This can be a very time-consuming process.

A marker-assisted breeding (MAB) strategy, advocated to be a fast-track approach in rice improvement for drought-prone environments, can be a suitable alternative strategy. The marker assisted backcrossing (MABC) approach has been used to improve the drought tolerance of high-yielding, popular, farmer-adapted varieties grown on a large scale. QTLs with large and consistent effects are worthy for use in marker-assisted selection (MAS) to improve the drought tolerance of presently cultivated varieties. The most suitable QTL for drought would be one that can overcome QTL×genetic background, QTL×environment, and QTL×ecosystem effects. One skilled in the art will recognize that the identification and introgression of QTLs in the background of elite rice varieties could be helpful in MAB and the generation of new drought-tolerant varieties.

### SUMMARY OF THE INVENTION

Described herein are methods and materials useful for improving lateral root growth, water uptake, and the yield of grain of cereal grasses grown under drought stress conditions. In particular, the present disclosure provides a quantitative trait locus associated with improved yield under drought stress. The disclosure further provides recombinant DNA for the generation of transgenic plants, transgenic plant cells, and methods of producing the same. The present disclosure further provides methods for generating transgenic seed that can be used to produce a transgenic plant having improved yield under drought stress, and methods for improving yield under drought stress in a cereal grass involving marker assisted selection and backcrossing.

In a particular embodiment described herein, is a method of improving lateral root growth and water uptake in a cereal grass comprising: a) crossing a crossing plant of one variety of cereal grass having chromosomal DNA that comprises a nucleic acid comprising qDTY_{12.1}, or a yield-improving part thereof, with a recipient plant of a distinct variety of cereal grass having chromosomal DNA that does not include a nucleic acid comprising qDTY_{12.1}, or a yield-improving part thereof; and b) selecting one or more progeny plants having chromosomal DNA that comprises a nucleic acid comprising qDTY_{12.1}, or a yield-improving part thereof, wherein qDTY_{12.1}, or a yield-improving part thereof, is detected in the crossing plant, recipient plant, or one or more progeny plants by analyzing genomic DNA from the crossing plant, the recipient plant, or one or more progeny plant, or germplasm, pollen, or seed thereof, for the presence of at least one molecular marker linked to qDTY_{12.1}, or a yield-improving part thereof, wherein the at least one molecular marker is selected from the group consisting of: RM28048; RM28076; RM28089; RM28099; RM28130; RM511; RM1261; RM28166; RM28199; and Indel-8, and wherein a selected one or more progeny plant having DNA that comprises a nucleic acid comprising qDTY_{12.1}, or a yield-improving part thereof, has improved lateral root growth and water uptake.

In another embodiment described herein, the method of improving lateral root growth and water uptake in a cereal grass further comprises the steps: a) backcrossing the one or more selected progeny plants to produce backcross progeny plants; and b) selecting one or more backcross progeny plants having chromosomal DNA that comprises a nucleic acid comprising qDTY_{12.1}, or a yield-improving part thereof, wherein qDTY_{12.1}, or a yield-improving part thereof, is detected in the one or more backcross progeny plants by analyzing genomic DNA from the one or more backcross progeny plants, or germplasm, pollen, or seed thereof, for the presence of at least one molecular marker linked to qDTY_{12.1}, or a yield-improving part thereof, wherein the at least one molecular marker is selected from the group consisting of: RM28048; RM28076; RM28089; RM28099; RM28130; RM511; RM1261; RM28166; RM28199; and Indel-8. In yet another embodiment described herein, these two steps are repeated one or more times to produce third or higher backcross progeny plants having chromosomal DNA that comprises a nucleic acid comprising qDTY_{12.1}, or a yield-improving part thereof, wherein qDTY_{12.1}, or a yield-improving part thereof, is detected in the one or more backcross progeny plants by analyzing genomic DNA from the one or more backcross progeny plants, or germplasm, pollen, or seed thereof, for the presence of at least one molecular marker linked to qDTY_{12.1}, or a yield-improving part thereof, wherein the at least one molecular marker is selected from the group consisting of: RM28048; RM28076; RM28089; RM28099; RM28130; RM511; RM1261; RM28166; RM28199; and Indel-8.

In certain embodiments, the physiological and morphological characteristics of the recipient plant, other than those of lateral root growth and water uptake, are retained. In other embodiments, at least one of the crossing plant and the recipient plant has chromosomal DNA comprising a nucleic acid having at least 70% sequence identity to Ulp1. In yet other embodiments, the selected one or more progeny plants is further selected for having increased lateral root growth relative to a control plant.

In another embodiment described herein, the selected one or more progeny plants is further selected for having increased lateral root growth relative to a control plant in both well watered and drought conditions. In other embodiments, the selected one or more progeny plants is further selected for having improved yield under drought conditions relative to a control plant. In yet other embodiments, the selected one or more progeny plants is further selected for having at least one trait associated with improved yield under drought conditions selected from the group consisting of: increased sucrose content in flag leaf relative to a control plant; increased sucrose content in spikelets relative to a control plant; increased starch content in spikelets relative to a control plant; and increased carbon reserves in roots relative to a control plant.

In another embodiment described herein, the cereal grass is selected from the group consisting of: rice; corn; wheat; barley; sorghum; millet; oats; and rye. In another embodiment, the cereal grass is rice. In yet another embodiment, the cereal grass is corn.

In other embodiments described herein, the crossing plant is a rice plant selected from the group consisting of: WayRarem; IR79971-B-102-B; and IR74371-46-1-1. In another embodiment, the recipient plant is a rice plant selected from the group consisting of: Vandana; Kalinga 3; Anjali; IR64; Swarna; Sambha Mahsuri; MTU1010, Lalat; Naveen; Sabitri; BR11; BR29; BR28; TDK1; TDK 9; and Chirang.

In another embodiment described herein, the yield improving part of qDTY_{12.1} comprises one or more nucleic acids sharing at least 70% identity with one or more nucleic acids of the group of nucleic acids consisting of: SEQ ID NO: 2 (OsNAM_{12.1}); SEQ ID NO: 3 (OsGPDP_{12.1}); SEQ ID NO: 4 (OsGPDP_{12.1}); SEQ ID NO: 5 (OsGPDP_{12.1}); SEQ ID NO: 6 (OsSTPK_{12.1}); SEQ ID NO: 7 (OsSTPK_{12.1}); SEQ ID NO: 8 (OsSTPK_{12.1}); SEQ ID NO: 9 (OsPOle_{12.1}); SEQ ID NO: 10 (OsMtN3_{12.1}); SEQ ID NO: 11 (OsWAK_{12.1}); SEQ ID NO: 12 (OsCesA_{12.1}); SEQ ID NO: 13 (OsGDP_{12.1}); SEQ ID NO: 14 (OsARF_{12.1}); SEQ ID NO: 15 (OsARF_{12.1}); SEQ ID NO: 16 (OsARF_{12.1}); SEQ ID NO: 17 (OsARF_{12.1}); SEQ ID NO: 18 (OsARF_{12.1}); SEQ ID NO: 20 (OsAmh_{12.1}); and SEQ ID NO: 21 (OsAmh_{12.1}). In yet another embodiment, the yield improving part of qDTY_{12.1} comprises a nucleic acid sharing an identity with SEQ ID NO: 2 (OsNAM_{12.1}) selected from the group consisting of: at least 70% identity; at least 75% identity; at least 80% identity; at least 85% identity; at least 90% identity; at least 95% identity; at least 96% identity; at least 97% identity; at least 98% identity; at least 99% identity; and 100% identity.

In another embodiment described herein, the crossing plant, in addition to having chromosomal DNA that comprises a nucleic acid comprising qDTY12.1, or a yield-improving part thereof, also comprises a nucleic acid comprising qDTY_{2.3}. In another embodiment, the recipient plant has chromosomal DNA that comprises a nucleic acid comprising qDTY_{2.3}.

In a particular embodiment described herein, is a method of improving lateral root growth and water uptake in a cereal grass comprising: a) crossing a crossing plant of one variety of cereal grass having chromosomal DNA that comprises a nucleic acid sharing an identity with SEQ ID NO: 2 (OsNAM_{12.1}) selected from the group consisting of: at least 70% identity; at least 75% identity; at least 80% identity; at least 85% identity; at least 90% identity; at least 95% identity; at least 96% identity; at least 97% identity; at least 98% identity; at least 99% identity; and 100% identity, with a recipient plant of a distinct variety of cereal grass having chromosomal DNA that does not include a nucleic acid sharing at least 70% identity with SEQ ID NO: 2 (OsNAM_{12.1}); and b) selecting one or more progeny plants having chromosomal DNA that comprises a nucleic acid sharing an identity with SEQ ID NO: 2 (OsNAM_{12.}) selected from the group consisting of: at least 70% identity; at least 75% identity; at least 80% identity; at least 85% identity; at least 90% identity; at least 95% identity; at least 96% identity; at least 97% identity; at least 98% identity; at least 99% identity; and 100% identity.

In certain embodiments described herein, the nucleic acid sharing an identity with SEQ ID NO: 2 (OsNAM_{12.}) selected from the group consisting of: at least 70% identity; at least 75% identity; at least 80% identity; at least 85% identity; at least 90% identity; at least 95% identity; at least 96% identity; at least 97% identity; at least 98% identity; at least 99% identity; and 100% identity is detected by RT-PCR.

In another embodiment described herein, the method of improving lateral root growth and water uptake in a cereal grass further comprising the steps: c) backcrossing the one or more selected progeny plants produce backcross progeny plants; and d) selecting one or more backcross progeny plants having chromosomal DNA that comprises a nucleic acid sharing an identity with SEQ ID NO: 2 (OsNAM_{12.}) selected from the group consisting of: at least 70% identity; at least 75% identity; at least 80% identity; at least 85% identity; at least 90% identity; at least 95% identity; at least 96% identity; at least 97% identity; at least 98% identity; at least 99% identity; and 100% identity. In certain embodiments, these steps are repeated one or more times to produce third or higher backcross progeny plants having chromosomal DNA that comprises a nucleic acid sharing an identity with SEQ ID NO: 2 (OsNAM_{12.}) selected from the group consisting of: at least 70% identity; at least 75% identity; at least 80% identity; at least 85% identity; at least 90% identity; at least 95% identity; at least 96% identity; at least 97% identity; at least 98% identity; at least 99% identity; and 100% identity.

In another embodiment described herein, the at least one of the crossing plant and the recipient plant has chromosomal DNA comprising a nucleic acid having at least 70% sequence identity to Ulp1, wherein Ulp1 encodes a functional deSUMOylating protease capable of deSUMOylating a polypeptide encoded by the nucleic acid sharing an identity with SEQ ID NO: 2 (OsNAM_{12.}) selected from the group consisting of: at least 70% identity; at least 75% identity; at least 80% identity; at least 85% identity; at least 90% identity; at least 95% identity; at least 96% identity; at least 97% identity; at least 98% identity; at least 99% identity; and 100% identity.

In another embodiment described herein, is a method for selecting a cereal grass plant having improved lateral root growth and water uptake relative to a control cereal grass plant, comprising: a) inducing expression or increasing expression in a cereal grass plant a nucleic acid comprising qDTY_{12.1}, or a yield-improving part thereof, wherein the induced or increased expression of the nucleic acid comprising qDTY_{12.1}, or a yield-improving part thereof, is obtained by transforming and expressing in the cereal grass plant the nucleic acid comprising qDTY_{12.1}, or a yield-improving part thereof; and b) selecting a cereal grass plant having improved lateral root growth and water uptake relative to a control cereal grass plant, wherein the cereal grass plant having improved lateral root growth and water uptake relative to a control cereal grass plant is selected by analyzing genomic DNA from the cereal grass plant, or germplasm, pollen, or seed thereof, and detecting therein at least one molecular marker linked to qDTY12.1, or a yield-improving part thereof, wherein the at least one molecular marker is selected from the group consisting of: RM28048; RM28076; RM28089; RM28099; RM28130; RM511; RM1261; RM28166; RM28199; and Indel-8. In another embodiment, the cereal grass plant has chromosomal DNAcomprising a nucleic acid having at least 70% sequence identity to Ulp1.

In another embodiment described herein, the induced or increased expression of the nucleic acid comprising qDTY12.1, or a yield-improving part thereof, is a result of introducing and expressing the nucleic acid comprising qDTY_{12.1}, or a yield-improving part thereof, in the cereal grass plant under control of at least one promoter functional in plants. In certain embodiments, the at least one promoter and the nucleic acid comprising qDTY_{12.1}, or yield improving part thereof, are operably linked.

In a particular embodiment described herein, is a method for generating a cereal grass plant having improved lateral root growth and water uptake relative to a control cereal grass plant comprising: a) transforming a cereal grass plant cell, cereal grass plant, or part thereof with a construct comprising: 1) a nucleic acid encoding a polypeptide having a sequence identity selected from the group consisting of: at least 70% identity; at least 75% identity; at least 80% identity; at least 85% identity; at least 90% identity; at least 95% identity; at least 96% identity; at least 97% identity; at least 98% identity; at least 99% identity; and 100% identity to nucleic acid sequence SEQ ID NO: 2 (OsNAM_{12.1}); 2) a promoter operably linked to the nucleic acid; and 3) a transcription termination sequence; and b) expressing the construct in a cereal grass plant cell, cereal grass plant, or part thereof, thereby generating a cereal grass plant having improved lateral root growth and water uptake relative to a control cereal grass plant.

In another embodiment described herein, the construct further comprises one or more nucleic acids sharing at an identity selected from the group consisting of: at least 70% identity; at least 75% identity; at least 80% identity; at least 85% identity; at least 90% identity; at least 95% identity; at least 96% identity; at least 97% identity; at least 98% identity; at least 99% identity; and 100% identity, with one or more nucleic acids of the group of nucleic acids consisting of: SEQ ID NO: 3 (OsGPDP_{12.1}); SEQ ID NO: 4 (OsGPDP_{12.1}); SEQ ID NO: 5 (OsGPDP_{12.1}); SEQ ID NO: 6 (OsSTPK_{12.1}); SEQ ID NO: 7 (OsSTPK_{12.1}); SEQ ID NO: 8 (OsSTPK_{12.1}); SEQ ID NO: 9 (OsPOle_{12.1}); SEQ ID NO: 10 (OsMtN3_{12.1}); SEQ ID NO: 11 (OsWAK_{12.1}); SEQ ID NO: 12 (OsCesA_{12.1}); SEQ ID NO: 13 (OsGDP_{12.1}); SEQ ID NO: 14 (OsARF_{12.1}); SEQ ID NO: 15 (OsARF_{12.1}); SEQ ID NO: 16 (OsARF_{12.1}); SEQ ID NO: 17 (OsARF_{12.1}); SEQ ID NO: 18 (OsARF_{12.1}); SEQ ID NO: 20 (OsAmh_{12.1}); and SEQ ID NO: 21 (OsAmh_{12.1}).

In another embodiment described herein, the construct further comprises a nucleic acid having at least 70% sequence identity to Ulp1, wherein the nucleic acid encoding a deSUMOylating protease encodes a functional deSUMOylating protease capable of deSUMOylating a polypeptide encoded by the nucleic acid sharing an identity with SEQ ID NO: 2 (OsNAM_{12.}) selected from the group consisting of: at least 70% identity; at least 75% identity; at least 80% identity; at least 85% identity; at least 90% identity; at least 95% identity; at least 96% identity; at least 97% identity; at least 98% identity; at least 99% identity; and 100% identity.

In a particular aspect described herein, is a method for the production of a transgenic cereal grass plant having improved lateral root growth and water uptake relative to a control cereal grass plant comprising: a) transforming and expressing in a cereal grass plant cell at least one nucleic acid having at a sequence identity selected from the group consisting of: at least 70% identity; at least 75% identity; at least 80% identity; at least 85% identity; at least 90% identity; at least 95% identity; at least 96% identity; at least 97% identity; at least 98% identity; at least 99% identity; and 100% identity with one or more nucleic acids of the group of nucleic acids consisting of: SEQ ID NO: 2 (OsNAM_{12.1}); SEQ ID NO: 3 (OsGPDP_{12.1}); SEQ ID NO: 4 (OsGPDP_{12.1}); SEQ ID NO: 5 (OsGPDP_{12.1}); SEQ ID NO: 6 (OsSTPK_{12.1}); SEQ ID NO: 7 (OsSTPK_{12.1}); SEQ ID NO: 8 (OsSTPK_{12.1}); SEQ ID NO: 9 (OsPOle_{12.1}); SEQ ID NO: 10 (OsMtN3_{12.1}); SEQ ID NO: 11 (OsWAK_{12.1}); SEQ ID NO: 12 (OsCesA_{12.1}); SEQ ID NO: 13 (OsGDP_{12.1}); SEQ ID NO: 14 (OsARF_{12.1}); SEQ ID NO: 15 (OsARF_{12.1}); SEQ ID NO: 16 (OsARF_{12.1}); SEQ ID NO: 17 (OsARF_{12.1}); SEQ ID NO: 18 (OsARF_{12.1}); SEQ ID NO: 20 (OsAmh_{12.1}); and SEQ ID NO: 21 (OsAmh_{12.1}); and b) cultivating the cereal grass plant cell under conditions promoting plant growth and development, and obtaining transformed plants expressing one or more of OsNAM_{12.1}, OsGPDP_{12.1}, OsSTPK_{12.1}, OsPOle_{12.1}, OsMtN3_{12.1}, OsWAK_{12.1}, OsCesA_{12.1}, OsGDP_{12.1}, OsARF_{12.1}, and OsAmh_{12.1}.

In another embodiment described herein, the method for the production of a transgenic cereal grass plant having improved lateral root growth and water uptake relative to a control cereal grass plant further comprises transforming and expressing in the cereal grass plant cell a nucleic acid having at least 70% sequence identity to Ulp1, wherein Ulp1 encodes a functional deSUMOylating protease capable of deSUMOylating a polypeptide encoded by the nucleic acid having at least 70% sequence identity with SEQ ID NO: 2 (OsNAM_{12.1}).

In another particular aspect described herein, is a transgenic plant cell comprising: a) at least one promoter that is functional in plants; and b) at least one nucleic acid having a sequence identity selected from the group consisting of: at least 70% identity; at least 75% identity; at least 80% identity; at least 85% identity; at least 90% identity; at least 95% identity; at least 96% identity; at least 97% identity; at least 98% identity; at least 99% identity; and 100% identity with one or more nucleic acids of the group of nucleic acids consisting of: SEQ ID NO: 2 (OsNAM_{12.1}); SEQ ID NO: 3 (OsGPDP_{12.1}); SEQ ID NO: 4 (OsGPDP_{12.1}); SEQ ID NO: 5 (OsGPDP_{12.1}); SEQ ID NO: 6 (OsSTPK_{12.1}); SEQ ID NO: 7 (OsSTPK_{12.1}); SEQ ID NO: 8 (OsSTPK_{12.1}); SEQ ID NO: 9 (OsPOle_{12.1}); SEQ ID NO: 10 (OsMtN3_{12.1}); SEQ ID NO: 11 (OsWAK_{12.1}); SEQ ID NO: 12 (OsCesA_{12.1}); SEQ ID NO: 13 (OsGDP_{12.1}); SEQ ID NO: 14 (OsARF_{12.1}); SEQ ID NO: 15 (OsARF_{12.1}); SEQ ID NO: 16 (OsARF_{12.1}); SEQ ID NO: 17 (OsARF_{12.1}); SEQ ID NO: 18 (OsARF_{12.1}); SEQ ID NO: 20 (OsAmh_{12.1}); and SEQ ID NO: 21 (OsAmh_{12.1}), wherein the promoter and polynucleotide are operably linked and incorporated into the plant cell chromosomal DNA.

In another embodiment described herein, a transgenic plant cell further comprising a nucleic acid having at least 70% sequence identity to Ulp1, wherein Ulp1 encodes a functional deSUMOylating proteas capable of deSUMOylating a polypeptide encoded by the nucleic acid having at least 70% sequence identity with SEQ ID NO: 2 (OsNAM_{12.1}).

In yet another aspect described herein, a transgenic plant cell is a plant cell selected from the group consisting of: rice plant cell; corn plant cell; wheat plant cell; barley plant cell; sorghum plant cell; millet plant cell; oats plant cell; and rye plant cell. In another embodiment, he plant cell is homozygous for the at least one nucleic acids.

In another embodiment described herein, is a transgenic plant comprising a plurality of transgenic plant cells described herein.

In another particular aspect described herein, is a transgenic plant comprising: a) at least one promoter that is functional in plants; and b) at least one nucleic acid having a sequence identity selected from the group consisting of: at least 70% identity; at least 75% identity; at least 80% identity; at least 85% identity; at least 90% identity; at least 95% identity; at least 96% identity; at least 97% identity; at least 98% identity; at least 99% identity; and 100% identity with one or more nucleic acids of the group of nucleic acids consisting of: SEQ ID NO: 2 (OsNAM_{12.1}); SEQ ID NO: 3 (OsGPDP_{12.1}); SEQ ID NO: 4 (OsGPDP_{12.1}); SEQ ID NO: 5 (OsGPDP_{12.1}); SEQ ID NO: 6 (OsSTPK_{12.1}); SEQ ID NO: 7 (OsSTPK_{12.1}); SEQ ID NO: 8 (OsSTPK_{12.1}); SEQ ID NO: 9 (OsPOle_{12.1}); SEQ ID NO: 10 (OsMtN3_{12.1}); SEQ ID NO: 11 (OsWAK_{12.1}); SEQ ID NO: 12 (OsCesA_{12.1}); SEQ ID NO: 13 (OsGDP_{12.1}); SEQ ID NO: 14 (OsARF_{12.1}); SEQ ID NO: 15 (OsARF_{12.1}); SEQ ID NO: 16 (OsARF_{12.1}); SEQ ID NO: 17 (OsARF_{12.1}); SEQ ID NO: 18 (OsARF_{12.1}); SEQ ID NO: 20 (OsAmh_{12.1}); and SEQ ID NO: 21 (OsAmh_{12.1}), wherein the promoter and polynucleotide are operably linked and incorporated into the plant cell chromosomal DNA.

In certain embodiments described herein, is a transgenic plant homozygous for the at least one nucleic acid. In another embodiment, is a seed of a transgenic plant described herein. In yet another embodiment is a plant part of a transgenic plant described herein.

In another particular aspect described herein, is a method for selecting transgenic plants having improved lateral root growth and water uptake relative to a control plant, comprising: a) screening a population of plants for increased lateral root growth and water uptake, wherein plants in the population comprise a transgenic plant cell having recombinant DNA incorporated into its chromosomal DNA, wherein the recombinant DNA comprises a promoter that is functional in a plant cell and that is functionally linked to an open reading frame of a nucleic acid having a sequence identity selected from the group consisting of: at least 70% identity; at least 75% identity; at least 80% identity; at least 85% identity; at least 90% identity; at least 95% identity; at least 96% identity; at least 97% identity; at least 98% identity; at least 99% identity; and 100% identity with one or more nucleic acids of the group of nucleic acids consisting of: SEQ ID NO: 2 (OsNAM_{12.1}); SEQ ID NO: 3 (OsGPDP_{12.1}); SEQ ID NO: 4 (OsGPDP_{12.1}); SEQ ID NO: 5 (OsGPDP_{12.1}); SEQ ID NO: 6 (OsSTPK_{12.1}); SEQ ID NO: 7 (OsSTPK_{12.1}); SEQ ID NO: 8 (OsSTPK_{12.1}); SEQ ID NO: 9 (OsPOle_{12.1}); SEQ ID NO: 10 (OsMtN3_{12.1}); SEQ ID NO: 11 (OsWAK_{12.1}); SEQ ID NO: 12 (OsCesA_{12.1}); SEQ ID NO: 13 (OsGDP_{12.1}); SEQ ID NO: 14 (OsARF_{12.1}); SEQ ID NO: 15 (OsARF_{12.1}); SEQ ID NO: 16 (OsARF_{12.1}); SEQ ID NO: 17 (OsARF_{12.1}); SEQ ID NO: 18 (OsARF_{12.1}); SEQ ID NO: 20 (OsAmh_{12.1}); and SEQ ID NO: 21 (OsAmh_{12.1}), wherein individual plants in said population that comprise the transgenic plant cell exhibit increased yield under drought conditions relative to control plants which do not comprise the transgenic plant cell; and b) selecting from said population one or more plants that exhibit lateral root growth and water uptake greater than the lateral root growth and water uptake in control plants which do not comprise the transgenic plant cell.

In another embodiment described herein, the method for selecting transgenic plants having improved lateral root growth and water uptake relative to a control plant further comprises selecting one or more plants that exhibit increased yield under drought conditions at a level greater than the yield under drought conditions in control plants that do not comprise the transgenic plant cell. In another aspect described herein, the method for selecting transgenic plants having improved lateral root growth and water uptake relative to a control plant further comprises a step of collecting seed from the one or more selected plants.

In particular example described herein, is a method of improving lateral root growth and water uptake in a cereal grass plant comprising modifying a nucleic acid encoding no-apical meristem (NAM) transcription factor in a cereal grass so that the nucleic acid encoding the NAM transcription factor shares an identity with SEQ ID NO: 2 (OsNAM₁₂) selected from the group consisting of: at least 90% identity; at least 95% identity; at least 96% identity; at least 97% identity; at least 98% identity; at least 99% identity; and 100% identity. In another embodiment, this method further comprises modifying one or more nucleic acids encoding one or more genes selected from the group consisting of GPDP; STPK; POle; MtN3; WAK, CesA; GDP; ARF; and Amh so that the one or more nucleic acids share an identity selected from the group consisting of: at least 95% identity; at least 96% identity; at least 97% identity; at least 98% identity; at least 99% identity; and 100% identity with one or more nucleic acids of the group of nucleic acids consisting of: SEQ ID NO: 3 (OsGPDP_{12.1}); SEQ ID NO: 4 (OsGPDP_{12.1}); SEQ ID NO: 5 (OsGPDP_{12.1}); SEQ ID NO: 6 (OsSTPK_{12.1}); SEQ ID NO: 7 (OsSTPK_{12.1}); SEQ ID NO: 8 (OsSTPK_{12.1}); SEQ ID NO: 9 (OsPOle_{12.1}); SEQ ID NO: 10 (OsMtN3_{12.1}); SEQ ID NO: 11 (OsWAK_{12.1}); SEQ ID NO: 12 (OsCesA_{12.1}); SEQ ID NO: 13 (OsGDP_{12.1}); SEQ ID NO: 14 (OsARF_{12.1}); SEQ ID NO: 15 (OsARF_{12.1}); SEQ ID NO: 16 (OsARF_{12.1}); SEQ ID NO: 17 (OsARF_{12.1}); SEQ ID NO: 18 (OsARF_{12.1}); SEQ ID NO: 20 (OsAmh_{12.1}); and SEQ ID NO: 21 (OsAmh_{12.1}). In another embodiment, the cereal grass comprises a nucleic acid comprising qDTY_{2.3}. In yet another embodiment, this method of improving lateral root growth and water uptake in a cereal grass plant, modifying the nucleic acid is performed using a technique selected from the group consisting of: transgenic method; crossing; backcrossing; protoplast fusion; doubled haploid technique; embryo rescue; zinc-finger nucleases; transcription activator-like effector nucleases; and clustered regularly interspaced short palindromic repeat (CRISPR)/Cas-based RNA-guided DNA endonucleases.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1A: Line graph showing yield and panicle branching results for qDTY_{12.1} NILs. Data show an additive effect of qDTY_{12.1} QTL+ and QTL− lines for grain yield over Vandana under varying severity of drought stress in six experiments conducted over three seasons at IRRI.

FIG. 1B: Photographs showing yield and panicle branching results for qDTY_{12.1} NILs. Increased panicle branching occurred in the NIL 481-B.

FIG. 1C: Bar graph showing yield and panicle branching results for qDTY_{12.1} NILs. Quantitative results are shown for increased panicle branching, total spikelets and spikelet fertility. Two to three panicles were sampled from 10 plants each of Vandana and 481-B.

FIGS. 2A-2D: Graphs showing morpho-physiological characters of qDTY_{12.1} NILs. The drought response of qDTY_{12.1} reflects drought-induced transpiration efficiency coupled with greater water uptake during reproductive stage drought due to increased lateral root growth qDTY_{12.1}. Nits showed higher transpiration efficiency according to FIG. 2A) carbon isotope discrimination in the youngest leaves sampled every 2 weeks during the drought stress period in the field (2012DS); FIG. 2B) instantaneously by gas exchange (photosynthesis rate/stomatal conductance; 2012WS); FIG. 2C) gravimetrically in a greenhouse seedling-stage study; and FIG. 2D) by soil moisture measurements in which 481-B showed more conservative water uptake patterns than Vandana during vegetative stage and higher water uptake during reproductive stage at a 40 cm depth (2012DS).

FIGS. 2E-2F: Photographs showing morpho-physiological characteristics of qDTY_{12.1} NIL 481-B. FIG. 2E) a higher proportion of lateral roots in 481-B than in Vandana; and FIG. 2F) increased LRN in vitro compared with Vandana.

FIG. 3A: A heat map showing the relative expression of candidate genes in root, leaf & panicles in NILs compared to Vandana during severe reproductive stage DC.

FIG. 3B: A bar graph representing qRT-PCR expression analysis of the five putative target CGs of OsNAM_{12.1}.

FIG. 3C: Photograph of EMSA for CG promoter binding by OsNAM_{12.1}. Lane 1, Negative control of DNA without the protein; Lane 2 DNA with GST protein, Lane 3 to 6 DNA with OsGDP_{12.1}, OsNOD_{12.1}, OsCesA_{12.1}, and OsNAM_{12.1} respectively.

FIG. 4A: Table showing characteristics of the parental rice varieties used.

FIG. 4B: Flow chart showing the marker assisted backcrossing (MAB) strategy for simultaneous fine mapping and development of NILs of recipient parent Vandana with fine-mapped segment of qDTY_{12.1}.

FIG. 5: Graphical genotype of IR84984-83-15-481-B (BC₂F₃) showing recipient genome recovery in the background and length of QTL region transferred.

FIG. 6A: Photographs of donor parent WayRarem (WR), recipient parent Vandana (V), and qDTY_{12.1} positive NIL IR84984-83-15-481-B (481-B) under drought. The NIL 481-B shows filled panicles while the other two do not.

FIG. 6B: Photographs showing grain type similarity between V and 481-B.

FIG. 6C: Line graph showing grain yield data from 11 trials over three years for yield.

FIGS. 7A-7C: FIG. 7A) Two NILs showed improved performance over Vandana in seedling stage trials for drought tolerance as measure by shoot biomass. FIGS. 7B) and 7C) NIL 481-B showed more root branching in seedlings in the greenhouse under drought conditions.

FIGS. 8A-8C: The qDTY_{12.1} NILs did not show different transpiration efficiency (TE) than Vandana under well-watered conditions according to FIG. 8A) successive carbon isotope discrimination measurements on the 3 youngest leaves in the field (DS), FIG. 8B) gravimetrically in the greenhouse, and FIG. 8C) instantaneously by gas exchange in the field (WS). Significant differences between +QTL and −QTL lines are indicated by a “**” for p<0.01 and “*” for p<0.05.

FIGS. 8D-8G: Additional seasons of data for the TE and root traits observed for qDTY_{12.1}: carbon isotope discrimination in FIG. 8D) drought stress and FIG. 8E) well-watered conditions (WS); FIG. 8F) genotypic differences in soil moisture levels (2012WS); FIG. 8G) Differences in root distribution among diameter classes as a proportion of total root length at a soil depth of 30-45 cm (2010DS). Significant differences between +QTL and −QTL lines are indicated by a “**” for p<0.01 and “*” for p<0.05.

FIG. 9A: Comparison of the QTL region in the Nipponbare and 9311 genomes.

FIG. 9B: Schematic representation of the relative position of the CG along the QTL.

FIG. 9C: Results for Southern hybridization of the OsCesA_{12.1} probe on genomic DNA of NB, WayRarem, +21, +83, −21, −83 and V (1-7). Plasmid DNA containing the OsCesA_{12.1} fragment was used as a positive control (P). Arrow indicates the 2.7 kb fragment absent in V, −21 & −83 but present in NB, WayRarem, +21 and +83.

FIG. 10A: Schematic of the different regions of the OsNAM_{12.1} protein from the N-terminal to the C-terminal: dimerization domain (DD), DNA binding domain (DBD), transactivation domain (TAD) and the position of the PEST motif that facilitates proteasome or calpain mediated protein cleavage. Numbers represent the amino acid position.

FIG. 10B: Primary amino acid sequence comparison between Vandana (SEQ ID NO: 182) and WayRarem (SEQ ID NO: 183) showing the three mutations encircled. The red arrows indicate potential SUMOylation sites and the blue arrow indicates the phosphorylation site change. The underlined region indicates the PEST sequence.

FIG. 10C: The effect of the AA differences on the 3D structure of Vanadana (V) and WayRarem (WR).

FIG. 10D: Schematic differences in drought responsive promoter cis-elements in the additional promoter sequence present in WR.

FIG. 11A-11B: Characteristics of transgenic plants I-OsNAM_{12.1}^{ox}. FIG. 11A) Photograph showing an increase in lateral root growth under PEG simulated water deficit in the three transgenic events of I-OsNAM_{12.1}^{ox} seedlings in vitro. FIG. 11B) Bar graph showing quantitative difference in LR between the three events compared to WT IR64.

FIGS. 11C-11D: Characteristics of transgenic plants I-OsNAM_{12.1}^{ox}. FIG. 11C) Photograph showing an increase in panicle branching under PEG simulated water deficit. FIG. 11D) Quantitative difference in panicle branching between the three events compared to WT IR64.

FIG. 11E: Characteristics of transgenic plants I-OsNAM_{12.1}^{ox}. Line graph showing gas exchange measurements (LICOR-6400) whereby transgenic events showed higher transpiration rates under drought stress.

FIG. 11F: Characteristics of transgenic plants I-OsNAM_{12.1}^{ox}. Bar graph showing field comparison between WT IR64 and I-OsNAM_{12.1}^{ox} as seen through number of spikelets and filled spikelets under drought.

FIGS. 12A-12B: FIG. 12A) Panel showing that plants mutated for the particular CGs led to higher lateral root growth than the WT. FIG. 12B) table showing percentage increase in LR in the mutants. Three to five tubes were sampled for each mutant and WT.

FIGS. 13A-13B: EMSA and SUMOylation results for OsNAM_{12.1}. FIG. 12A) EMSA for CG promoter binding by OsNAM_{12.1}. Lane 1, negative control of OsGDP_{12.1} promoter DNA without the protein; Lane 2 negative control OsGDP_{12.1} promoter DNA with GST protein, which was part of the recombinant OsNAM_{12.1} protein; Lanes 3 to 6, respective promoter DNA of OsGDP_{12.1}, OsNOD_{12.1}, OsCesA_{12.1}, and OsARF_{12.1} showing binding to the recombinant OsNAM_{12.1} through band shift represented by the white arrowheads. FIG. 12B) An immunoblot using anti-NAM antibody from rabbit shows higher M_{w} bands of OsNAM_{12.1.} (Lanes 1-5: Vandana, WayRarem, NIL, WT-IR64 & I-OsNAM_{12.1}^{ox}).

FIG. 13C: An immunoblot showing the action of deSUMoylating protease Ulp1 on the di-sumoylated OsNAM_{12.1} (˜53 kDa). The column graph shows the subsequent deSUMOylation of di-SUMOylated form and progressive accumulation of mono-SUMOylated (˜41 kDa) OsNAM_{12.1}.

FIG. 13D: SUMO protein interaction with recombinant OsNAM_{12.1}. Lane 1 Marker; Lane 2, SUMO1; Lane 3, SUMO2; Lane 4, SUMO3; P is positive control, N is negative control and NAM is untreated NAM.

FIG. 13E: 2D-immunoblot result for deSUMOylation with Ulp1 shows clear changes in spots marked with arrows, after the enzyme treatment.

FIG. 13F: 2D-immunoblot successively on the same blot with anti-NAM and anti-SUMO antibody shows identical spots confirming in vivo SUMOylation of the NAM protein.

FIG. 14: The effect of OsNAM_{12.1} on in vitro root phenotype of different genotypes.

FIG. 15: Haplotype Structure at OsNAM12.1. Figure depicts SNP calls from the 125 genomes data across the LOC_Os12g29330.1 locus, including the 5′ and 3′ untranslated region (UTR). Seven major haplotypes were identified by clustering the domesticated varieties at both synonymous and non-synonymous SNPs that differ from the Nipponbare reference genome. The temperate japonica subpopulation is comprised of one haplotype, while the indica, aus, tropical japonica, and aromatic subpopulations each have two haplotypes (labeled along the y-axis of the graph). Nipponbare SNPs are labeled in dark blue. Synonymous SNPs that differ from the reference genome are labeled in white. Four Non-synonymous SNPs were identified within the coding sequence. Two of these non-synonymous SNPs are highly prevalent within the indica subpopulation (labeled yellow), and are present within WayRarem. The other two non-synonymous SNPs identified are relatively rare within this collection of germplasm, only appearing three times within all 125 genetic lines (labeled red). Missing SNP calls are labeled with a double period (..).

FIG. 16A: SSR marker and CG-allele based genotyping of Intra-QTL recombinant lines.

FIG. 16B: Effect on root growth under presence/absence of genetic regions in IQRLs.

FIG. 16C: Field-based yield data on line 937-B and 917-B. The colored numbers represent highest yield.

FIG. 17: Field performance and grain characteristics of BC₂F_{3:4} and BC₃F_{3:7} NILs, parents, and drought-tolerant checks in advanced yield trials under upland severe stress and non-stress conditions with respective percentage recovery of Vandana allele in the background (% BG).

FIGS. 18A-18B: Fine mapping results. FIG. 17A) Top—Using five SSR markers RM28099, RM28130, RM511, RM1261 and RM28166 represented as alphabets respectively or Bottom—the nine candidate genes OsGPDP_{12.1}, OsAmH_{12.1} OsPOLe_{12.1} OsMtN3_{12.1}, OsCesA_{12.1}, OSNAM_{12.1}, OsGDP_{12.1}, OsWAK_{12.1} and OsARF_{12.1} represented as numerals 1-9 respectively on the x-axis. FIG. 187B) In qDTY_{12.1} recombinant plants, yield was reduced as the percentage of WR alleles decreased.

FIG. 19A-19F: Heat map style representation of the observation that higher the number of WayRarem alleles of markers (genes) along qDTY_{12.1}, better the yield. The figure represents raw data for yield (Y-axis-right; Kg/h) in different recombinant lines (Y-axis-left) containing markers (X-axis top) for Vandana (yellow), WayRarem (Green) and heterozygote (light green) alleles under no stress (FIG. 19A), mild stress (FIG. 19B), moderate stress (FIG. 19C), severe stress-I (FIG. 19D), severe stress-II (FIG. 19E). The sixth panel (FIG. 19F) represents percent yield loss under the two severe stress conditions combined.

FIGS. 20A-20B: Differences between Vandana and WayRarem in the potential post-translational modification of OsNAM_{12.1} in FIG. 20A) SUMOylation and FIG. 20B) phosphorylation sites. Figures disclose SEQ ID NOS 182, 184-187, 183-184, 187, and 186, respectively, in order of appearance.

FIGS. 21A-21C: Differences in LRN and SUMOylation of OsNAM_{12.1} in different plant lines. FIG. 21A) 2D-immunodetection of OsNAM_{12.1} SUMOylation pattern under drought in various plant lines. The arrows indicate spots that are substantially reduced under drought in the tolerant lines of 481-B, WR50-6-B4 (2.3), V-OsNAM_{12.1}^{ox} (V-Ox) and I-OsNAM_{12.1}^{ox} (IR-Ox) but not in Vandana or WayRarem. Multiple spots are most likely the various combinations of multiple phosphorylation and SUMOylation of OsNAM_{12.1}. FIG. 21B) The effect of OsNAM_{12.1} on in vitro root phenotype of different ‘genotypes’. FIG. 21C) 3D scatterplot for total root length, maximum root depth and root surface area of Vandana, WayRarem, V-OsNAM_{12.1}^{ox}, 481-B, and WR-50-6-B4 plants at 15 days after germination. Data was collected with ImageJ root analyzer from 8 to 15 seedlings under normal and PEG-simulated drought conditions. Statistical analysis and graph plotting were performed using R software. Multiple stalks of a single color represent mean values for multiple samples within a ‘genotype’, which represent different SD within the group in the three root traits. A single stalk for 481-B in well-watered conditions is masked by one of the stalks of WayRarem indicating similar values of the two.

FIG. 22: Proteins related to cell growth and cell wall formation, along with amino acid metabolism in the roots during stress. Specific locus IDs in the MSU database are shown, along with the protein description and the comparative amounts in NILs compared to Vandana.

FIG. 23: Proteins related to glycolysis and the TCA cycle in the flag leaf, spikelets and roots during stress.

FIG. 24: Proteins related to carbohydrate metabolism, specifically sucrose and starch metabolism in the flag leaf and spikelets during stress. Specific locus IDs in the MSU database are shown, along with the protein description and the comparative amounts in the NILs compared to Vandana.

FIG. 25: Proteins related to photosynthesis and photorespiration in the flag leaf during stress. Specific locus IDs in the database are shown, along with the protein description and the comparative amounts in the NILs compared to Vandana.

FIG. 26: Proteins related to redox systems in the flag leaf during stress. Specific locus IDs in the MSU database are shown along with the protein description and the comparative amounts in the NILs compared to Vandana.

FIGS. 27A-27B: FIG. 28A) A Venn diagram depicting the unique and common proteins identified in all the three tissues from the parent Vandana and the 481-B during drought stress treatment. FIG. 28B) Percentage overview of the proteins mapped onto the gene ontologies through MapMan in flag leaf, spikelets and roots during stress represented as in the 481-B compared to Vandana.

FIGS. 28A-28L: Content of sugars and starch (FIGS. 28A-28D), N %-C/N ratio (FIGS. 28E-28F), free amino acids (FIGS. 28G-28J) and the quantitative PCR data of 3 phosphoglycerate dehydrogenase and aldehyde dehydrogenase (FIGS. 28K-28L) in the roots of the parents (Vandana and WayRarem) and the 481-B. Green bars represent the well-watered treatment (control) while red bars represent the drought (stress) treatment. The average measurement and standard error is shown for each of the samples (n=3).

FIGS. 29A-29D: Content of sugars and starch (FIGS. 29A-29D) in the flag leaf of the parents (Vandana and WayRarem) and the 481-B. Green bars represent the well-watered treatment (control) while red bars represent the drought (stress) treatment. The average measurement and standard error is shown for each of the samples (n=3).

FIGS. 30A-30G: Content of sugars and starch (FIGS. 30A-30D) and the quantitative PCR data of glucose-1-phosphate adenylyltransferase, sucrose synthase and adenylate kinase (FIGS. 30E-30G) in the spikelets of the parents (Vandana and WayRarem) and the 481-B. Green bars represent the well-watered treatment (control) while red bars represent the drought (stress) treatment. The average measurement and standard error is shown for each of the samples (n=3).

FIGS. 31A-31F: Content of total free amino acid in the spikelets (FIGS. 31A-31C) and in the flag leaf (FIGS. 31D-31F) of the parents (Vandana and WayRarem) and the 481-B. Green bars represent the well-watered treatment (control) while red bars represent the drought (stress) treatment.

FIGS. 32A-32E: Quantitative PCR data of pyrroline-5-carboxylate reductase and pyrroline-5-carboxylate synthase gene in the flag leaf (FIGS. 32A-32B), the roots (FIGS. 32C-32D) of the parents (Vandana and WayRarem) and the 481-B. Green bars represent the well-watered treatment (control) while red bars represent the drought (stress) treatment. The average measurement and standard error is shown for each of the samples (n=3). Colorimetric estimation of the proline content (FIG. 32E) in the parents (Vandana and WayRarem) and the 481-B under drought (stress) conditions. The average measurement and standard error is shown for each of the samples (n=3).

FIG. 33: Complete set of primers used for quantitative PCR analysis in Example 3 (SEQ ID NOS 188-197, respectively, in order of appearance).

FIGS. 34A-34F: Data representing the N %, C/N ratio and total free amino acid content in the flag leaf and the spikelets of the parents (Vandana and WayRarem) and the 481-B. Green bars represent the well-watered treatment (control) while red bars represent the drought treatment. The average measurement and standard error is shown for each of the samples (n=3).

FIGS. 35A-35B: Photosynthesis rate in which the 481B and Vandana showed no difference during control conditions, but during stress 481B had a lower photosynthesis rate than Vandana. The average measurement and standard error is shown for each of the samples (n=3).

FIG. 36: Transpiration efficiency in which the 481B showed better TE than Vandana during stress. The average measurement and standard error is shown for each of the samples (n=3).

FIG. 37: Stomatal conductance in which the 481B showed lower stomatal conductance than Vandana during stress. The average measurement and standard error is shown for each of the samples (n=3).

### DETAILED DESCRIPTION OF THE INVENTION

Throughout this disclosure, various publications, patents and published patent specifications are referenced. The disclosures of these publications, patents and published patent specifications are hereby incorporated by reference into the present disclosure to more fully describe the state of the art to which this invention pertains.

Drought killed 11 million and adversely affected two billion people in the last century. Expected climato-demography changes predict exacerbated drought scenarios. Rice, a food and livelihood crop for the poor inhabits drought prone areas. Accordingly, drought tolerant rice may alleviate poverty and hunger. Literature reporting drought tolerant rice has consequently increased. Yet, breeding lines, QTLs, genes or omics/networks-based attempts fall short of the tolerant landraces.

Generating drought tolerant rice genotypes is a highly desirable goal for hunger and poverty amelioration. Single gene transgenic approaches or QTL research have yet not resulted in tolerance levels better than in the available landraces. Disclosed herein are methods of improving upon the tolerance level of a commercial rice genotype through a QTL. This result is of future significance to rice farmers. Also disclosed is evidence that success with this QTL is due to a gene-complex of different genes co-localized at this region. These genes explained multiple morpho-physiological traits altered under drought. This is the first such validation. Ideally expected but rarely demonstrated, field- and lab-based results are corroborative.

The present invention provides methods and materials useful for improving lateral root growth, water uptake, and the yield of grain of cereal grasses grown under drought stress conditions.

### DEFINITIONS

“Yield” describes the amount of grain produced by a plant or a group, or crop, of plants. Yield can be measured in several ways, e.g. t ha⁻¹, average grain yield per plant.

As used herein, “drought stress” means a period of insufficient water supply for normal plant development and growth.

“Improved yield under drought stress” means an increase in the yield of a plant or a group, or crop, of plants compared to corresponding plant or a group, or crop, of plants.

As used herein a “phenotypic trait” is a distinct variant of an observable characteristic, e.g., yield under drought conditions, of a plant that may be inherited by a plant or may be artificially incorporated into a plant by processes such as transfection.

As used herein, “introgression” means the movement of one or more genes, or a group of genes, from one plant variety into the gene complex of another as a result of backcrossing.

As used herein a “plant cell” means a plant cell that is transformed with stably-integrated, non-natural, recombinant DNA, e.g. by Agrobacterium-mediated transformation, bombardment using microparticles coated with recombinant DNA, or other method, or by programmable site-specific nucleases, e.g. zinc-finger nucleases (ZFNs), transcription activator-like effector nucleases (TALENs), clustered regulator interspaced short palindromic repeat (CRISP)/Cas-based RNA-guided DNA endonucleases, or other nuclease. A plant cell of this invention can be an originally-transformed or nucleases-modified plant cell that exists as a microorganism or as a progeny plant cell that is regenerated into differentiated tissue, e.g. into a transgenic plant with stably-integrated, non-natural recombinant DNA, or seed or pollen derived from a progeny transgenic plant.

As used herein a “transgenic plant” means a plant whose genome has been altered by the stable integration of recombinant DNA. A transgenic plant includes a plant regenerated from an originally-transformed plant cell and progeny transgenic plants from later generations or crosses of a transformed plant.

As used herein “recombinant DNA” means DNA which has been a genetically engineered and constructed outside of a cell including DNA containing naturally occurring DNA or cDNA or synthetic DNA.

“Percent identity” describes the extent to which the sequences of DNA or protein segments are invariant throughout a window of alignment of sequences, for example nucleotide sequences or amino acid sequences. Percent identity is calculated over the aligned length preferably using a local alignment algorithm, such as BLASTp. As used herein, sequences are “aligned” when the alignment produced by BLASTp has a minimal e-value.

As used herein “promoter” means regulatory DNA for initializing transcription. A “promoter that is functional in a plant cell” is a promoter capable of initiating transcription in plant cells whether or not its origin is a plant cell, e.g. is it well known that Agrobacterium promoters are functional in plant cells. Thus, plant promoters include promoter DNA obtained from plants, plant viruses and bacteria such as Agrobacterium and Bradyrhizobium bacteria.

As used herein “operably linked” means the association of two or more DNA fragments in a recombinant DNA construct so that the function of one, e.g. protein-encoding DNA, is controlled by the other, e.g. a promoter.

As used herein “expressed” means produced, e.g. a protein is expressed in a plant cell when its cognate DNA is transcribed to mRNA that is translated to the protein.

As used herein a “control plant” means a plant that does not contain the recombinant DNA that imparts enhanced yield under drought stress. A control plant is used to identify and select a transgenic plant that has enhanced yield under drought stress. A suitable control plant can be a non-transgenic plant of the parental line used to generate a transgenic plant, i.e. devoid of recombinant DNA. A suitable control plant may in some cases be a progeny of a hemizygous transgenic plant line that does not contain the recombinant DNA, known as a negative segregant.

Recombinant DNA constructs are assembled using methods well known to persons of ordinary skill in the art and typically comprise a promoter operably linked to DNA, the expression of which provides the enhanced agronomic trait. Other construct components may include additional regulatory elements, such as 5′ leaders and introns for enhancing transcription, 3′ untranslated regions (such as polyadenylation signals and sites), DNA for transit or signal peptides.

Numerous promoters that are active in plant cells have been described in the literature. These include promoters present in plant genomes as well as promoters from other sources, including nopaline synthase (NOS) promoter and octopine synthase (OCS) promoters carried on tumor-inducing plasmids of Agrobacterium tumefaciens and the CaMV35S promoters from the cauliflower mosaic virus as disclosed in U.S. Pat. Nos. 5,164,316 and 5,322,938. Useful promoters derived from plant genes are found in U.S. Pat. No. 5,641,876 which discloses a rice actin promoter, U.S. Pat. No. 7,151,204 which discloses a maize chloroplast aldolase promoter and a maize aldolase (FDA) promoter, and US Patent Application Publication 2003/0131377 A1 which discloses a maize nicotianamine synthase promoter. These and numerous other promoters that function in plant cells are known to those skilled in the art and available for use in recombinant nucleic acids of the present invention to provide for expression of desired genes in transgenic plant cells.

Furthermore, the promoters may be altered to contain multiple “enhancer sequences” to assist in elevating gene expression. Such enhancers are known in the art. By including an enhancer sequence with such constructs, the expression of the selected protein may be enhanced. These enhancers often are found 5′ to the start of transcription in a promoter that functions in eukaryotic cells, but can often be inserted upstream (5′) or downstream (3′) to the coding sequence. In some instances, these 5′ enhancing elements are introns. Particularly useful as enhancers are the 5′ introns of the rice actin 1 (see U.S. Pat. No. 5,641,876) and rice actin 2 genes, the maize alcohol dehydrogenase gene intron, the maize heat shock protein 70 gene intron (U.S. Pat. No. 5,593,874) and the maize shrunken 1 gene. See also US Patent Application Publication 2002/0192813A1 which discloses 5′, 3′ and intron elements useful in the design of effective plant expression vectors.

The term “quantitative trait locus” or “QTL” refers to a polymorphic genetic locus with at least two alleles that reflect differential expression of a continuously distributed phenotypic trait.

The term “associated with” or “associated” in the context of this invention refers to, for example, a nucleic acid and a phenotypic trait, that are in linkage disequilibrium, i.e., the nucleic acid and the trait are found together in progeny plants more often than if the nucleic acid and phenotype segregated independently.

The term “marker” or “molecular marker” or “genetic marker” refers to a genetic locus (a “marker focus”) used as a point of reference when identifying genetically linked loci such as a quantitative trait locus (QTL). The term may also refer to nucleic acid sequences complementary to the genomic sequences, such as nucleic acids used as probes or primers. The primers may be complementary to sequences upstream or downstream of the marker sequences. The term can also refer to amplification products associated with the marker. The term can also refer to alleles associated with the markers. Allelic variation associated with a phenotype allows use of the marker to distinguish germplasm on the basis of the sequence.

The term “interval” refers to a continuous linear span of chromosomal DNA with termini defined by and including molecular markers.

The term “crossed” or “cross” in the context of this invention means the fusion of gametes via pollination to produce progeny (i.e., cells, seeds or plants). The term encompasses both sexual crosses (the pollination of one plant by another) and selling (self-pollination, i.e., when the pollen and ovule are from the same plant or from genetically identical plants).

The phrase “stringent hybridization conditions” refers to conditions under which a probe or nucleic acid will hybridize to its target subsequence, typically in a complex mixture of nucleic acids, but to essentially no other sequences. Stringent conditions are sequence-dependent and will be different in different circumstances. Longer sequences hybridize specifically at higher temperatures. An extensive guide to the hybridization of nucleic acids is found in Thijssen (Thijssen, 1993). Generally, stringent conditions are selected to be about 5-10° C. lower than the thermal melting point (Tm) for the specific sequence at a defined ionic strength pH. The Tm is the temperature (under defined ionic strength, pH, and nucleic acid concentration) at which 50% of the probes complementary to the target hybridize to the target sequence at equilibrium (as the target sequences are present in excess, at Tm, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. For selective or specific hybridization, a positive signal is at least two times background, preferably 10 times background hybridization. Exemplary stringent hybridization conditions are often: 50% formamide, 5×SSC, and 1% SDS, incubating at 42° C., or, 5×SSC, 1% SDS, incubating at 65° C., with wash in 0.2×SSC, and 0.1% SDS at 65° C. For PCR, a temperature of about 36° C. is typical for low stringency amplification, although annealing temperatures may vary between about 32° C. and 48° C. depending on primer length. Additional guidelines for determining hybridization parameters are provided in numerous references, e.g. Current Protocols in Molecular Biology, eds. Ausubel, et al. 1995).

General Description

Preferably, a QTL of the present invention comprises at least one marker associated with the QTL of the present invention selected from the group consisting of: RM28048 (forward primer: SEQ ID NO: 22; reverse primer: SEQ ID NO: 23); RM28076 (forward primer: SEQ ID NO: 24; reverse primer: SEQ ID NO: 25); RM28089 (forward primer: SEQ ID NO: 26; reverse primer: SEQ ID NO: 27); RM28099 (forward primer: SEQ ID NO: 28; reverse primer: SEQ ID NO: 29); RM28130 (forward primer: SEQ ID NO: 30; reverse primer: SEQ ID NO: 31); RM511 (forward primer: SEQ ID NO: 32; reverse primer: SEQ ID NO: 33); RM1261 (forward primer: SEQ ID NO: 34; reverse primer: SEQ ID NO: 35); RM28166 (forward primer: SEQ ID NO: 36; reverse primer: SEQ ID NO: 37); RM28199 (forward primer: SEQ ID NO: 38; reverse primer: SEQ ID NO: 39); and Indel-8 (forward primer: SEQ ID NO: 60; reverse primer: SEQ ID NO: 61). Because the nucleic acid sequence of the QTL that is responsible for conferring the improved yield under drought stress may only be a fraction of the entire QTL herein identified, the markers indicate linked inheritance of genetic regions or the absence of observed recombination within such genetic regions. Therefore, it is noted that the markers listed herein indicate the chromosomal region where a QTL of the invention is located in the genome of the specified rice varieties and that those markers do not necessarily define the boundaries or the structure of that QTL. Thus, the part of the QTL that comprises the essential yield-improving nucleic acid sequence(s) may be considerably smaller than that indicated by the contiguous markers listed for a particular QTL. Such a part is herein referred to as a “yield-improving part” of a QTL. As a result, a yield-improving part of a QTL need not necessarily comprise any of the listed markers. Also, other markers may be used to indicate the various QTLs, provided that such markers are genetically linked to the QTLs.

A yield-improving part of a QTL for improving yield under drought stress in cereal grasses may be identified by using a molecular marker technique, for instance, with one or more of the markers for a QTL disclosed herein as being linked to said QTL, preferably in combination with a yield bioassay. Cereal grass plants that do not comprise a yield-improving part of a QTL of the present invention have a relatively lower yield. The markers provided by the present invention may be used for detecting the presence of one or more QTLs of the invention in a cereal grass plant suspected of having improved yield under drought stress, and may therefore be used in methods involving marker-assisted breeding and selection of cereal grass plants having improved yield under drought stress. Preferably, detecting the presence of a QTL of the invention is performed with at least one of the markers for a QTL described herein as being linked to the QTL. The present invention therefore relates in another aspect to a method for detecting the presence of a QTL for improved yield under drought stress, comprising detecting the presence of a nucleic acid sequence of the QTL in a cereal grass plant suspected of having improved yield under drought stress, wherein the presence of the nucleic acid sequence may be detected by the use of the said markers.

The nucleic acid sequence of a QTL of the present invention may be determined by methods known to the skilled person. For instance, a nucleic acid sequence comprising the QTL or a yield-improving part thereof may be isolated from a donor plant by fragmenting the genome of said plant and selecting those fragments harboring one or more markers indicative of the QTL. Subsequently, or alternatively, the marker sequences (or parts thereof) indicative of the QTL may be used as PCR amplification primers, in order to amplify a nucleic acid sequence comprising said QTL from a genomic nucleic acid sample or a genome fragment obtained from said plant. The amplified sequence may then be purified in order to obtain the isolated QTL. The nucleotide sequence of the QTL, and/or of any additional markers comprised therein, may then be obtained by standard sequencing methods.

The present invention therefore also relates to an isolated nucleic acid (preferably DNA) sequence that comprises a QTL of the present invention, or a yield-improving part thereof. Thus, the markers that pinpoint the various QTLs described herein may be used for the identification, isolation and purification of one or more genes from cereal that encode for yield improvement under drought stress.

The nucleotide sequence of a QTL of the present invention may, for instance, also be resolved by determining the nucleotide sequence of one or more markers associated with the QTL and designing internal primers for the marker sequences that may then be used to further determine the sequence of the QTL outside of the marker sequences. For instance, the nucleotide sequence of the markers disclosed herein may be obtained by isolating the markers from the electrophoresis gel used in the determination of the presence of the markers in the genome of a subject plant, and determining the nucleotide sequence of the markers by, for instance, dideoxy chain terminating methods, which are well known in the art.

In embodiments of such methods for detecting the presence of a QTL in a cereal grass plant, the method may also comprise the steps of providing a oligonucleotide or nucleic acid capable of hybridizing under stringent hybridization conditions to a nucleic acid sequence of a marker linked to the QTL, preferably selected from the markers disclosed herein as being linked to said QTL, contacting the oligonucleotide or nucleic acid with a genomic nucleic acid of a cereal grass plant suspected of possessing relatively higher yield during drought stress, and determining the presence of specific hybridization of the oligonucleotide or nucleic acid to said genomic nucleic acid. Preferably, said method is performed on a nucleic acid sample obtained from the cereal grass plant suspected of possessing relatively higher yield during drought, although in situ hybridization methods may also be employed. Alternatively, and in a more preferred embodiment, the skilled person may, once the nucleotide sequence of the QTL has been determined, design specific hybridization probes or oligonucleotides capable of hybridizing under stringent hybridization conditions to the nucleic acid sequence of said QTL and may use such hybridization probes in methods for detecting the presence of a QTL of the invention in a cereal grass plant suspected of possessing relatively higher yield during drought stress.

Production of Cereal Grass Plants with Improved Yield under Drought Stress by Transgenic Methods.

According an aspect of the present invention, a nucleic acid (preferably DNA) sequence comprising at least one QTL of the present invention or a yield-improving part thereof, may be used for the production of a cereal grass plant with improved yield under drought stress. In this aspect, the invention provides for the use of a QTL of the present invention or yield-improving parts thereof, for producing a cereal grass plant with improved yield under drought stress, which use involves the introduction of a nucleic acid sequence comprising said QTL in a cereal grass plant having relatively low yield under drought stress. As stated, said nucleic acid sequence may be derived from a suitable donor cereal grass plant. Suitable donor rice plants capable of providing a nucleic acid sequence comprising at least one of the hereinbefore described QTLs, or yield-improving parts thereof, are WayRarem, and the WayRarem-derived hybrids IR74371-46-1-1 and IR79971-B-102-B. Other related rice plants that exhibit relatively high yield under drought stress and comprise one or more genes that encode for improved yield under drought stress may also be utilized as donor plants as the present invention describes how this material may be identified.

Once identified in a suitable donor cereal grass plant, the nucleic acid sequence that comprises a QTL for improve yield under drought stress according to the present invention, or a yield-improving part thereof, may be transferred to a suitable recipient plant by any method available. In certain embodiments, a suitable recipient cereal grass plant is a rice plant that does not comprise a yield-improving QTL described herein, or a yield-improving part thereof, including but not limited to Vandana; Kalinga 3; Anjali; IR64; Swarna; Sambha Mahsuri; MTU1010, Lalat; Naveen; Sabitri; BR11; BR29; BR28; TDK1; TDK 9; and Chirang.

For instance, the said nucleic acid sequence may be transferred by crossing a donor cereal grass plant with a susceptible recipient cereal grass plant (i.e. by introgression), by transformation, by protoplast fusion, by a doubled haploid technique, by embryo rescue, or by any other nucleic acid transfer system, optionally followed by selection of offspring plants comprising the QTL and exhibiting improved yield under drought stress. For transgenic methods of transfer a nucleic acid sequence comprising a QTL for improved yield under drought stress according to the present invention, or a yield-improving part thereof, may be isolated from said donor plant by using methods known in the art and the thus isolated nucleic acid sequence may be transferred to the recipient plant by transgenic methods, for instance by means of a vector, in a gamete, or in any other suitable transfer element, such as a ballistic particle coated with said nucleic acid sequence.

Plant transformation generally involves the construction of an expression vector that will function in plant cells. In the present invention, such a vector comprises a nucleic acid sequence that comprises a QTL for improved yield under drought stress of the present invention, or a yield-improving part thereof, which vector may comprise a yield-improving gene that is under control of, or operatively linked to, a regulatory element such as a promoter. The expression vector may contain one or more such operably linked gene/regulatory element combinations, provided that at least one of the genes contained in the combinations encodes for improved yield under drought stress. The vector(s) may be in the form of a plasmid, and can be used alone or in combination with other plasmids to provide transgenic plants that have improved yield under drought stress, using transformation methods known in the art, such as the Agrobacterium transformation system.

Expression vectors may include at least one marker gene, operably linked to a regulatory element (such as a promoter) that allows transformed cells containing the marker to be either recovered by negative selection (by inhibiting the growth of cells that do not contain the selectable marker gene), or by positive selection (by screening for the product encoded by the marker gene). Many commonly used selectable marker genes for plant transformation are known in the art, and include, for example, genes that code for enzymes that metabolically detoxify a selective chemical agent which may be an antibiotic or a herbicide, or genes that encode an altered target which is insensitive to the inhibitor. Several positive selection methods are known in the art, such as mannose selection. Alternatively, marker-less transformation can be used to obtain plants without mentioned marker genes, the techniques for which are known in the art.

One method for introducing an expression vector into a plant is based on the natural transformation system of Agrobacterium. A. tumefaciens and A. rhizogenes are plant pathogenic soil bacteria that genetically transform plant cells. The Ti and Ri plasmids of A. tumefaciens and A. rhizogenes, respectively, carry genes responsible for genetic transformation of the plant. Methods of introducing expression vectors into plant tissue include the direct infection or co-cultivation of plant cells with Agrobacterium tumefaciens. Descriptions of Agrobacterium vectors systems and methods for Agrobacterium-mediated gene transfer are provided by Gruber and Crosby, 1993 and Moloney et al., 1989. See also, U.S. Pat. No. 5,591,616. General descriptions of plant expression vectors and reporter genes and transformation protocols and descriptions of Agrobacterium vector systems and methods for Agrobacterium-mediated gene transfer can be found in Gruber and Crosby, 1993. General methods of culturing plant tissues are provided, for example, by Miki et al., 1993 and by Phillips, et al., 1988. A reference handbook for molecular cloning techniques and suitable expression vectors is Sambrook and Russell (2001).

Another method for introducing an expression vector into a plant is based on microprojectile-mediated transformation wherein DNA is carried on the surface of microprojectiles. The expression vector is introduced into plant tissues with a biolistic device that accelerates the microprojectiles to speeds of 300 to 600 m/s which is sufficient to penetrate plant cell walls and membranes. Another method for introducing DNA to plants is via the sonication of target cells. Alternatively, liposome or spheroplast fusion has been used to introduce expression vectors into plants. Direct uptake of DNA into protoplasts using CaCl₂ precipitation, polyvinyl alcohol, or poly-L-ornithine may also be used. Electroporation of protoplasts and whole cells and tissues has also been described.

Following transformation of target tissues, expression of the above described selectable marker genes allows for preferential selection of transformed cells, tissues and/or plants, using regeneration and selection methods now well known in the art. The markers described herein may also be used for that purpose.

Production of Cereal Grass Plants with Improved Yield under Drought Stress by Programmable Site-Specific Nucleases.

Zinc-finger nucleases (ZFNs), transcription activator-like effector nucleases (TALENs), and clustered regularly interspaced short palindromic repeat (CRISPR)/Cas-based RNA-guided DNA endonucleases comprise a powerful class of tools useful in genome engineering. The chimeric nucleases of ZFNs and TALENs are composed of programmable, sequence-specific DNA-binding modules linked to a nonspecific DNA cleavage domain. ZFNs and TALENs enable a broad range of genetic modifications by inducing DNA double-strand breaks that stimulate error-prone non-homologous end joining or homology-directed repair at specific genomic locations.

Site-specific nucleases induce DNA double-strand breaks that stimulate non-homologous end joining and homology directed repair at targeted genomic loci. A thorough review of the ZFN, TALEN, and CRISPR/Cas-based RNA-guided DNA endonuclease is available (Gaj et al., 2013). Further discussion of ZNFs may be found in U.S. Pat. Nos. 8,106,255, 8,399,218, and 8,592,645. Further discussion of TALENs may be found in U.S. Pat. No. 8,697,853. Further discussion of CRISPR/Cas-based RNA-guided DNA endonucleases may be found in U.S. Pat. No. 8,697,359, and in J. D. Sander & J. K. Juong (2014).

In certain aspects, any one of these technologies (ZFNs, TALENs, and CRISPR/Cas-based RNA guided DNA endonucleases) may be used to modify the genome of a cereal grass plant. Such modification may include modification, insertion, or deletion of a QTL or one or more individual genes associated with improved lateral root growth, water uptake, and increased yield under drought conditions. For example, the Vandana genome, which already includes qDTY_{2.3}, or a functional part thereof, may be modified from the Vandana allele to the WayRarem allele at OsNAM_{12.1}.

Production of Cereal Grass Plants with Improved Yield under Drought Stress by Non-Transgenic Methods.

In an alternative embodiment for producing a cereal grass plant with improved yield under drought stress, protoplast fusion can be used for the transfer of nucleic acids from a donor plant to a recipient plant. Protoplast fusion is an induced or spontaneous union, such as a somatic hybridization, between two or more protoplasts (cells of which the cell walls are removed by enzymatic treatment) to produce a single bi- or multi-nucleate cell. The fused cell, which may even be obtained with plant species that cannot be interbred in nature, is tissue cultured into a hybrid plant exhibiting the desirable combination of traits. More specifically, a first protoplast can be obtained from a cereal grass plant or other plant line that exhibits improved yield under drought stress. For example, a protoplast from rice WayRarem can be used. A second protoplast can be obtained from rice or other plant variety, preferably a variety that comprises commercially desirable characteristics, such as, but not limited to disease resistance, insect resistance, weed resistance, etc. The protoplasts are then fused using traditional protoplast fusion procedures, which are known in the art.

Alternatively, embryo rescue may be employed in the transfer of a nucleic acid comprising one or more QTLs of the present invention from a donor plant to a recipient plant. Embryo rescue can be used as a procedure to isolate embryo's from crosses wherein plants fail to produce viable seed. In this process, the fertilized ovary or immature seed of a plant is tissue cultured to create new plants (Pierik, 1999).

The present invention also relates to a method of producing a cereal grass plant having improved yield under drought stress comprising the steps of performing a method for detecting the presence of a quantitative trait locus (QTL) associated with improved yield under drought stress in a donor cereal grass plant according to invention as described above, and transferring a nucleic acid sequence comprising at least one QTL thus detected, or a yield-improving part thereof, from said donor plant to a cereal grass plant having a relatively lower yield under drought stress. The transfer of said nucleic acid sequence may be performed by any of the methods previously described herein.

A preferred embodiment of such a method comprises the transfer by introgression of said nucleic acid sequence from a cereal grass plant having improved yield under drought stress into a cereal grass plant having a relatively lower yield under drought stress by crossing said plants. This transfer may thus suitably be accomplished by using traditional breeding techniques. QTLs are preferably introgressed into commercial cereal grass varieties by using marker-assisted breeding (MAS). Marker-assisted breeding or marker-assisted selection involves the use of one or more of the molecular markers for the identification and selection of those offspring plants that contain one or more of the genes that encode for the desired trait. In the present instance, such identification and selection is based on selection of QTLs of the present invention or markers associated therewith. MAS can also be used to develop near-isogenic lines (NIL) harboring the QTL of interest, allowing a more detailed study of each QTL effect and is also an effective method for development of backcross inbred line (BIL) populations (see, e.g., Nesbitt et al., 2001; van Berloo et al., 2001). Cereal grass plants developed according to this preferred embodiment can advantageously derive a majority of their traits from the recipient plant, and derive improved yield under drought stress from the donor plant.

As discussed briefly above, traditional breeding techniques can be used to introgress a nucleic acid sequence encoding for improved yield under drought stress into a recipient cereal grass plant having a relatively lower yield under drought stress. In one method, which is referred to as pedigree breeding, a donor cereal grass plant comprising a nucleic acid sequence encoding for improved yield under drought stress is crossed with a cereal grass plant having a relatively lower yield under drought stress that preferably exhibits commercially desirable characteristics, such as, but not limited to, disease resistance, insect resistance, weed resistance, etc. The resulting plant population (representing the F1 hybrids) is then self-pollinated and set seeds (F2 seeds). The F2 plants grown from the F2 seeds are then screened for improved yield under drought stress. The population can be screened for improve yield under drought stress in a number of different ways. For example, the population can be screened by field evaluation over several seasons. Yield may be determined by weight of grain per hectare (e.g., t ha⁻¹, kg ha⁻¹), average grain weight per plant, or any other method known in the art.

A Cereal Grass Plant Having Improved Yield under Drought Stress, or a Part Thereof, Obtainable by a Method of the Invention is Also an Aspect of the Present Invention.

Another aspect of the present invention relates to a cereal grass plant having improved yield under drought stress, or part thereof, comprising within its genome at least one QTL, or a yield-improving part thereof, consisting at least in part of the QTL on chromosome 12 of WayRarem associated with improved yield under drought stress, wherein the QTL or the yield improving part thereof is not in its natural genetic background. The cereal grass plants having improved yield under drought stress of the present invention can be of any genetic type such as inbred, hybrid, haploid, dihaploid, parthenocarp, or transgenic. Further, the plants of the present invention may be heterozygous or homozygous for the improved yield under drought stress trait, preferably homozygous. Although the QTLs of the present invention, as well as those QTLs obtainable by a method of the invention, as well as yield-improving parts thereof, may be transferred to any plant in order to provide for a plant having improved yield under drought stress, the methods and plants of the invention are preferably related to the cereal grasses family, more preferably rice.

Inbred cereal grass lines having improved yield under drought stress can be developed using the techniques of recurrent selection and backcrossing, selfing and/or dihaploids or any other technique used to make parental lines. In a method of selection and backcrossing, improved yield under drought stress can be introgressed into a target recipient plant (which is called the recurrent parent) by crossing the recurrent parent with a first donor plant (which is different from the recurrent parent and referred to herein as the “non-recurrent parent”). The recurrent parent is a plant that has relatively low yield under drought stress and possesses commercially desirable characteristics, such as, but not limited to disease resistance, insect resistance, weed resistance, etc. The non-recurrent parent comprises a nucleic acid sequence that encodes for improved yield under drought stress. The non-recurrent parent can be any plant variety or inbred line that is cross-fertile with the recurrent parent. The progeny resulting from a cross between the recurrent parent and non-recurrent parent are backcrossed to the recurrent parent. The resulting plant population is then screened. The population can be screened in a number of different ways. F1 hybrid plants that exhibit improved yield under drought stress, comprise the requisite nucleic acid sequence encoding for improved yield under drought stress, and possess commercially desirable characteristics, are then selected and selfed and selected for a number of generations in order to allow for the cereal grass plant to become increasingly inbred. This process of continued selfing and selection can be performed for two to five or more generations. The result of such breeding and selection is the production of lines that are genetically homogenous for the genes associated with improved yield under drought stress as well as other genes associated with traits of commercial interest. Instead of using phenotypic pathology screens of bioassays, MAS can be performed using one or more of the herein described molecular markers, hybridization probes or nucleic acids to identify those progeny that comprise a nucleic acid sequence encoding for improved yield under drought stress. Alternatively, MAS can be used to confirm the results obtained from the quantitative bioassays. Once the appropriate selections are made, the process is repeated. The process of backcrossing to the recurrent parent and selecting for improved yield under drought stress is repeated for approximately five or more generations. The progeny resulting from this process are heterozygous for one or more genes that encode for improve yield under drought stress. The last backcross generation is then selfed in order to provide for homozygous pure breeding progeny for improved yield under drought stress.

The cereal grass lines having improved yield under drought stress described herein can be used in additional crossings to create hybrid plants having improved yield under drought stress. For example, a first inbred cereal grass plant having improved yield under drought stress of the invention can be crossed with a second inbred cereal grass plant possessing commercially desirable traits such as, but not limited to, disease resistance, insect resistance, weed resistance, etc. This second inbred cereal grass line may or may not have relatively improved yield under drought stress.

Marker Assisted Selection and Backcrossing.

qDTY_{12.1} MAS and MABC are described herein.

As is known to those skilled in the art, there are many kinds of molecular markers. For example, molecular markers can include restriction fragment length polymorphisms (RFLP), random amplified polymorphic DNA (RAPD), amplified fragment length polymorphisms (AFLP), single nucleotide polymorphisms (SNP) or simple sequence repeats (SSR). Simple sequence repeats (SSR) or microsatellites are regions of DNA where one to a few bases are tandemly repeated for few to hundreds of times. For example, a di-nucleotide repeat would resemble CACACACA and a trinucleotide repeat would resemble ATGATGATGATG (SEQ ID NO: 141). Simple sequence repeats are thought to be generated due to slippage mediated errors during DNA replication, repair and recombination. Over time, these repeated sequences vary in length between one cultivar and another. An example of allelic variation in SSRs would be: allele A being GAGAGAGA (4 repeats of the GA sequence) and allele B being GAGAGAGAGAGA (6 repeats of the GA sequence) (SEQ ID NO: 142). When SSRs occur in a coding region, their survival depends on their impact on structure and function of the encoded protein. Since repeat tracks are prone to DNA-slippage mediated expansions/deletions, their occurrences in coding regions are limited by non-perturbation of the reading frame and tolerance of expanding amino acid stretches in the encoded proteins. Among all possible SSRs, tri-nucleotide repeats or multiples thereof are more common in coding regions.

A single nucleotide polymorphism (SNP) is a DNA sequence variation occurring when a single nucleotide—A, T, C or G—differs between members of a species (or between paired chromosomes in an individual). For example, two sequenced DNA fragments from two individuals, AAGCCTA to AAGCTTA, contain a difference in a single nucleotide. In this case, there are two alleles: C and T.

A primary motivation for development of molecular markers in crop species is the potential for increased efficiency in plant breeding through marker assisted selection (MAS) and marker assisted backcrossing (MABC). Genetic marker alleles, or alternatively, identified QTL alleles, are used to identify plants that contain a desired genotype at one or more loci and that are expected to transfer the desired genotype, along with a desired phenotype to their progeny. Genetic marker alleles can be used to identify plants that contain a desired genotype at one locus or at several unlinked or linked loci (e.g., a haplotype) and that would be expected to transfer the desired genotype, along with a desired phenotype to their progeny. The present invention provides the means to identify cereal grass plants, particularly rice, that are able to improve the yield of grain under drought stress by identifying plants having a specified quantitative trait locus or gene, e.g., qDTY_{12.1}, OsNAM_{12.1}, and homologous or linked markers. Similarly, by identifying plants having poor yield under drought stress, such low-yielding plants can be identified and, e.g., eliminated from subsequent crosses.

After a desired phenotype, e.g., improved yield under drought stress and a polymorphic chromosomal locus, e.g., a marker locus or QTL, are determined to segregate together, it is possible to use those polymorphic loci to select for alleles corresponding to the desired phenotype: a process called marker-assisted selection (MAS). In brief, a nucleic acid corresponding to the marker nucleic acid is detected in a biological sample from a plant to be selected. This detection can take the form of hybridization of a probe nucleic acid to a marker, e.g., using allele-specific hybridization, Southern analysis, northern analysis, in situ hybridization, hybridization of primers followed by PCR amplification of a region of the marker or the like. A variety of procedures for detecting markers are described herein. After the presence (or absence) of a particular marker and/or marker allele in the biological sample is verified, the plant may be selected, i.e., used to make progeny plants by selective breeding.

Rice breeders combine modern irrigated rice varieties, e.g. Vandana and Sabitri, with genes for improved yield under drought stress and other desirable traits to develop improved rice varieties. Screening a large number of plants for improved yield under drought stress can be expensive, time consuming and unreliable. Use of the polymorphic loci described herein, and genetically-linked nucleic acids, as genetic markers for the improved yield under drought stress locus is an effective method for selecting varieties capable of fertility restoration in breeding programs. For example, one advantage of marker-assisted selection over field evaluations for improved yield under drought stress is that MAS can be done at any time of year regardless of the growing season. Moreover, environmental effects are irrelevant to marker-assisted selection.

Another use of MAS in plant breeding is to assist the recovery of the recurrent parent genotype by backcross breeding. Backcross breeding is the process of crossing a progeny back to one of its parents. Backcrossing is usually done for the purpose of introgressing one or a few loci from a donor parent into an otherwise desirable genetic background from the recurrent parent. The more cycles of backcrossing that are done, the greater the genetic contribution of the recurrent parent to the resulting variety. This is often necessary, because donor parent plants may be otherwise undesirable. In contrast, varieties which are the result of intensive breeding programs may have excellent yield, fecundity or the like, merely being deficient in one desired trait such as yield under drought stress. As a skilled worker understands, backcrossing can be done to select for or against a trait.

Markers corresponding to genetic polymorphisms between members of a population can be detected by numerous methods, well-established in the art (e.g., restriction fragment length polymorphisms, isozyme markers, allele specific hybridization (ASH), amplified variable sequences of the plant genome, self-sustained sequence replication, simple sequence repeat (SSR), single nucleotide polymorphism (SNP) or amplified fragment length polymorphisms (AFLP)).

The majority of genetic markers rely on one or more properties of nucleic acids for their detection. For example, some techniques for detecting genetic markers utilize hybridization of a probe nucleic acid to nucleic acids corresponding to the genetic marker. Hybridization formats include but are not limited to, solution phase, solid phase, mixed phase or in situ hybridization assays. Markers which are restriction fragment length polymorphisms (RFLP), are detected by hybridizing a probe (which is typically a sub-fragment or a synthetic oligonucleotide corresponding to a sub-fragment of the nucleic acid to be detected) to restriction digested genomic DNA. The restriction enzyme is selected to provide restriction fragments of at least two alternative (or polymorphic) lengths in different individuals and will often vary from line to line. Determining a (one or more) restriction enzyme that produces informative fragments for each cross is a simple procedure, well known in the art. After separation by length in an appropriate matrix (e.g., agarose) and transfer to a membrane (e.g., nitrocellulose, nylon), the labeled probe is hybridized under conditions which result in equilibrium binding of the probe to the target followed by removal of excess probe by washing. Nucleic acid probes to the marker loci can be cloned and/or synthesized. Detectable labels suitable for use with nucleic acid probes include any composition detectable by spectroscopic, radioisotopic, photochemical, biochemical, immunochemical, electrical, optical or chemical means. Useful labels include biotin for staining with labeled streptavidin conjugate, magnetic beads, fluorescent dyes, radiolabels, enzymes and colorimetric labels. Other labels include ligands which bind to antibodies labeled with fluorophores, chemiluminescent agents and enzymes. Labeling markers is readily achieved such as by the use of labeled PCR primers to marker loci.

The hybridized probe is then detected using, most typically, autoradiography or other similar detection technique (e.g., fluorography, liquid scintillation counter, etc.). Examples of specific hybridization protocols are widely available in the art.

Amplified variable sequences refer to amplified sequences of the plant genome which exhibit high nucleic acid residue variability between members of the same species. All organisms have variable genomic sequences and each organism (with the exception of a clone) has a different set of variable sequences. Once identified, the presence of specific variable sequence can be used to predict phenotypic traits. Preferably, DNA from the plant serves as a template for amplification with primers that flank a variable sequence of DNA. The variable sequence is amplified and then sequenced.

In vitro amplification techniques are well known in the art. Examples of techniques sufficient to direct persons of skill through such in vitro methods, including the polymerase chain reaction (PCR), the ligase chain reaction (LCR), O,β-replicase amplification and other RNA polymerase mediated techniques (e.g., NASBA), are readily found in the art. One of skill will appreciate that essentially any RNA can be converted into a double stranded DNA suitable for restriction digestion, PCR expansion and sequencing using reverse transcriptase and a polymerase.

Oligonucleotides for use as primers, e.g., in amplification reactions and for use as nucleic acid sequence probes, are typically synthesized chemically according to the solid phase phosphoramidite triester method, or can simply be ordered commercially.

Alternatively, self-sustained sequence replication can be used to identify genetic markers. Self-sustained sequence replication refers to a method of nucleic acid amplification using target nucleic acid sequences which are replicated exponentially in vitro under substantially isothermal conditions by using three enzymatic activities involved in retroviral replication: (1) reverse transcriptase, (2) Rnase H and (3) a DNA-dependent RNA polymerase. By mimicking the retroviral strategy of RNA replication by means of cDNA intermediates, this reaction accumulates cDNA and RNA copies of the original target.

As mentioned above, there are many different types of molecular markers, including amplified fragment length polymorphisms (AFLP), allele-specific hybridization (ASH), single nucleotide polymorphisms (SNP), simple sequence repeats (SSR), and isozyme markers. Methods of using the different types of molecular markers are known to those skilled in the art.

The qDTY_{12.1} QTL and genes NAM_{12.1}; GPDP_{12.1}; STPK_{12.1}; POLe_{12.1}; MtN3_{12.1}; WAK_{12.1}; CesA_{12.1}; GDP_{12.1}; ARF_{12.1}; Nod_{12.1}; and AmH1_{12.1}, or homologs thereof, in the genome of a plant exhibiting a preferred phenotypic trait is determined by any method listed above, e.g., RFLP, AFLP, SSR, etc. If the nucleic acids from the plant are positive for one or more desired genetic markers, the plant can be selfed to create a true breeding line with the same genotype or it can be crossed with a plant with the same marker or with other desired characteristics to create a sexually crossed hybrid generation.

It will be recognized by one skilled in the art that the materials and methods of the present invention may be similarly used to confer improved yield under drought stress in cereal grasses other than rice, such as corn, wheat, barley, sorghum, millet, oats, and rye

### EXAMPLES

### Example 1

### A Gene-Complex Affecting Multiple Component Traits Underpins a Large-Effect QTL for Rice Yield Under Drought

Materials and Methods.

Plant Material.

qDTY_{12.1} was identified in an F_{3:4} population derived from the cross Vandana/WayRarem. Vandana is an upland-adapted cultivar derived from a cross between C22 and Kalakeri. This cultivar is early to mature, and low yielding but tolerant of drought, and is grown in drought-prone areas of Jharkhand and Orissa (eastern India). WayRarem is a high-yielding, drought-susceptible upland rice cultivar from Indonesia. The yield-increasing allele in this study was derived from the susceptible parent, WayRarem, making the tolerant parent Vandana the recipient parent for a MAB program. IR79971-B-102-B, one of the F₃-derived lines from the original population, was used as the donor for qDTY_{12.1}. This line was backcrossed to Vandana to develop BC₂- and BC₃-derived populations for the identification of NILs with qDTY_{12.1} showing improved tolerance of drought compared with Vandana. A set of such contrasting+QTL and −QTL BC₂F₃-derived lines was used for the qDTY_{12.1} physiology studies.

Generation of Genotypic Data.

Young leaves were collected from 2-week-old plants and freeze-dried. Freeze-dried leaf samples were ground using a Geno/Grinder® (SPEX CertiPrep) and DNA was extracted by the modified CTAB method in deep-well plates. The quality and quantity of DNA were then checked on 0.8% agarose gel and diluted to a final concentration of 20 ng μL⁻¹ with TE (Tris-EDTA) buffer. Polymerase chain reaction (PCR) was performed in 96-well plates. After the PCR was completed, 4 μL of 6× loading dye was added to each well. Four μL of the resulting solution mix was then loaded into an 8% (w/v) polyacrylamide gel for size separation of the amplified DNA fragments using a mini vertical electrophoresis system (CBS Scientific, model MGV-202-33). DNA fragments were then stained with SYBR® Safe gel stain (Invitrogen) and visualized with a UV trans-illuminator.

Rice SSR markers (RM28076, RM28089, RM28099, RM28130, RM511, RM1261, RM28166, RM28199, RM28048, and Indel-8) were used for foreground, recombinant, and background selection. All markers described by Bernier et al. (2007) were used for selection. Three other markers, RM28076, RM28089, and RM28099, were also included for foreground and recombinant selection. The cM position was used for constructing chromosome maps. Graphical genotyping software GGT2 was used for the construction of chromosome maps of the selected lines.

Molecular Marker Analysis and Crossing Scheme.

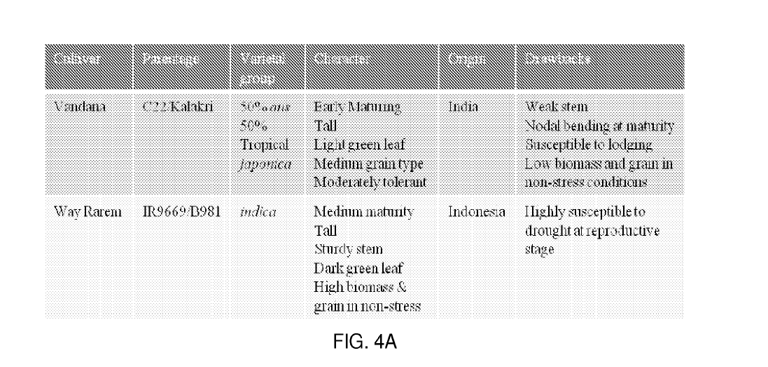

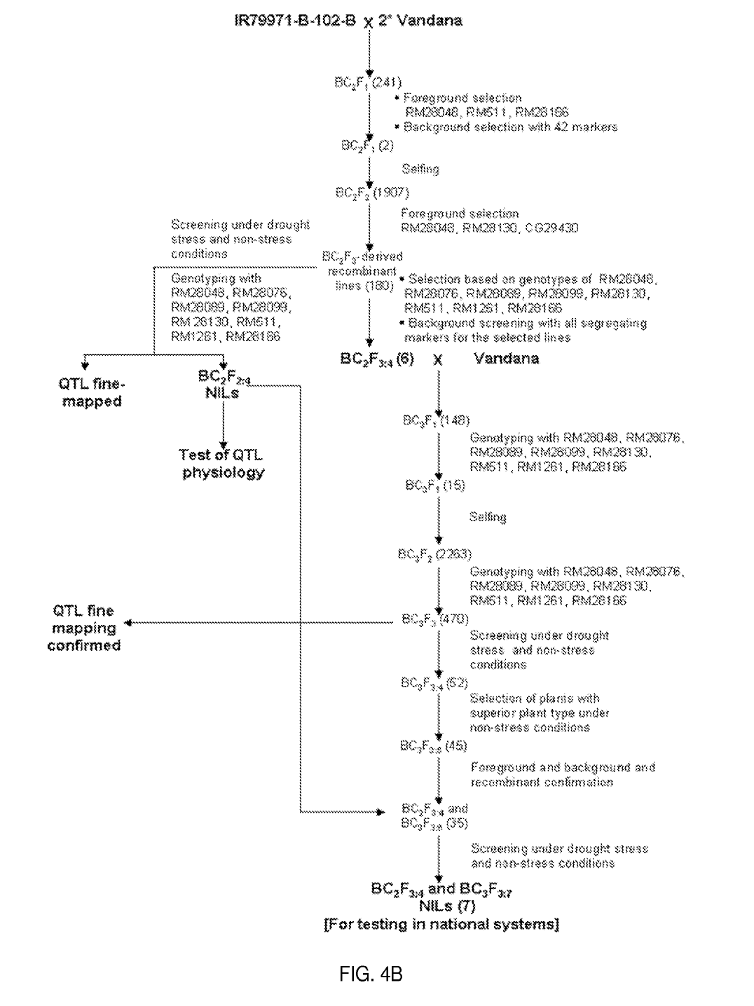

The MAB scheme for the transfer of qDTY_{12.1} into Vandana is shown in FIG. 4B. qDTY_{12.1} spans between RM28048 and RM28166 on chromosome 12 of the rice genome. IR79971-B-102-B, an F_{3:4} line with the full segment of the QTL, was crossed twice to Vandana to develop a BC₂F₁ (241 plants). The population was screened with RM28048, RM511, and RM28166 to identify individual plants segregating for qDTY_{12.1} (foreground selection). The selected plants were then screened with 42 SSR markers for the presence of the Vandana allele across the background (background selection) and two BC₂F₁ plants (IR84984-21-19 and IR84984-83-15) segregating for qDTY_{12.1} and maximum background recovery were identified to develop the BC₂F₂ population. A large BC₂F₂ population (1907 plants) was developed from the identified BC₂F₁ plants and was genotyped with foreground markers RM28048, RM28130, and CG29430 to identify lines segregating for the qDTY_{12.1} locus. The 180 BC₂F₃ lines identified through this process were then saturated with six additional SSR markers, RM28076, RM28089, RM28099, RM511, RM1261, and RM26166, within the region and were screened under varying drought-stress conditions to identify high-yielding BC₂F₃-derived NILs. Lines from this population with and without the full region of the QTL (RM28048-RM28166) were also used for the study of QTL physiology. Six BC₂F_{3:4} lines, IR84984-21-19-861-B, IR84984-21-19-158-B, IR84984-21-19-177-B, IR84984-21-19-917-B, IR84984-83-15-805-B, and IR84984-83-15-937-B, were backcrossed to Vandana to generate 148 BC₃F₁ plants. These lines were also screened with all background markers segregating in the BC₂F₁ background screening. The BC₃F₁ plants were screened with all eight markers within the QTL, and 15 BC₃F₁ plants segregating for different segments of qDTY_{12.1} were selected for developing a large BC₃F₂ population (2263 plants). Since this population came from 15 different F₁ plants, the inventors were able to divide it into 15 sub-populations, each segregating for a different segment of the QTL. These sub-populations were then genotyped with all segregating markers within the QTL based on the respective F₁ information available and 470 BC₃F₂ plants were identified. The genotypes of these plants were confirmed with all eight markers within the QTL region and BC₃F₃ lines from these plants were screened under drought stress and non-stress conditions. The 52 best BC₃F_{3:4} lines were selected based on their field performance under stress. These lines were then evaluated under non-stress conditions for superior plant type and yield and single panicle selections were conducted. Seeds from 62 different lines coming from BC₂- and BC₃-derived populations were multiplied under non-stress conditions and confirmed for the presence of qDTY_{12.1} along with background screening with segregating markers. Thirty-five selected lines with the qDTY_{12.1} segment were evaluated in advanced yield trials (AYTs) under upland stress and non-stress conditions and four BC₂-derived and three BC₃-derived lines with qDTY_{12.1}, superior plant type, the highest Vandana genome recovery, and highest yield under non-stress conditions were identified.

Reproductive-Stage Drought Screening Experiments.

Field experiments were conducted from at the International Rice Research Institute (IRRI), Los Baños, Laguna, Philippines, located at 14° 13′N latitude, 121° 15′E longitude, at an elevation of 21 m.

Population screening and physiological characterization were conducted in upland conditions under drought and non-stress treatments in either the open field or rainout shelter. Throughout this study, the term ‘upland’ refers to field trials conducted under direct-seeded, non-puddled, non-flooded aerobic conditions in leveled upland fields. Screening of BC₂F_{3:4} and BC₃F_{3:4} lines with qDTY_{12.1} developed through MAB, and subsequent AYTs and physiology studies, were conducted using an α-lattice or randomized complete block design along with Vandana, WayRarem, in 2-4 replications of 1-4 row plots 1.5-3 m in length, 0.25 m row-to-row spacing and 2.0-2.5 g seed per linear meter. Fertilizer and crop management practices were followed as described by Bernier et al. (2007). In all stress experiments, trials were sprinkler-irrigated twice a week during establishment and early vegetative growth. A line source sprinkler was used to create a gradient with 3 distinct treatments. At 35 days after seeding, stress was initiated by withholding irrigation and plots were irrigated only when the soil water tension fell below −50 kPa at 30-cm soil depth and most lines had wilted and exhibited leaf drying. Upland non-stress trials received the same cultural practices as the stress trials except that irrigation was continued twice a week up to 10 days before harvest.

Morphophysiology Measurements.

Genetic variation for water uptake was determined by volumetric soil moisture at 10 cm depth increments (Diviner 2000, Sentek Sensor Technologies, Stepney SA, Australia) in 2012DS and 2012WS, where PVC tubes were installed in all plots at the mid-point between rows and hills, ˜30 cm from the edge of the plot. Root samples were taken between 58 and 85 DAS with 3 subreplicates per plot using a 4-cm-diameter core sampler to a depth of 60 cm, washed, scanned, and analyzed according to Henry et al. 2011. TE was assessed in 2012DS and WS by carbon isotope analysis of the 2 youngest leaves sampled from 3 plants per plot at 2-week intervals from 21-70 DAS. Δ¹³C was calculated as (−8−leaf ¹³C conc)/(1+(leaf ¹³C conc/1000)) according to Farquhar et al. 1989.

Instantaneous transpiration efficiency (TE) was determined at 44 DAS in 2012 WS by LI-6400 portable gas exchange system (Li-Cor Inc., Lincoln, Nebr., USA). In all field trials, days to 50% flowering (DTF), mean plant height at maturity (PH), grain yield, and biomass were recorded according to Venuprasad et al. (2009) from a 2-m² (stress) and 0.125-2.0 m² (non-stress) area of each plot. Statistical analysis approach is outlined in Table 1.

TABLE 1
Statistical analysis used.

| Pᵢⱼₖ = M + Rᵢ + Bⱼ (Rᵢ) + Lₖ + eᵢⱼₖ | |
|---|---|
| Add. (%) = [(T_{L} − T_{V}/2)/Tv]*100 | |
| Symbol | Description |
| P | measurement recorded on a plot |
| M | mean over all plots |
| R | replications |
| B | blocks |
| L | lines |
| e | error |
| Additive (%) | percentage additive effect of the line |
| T_{L} | trait value for the line with the QTL |
| T_{V} | trait value of the recipient parent |
| Statistical Package | Purpose |
| CROPSTAT v 7.2 | Yield trials |
| (63) | |
| R v. 2.8.0 (64) | Physiology expts. |

Seedling Stage Trials.

Seedling stage stress trials were established in upland fields in both dry and wet seasons as described above, except that the drought stress treatment was initiated 7 DAS and plants were harvested at 32 DAS to determine biomass. A seedling greenhouse study was conducted in 4-cm-diam 40-cm deep soil-filled tubes according to Henry et al. 2012. Soil moisture treatments included well-watered (WW; maintained at field capacity) and dry down from field capacity (DD), with five replicates per genotype planted in an RCBD. Water uptake, shoot mass, and root length were determined, including one nodal root from each plant with all lateral roots carefully spread apart in order to detect the number of root branches.

Root Phenotyping.

Mature dehusked seeds of Vandana, WayRarem, NIL, 6 recombinant lines, IR64 (Parent for the transgenic line) and 3 transgenic events were sterilized in 1% Sodium Hypochlorite and were germinated in MS₀ media (KNO₃ —1.9 g/L, (NH₄)₂SO₄ —1.65 g/L, MgSO₄.H2O—0.37 g/L, MnSO₄.4H₂O—22.3 mg/L, ZnSO₄.7H₂O—8.6 mg/L, CuSO₄.5H₂O—0.025 mg/L, CaCl₂.2H₂O—0.44 g/L, KI—0.83 mg/L, CoCl₂.6H₂O—0.025 mg/L, KH₂PO₄—0.17 g/L, H₃BO₃—6.2 mg/L, Na₂MoO₄.2H₂O—0.25 mg/L, FeSO₄.7H₂O—27.8 mg/L, Na₂EDTA.2H₂O—37.3 mg/L, Nicotinic acid—0.5 mg/L, Pyridoxine HCl—0.5 mg/L, Thiamine HCl—0.1 mg/L,Glycine—2 mg/L, myoinositol—100 mg/L, sucrose—30 g, gelrite—0.2% (w/v), pH to 5.8, sterilized at 121° C./15 psi for 15 minutes) in dark at 25-29° C. for 3 days. Ten pregerminated seeds per line were transferred into MS₀ with and without 10% (w/v) PEG (MW: 8,000) in test tubes and were grown under light at 29° C. The root morphology was observed after 8 days and was documented using Nikon D90 camera under diffused light.

Statistical Analysis and Construction of Linkage Maps.

The model used for analysis of variance for an α-lattice design was Pᵢⱼₖ=M+Rᵢ+Bⱼ(Rᵢ)+Lₖ+eᵢⱼₖ where Pᵢⱼₖ is the measurement recorded on a plot, M is the mean over all plots, and R, B, L, and e refer to replications, blocks, lines, and error, respectively. Data of yield trials for computation of means were analyzed using CROPSTAT v. 7.2 (IRRI, 2007) taking the effect of replications and block within replications as random. Additive effect of the line with the QTL was computed as Add. (%)=[(T_{L}−T_{V}/2)/T_{V}]*100 where Add. (%) is the percentage additive effect of the line with the QTL over the recipient parent (Vandana), T_{L}, is the trait value for the line with the QTL, and T_{V} is the trait value of the recipient parent (Vandana).

Statistical analyses for the physiology experiments were performed in R v. 2.8.0 (R Development Core Team, 2008) using ANOVA and Tukey's HSD test.

SNPs in the 10 Candidate Genes.

Targeted sequencing of the QTL region from Vandana, WayRarem and IR64 genomes were performed. Sequencing libraries for the three samples were created using Agilent SureSelect protocol for paired-end Illumina platform (Agilent Technologies: SureSelectXT Target Enrichment System for Illumina Paired-End Sequencing Library, V1.4.1; publication number G7530-90000). 3 μg of high quality genomic DNA was used per sample as starting material and sheared. Concentration of the sheared DNA were analyzed and checked for quality control. The libraries were then PCR amplified and hybridized with SureSelect custom made baits. Post-capture-PCR was performed on the hybridized samples to incorporate the sequencing Index Tag followed by another round of quality control for PCR efficiency in Bioanalyser. Each tag added library was quantified using qPCR with Agilent Technologies QPCR NGS library quantification kit (Illumina GA) on an Agilent Technologies MX3005 qPCR machine.

Once done, sequencing was performed by Ambry Genetics (15 Argonaut, Aliso Viejo, Calif. 92656, United States) using Illumina HiSeq2000. Initial data processing and base calling, including extraction of cluster intensities, was done using RTA 1.12.4 (HiSeq Control Software 1.4.5). Sequence quality filtering script was executed in the Illumina CASAVA software (ver 1.8.2, Illumina, Hayward, Calif.).

The raw Fastq reads were mapped/aligned to the IRGSP Build 5 reference sequence using the Bowtie2 aligner. After the alignment, typically a round of quality control removal on reads with poor quality score was performed using Genespring 12.5 software (Agilent Technologies). Reads with average score less than 30 and with ambiguous (N) bases were removed. After filtering of reads on quality score, another filter was performed to address the duplicate artifact introduced due to the Polymerase Chain Reaction (PCR) bias for certain regions. In this setup, all duplicate status reads were removed leaving behind only 1 copy. Lastly, due to the enrichment done before the sequencing, reads outside the SureSelect region were removed. Finally, Single Nucleotide Polymorphisms (SNPs) were identified in Genespring 12.5.

Candidate Gene Selection.

qDTY12.1 covers a region of 1.75 Mb spanning the area between the markers RM28099 and RM28166. A total of 248 genes were collected from Gramene database. Since this region was closer to the centromere, there were a lot of transposons and retro-transposons. As a first step, all the transposons and retro-transposons were removed.

There were 118 genes in 1.75 Mb spanning the QTL after dropping the (retro)transposons These were analyzed in silico for expression under drought from three experiments, (GSE26280, GSE24048 and GSE6901). Manual differential expression analysis in an Excel sheet using the relative values; SAM module in Excel); SAM module in TIGR MeV4 and Genevestigator were used. There were 28 recurrent genes in these analyses. This information was combined with sequence polymorphism to narrow down the potential CGs. After literature survey, 10 CGs were picked which were analyzed for promoter cis-elements and gene ontology (GO) analysis which supported the selection of the 10 genes as putative CGs. In another analysis, putative NAM binding sites were predicted. The NAM binding motif was generated from literature and sites predicted using an in house perl script. This allowed the selection of OsAmH_{12.1}.

In order to understand the cis-elements governing the expression of these 10 CGs, promoters of these genes were submitted to PLACE database (dna.affrc.go.jp/PLACE). Drought specific cis-elements were observed in all the 10 CGs. To gain a clearer insight into the functional characteristic of the genes and their role, gene ontologies were predicted in ARGOT² (medcomp.medicina.unipd.it/Argot2/index.php).

In another separate analysis, putative NAM binding sites were predicted in the genes of the QTL. The NAM binding motif was generated from various literature sources and those sites were predicted using an in house built perl script.

Fine Mapping.

Nearly 1900 BC2F3 lines were phenotyped for drought tolerance through yield analysis and these lines were then genotyped using SSR markers described herein. Fifty six of these lines were genotyped with 9 candidate gene-based markers using primers designed (Table 2) after comparing the Vandana and WayRarem sequence obtained from the NGS data.

TABLE 2
Primers used for Fine mapping

| GENE | PRIMER NAME | SEQUENCE | SEQ ID NO: |
|---|---|---|---|
| OsAmh_{12.1} | AMI_C2_F2 | TTTGCCAGCTTTGACCTTCA | 143 |
| OsAmh_{12.1} | AMI_C2_R2 | CCATCGACCGTTGCACATTA | 144 |
| OsARF_{12.1} | ARF_C3_F2 | ACCTCCCGTTGCTTCTCTC | 145 |
| OsARF_{12.1} | ARF_C3_R2 | TCGGAGAGAATTTCGGGCTC | 146 |
| OsNAM_{12.1} | NAM12_PF + 1656 | CCACATCGGTTATGACCA | 147 |
| OsNAM_{12.1} | 5UNAM12-R1 | CGTCTCCATCGATACACCTC | 148 |
| OsGDP_{12.1} | GRAM_C2_F2 | CACCATCTGTCCAAAGTCCA | 149 |
| OsGDP_{12.1} | GRAM_C2_R | GTAACTCTGCTCCGGCAACT | 150 |
| OsGPDP_{12.1} | W-GPDP-C3-F1 | TCGTTTATTCTATTGTTTGCCA | 151 |
| OsGPDP_{12.1} | W-GPDP-C3-R1 | CCATCTCCTTGGCGTGTACAA | 152 |
| OsCesA_{12.1} | Cesa_CF1 | GTGCTGTCCATATATCCTCGC | 153 |
| OsCesA_{12.1} | Cesa_CR1 | CAACCTGGACCATAGCCGCT | 154 |
| OsMtN3_{12.1} | NOD CF2 | CTACCGGATCTACAAGAGCAAG | 155 |
| OsMtN3_{12.1} | NOD CR2 | GATCTTCGTCGTGAACACC | 156 |
| OsPOle_{12.1} | POLe_C2_F | GCGCCAAAATTTCTTGGT | 157 |
| OsPOle_{12.1} | POLe_C2_R2 | CTTCTCGGCGGTGATCTTGA | 158 |
| OsWAK_{12.1} | WAK_Pro_F | CTCTCTACTCGCCAACCACC | 159 |
| OsWAK_{12.1} | WAK_Pro_R | CATGAACAGCCTGGTGTCGT | 160 |

Preparation of c-DNA for Expression Study.

The seeds of recipient parent Vandana, NIL and the donor parent WayRarem were sown in staggered manner so that they reach the stage of booting at the same time in four replicates. Excess water was removed from the pots in two of the replicates and further watering was stopped after 52 days for sowing of Vandana while maintaining the other two replicates in well-watered condition. Explants were collected for RNA isolation after the complete rolling of leaves which occurred after 3 days of dewatering. Similarly roots and leaves of two homozygous transgenic lines and a non-transformed control IR64 plants were collected from the physiology experiment conducted to screen the drought tolerance of the OsNAM transgenic lines. Total RNA was isolated from the leaf, root and panicle of the stressed and the well watered control plants by using Trizol reagent (Ambion, Austin, Tex., USA). The cDNA was synthesized using the ImProm-II Reverse transcription system (Promega Corporation, Madison, USA) as per the manufacturer's instruction.

RT-PCR Protocol.

Primer pairs for use in RT-PCR experiments were designed based on the coding sequence of candidate genes (sequences obtained from Gramene) (Table 3).

TABLE 3
Primers used in studies with qDTY121

| | | | | SEQ | |
|---|---|---|---|---|---|
| | | | | ID | |
| Gene Name | Locus ID | Primer | Sequence information | NO: | Purpose |
| Nodulin MtN3 | LOC_Os12g29220 | NOD_CF2 | 5′-ctaccggatctacaagagcaag-3′ | 155 | RT-PCR |
| family protein | | NOD_CR2 | 5′-gatcttcgtcgtgaacacc-3′ | 156 | RT-PCR |
| Amidohydrolase | LOC_Os12g28270 | AMDH_CF3 | 5′-ggcgtgaggcgtacatcac-3′ | 161 | RT-PCR |
| | | AMDH_CR1 | 5′-ggagtactgattggtagtgtcg-3′ | 162 | RT-PCR |
| No apical | LOC_Os12g29330 | NAM_CF2 | 5′-gagtgagcaggagaggta-3′ | 163 | RT-PCR |
| meristem protein | | NAM_CR2 | 5′-catgacccagtccgtcttct-3′ | 164 | RT-PCR |
| Serine/threonine | LOC_Os12g27520 | SER_CF2 | 5′-tagtgcaaagccttccctgt-3′ | 165 | RT-PCR |
| protein kinase | | SER_CR2 | 5′-cctgcggtagctttcgtaac-3′ | 166 | RT-PCR |
| Cellulose | LOC_Os12g29300 | CESA_F | 5′-gcgtcttcttcgactgcac-3′ | 167 | RT-PCR |
| synthase | | CESA_R | 5′-caacctggaccatagccgct-3′ | 154 | RT-PCR |
| Expressed | LOC_Os12g29340 | EXP_CF1 | 5′-gctgaaagcctctccatgtt-3′ | 168 | RT-PCR |
| protein | | EXP_CR1 | 5′-gcatgatgcatagtggatgg-3′ | 169 | RT-PCR |
| GRAM domain | LOC_Os12g29400 | GDP_CF2 | 5′-tactcacccagccatggac-3′ | 170 | RT-PCR |
| containing | | GDP_CR2 | 5′-cggcgaacgtctgcttgta-3′ | 171 | RT-PCR |
| protein | | | | | |
| Auxin response | LOC_Os12g29520 | ARF_CF2 | 5′-tggcacatggtctcttatgc-3′ | 172 | RT-PCR |
| factor | | ARF_CR2 | 5′-agaggagcgctgatctatgc-3′ | 173 | RT-PCR |
| Wall associated | LOC_Os12g29430 | WPK_CF2 | 5′-gcctcactactggaagaagg-3′ | 174 | RT-PCR |
| protein kinases | | WPK_CR2 | 5′-tcccctctagctgatatgc-3′ | 175 | RT-PCR |
| Pollen Ole | LOC_Os12g28770 | OLE_CF2 | 5′-cgtcatcgacaacccctcgc-3′ | 176 | RT-PCR |
| | | OLE_CR2 | 5′-cagtagaacagcgacgtgtt-3′ | 177 | RT-PCR |
| No apical | LOC_Os12g29330 | NAM101- | 5′-ttgatittgccgaggtgta-3′ | 178 | Cloning |
| meristem protein | | 119F | | | |
| | | NAM168- | 5′cctgctcactccaccctggaggaag | 179 | Cloning |
| | | 186R1 | caggtcgga-3′ | | |
| GRAM domain | LOC_Os12g29400 | ProGDP01 | 5′-ggcctccaaaatttatagtccca-3′ | 133 | EMSA |
| protein | | ProGDP02 | 5′-gtggagaggcctcctgtttac-3′ | 134 | EMSA |
| (Promoter) | | | | | |
| Cellulose | LOC_Os12g29300 | ProCESA01 | 5′-gaggcttcctgttgactggt-3′ | 135 | EMSA |
| synthase | | ProCESA02 | 5′-attgcctccgttggtgttga-3′ | 136 | EMSA |
| (Promoter) | | | | | |
| Auxin response | LOC_Os12g29520 | ProARF01 | 5′-tctgtagccccgctattctt-3′ | 137 | EMSA |
| factor (Promoter) | | ProARF02 | 5′-aggtagagcggtgaggtcac-3′ | 138 | EMSA |
| Nodulin MtN3 | LOC_Os12g29220 | ProNOD01 | 5′-tacccgtgcaaacaaagaacag-3′ | 139 | EMSA |
| family protein | | ProNOD02 | 5′-ggaaagtcttttggacacgc-3′ | 140 | EMSA |
| (Promoter) | | | | | |

Real-Time PCR Protocol (qRT-PCR).

The reaction was set in 20 μl volume consisting of 5.0 μl of normalized cDNA, 10 μl of 2×SYBR green PCR master mix (Roche Diagnostics GmbH, Germany), and 1 μl each of 10× primer pair. Reactions were run in duplicate in a 7500 Fast Real-Time PCR System (Applied Biosystems, Foster City, USA). The amplification conditions maintained were 95° C. for 15 min, 40 cycles of denaturing at 94° C. for 15 s, annealing at 55° C. for 30 s, and extension at 72° C. for 30 s, followed by a disassociation stage (melting curve analysis). The comparative threshold cycle (ΔΔCt) method was used to quantify the relative expression levels.

Haplotype Analysis.

Genomic DNA was extracted from young leaf tissue of 125 purified (homozygous) rice varieties and wild ancestors using Qiagen DNeasy columns, made into paired-end libraries and sequenced on an Illumina Genome Analyser II, providing reads of 88, 100 and 120 bp lengths. Short reads were aligned to the Nipponbare reference genome and SNP genotypes were called using “Panati” (Mark Wright, Cornell University). Genome coverage was >7× genome equivalents in each case. Fastq data has been deposited in the Short Read Archive at NCBI as Acc_ID SRA# SAMN02142729-SAMN02142853. Subpopulation identity of the O. sativa varieties was analyzed using Principle Component Analysis (PCA). The haplotype structure of a ˜6 kB region surrounding the OsNAM12.1 gene was analyzed, including 2 kB upstream and downstream of the 5′ and 3′ untranslated regions (UTRs).

Immunoblot Assays.

Proteins were extracted from the roots of the rice plants from both the treatments (drought stress & well-watered) using the trichloroacetic acid-acetone method and dissolved in solubilisation buffer containing 9M urea, 4% CHAPS, 1% DTT, and 1% Biolyte Amphotytes (pH 3-10; BIO-RAD Laboratories, Inc, Hercules, Calif., USA). Protein concentration of the extracts was determined using the Bradford method. Protein samples (125 μg) were rehydrated overnight at room temperature on an IPGphor using IPG Dry-Strips (7 cm, pH 3-10; non-linear gradient, GE Healthcare, USA), followed by iso-electric focusing at 10.8 kVhrs. After two-step equilibration, the IPG strips were loaded on a 10% w/v SDS-PAGE gel using the 4-gel Mini-PROTEAN® Tetra cell (BIO-RAD Laboratories, Inc, Hercules, Calif., USA). The gels were run at 20V/gel for 2 h and kept until the dye front reached the bottom of the gel. The gels were visualized using the CBB-G250 and scanned using Quantity One software (BIO-RAD) at a resolution of 600 dpi. After electrophoresis, proteins were blotted onto a nitrocellulose membrane (Hybond-C Extra GE Healthcare Amersham Biosciences, USA) using the “semi-dry method” with a discontinuous buffer system. The blotting procedure was carried out for 1 h with a constant voltage of 17V. Subsequently, the membrane was blocked with 5% (w/v) dried skimmed milk in PBS-T (0.1% Tween 20 in phosphate buffered saline (PBS), 10 mM Na2HPO4, 1.75 mM KH2PO4, 13.7 mM NaCl and 2.86 mM KCl) at room temperature for 1 h. Incubation with primary antibody was performed in 5% (w/v) dried skimmed milk in PBS-T (0.01% Tween 20 in PBS) overnight at 4° C. The primary antibodies used for these experiments were anti-NAM antibody (1:1000 dilution of NAM polyclonal (0.39 mg)—Abexome Biosciences, India) and anti-SUMO (1:1000 dilution of Sumo 1 antibody (ab5316), Abcam PLC, Cambridge, England). Subsequently, incubation with HRP-conjugated secondary antibody (Goat Anti-Rabbit Antibody Conjugated to Horseradish Peroxidase—166-2408EDU-BIO-RAD Laboratories, Inc, Hercules, Calif., USA) was performed for detection. Incubation was done in 5% (w/v) dried skimmed milk in PBS-T (0.01% Tween 20 in PBS) at room temperature for 2 h. Chemiluminescence was detected by Novex® ECL Chemiluminescent Substrate Reagent Kit (Invitrogen, UK). Blots were exposed to autoradiographic films (Amersham Hyperfilm ECL) for chemiluminescent imaging.

Recombinant OsNAM_{12.1}.

Recombinant OsNAM_{12.1} was expressed in BL21 E. coli cells from a BamHI and XhoI construct in pGEX-4T1 (Promega) amplified from pCAMBIANAM using the primers NAMpGEXFor (CCCCGGATCCATGGAGACGACGGCG) (SEQ ID NO: 131) and NAMpGEXRev (GCGCCTCGAGTTAGTCGGAGGCGTCGCC) (SEQ ID NO: 132). Transformed cells were induced with 1 mM IPTG at 25° C. and induced cultures lysed in PBS buffer pH 7.4 by sonication. The soluble fraction was extracted by centrifugation at 1000×g for 30′. Protein was obtained after glutathione sepharose column chromatography (GE healthcare), by eluting with 10 mM glutathione, 50 mM Tris-Hcl pH 8.0. EMSA.

Electrophoretic Mobility Shift Assay

EMSA was performed using LightShift Chemiluminescent EMSA Kit (Thermo Scientific, USA). Around 400 bp to 450 bp promoters region of the target genes were Amplified by PCR using the following pairs primers: OsGDP12.1 (ProGDP01 and ProGDP02), OsCesA12.1 (ProCESA01 and ProCESA02), OsARF12.1 (ProARF01 and ProARF02) and OsNod12.1 (ProNOD01 and ProNOD02) (Table 1). Initially the DNA were labeled with biotin using Biotin 3′ End DNA Labeling Kit (Thermo Scientific, USA) followed by PCR purification using QIAquick PCR Purification Kit (QIAGEN). The labeled DNA was incubated with different concentration of OsNAM12.1GST for 20 min at 30° C. and were run on 4% native PAGE. The signals were detected according to the manufacturer's protocol.

SUMOylation Assay.

In vitro SUMOylation assay was carried out using SUMOylation kit (ENZO Life Sciences) by following the manufacturer's protocol.

TRIM Mutant Analysis.

TRIM lines used in the current study were M0074686, M0092628, M0111080, M0093267, M0032667 and M0066205 which were AT lines for OsCesA12.1, OsWAK12.1, OsGDP12.1, OsARF12.1, OsSTPK12.1 and OsPOLe12.1, respectively (Table 4). An additional AT line M0115183 was used for OsPOLe12.1. A KO line M0039637 was used for OsAmH12.1. Thirty T2 seeds were received for each line. They were imbibed for two days before subjected to the Yoshida medium with gerite in the presence (+) or absence (−) of 23% PEG. Genotyping was performed at Day 10 to identify homozygous, heterozygous, and wild type (no T-DNA integration) plants. qRT-PCR of the root tissue was then performed to confirm the AT/KO nature of each line. The photos of roots of each genotype were taken at Day 14. The roots were fixed at Day 20 and several root parameters were then assayed by scanner coupled with WinRhizo program.

Plant Transformation—Designing Overexpression Construct.

Binary vector IRS 537, derivative of pCambia 1300 vectors, was provided by Rice Biotech Lab, PBGB, IRRI. The expression of the IRS537 transgene is driven by the maize Ubiquitin promoter and terminated with Nos terminator. Since both of the binary vector and insert (OsNAM12.1 coding sequence) have BamHI and KpnI sites, about 2 ug of plasmid DNA from both vector and insert were digested using 1 ul each of BamHI and KpnI restriction enzyme (Invitrogen) with double digestion buffer, 0.5× Buffer K, for 2 hours at 37° C. Then the above two linearized fragments were purified and ligated using the T4 DNA ligase (Promega, USA) to produce the overexpression construct. The concentration of vector to insert was maintained in 1:3 ratio to avoid false positive by rejoining of vectors without any insert. Briefly, about 30 fM (20 ng) of plasmid vector and 90 fM (60 ng) of insert was ligated together using 1 U of T4 DNA ligase at 14° C. over night. The next day, the reaction was stopped by adding 0.5 μl of 50 mM EDTA before transformation. The ligated binary vector was transformed into E. coli DH5 alpha competent cells by heat shock method. Fusion result was confirmed by colony PCR and restriction digestion with BamHI and KpnI. Only the plasmid with double confirmed by PCR and restriction digestion was used for transformation into Agrobacterium tumefaciens strain LBA 4404.

Plant Transformation—Mobilization of the Construct into Agrobacterium tumefaciens.

Required number of vials containing 100 μl of Agrobacterium tumefaciens competent cells was thawed on ice for 10-15 min till the cells come into liquid phase. About 1 μg of supercoiled plasmid DNA was slowly added to the vial and swirled to mix the plasmid DNA. The cells were frozen in liquid N2 for 1-2 min for complete freezing and thawed by placing in water bath preheated to 37° C. for about 5 min (till the cell suspension is liquefied) and immediately 900 μl of YEB media will be added to each tube and mixed by inversion. The vials were incubated at 28° C. at 150 rpm for 3 hours. The cells were harvested by spinning for 30 seconds at 3000 rpm in a micro-centrifuge. Approximately 3/4 of the supernatant was decanted in the laminar air flow cabinet and the pellet was re-dissolved in the solution that was left behind by slow tapping. These cells were plated in 2 SOB media plates containing 10 mg/L of rifampicin, 50 mg/L kanamycin for which the resistance gene is conferred on the plasmid and incubated at 28° C. for 48 hours until the bacterial colonies became visible and big enough for streaking and colony PCR. Plasmid DNA was extracted from colony PCR positive clones and restricted by BamHI and KpnI to release the insert DNA. Only the colony confirmed by PCR and restriction digestion was used for transformation into immature embryo of IR64.

Plant Transformation—Transformation by Agrobacterium Mediated Transformation Method.

The overexpression construct was transformed into mega rice variety IR64 according to modified protocols of Hiei et al., (1997) with some modification. Roughly, about 250 of 10-12 days old immature embryos (IE) were isolated in sterilized condition. The IEs were transferred in a petri dish containing agar culture medium A201 with the scutellar side up (i.e. plumule-radicle axis side in contact with the medium). Fifty immature embryos were arranged per plate. Agrobacterium cells were harvested from 2 day old culture plate and added to liquid infection medium (A200) and the density of the culture was adjusted to OD595=0.3. The culture was incubated at 25° C. for 1 hour in dark. About 5 μl of the Agrobacterium culture was added to each embryo and the plate was incubated in dark at 25° C. in dark for one week. The shoot was carefully removed from the germinating embryo and it was blotted on sterile filter paper to remove the Agrobacterium. The embryo was placed back to resting medium A202 with 16 embryos arranged per plate for five days. The growing embryos were divided 4 equal parts and were incubated on selection medium (A203) containing 30 mg/L of hygromycin for 10 days. This was repeated three times. A hundred percent transformation efficiency was considered if three plants were produced from ¼ part of the IE. Resistant calli were transferred to A204 pre-regeneration medium (8 callus lines per Petri dish) containing 50 mg/L of hygromycin and were cultured for 10 days. The greenish embryogenic calli were transferred into A205regeneration medium (4 callus lines per Petri dish) with 50 mg/L of hygromycin for 10 days. After 10 days, growing shoots were selected and transferred to test tubes with solid MS medium for rooting 2 weeks. The well rooted plantlets were washed in tap water and were grown hydroponically in Yoshida culture solution. The composition and details of the media used for Agrobacterium mediated transformation are provided in Appendix 3 and 4.

Results.

The qDTY_{12.1} NILs Show Yield Advantage Under Drought in the Fields.

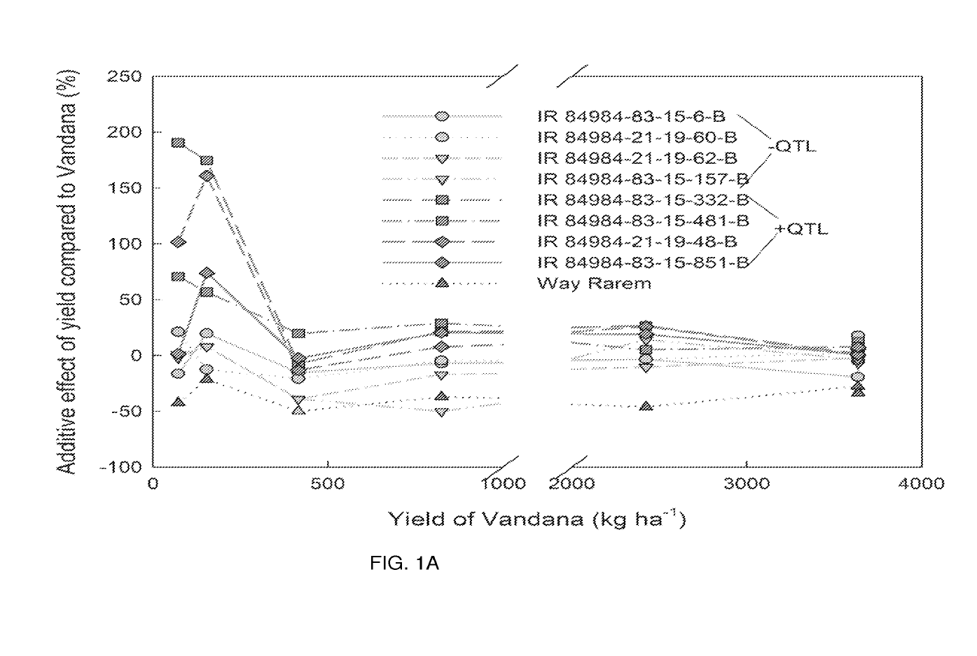

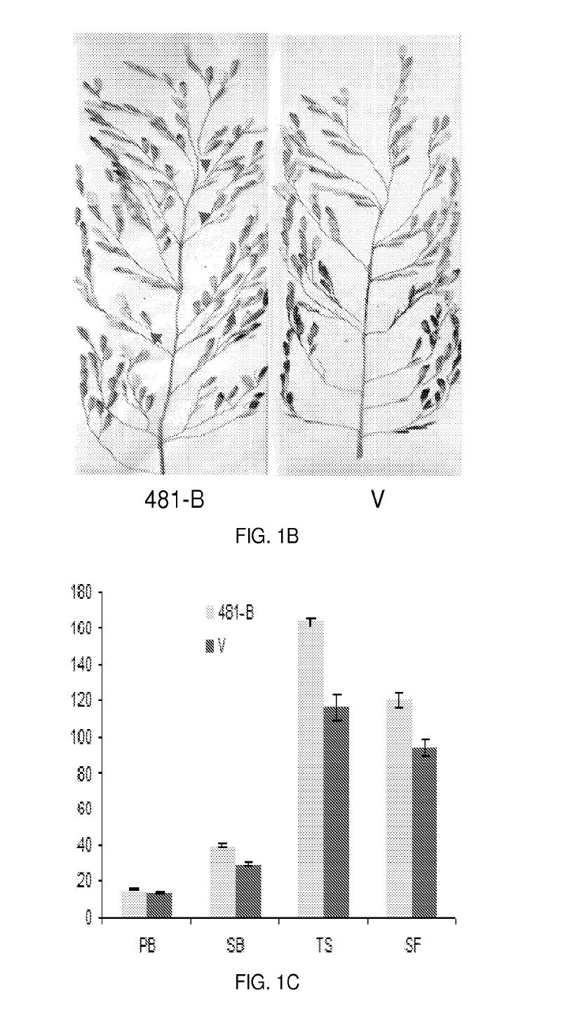

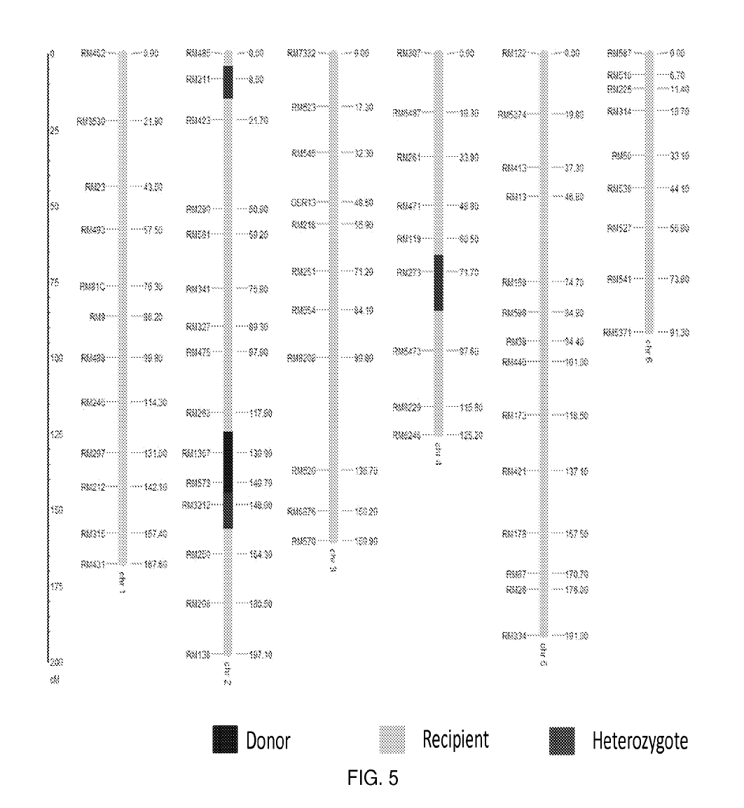

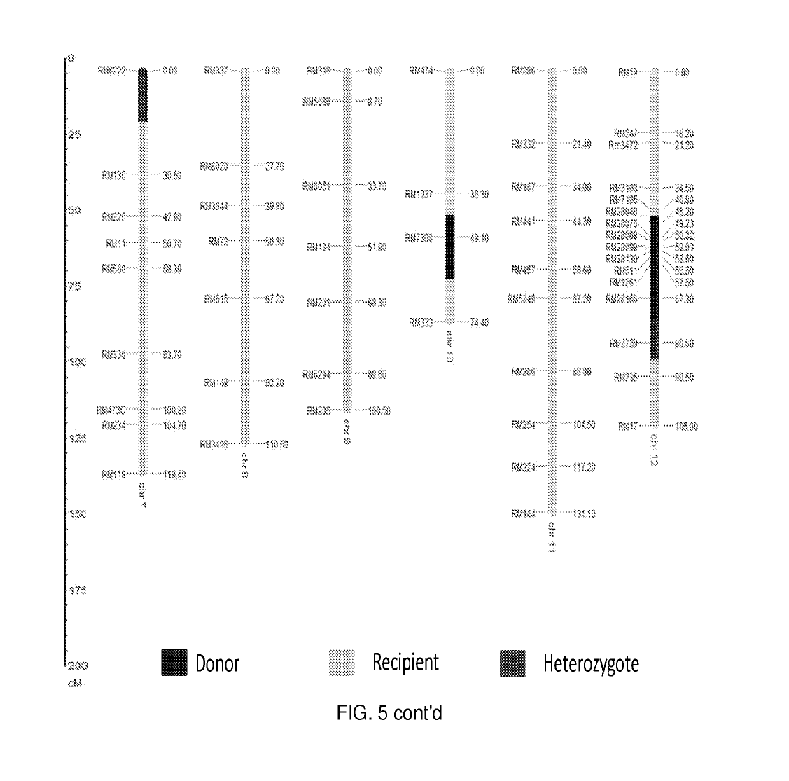

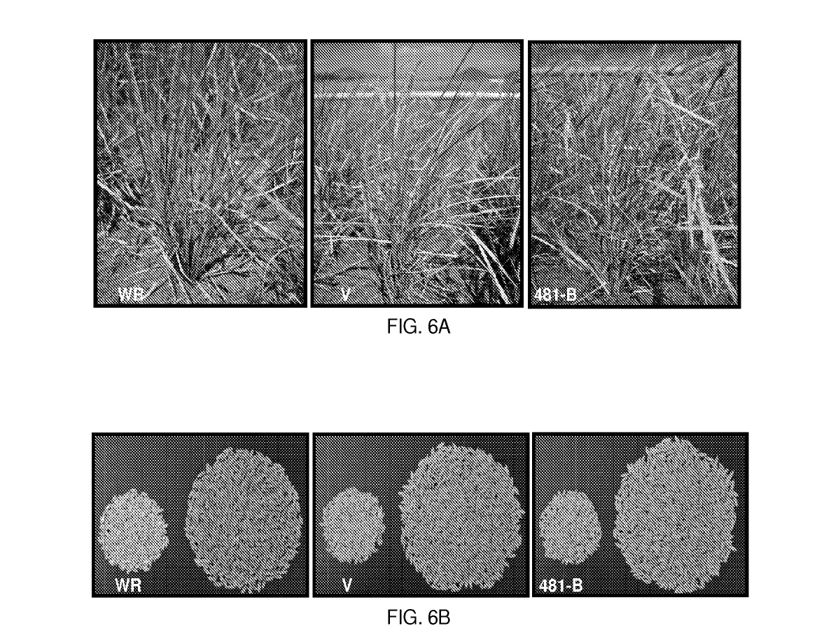

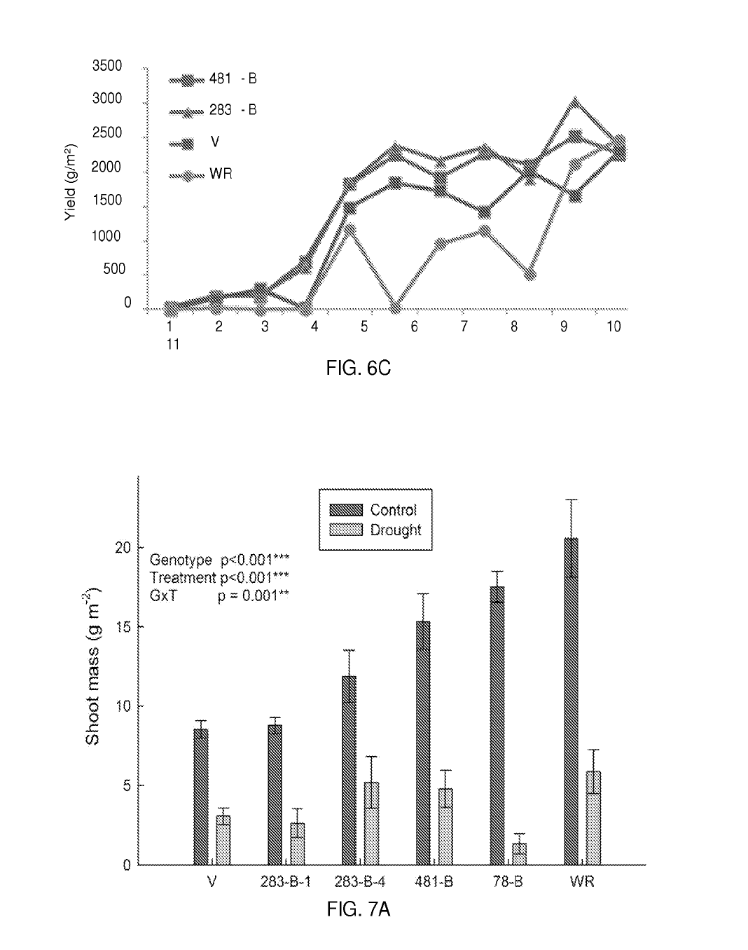

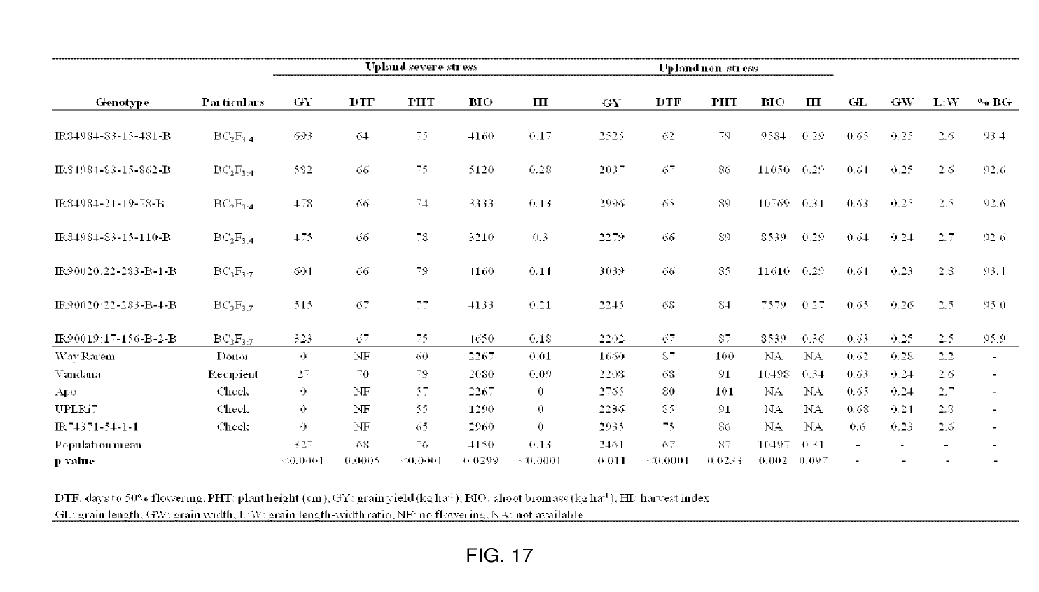

Using marker assisted backcrossing, NILs carrying the WayRarem qDTY_{12.1} were generated in the Vandana background with 93.4 to 95.9% recovery of the Vandana-genome (FIGS. 4A-4B, 5, 17). All NILs had a yield advantage of 300-500 kg/ha over Vandana under field drought (FIGS. 1A, 17). Mean grain yield was 323 kg/ha, with NIL IR84984-83-15-481-B (481-B) showing the largest increase in grain yield (693 kg/ha vs. 27 kg/ha for Vandana). Field evaluation over six trials in three seasons demonstrated that the additive effect of qDTY_{12.1} was proportional to drought severity. Average additive effect ranged from 4% under irrigation to 104% under severe drought (FIG. 1A). Greater mean yield was consistently observed in the NILs across 11 field trials over three years at a separate location and there was no significant change in length to width ratio of grains (FIG. 6). Also, the NILs exhibited seedling stage drought tolerance, measured over two seasons in the form of increase in shoot biomass (FIG. 6D).

Multiple Morpho-Physiological Component Traits are Improved in the NILs.

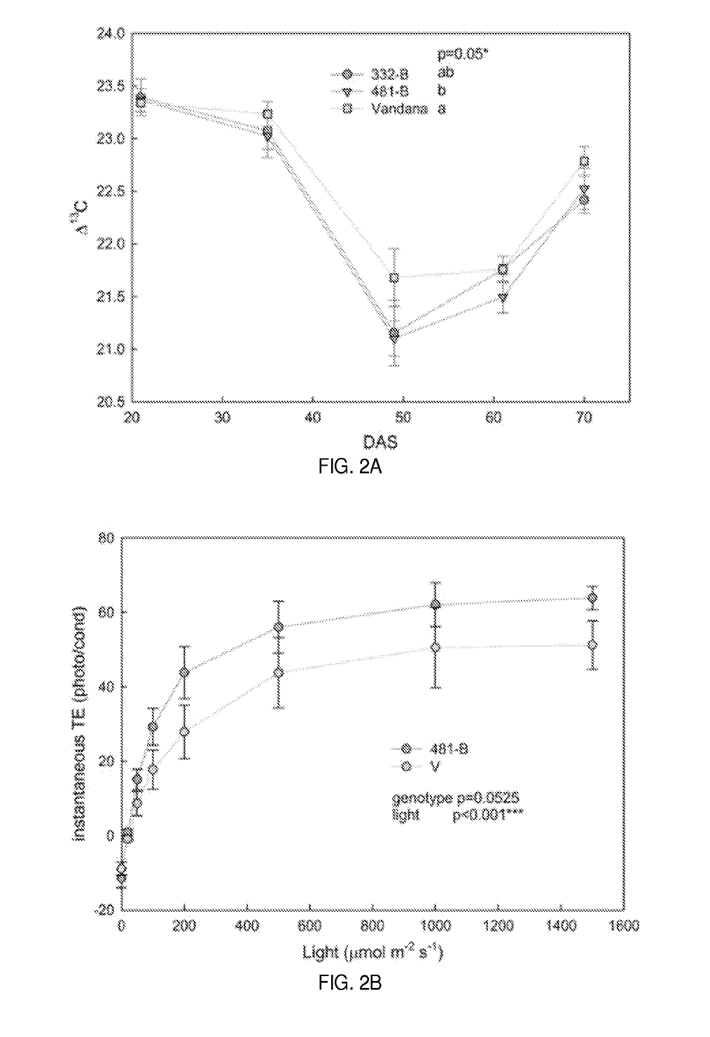

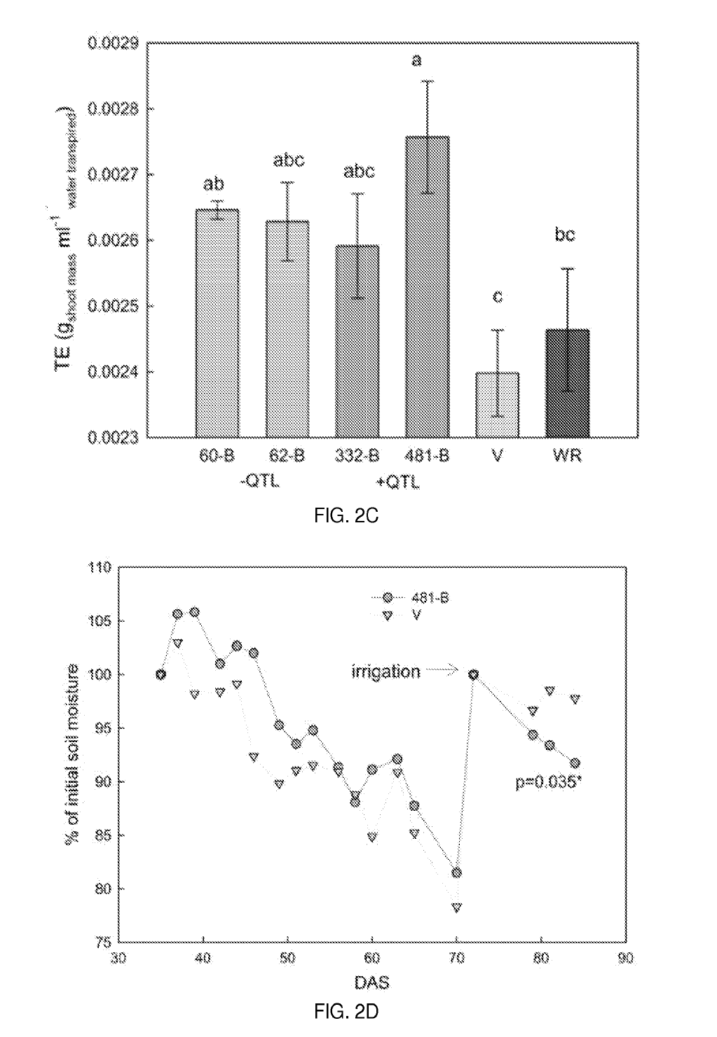

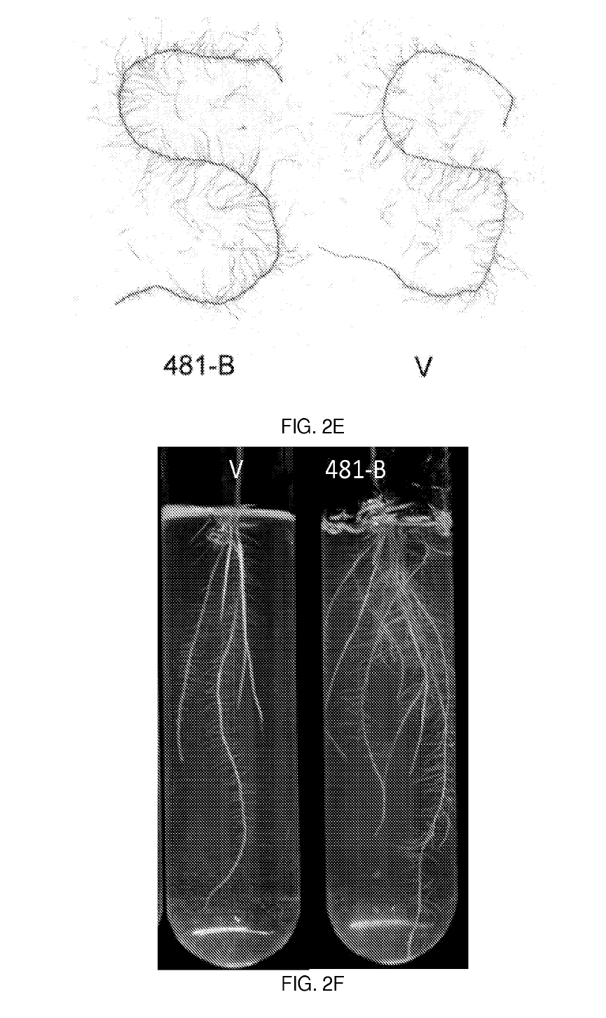

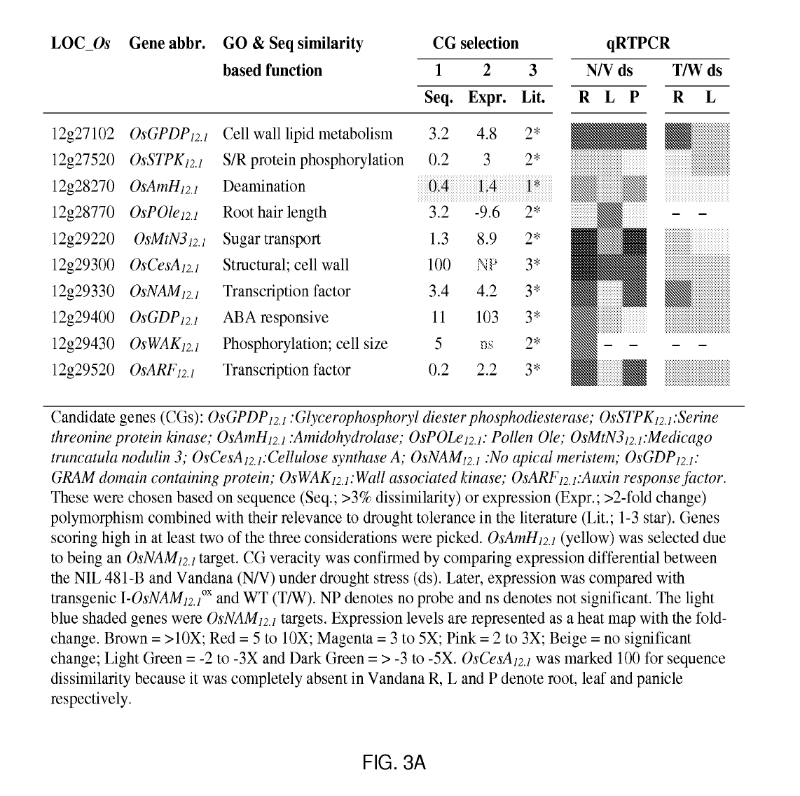

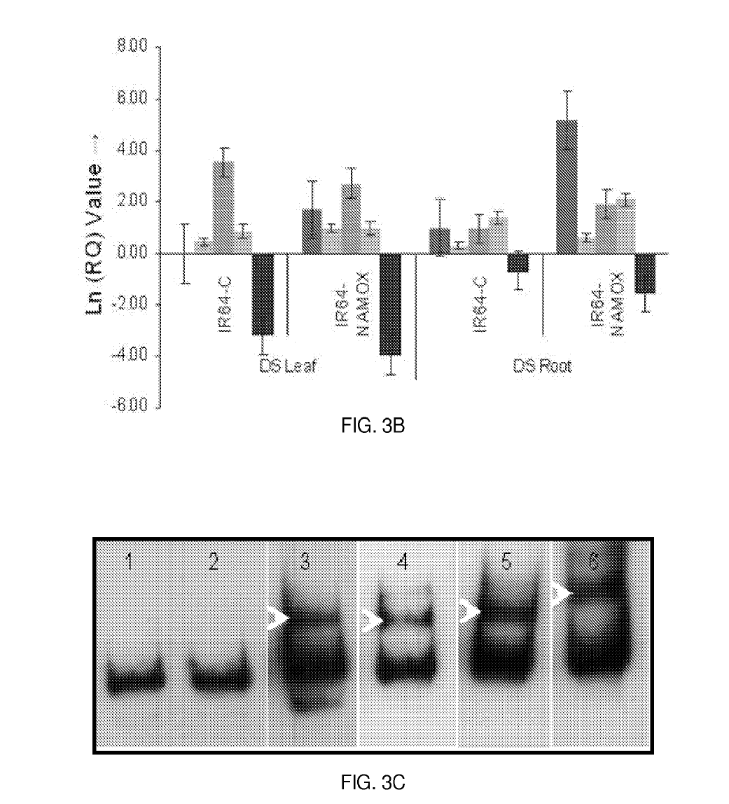

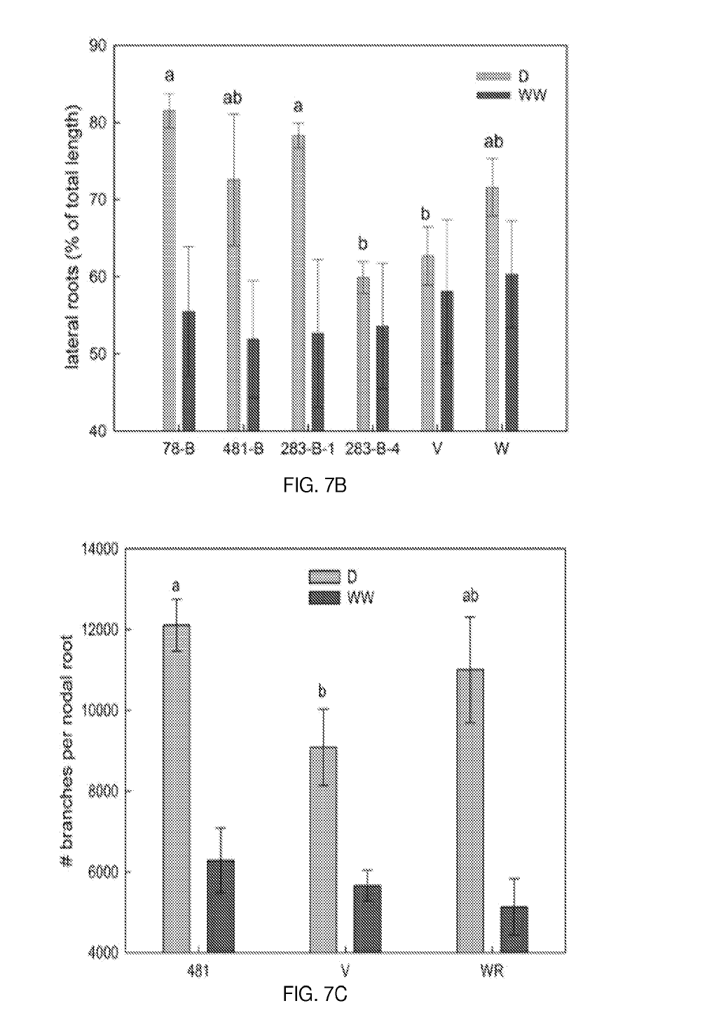

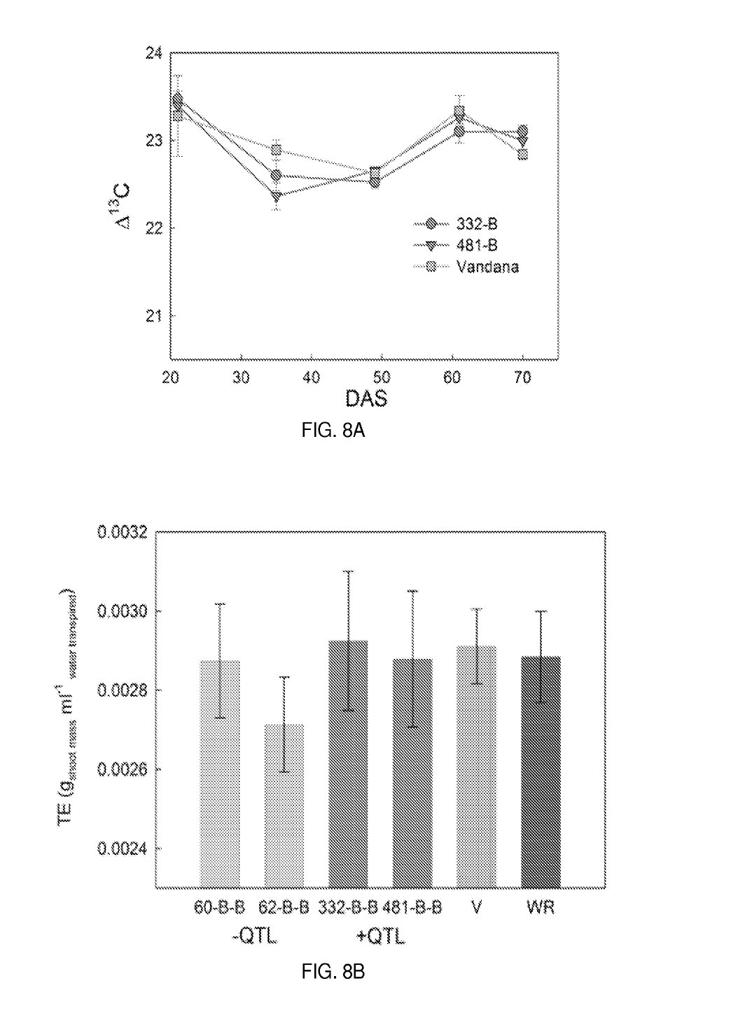

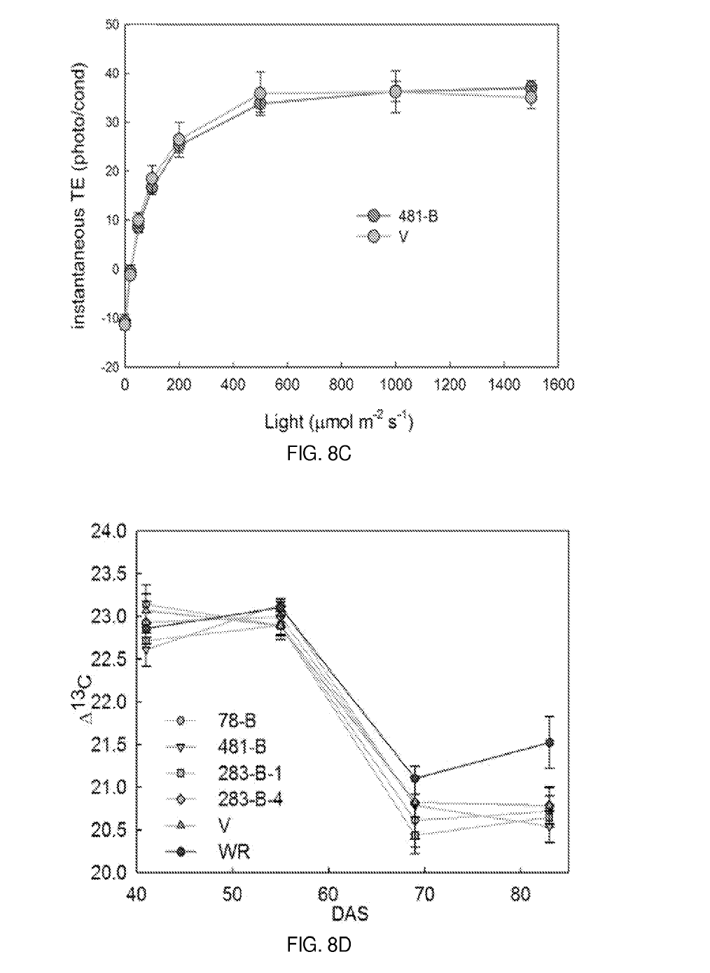

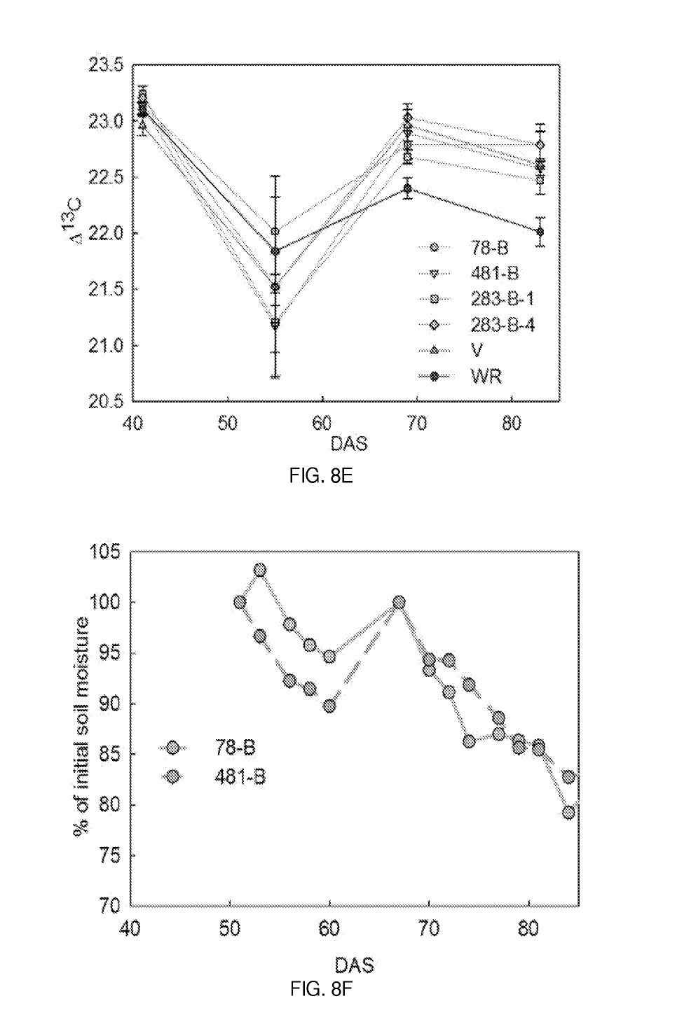

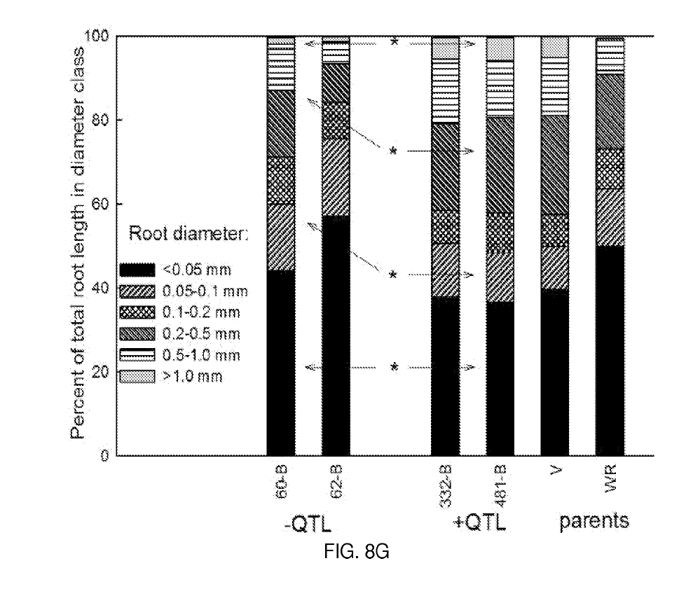

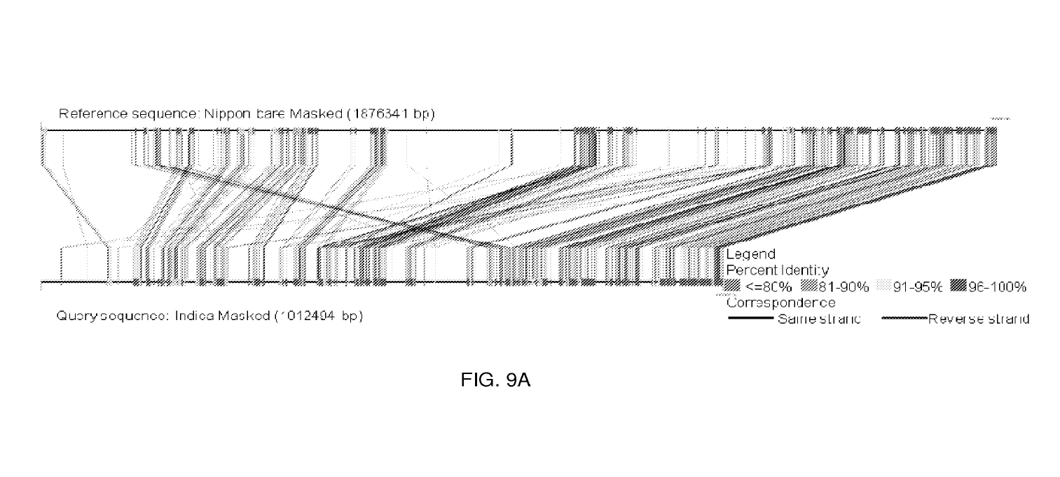

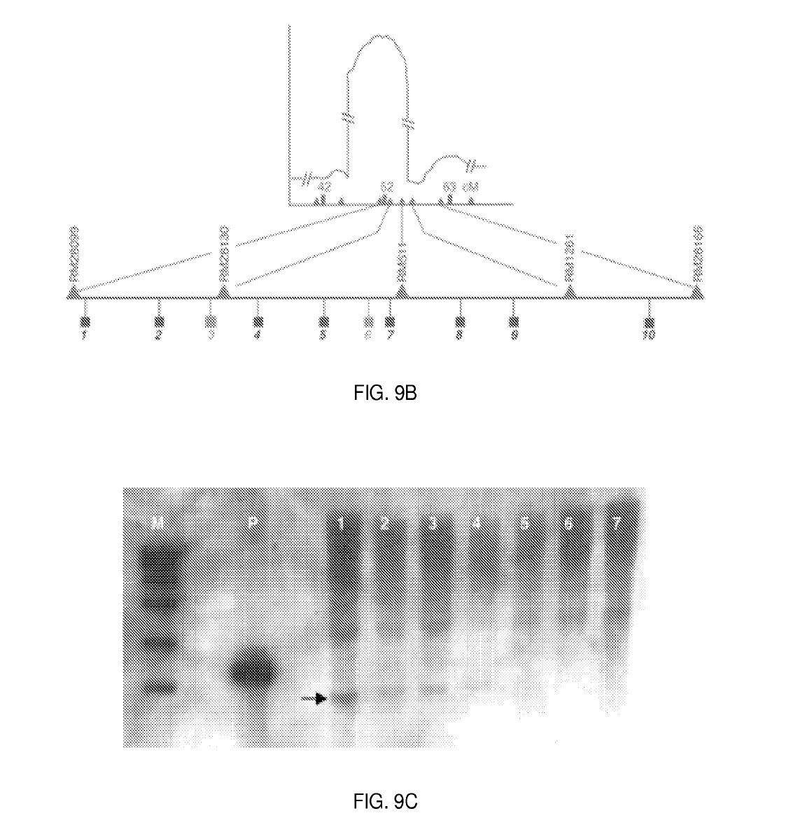

Under field drought conditions, all NILs exhibited increased biomass and harvest index, similar plant height and decreased number of days to flowering (DTF) compared to Vandana (FIG. 17). qDTY_{12.1} was shown to increase water uptake under drought. Under field drought conditions, NIL 481-B exhibited greater transpiration efficiency (TE) than Vandana through four different methods of analysis (FIGS. 2A-2D, 8) and demonstrated increased LRN (FIGS. 2E; 7B, 8D). Differences in LRN in Vandana and NILs were also observed at the seedling-stage in greenhouse studies (FIG. 7C) and even under polyethylene glycol (PEG) simulated water deficit in agar tubes (FIG. 2F). NILs also exhibited increased numbers of secondary branches and filled grains in the panicles (FIGS. 1B-1C). Taken together, NILs performed better under drought for multiple morpho-physiological component traits of yield including biomass, DTF, TE, LRN, number of panicle branches, total number of spikelets, and number of filled spikelets.

The Large-Effect qDTY_{12.1} is Composed of Sub-QTLs: The Search for Candidate Genes.

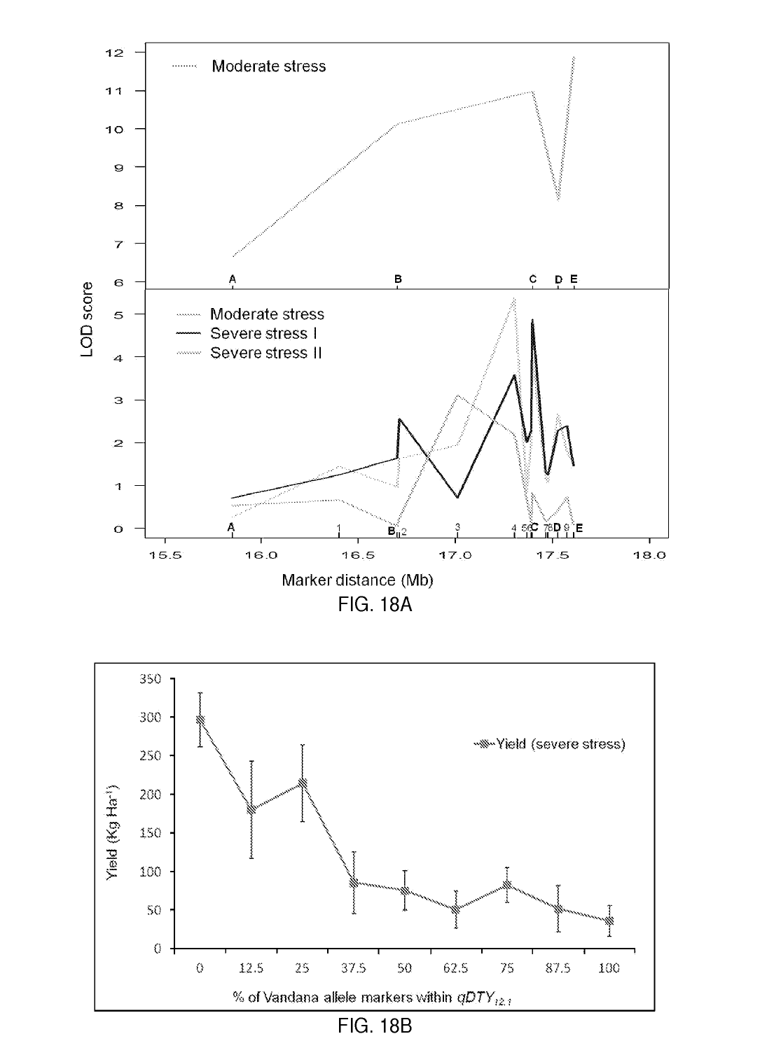

1900 BC₂F₃ lines were genotyped to identify 52 that lacked the WayRarem allele for one or more of the SSR markers used across a 1.7-Mb region. R/QTL mapping revealed high LOD scores for the flanking markers and the two internal weak peaks indicated at least three fractions for qDTY_{12.1} (FIG. 18A). The LOD score trend reiterated the importance of the entire region. Hence, the Nipponbare-bait-based targeted NGS data (100×) was obtained for the 1.7-Mb region from Vandana and WayRarem. The Nipponbare qDTY_{12.1} region contained 45 genes other than those for retrotransposons, expressed and hypothetical proteins. Both Vandana and WayRarem lacked three ATPases and a Tetratricopeptide gene. Vandana lacked an additional gene (Cellulose synthase A; CesA10) that WayRarem retained. Considering the promoter and CDS, the sequence of 16 genes was >3% dissimilar between Vandana and WayRarem.

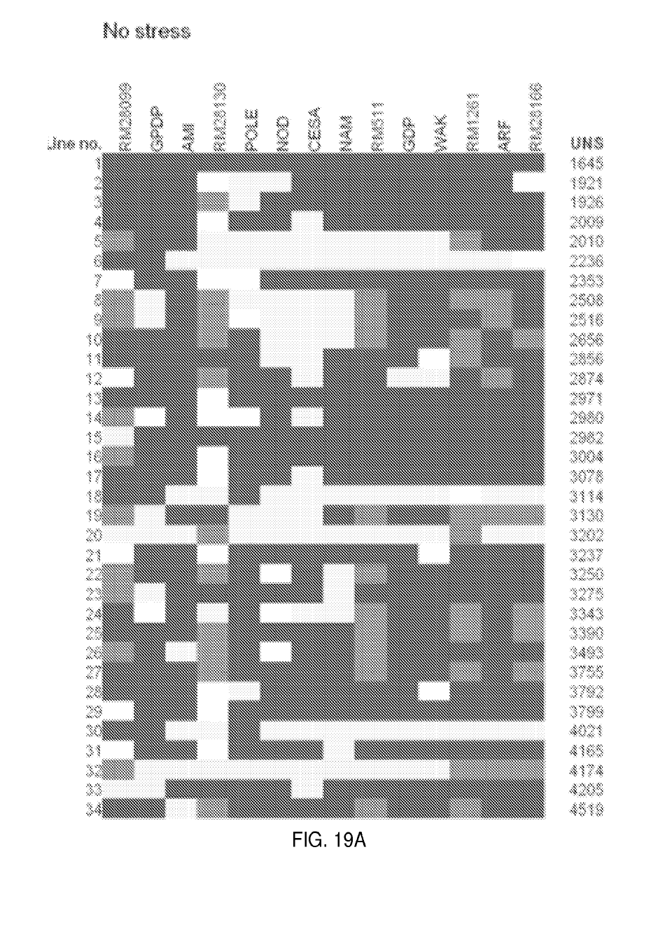

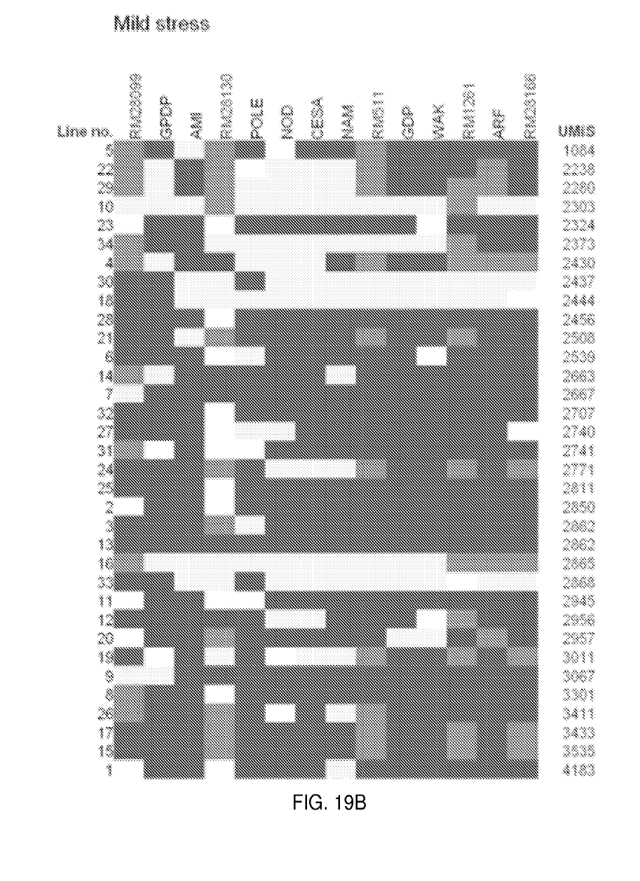

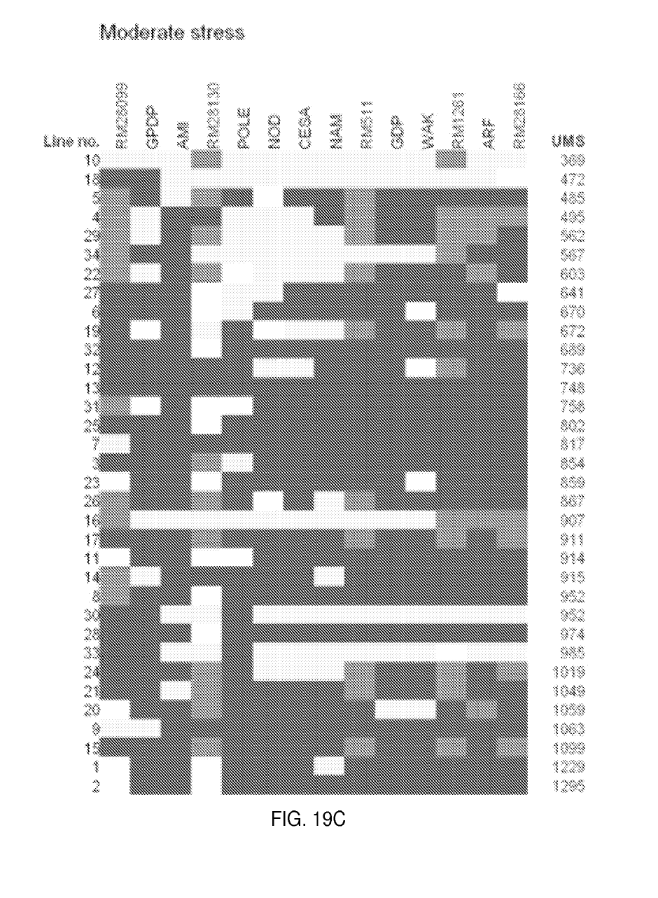

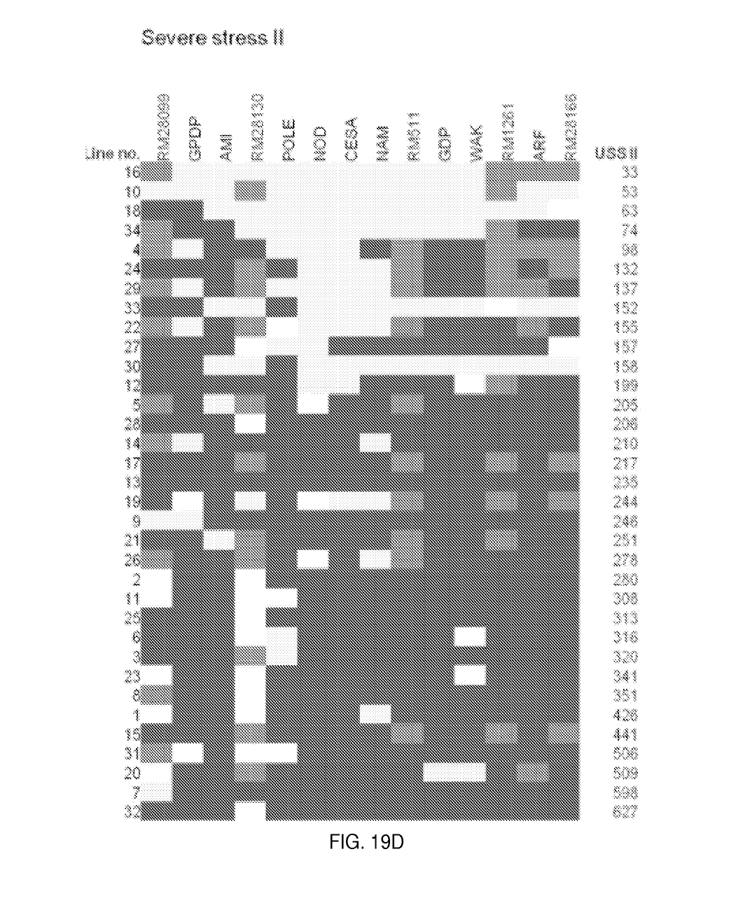

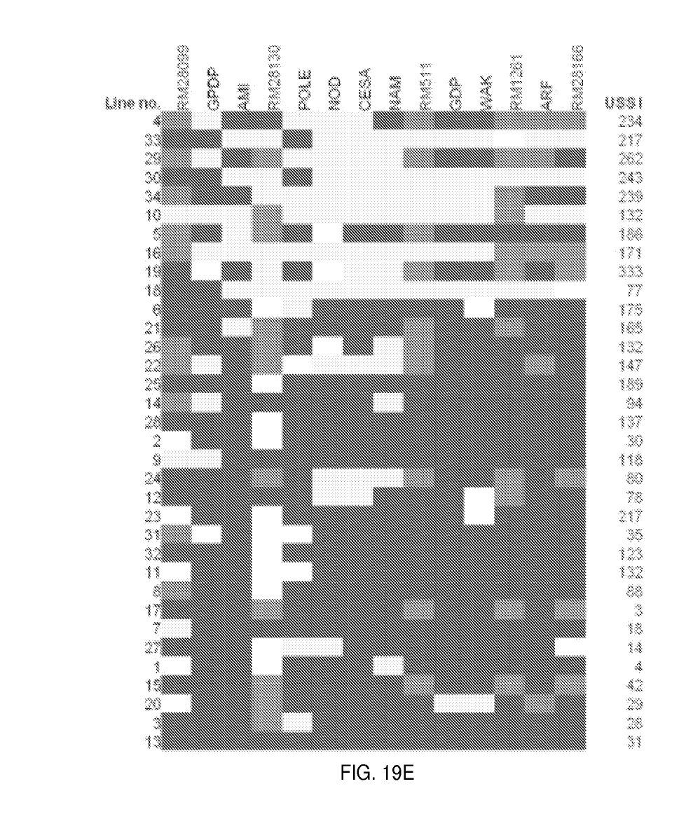

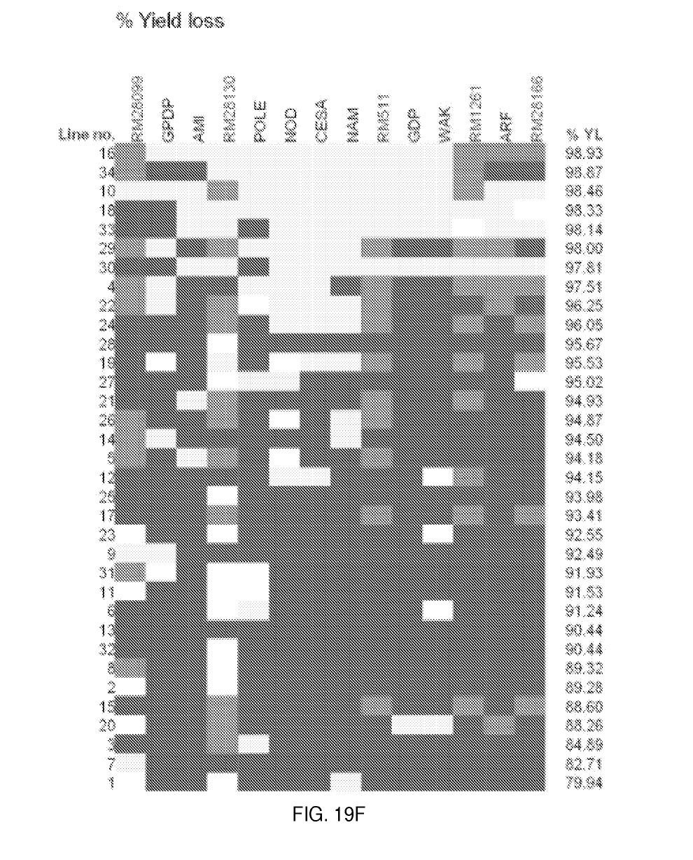

Genevestigator-mediated analysis for differential expression of the 45 genes under drought revealed 11 that were ≧2-fold up- or down-regulated. Four of these were also ≧3% dissimilar and thus potential candidate genes. Based on substantial sequence or expression polymorphism, combined with compelling relevance from literature on drought tolerance, six additional putative candidate genes were selected (FIG. 19). Quantitative transcript analysis of these 10 genes in Vandana and 481-B roots, leaves and panicles under normal and drought conditions revealed all 10 to be >2-fold up- or down-regulated in 481-B, in at least one of the three tissues (FIG. 19).

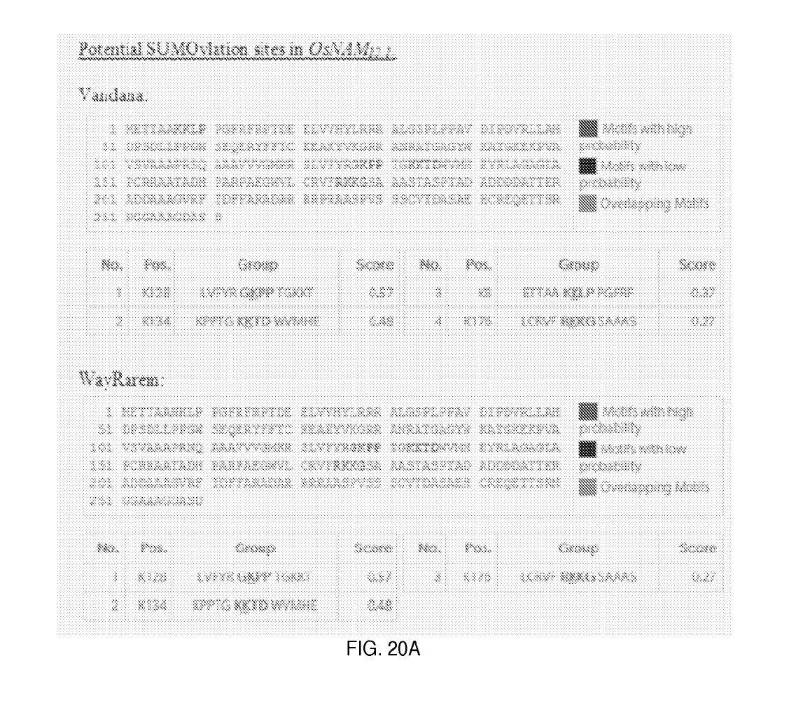

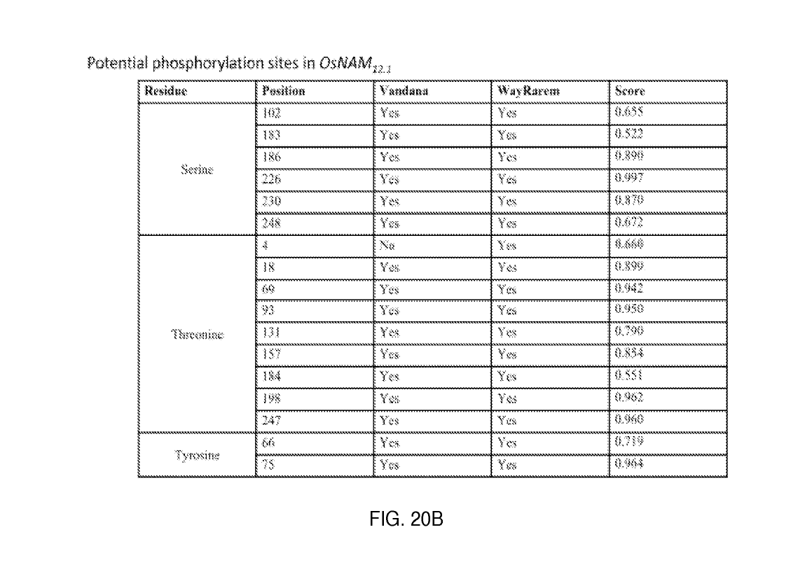

To identify strong candidate genes, a subset of 34 recombinant lines were genotyped for the Vandana/WayRarem allele of the nine putative candidate genes. This gene-based fine mapping fractionated qDTY_{12.1} into 4 regions (FIG. 18B). As the content of WayRarem alleles decreased, so did the yield (FIG. 18C). Also, as the stress level increased, more WayRarem alleles were required for better yield (FIG. 20). These results reiterated a role for multiple genes, spread along the 4 regions, for the full impact of qDTY_{12.1} Additionally, OsMtN3_{12.1} and OsNAM_{12.1} subtending the major peak were identified as candidate genes with higher probability of exerting stronger effects (FIG. 18B).

OsNAM_{12.1}.

The OsNAM_{12.1} protein contains a protein cleavage PEST motif (177-KGSAAASTASPTADADDDDATTER-200 (SEQ ID NO: 180); score 14.1) as in the negative regulatory domain of another drought responsive Arabidopsis transcription factor DREB2A. The lysine bordering the PEST motif can accept ubiquitin or SUMO, and such a modification can alter PEST-targeted protein cleavage, thus affecting protein stability. Along with the PEST motif-mediated protein cleavage, which can be affected by the modification of the bordering lysine, the post-translational modifications of OsNAM_{12.1} are revealed as multiple immuno-detectable bands under well-watered conditions.

For Ulp1-mediated deSUMOylation of OsNAM_{12.1} putative di-SUMOylated (—53 kD; 29+12+12) rather than the putative mono-SUMOylated (—41 kD; 29+12) OsNAM_{12.1} was preferentially deSUMOylated. Such differential/preferential activity is known for Ulp1. DeSUMOyltion visualization on 2D gel provided further evidence that OsNAM_{12.1} was SUMOylated in vivo.

Despite transcriptional upregulation, relative down-regulation of OsNAM_{12.1} protein under drought indicated conditional balance between the transcript, protein and the modified protein, necessary for drought response.

The Role of OsNAM_{12.1} in qDTY_{12.1}.

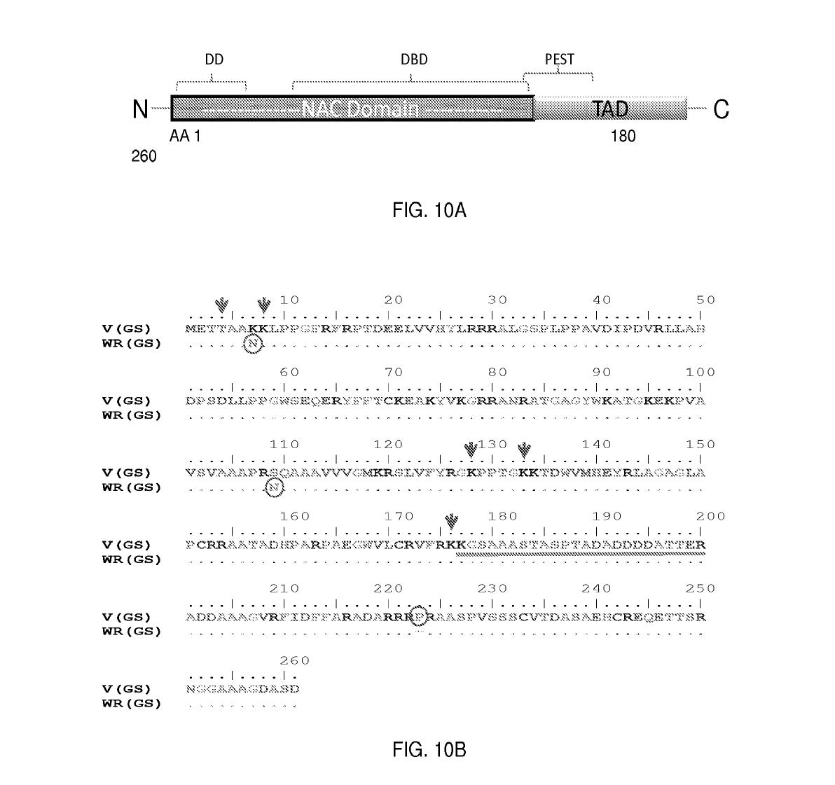

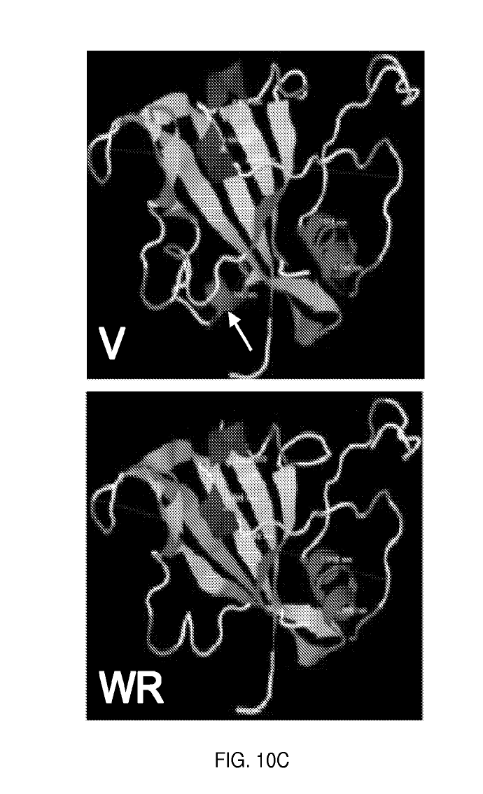

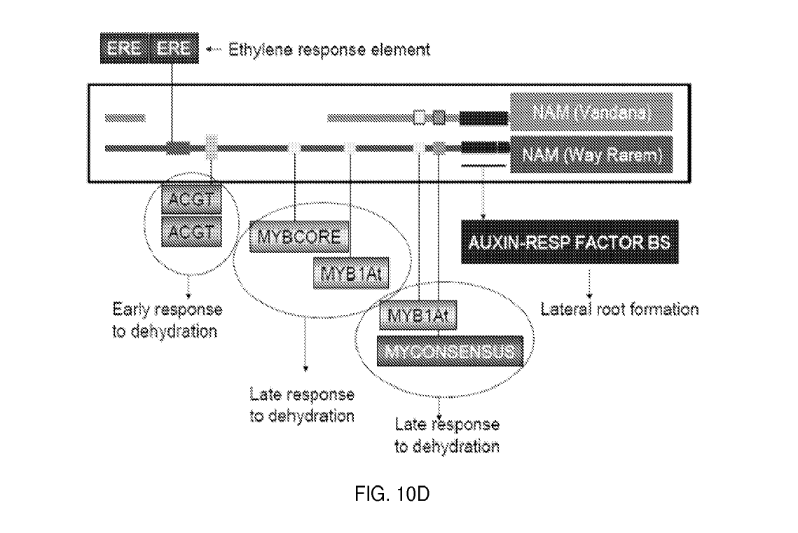

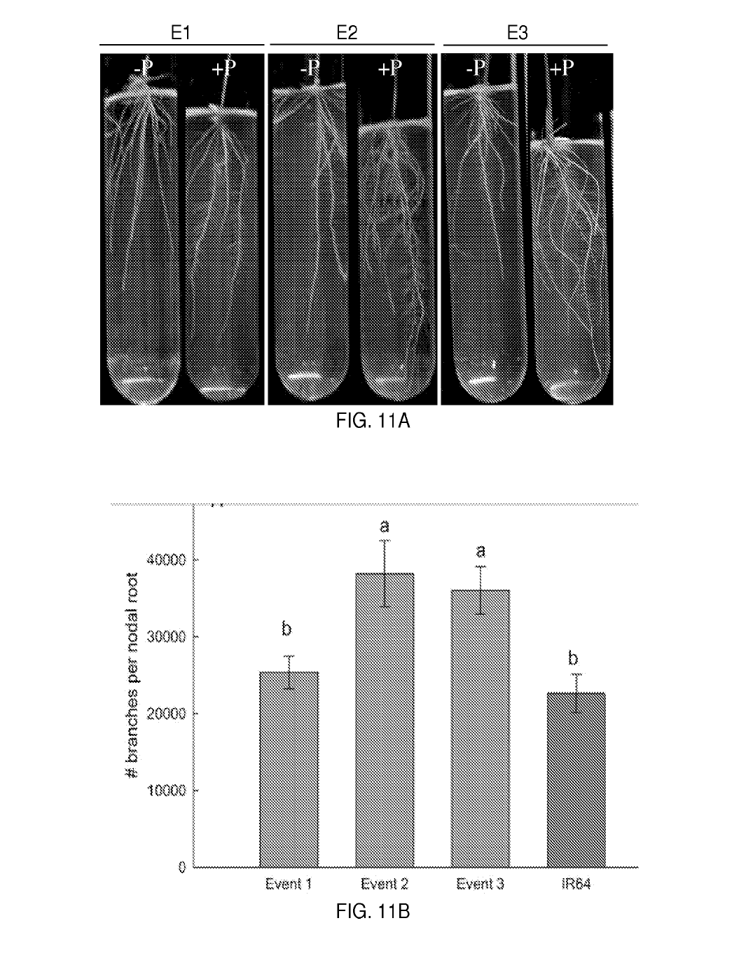

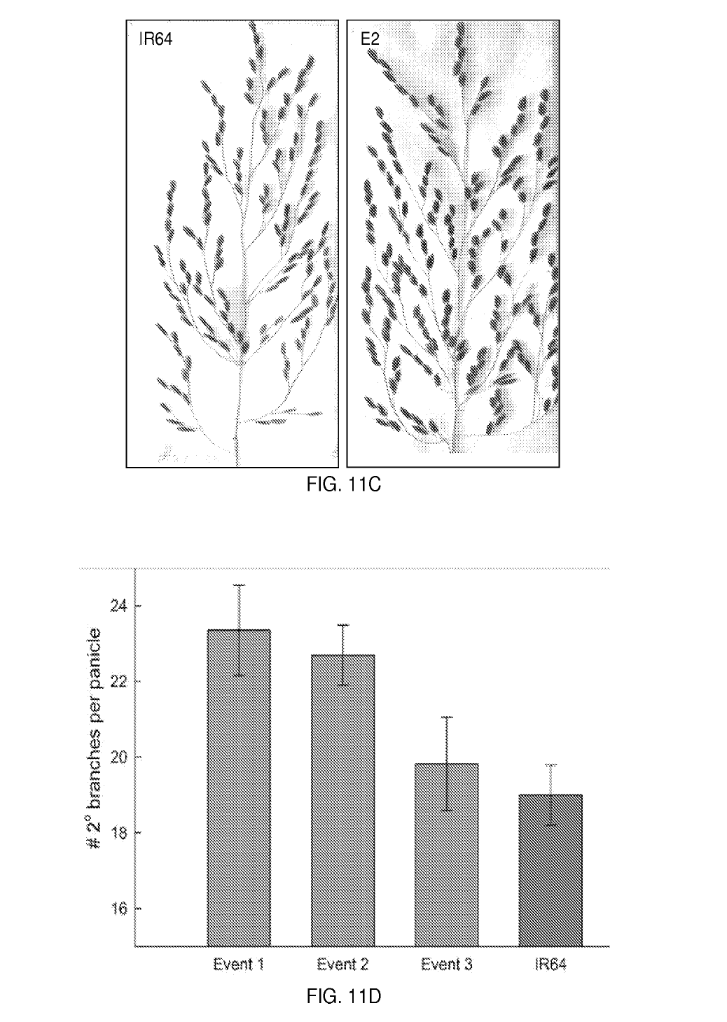

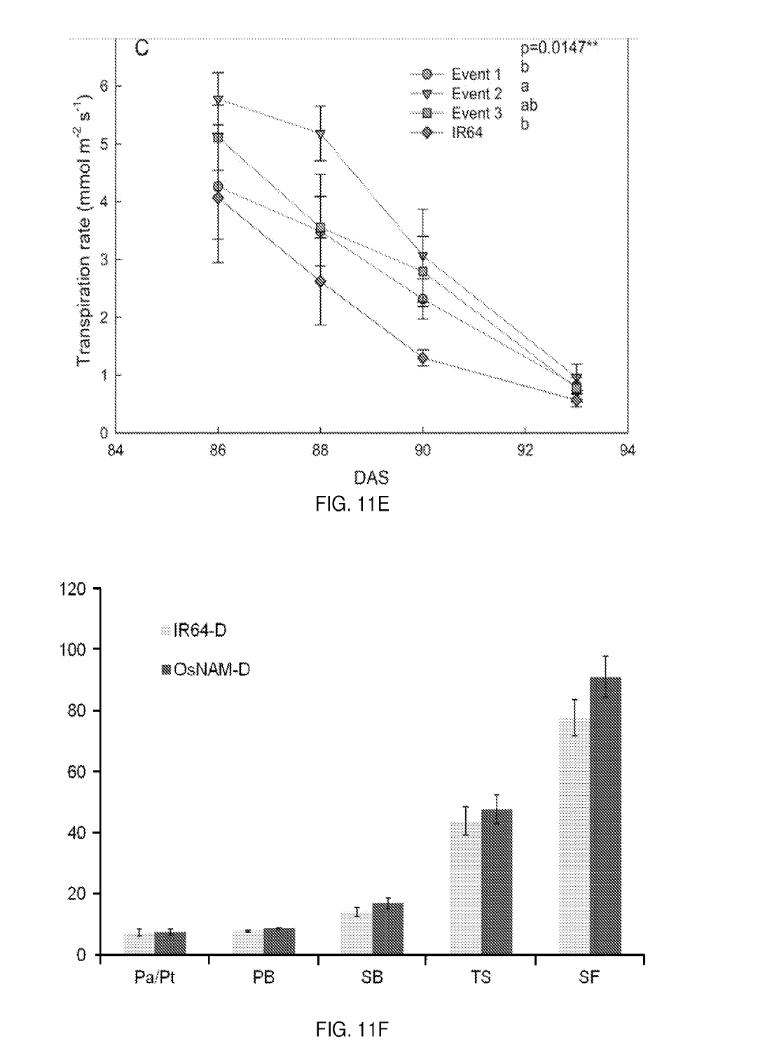

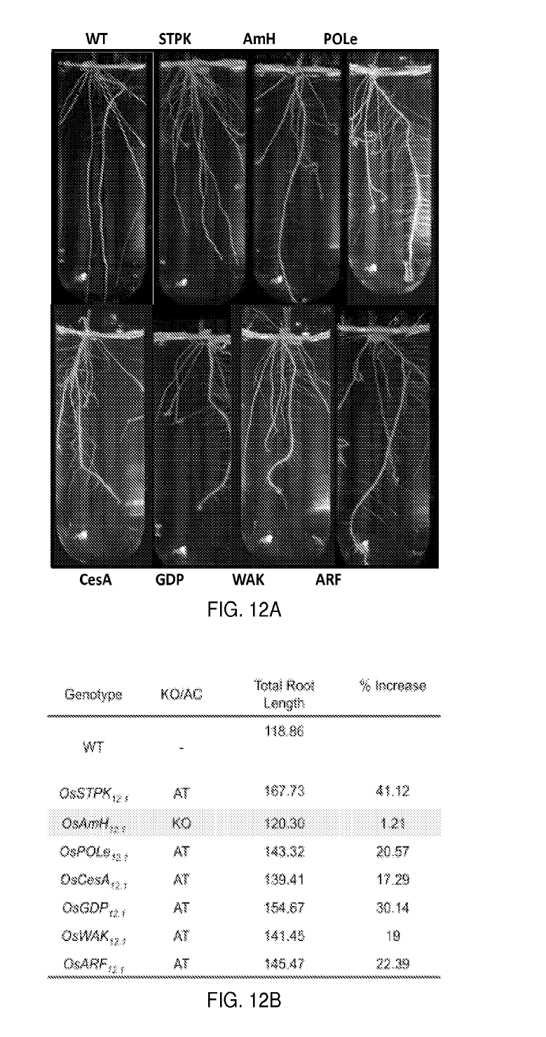

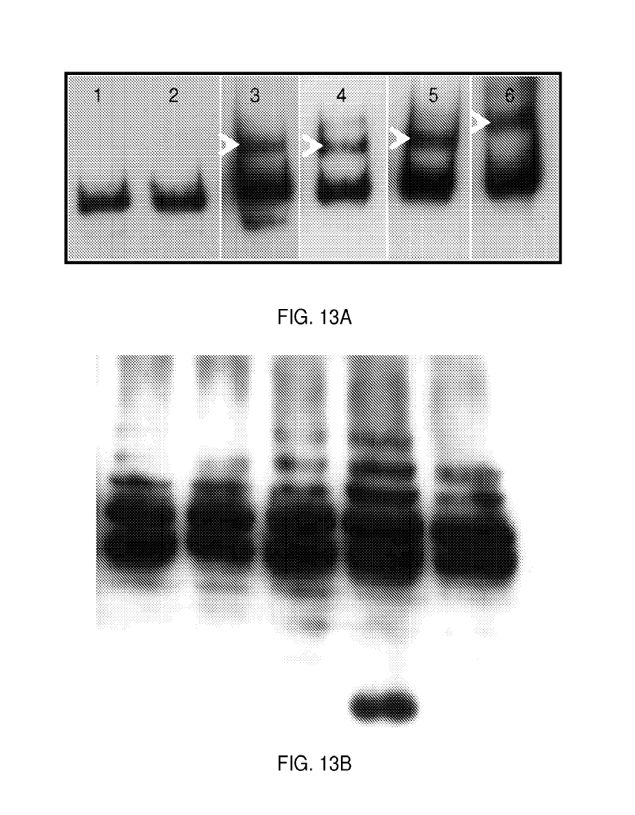

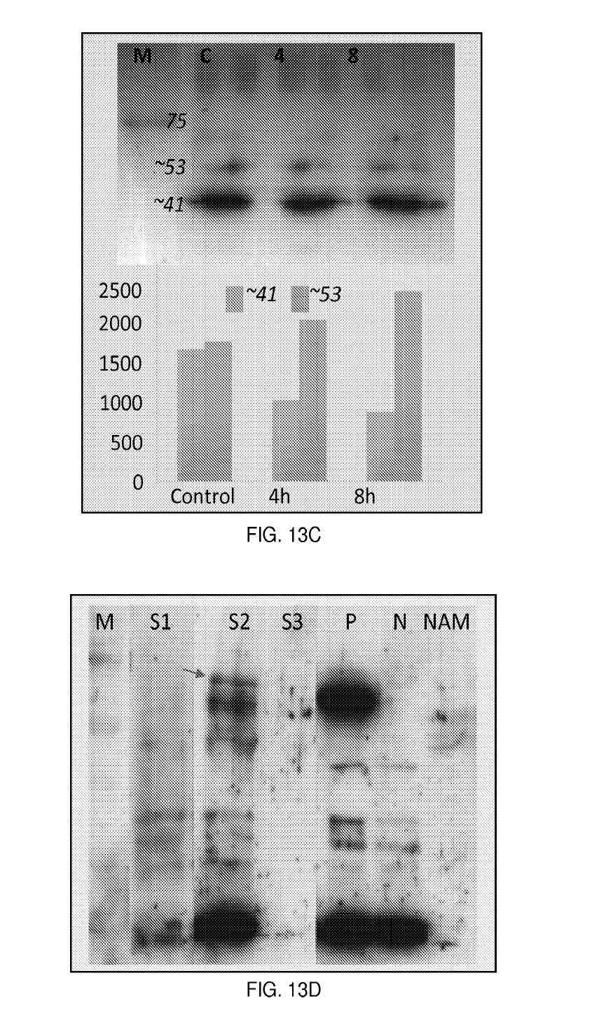

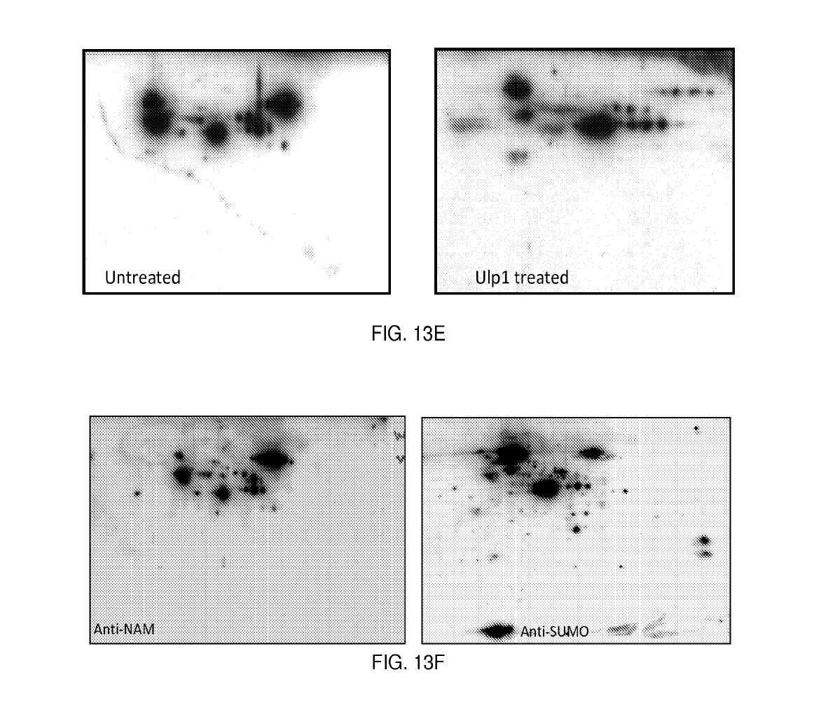

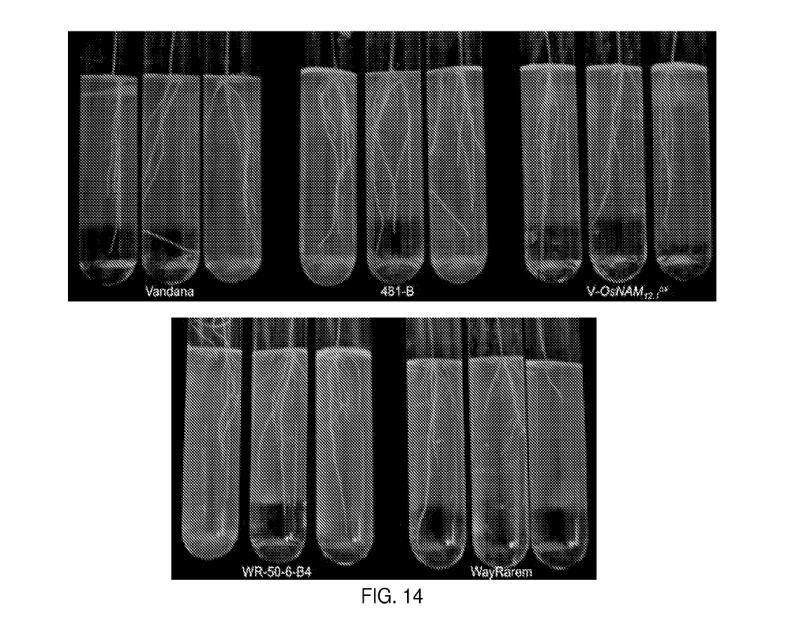

Promoter polymorphism in OsNAM_{12.1} was highly relevant to drought response and LRN, while non-synonymous CDS SNPs predicted protein structure variation (FIG. 10). Moreover, Arabidopsis CesA genes might be targets of NAC domain proteins (24). Thus, candidate gene promoters when queried for NAM/NAC binding sites revealed OsAmH_{12.1}, OsMtN3_{12.1}, OsCesA_{12.1}, OsGDP_{12.1} and OsARF_{12.1} as putative targets of OsNAM_{12.1}. Recombinant WayRarem OsNAM_{12.1} binding to these promoters was confirmed with electrophoretic mobility shift assay (EMSA), except for OsAmH_{12.1} (FIG. 13A). However, separate evidence supported OsAmH_{12.1} regulation by OsNAM_{12.1}. For example, WayRarem OsNAM_{12.1} when constitutively over-expressed in IR64 (I-OsNAM_{12.1}^{ox}), led to upregulation of OsCesA_{12.1}, OsMtN3_{12.1}, OsARF_{12.1} and OsGDP_{12.1} and down-regulation of OsAmH_{12.1} in T₂ homozygous plants under drought. The up- and down-regulation of these particular genes was similar to the observations in 481-B (FIG. 19). Similarly, the I-OsNAM_{12.1}^{ox} plants exhibited increased root and panicle branching, spikelet number and transpiration rates under drought, as in 481-B (FIGS. 11A-11E). WT IR64 and I-OsNAM_{12.1}^{ox} plants had similar TE and yield-under-drought in pot studies but under field conditions the I-OsNAM_{12.1}^{ox} plants exhibited increase in yield as seen through the number of filled spikelets (FIG. 11F). I-OsNAM_{12.1}^{ox} plants thus largely recapitulated the performance of 481-B under drought but not to similar extents, as discussed below.

The OsNAM_{12.1} is Differentially SUMOylated Under Drought.

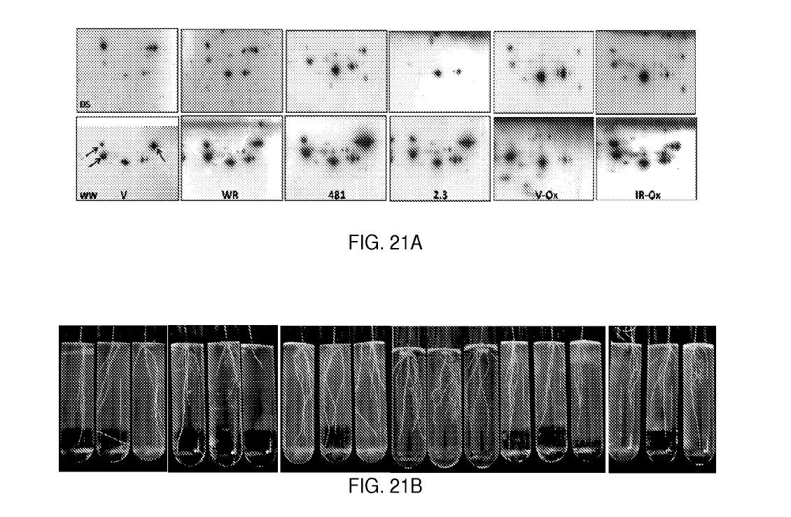

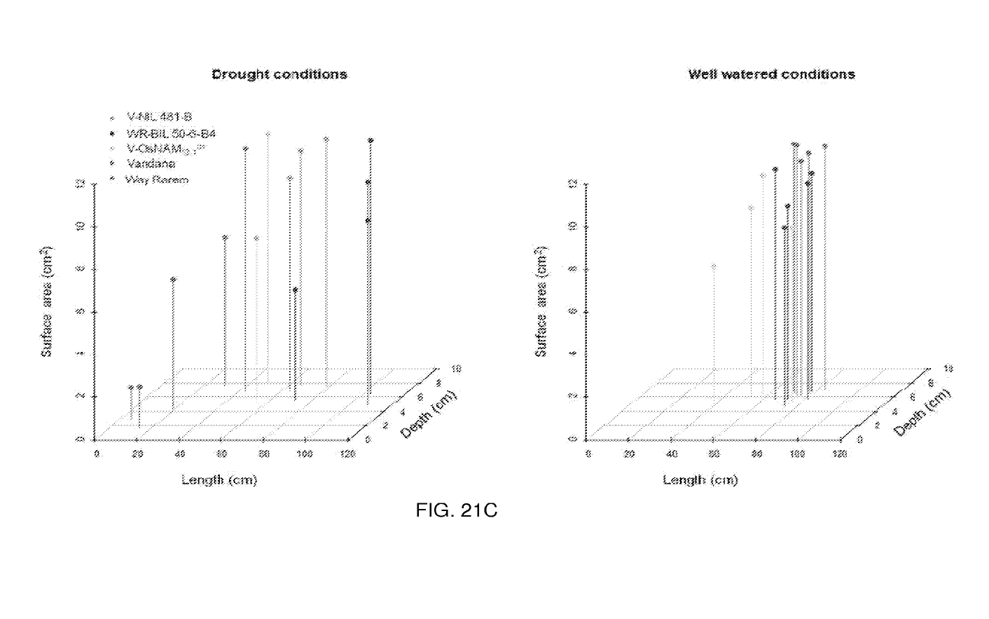

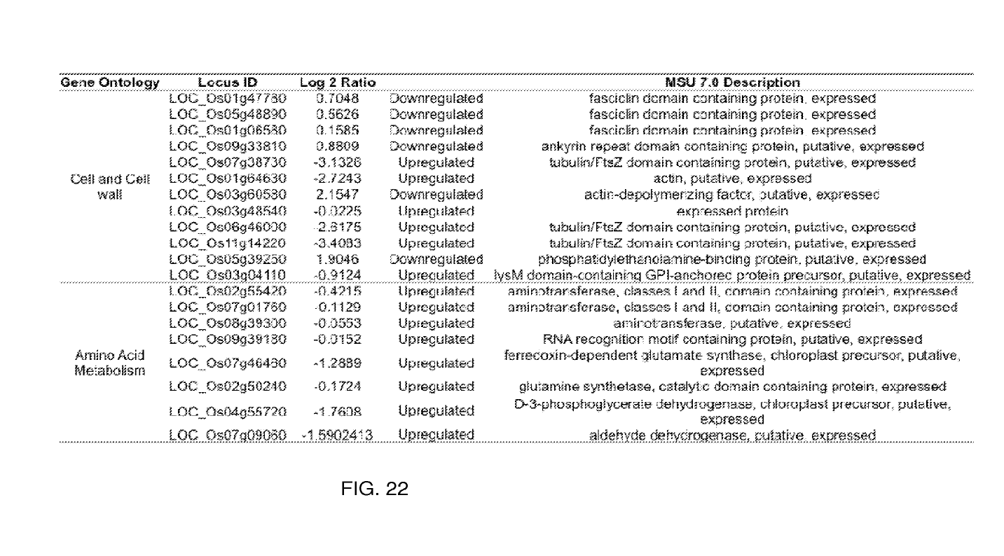

TFs act as negative and positive regulators, like OsNAM_{12.1} most likely does for OsAmH_{12.1} and the four other co-localized target genes respectively, through post-translational modification (PTM). Vandana and WayRarem OsNAM_{12.1} lacked a phosphorylation and a SUMOylation site respectively from the potential multiple sites for the two correlated PTMs (FIG. 21). Potential multiple SUMOylation may explain the multiple immunodetection bands for OsNAM_{12.1} (FIG. 13B). SUMOylation of the recombinant OsNAM_{12.1} and deSUMOylation of the plant OsNAM_{12.1} in vitro confirmed that it could be SUMOylated (FIGS. 13C-13D) while it's in vivo SUMOylation was confirmed through 2D-immunodetection before and after treatment with SUMO protease Ulp1 (FIG. 13E) and by the use of anti-OsNAM_{12.1} and antiSUMO antibody on the same blot (FIG. 13F). Differential SUMOylation of OsNAM_{12.1} under drought was also confirmed through 2D-immunodetection (FIG. 22A). The importance of differential SUMOylation of OsNAM_{12.1} and its potential role in explaining the epistasis noted for a functional qDTY_{12.1} is discussed below. Of the various modified moieties noted for OsNAM_{12.1}, drought-mediated deSUMOylation of some (FIG. 22A) was noteworthy.

The Functional OsNAM_{12.1} Haplotype is Specific to Susceptible Genotypes.

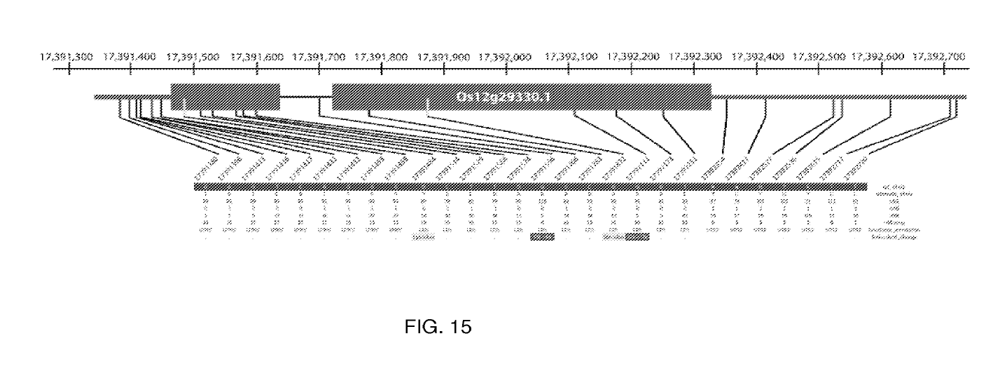

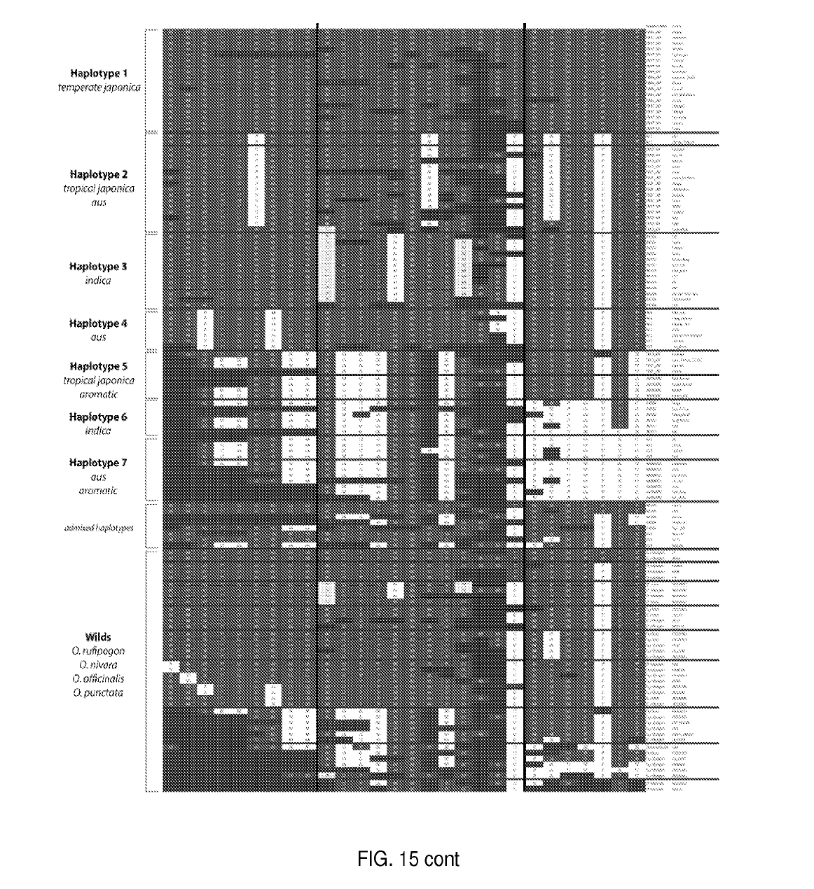

To associate the WayRarem OsNAM_{12.1} allele with drought tolerance across genotypes, SNP composition of a 6-Kb region surrounding OsNAM_{12.1} was examined in 125 re-sequenced rice lines. Substantial SNP and indel variation was noted and 7 major haplotypes were identified at different frequencies within the five O. sativa subpopulations and wild rice (FIG. 15). The haplotype containing the two non-synonymous genic SNPs as in WayRarem was absent in the drought tolerant lines but present in 9 drought sensitive, irrigated and high-yielding indica lines. De-novo OsNAM_{12.1} sequencing from known drought tolerant lines such as Apo, N22 and Dular also did not contain the WayRarem haplotype. The WayRarem OsNAM_{12.1} haplotype, as it existed in the sturdier wild rice O. nivara and O. rufipogon (FIG. 15) was most likely selected away from an epistatic locus when selecting/breeding for high yielding, irrigated genotypes, which are mostly drought susceptible. Indeed, epistatic interaction of qDTY_{12.1} was noted with the Vandana qDTY_{2.3} and its introgression into WayRarem increased the yield of the backcross-inbred lines (BIL; WR50-6-B4) under drought.

SUMOylation of OsNAM_{12.1} May Underlie qDTY_{12.1} Epistasis.

The OsNAM_{12.1} 2 D-immunodetection patterns of WR50-6-B4, 481-B, I-OsNAM_{12.1}^{ox} and the Vandana line transformed with the WayRarem OsNAM_{12.1} (V-OsNAM_{12.1}^{ox}) revealed down-regulation of certain OsNAM_{12.1} moieties under drought, which did not happen in Vandana and WayRarem (FIG. 22A). The change in the pattern of 2D-GE spots indicated deSUMOylation when compared to the pattern in FIG. 13E, wherein the latter demonstrated the action of the deSUMOylating protease Ulp1 on OsNAM_{12.1}. These results demonstrated that drought-mediated deSUMOylation of OsNAM_{12.1} was upregulated when qDTY_{2.3}, or a functional part thereof, was present. This indicated that OsNAM_{12.1} (de)SUMOylation status at qDTY_{12.1} might be affected by the epistatic qDTY_{2.3}, or a functional part thereof. These results also indicated that although OsNAM_{12.1} transcript, protein and protein-SUMOylation were detected in WayRarem, the requisite putative deSUMOylated moieties under drought were not achieved, most likely due to the lack of qDTY_{2.3}. In Vandana, a similar lack of deSUMOylated moieties under drought, despite the presence of qDTY_{2.3}, was due to the mutations in its OsNAM_{12.1} (FIG. 21). Vandana is thus an OsNAM_{12.1} functional knock out (KO) line, amenable to complementation with the WayRarem OsNAM_{12.1} as shown through the change in the OsNAM_{12.1} 2 D pattern (FIG. 22A) and in the drought responsive morpho-physiology of 481-B and V-OsNAM_{12.1}^{ox} (FIGS. 11; 22B-22C).

Relating Drought-Specific Lateral Root Branching and OsNAM_{12.1}.

Root architecture plays an important role in drought tolerance. In order to relate LRN to OsNAM_{12.1}, the parental genotypes Vandana and WayRarem; 481-B; WR50-6-B4; and V-OsNAM_{12.1}^{ox}; plants were assessed for total root length, maximum root depth and root surface area. Under normal conditions root characteristics of the different lines were largely similar. However under simulated drought, WR50-6-B4, 481-B and V-OsNAM_{12.1}^{ox}, in that order, exhibited significant increases over Vandana and WayRarem (FIGS. 22B-22C). These results showed that the WayRarem OsNAM_{12.1} by itself, but more in concert with additional WayRarem alleles, affected root architecture under drought and that qDTY_{2.3} had a role in the process.

Candidate Genes Other than OsNAM_{12.1} have a Role in qDTY_{12.1}

T-DNA insertion-mediated knock out (KO) line for OsAmH_{12.1} and activation-tag (AT) lines for OsCesA_{12.1}, OsGDP₁₂ and OsARF_{12.1}, along with those for OsSTPK_{12.1}, OsPOle_{12.1} and OsWAK_{12.1} were identified in the TRIM collection. LRN was enhanced in all mutants (FIG. 11) but panicle branching and spikelet number was not affected (Table 4). However, AT-OsSTPK_{12.1} exhibited significantly higher number of filled grains than the WT (Table 4). These findings, in combination with the candidate gene transcript expression patterns (FIG. 19) confirm OsAmH_{12.1} as a negative and other tested candidate genes as positive regulators of LRN.

TABLE 4
Panicle branching, spikelet number, and fertility of AT/KO mutants.

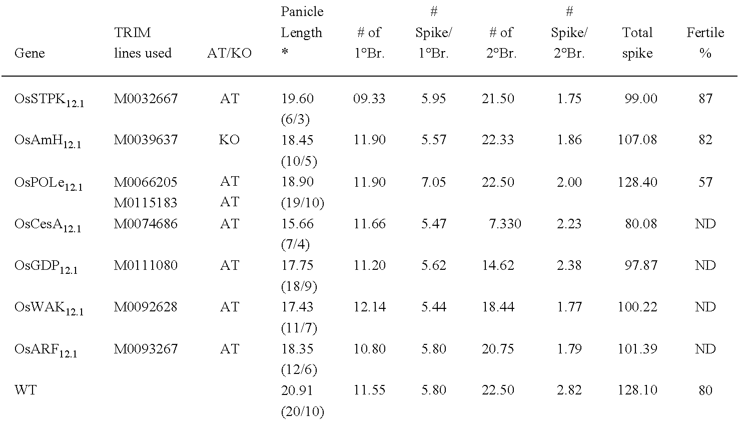

*Panicle length measured in cm. All numbers, except in parentheses, are average values based on the number of panicles sampled from the number of plants (the number in parentheses).
Activation tag (AT) or knock out (KO) status was confirmed through qRTPCR.
1°Br., indicates the average number of primary branches per panicle.
2°Br., indicates the average number of secondary branches per panicle.
# Spike, indicates the average number of spikelets per primary or secondary branches.
ND indicates ‘Not Determined’ due to low fertility.
Light grey shading denotes values significantly less than the WT and dark grey shading denotes values significantly higher than the WT.
This data from the greenhouse pot studies is an indication that OsSTPK_{12.1} has a role in increasing the fertility.

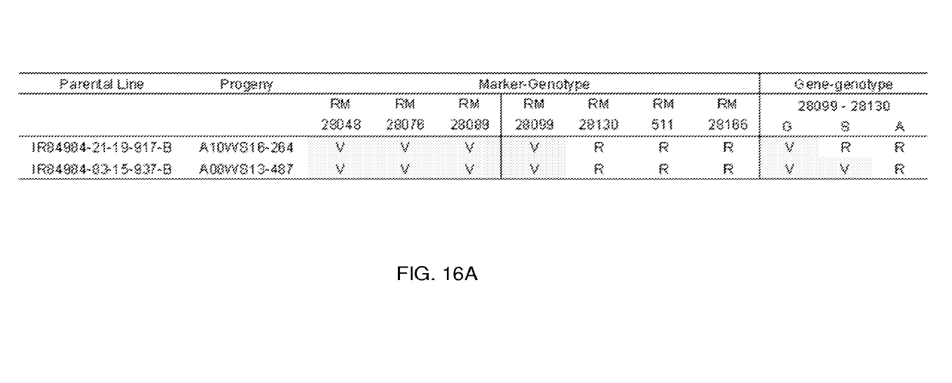

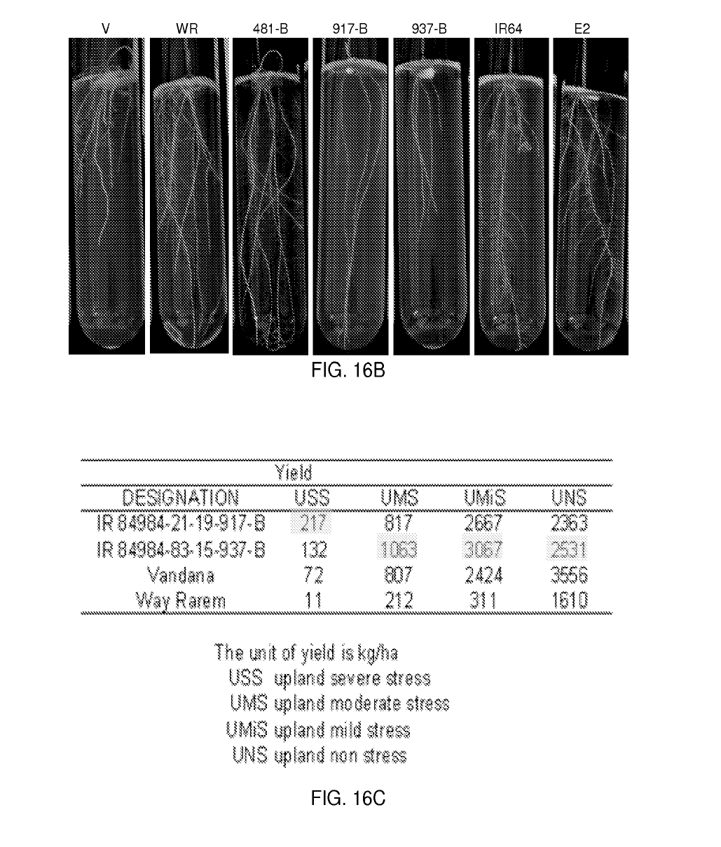

Yet another evidence for the multigenic nature of wDTY_{12.1} was the lack of increase in LRN under drought in the intra-QTL recombinant plants. Allele-specific genotyping for candidate genes revealed that intra-QTL recombinant plants of lines 917-B and 937-B were similar to 481-B except for missing the WayRarem allele of OsGPDP_{12.1} in both the lines while 937-B also lacked the WayRarem allele for OsSTPK_{12.1} (FIG. 16A). Both lines showed significant reduction in lateral root growth compared to 481-B (FIG. 16B). Field-based analysis revealed better yield for 937-B under no, mild, and moderate drought, but increased performance of 917-B, which contained the OsSTPK_{12.1}, under severe drought (FIG. 16C). These data indicated that OsGPDP_{12.1} is also involved in lateral root growth, while OsSTPK_{12.1} contributes not just to lateral root growth but also stabilizes yield under severe drought. Such a role for OsSTPK_{12.1} is supported by higher grain filling in the OsSTPK_{12.1} AT-mutant.

Unlike most QTLs that suffer from lack of validity in multiple locations and genotypes, qDTY1_{12.1} was valid in multiple locations, genotypes, eco-systems, and development stages. Reiterative field validation of the line 481-B over multiple years and seasons confirmed its stability and revealed yield advantage even under well-watered conditions.

Field-based characterization of 481-B revealed that multiple morpho-physiological traits were favorably affected including critical traits of days to flowering, transpiration efficiency and spikelet number and fertility. However, the one trait highly favored for drought tolerance, i.e. deeper roots, was not seen in 481-B. Instead, profuse root branching to increase LRN was observed. Various root traits explored for drought tolerance in rice have been reviewed in the literature, but LRN was never a main contender. Results presented herein show drought-specific increase in LRN was a distinguishing feature of 481-B in soil and even in PEG simulated water deficit in vitro (FIGS. 2E-2F, 16B, 22B).

The large-effect of qDTY_{12.1}, combined with the evidence of its influence on multiple traits, showed a role for multiple genes, distributed in sub-QTLs. Fine mapping studies with the SSR and putative candidate gene-based markers demarcated 4 fractions within qDTY_{12.1} (FIG. 2A) and showed the importance of the underlying candidate genes. Initial validity of the candidate gene selection was shown by results on sequence polymorphism and drought-mediated changes in the expression of these genes (FIG. 19).

OsNAM_{12.1} was considered a prime candidate gene herein because i) NAM/NAC TFs affect root architecture and drought tolerance; ii) phylogenetically it belonged to the ONAC1 clade, none of the eight members of which have been studied; iii) its promoter indel contained auxin and ethylene response elements important in drought response and root growth (FIG. 10D); and iv) its CDS SNPs changed a lysine (K7N) and a serine (5109N) which predicted altered SUMOylation and phosphorylation and a weaker fitting structural RMSD for Vandana than WayRarem to the rice stress-inducible NACl (FIGS. 10B-10C). Selection of OsNAM_{12.1} as the major candidate gene was justified when I-OsNAM_{12.1}^{ox} plants largely recapitulated the morpho-physiology of 481-B (FIG. 11). Additionally, V-OsNAM_{12.1}^{ox} plants also exhibited the expected changes in LRN (FIGS. 22A-22B) and in panicle branching.

Multiple glycosylation, phosphorylation, and SUMOylation sites were present in OsNAM_{12.1} (FIG. 21). Recombinant OsNAM_{12.1} was SUMOylated with SUMO2 in vitro but not with SUMO1 or SUMO3 (FIG. 13D). Protein SUMOylation under stress by SUMO1/2 but not SUMO3 was earlier noted in Arabidopsis indicating heterogeneity and plant-, trait- or protein-specificity for the protein: SUMO interaction. Further, the two-dimensional immunodetection results with anti-OsNAM_{12.1} and anti-SUMO antibodies showed identical spots and supported in vivo SUMOylation of OsNAM_{12.1} (FIG. 13F). Multiple immunodetectable OsNAM_{12.1} bands/spots seen in Vandana and WayRarem under well-watered conditions represented various PTM forms of OsNAM_{12.1}. Certain spots (indicated by arrows in FIG. 22A) were down-regulated under drought in a manner that indicated deSUMOylation when compared to FIG. 13E, when the latter shows SUMO protease Ulp1 treatment of OsNAM_{12.1}.

Transcription factor (de)SUMOylation is known to alter its role between activator and repressor. Results of candidate gene expression, EMSA, and SUMOylation combined with TRIM mutant analyses showed such a dual role for OsNAM_{12.1}, such that it repressed OsAmH_{12.1} but activated four other co-localized target genes. Since OsNAM_{12.1} SUMOylation was observed in all samples (FIG. 13B), the effective content of one or more particular forms of OsNAM_{12.1} is critical. However, results demonstrated that SUMOylation notwithstanding, along with the known effect of NAM/NAC TFs on increasing lateral roots under drought, OsNAM_{12.1} also affected panicle branching and spikelet number.

Two major haplotypes were identified for OsNAM_{12.1} in each of the subpopulations, with the exception of temperate japonica where all 19 genotypes carried a single OsNAM_{12.1} haplotype. This level of variation is significantly higher than that reported in other genes and demarks rapid and recent evolution across this locus and a high level of evolutionary plasticity in response to variable selection pressures. One of the indica-specific haplotypes displayed two non-synonymous SNPs in the CDS that are conserved between WayRarem and 10 indica genotypes, including IR64, indicating that the favorable qDTY_{12.1} allele is of indica origin. These results showed that qDTY_{12.1} originated from the high-yielding but drought susceptible WayRarem genotype. IR64 does not display drought tolerance characters of qDTY_{12.1}. The lagging effect on yield in I-OsNAM_{12.1}^{ox} plants compared to 481-B implicated one or more of the other nine genes in increasing the number of filled spikelets as opposed to OsNAM_{12.1} increasing the number of spikelets per se under drought. Indeed, AT-OsSTPK_{12.1} exhibited larger number of filled grains despite no changes in the number of branches in the panicle.

The functional OsNAM_{12.1} being restricted to susceptible genotypes indicated epistasis, which was identified with the Vandana qDTY_{2.3}, or a functional part thereof. The qDTY_{2.3} locus contained an ubiquitin protease, which acts as a deSUMOylating protein. Without wishing to be bound to any particular theory, the functional model for qDTY_{12.1} is that the Vandana OsNAM_{12.1} does not work due to the SNPs that cause K7N and 5109N alterations and the P223 insertion (this latter P insertion was not noticed in any of the 125 re-sequenced genomes), while the WayRarem OsNAM_{12.1} does not work, due to altered ubiquitin protease or any other gene at qDTY_{2.3} that facilitates deSUMOylation. Further, V-OsNAM_{12.1}^{ox} and WR50-6-B4 plants exhibited drought-mediated deSUMOylation of OsNAM_{12.1} and LRN increased similar to that in 481-B and I-OsNAM_{12.1}^{ox} plants (FIGS. 11A-10B, 16B, 22B-22C), showing a direct relationship between OsNAM_{12.1} deSUMOylation and LRN increase.

Low level of intra-QTL recombination of 2.7% (52/1900) instead of the expected 7% for a 1.75 Mb region (1 Mb=4 cM), due to its proximity to the centromere, underscores the practical value of qDTY_{12.1}.

A breeding strategy based on SNP markers for the 10 CGs will fast track ongoing efforts to introgress drought tolerance into popular, local varieties. Success with this robust LEQ, as opposed to the search for a master-regulator, to address the complex trait of drought tolerance underscores its practical value. It supports the much espoused meaningful complementation of field-based classical breeding and physiology with molecular biology to ameliorate food scarcity, hunger and poverty through rice science.

The present invention provides molecular markers, (i.e. including marker loci and nucleic acids corresponding to (or derived from) these marker loci, such as probes and amplification products) useful for genotyping plants, correlated with the qDTY_{12.1} QTL in rice. Such molecular markers are useful for selecting plants that carry the drought tolerance QTL or that do not carry the drought tolerance QTL. Accordingly, these markers are useful for marker assisted selection (MAS) and breeding of drought tolerant lines and identification of non-tolerant lines. Markers which may be used include: RM28048 (forward primer: SEQ ID NO: 22; reverse primer: SEQ ID NO: 23); RM28076 (forward primer: SEQ ID NO: 24; reverse primer: SEQ ID NO: 25); RM28089 (forward primer: SEQ ID NO: 26; reverse primer: SEQ ID NO: 27); RM28099 (forward primer: SEQ ID NO: 28; reverse primer: SEQ ID NO: 29); RM28130 (forward primer: SEQ ID NO: 30; reverse primer: SEQ ID NO: 31); RM511 (forward primer: SEQ ID NO: 32; reverse primer: SEQ ID NO: 33); RM1261 (forward primer: SEQ ID NO: 34; reverse primer: SEQ ID NO: 35); RM28166 (forward primer: SEQ ID NO: 36; reverse primer: SEQ ID NO: 37); RM28199 (forward primer: SEQ ID NO: 38; reverse primer: SEQ ID NO: 39); and Indel-8 (forward primer: SEQ ID NO: 60; reverse primer: SEQ ID NO: 61).

Epistasis occurs where the expression of one gene depends on the presence of one or more modifier genes. Under DS, epistasis between WayRarem qDTY_{12.1} and Vandana qDTY_{2.3} increased yield. Several candidate genes at the epistatic QTL_{2.3} on chromosome 2 have been identified. These include LOC_Os02g45580, LOC_Os02g45670, LOC_Os02g45700, LOC_Os02g45710, LOC_Os02g45750, LOC_Os02g45770, LOC_Os02g45810, LOC_Os02g46100, LOC_Os02g46140, LOC_Os02g46260, LOC_Os02g46320, LOC_Os02g46340, LOC_Os02g46350, LOC_Os02g46360, LOC_Os02g46600, LOC_Os02g46650, LOC_Os02g46690, LOC_Os02g46700, LOC_Os02g46720, LOC_Os02g46770, LOC_Os02g46780, LOC_Os02g46910, and LOC_Os02g46940.

In particular embodiments, qDTY_{12.1}, or a functional part thereof, is bred into a variety of rice having a functional qDTY_{2.3}. In another embodiment, both qDTY_{12.1} and qDTY_{2.3}, or functional parts thereof, are bred into a recipient variety. In yet other embodiments, one or more of the candidate genes at the epistatic QTL_{2.3} are expressed in a rice plant along with QTL_{12.1}, or one or more of the candidate genes at QTL_{12.1} identified above.

### Example 2

### Complexity of Drought Tolerance—Proteomic and Targeted Metabolite Analysis of Field Proven Near Isogenic Lines of a QTL for Rice Yield Under Stress

Plant Material and Growing Conditions

Field experiments were conducted at the International Rice Research Institute (IRRI, Los Baños, Laguna, 14° 10′11.81″N, 121° 15′39.22″E) during a dry season. Seeds of qDYT_{12.1} 481-B, generated as described above, and those of the parents Vandana and WayRarem were directly sown into rotovated soil at a rate of 2.0 g m-1 into plots of 3 rows×3 m, with 3 replicates per genotype in a randomized complete block design. The late flowering WayRarem was sown 20 days before the other genotypes in order to synchronize drought stress with flowering stage. Two treatments were included: a well-watered (WW) treatment in which sprinkler irrigation was applied 3 times per week throughout the study, and a drought stress (DS) treatment in an automated field rainout shelter in which irrigation was stopped at 35 days after the qDTY_{12.1} NILs were sown. At 71 days after sowing, developing spikelets, flag leaves, and root crowns of 481-B and Vandana were sampled for metabolomics and proteomics analysis, wrapped in aluminum foil, and placed directly into liquid nitrogen before storing at −80° C. Developing spikelets, flag leaves, and root crowns of WayRarem were subsequently sampled at 113 days after it was sown. Soil water potential in the DS treatment was monitored by tensiometers (Soilmoisture Equipment Corp., CA, USA; one per replicate) installed at a depth of 30 cm. From the date that WayRarem plots were sown until harvest, the ambient temperature averaged 23.4-30.8° C. (min-max), relative humidity averaged 85.8%, the crop received 1750 MJ m-2 solar radiation, and pan evaporation totaled 552 mm.

Measurements of Photosynthesis and Stomatal Conductance.

The field experiment was conducted at the International Rice Research Institute (IRRI, Los Baños, Laguna, 14° 10′11.81″N, 121° 15′39.22″E) during the dry season. Using a LI-6400 portable gas exchange system (Li-Cor Inc., Lincoln, Nebr.), light response curves were conducted in the stress and control treatments for Vandana and 481-B. The CO2 response curves were also conducted on these two treatments. Settings for all measurements were based on ambient conditions and included a leaf temperature of 30° C. and a flow rate to maintain relative humidity at 65%. The CO2 level was set to 400 ppm for the light response curves, and the light level was set to 1000 μmol m-2 s-1 for the CO2 response curves.

Protein Extraction and Separation by 1-DE.

Protein samples were extracted from the plant material using the Tris method. Flag leaf, root and spikelets (100 mg) from the three genotypes were pulverized with liquid nitrogen into fine powder to which 0.7 ml of Tris-Cl buffer (pH 8.0) was added. Seven (7) μl of protease inhibitor cocktail (Sigma-Aldrich) was added to prevent endogenous protease digestions. Samples were then allowed to incubate on ice while shaking for 2 hours. After incubation, they were spun at 17900×g (13000 rpm) for 15 minutes and the supernatant was collected. Quantification for all the samples was done using the Bradford method. Samples were run through SDS-PAGE under denaturing conditions as described in the Laemmli method (Laemmli, 1970). A total of 20 μm of protein sample was loaded per well. Loading dye with SDS and β-mercaptoethanol was added to each sample. They were then placed in a hot water bath for 5 minutes, and cooled to room temperature before loading. Gels were run at constant current of 15 mA for 2 hours per gel, and stained with Coomassie Brilliant Blue (G-250) for 24 hours, and destained for another 2 hours before tryptic digestion.

In-Gel Digestion of Protein & Tandem Mass Tag (TMT) Labeling.

Protein bands were excised and collected from the three independent replicate gels manually, and cut into small pieces. The gel pieces were washed twice with 50 μL of 50% acetonitrile (ACN)/50% 200 mM ammonium bicarbonate (ABC) for 5 min and shrunk with 100% ACN until the gels turned white; the gels were then dried for 5 min in a concentrator (miVac, Genevac, UK). The gel pieces were rehydrated at room temperature in 15 μL of 50 mM ABC (37° C., 4 min). An equivalent volume (15 μL) of trypsin (Promega, USA) solution (20 ng/4 in 50 mM ABC) was then added, and the gel pieces were incubated at 37° C. for at least 16 h. After digestion, the digests were extracted from gel slices by using 0.1% formic acid in 50% ACN. All extracts were dried in concentrator. TMT labeling was performed on each aliquot with Tandem Mass Tags (TMT) with respective reporters at m/z=126.1, 127.1, 128.1, 129.1, 130.1 and 131.1 Thomson (Th) in 40.2 μL CH3CN. After 60 min of reaction at RT, 8 μL hydroxylamine 5% (w:v) was added in each tube, and mixed for 15 min. The aliquots were then combined and the pooled sample was evaporated under vacuum. The sample was then dissolved in 1894 μL H2O/TFA 99.9%/0.1% before LC-MS analysis.

Nano LC-MS/MS Analysis.

Each digested peptide mixture (5 μL) for nano-LC/MS/MS analyses were introduced into the mass spectrometer via high-performance liquid chromatography using a 1200 series binary HPLC pump (Agilent, CA, USA) and a FAMOSTM well-plate microautosampler (LC Packings). For each analysis, sample was loaded into a 2 cm×75 μm i.d. trap column packed in-house with C18 resin (Magic C18AQ, 5 mm, 200 Å; Michrom, Bioresources, CA, USA). The trap column was connected to an analytical column (11 cm×75 mm i.d.) and the columns were rigidly packed in-house with C18 resin (Magic C18AQ, 5 μm, 100 Å). Mobile phase A consisted of 0.1% formic acid and mobile phase B consisted of 0.1% formic acid in 100% ACN. The flow rate was ˜250 nL/min under an in-house split flow system. Each reversed-phase step began with 5% ACN for 10 min, a gradient of 5%-40% ACN for 75 min, 40%-85% ACN for 5 min, 85% ACN for 10 min, and then re-equilibrated with 5% ACN for 20 min. Mass spectrometric analyses were performed on a LTQ XL linear ion trap mass spectrometer (ThermoFisher Scientific, San Jose, Calif., USA). A full-mass scan was performed between m/z 350 and 2000, followed by MS/MS scans of the five highest-intensity precursor ions at 35% relative collision energy. Dynamic exclusion was enabled with a repeat count of 1, exclusion duration of 3 min, and a repeat duration of 30 s.

Protein Identification.

The acquired MS/MS spectra were searched against SwissProt protein database 56.8 (release of 10 Feb. 2009) using the Mascot Daemon version 2.2.2 and Oryza sativa was chosen for the taxonomic category. Peptide mass tolerance and fragment tolerance were set at 2 Da and 0.5 Da, respectively. The initial search was set to allow for up to two missed tryptic cleavages. A Decoy database was performed to determine false positive rates. The false positive rates were controlled below 5% by setting p value at 0.025.

Functional Annotation.

Identified proteins with one or more than one peptide with MASCOT score greater than 40 were immediately accepted. Single peptides with MASCOT score less than 40 were deleted from the analysis to avoid false positives; 23, 21 and 15 single peptides were deleted from flag leaf, panicle and root respectively. The MSU TIGR v7.0 locus identifiers of the remaining proteins were retrieved from ID mapping tool in UniProtKB for giving them as input in MAPMAN. Finally a total of 915 proteins, of which 304, 407 and 204 proteins with TIGR locus IDs from flag leaf, panicle and root were used for further functional annotation using MAPMAN. The proteins were mapped against the already available rice mapping file and mapped proteins were classified into 24 functional categories based on MAPMAN BINS described by Thimm et al. (2004).

Starch Estimation.

Starch was estimated by measuring the NADH absorption at 340 nm which was generated during the conversion of glucose 6 phosphate to 6-phosphogluconate by the enzyme, glucose 6 phosphate dehydrogenase (Ernst and Arditti, 1972). The pellet obtained (from 15-20 mg of seed or leaf) after ethanolic extraction was used for starch estimation by HCl (Hydrochloric acid). The pellet was dissolved in 2N HCl (1.5 mL) and incubated at 95° C. for 1 h. The resulting mixture was directly used for glucose estimation after centrifugation at 13,000 g for 5 min. A mixture of 750 μL Imidazole buffer (pH 6.9) consisting of 2 mM NAD and 1 mM ATP was incubated at room temperature for 10 minutes in a disposable plastic cuvette along with 5-10 μl of the extract and 2 μL of glucose 6 phosphate dehydrogenase (2 units). After recording the initial absorbance of the mixture at 340 nm, 10 μL of hexokinase (8 units) solution was added to the mixture and incubated for further 25 min and the absorbance was recorded at 340 nm. A standard curve was prepared using starch (standards) from maize kernel.

Sugar Estimation

Lyophilised powdered plant sample was extracted three times with 80% ethanol by incubating at 60° C. for 30 min in a thermomixer. The supernatant obtained after centrifugation at 13,000 g for 10 min at 4° C. was evaporated to dryness using a centrifuge vacuum evaporator. The dried material was re-dissolved in deionized water and vortexed thoroughly. Contents were then filtered (Ultrafree-MC Membranes; Millipore) and the filtrate obtained was used for estimation of sugar analysis by HPAEC method.

Soluble sugars were analyzed by ion chromatography, HPAEC-PAD (High Performance Anion Exchange Chromatography-Pulsed Amperometric Detection). Chromatographic analysis was conducted with a Dionex IC system consisting of an autosampler AS 50, a gradient pump GP 50, and an electrochemical detector ED 40 equipped with a thin-layer-type amperometric cell. The cell comprised a gold working electrode and an Ag/AgCl reference electrode. Data acquisition and processing were accomplished with the Dionex Chromeleon 6.70 software. Chromatographic separation was carried out with the analytical column, CarboPac PA 20 in conjunction with a guard column and an Ion 1 Pac trap guard column. Column temperature was maintained at 35° C. in a column oven (STH-585). Analytes were separated with isocratic elution using 50% A (150 mM NaOH) and 50% B (water) as eluents at a flow rate of 0.3 mL min-1 for 15 min. Analyte detection was achieved by applying a quadrupole-potential waveform on the gold electrode (E1=0.1 V from 0 to 0.4 ms; E2=2.0 V from 0.41 to 0.42 ms; E3=0.6 V from 0.42 to 0.43 ms; E4=−0.1 V from 0.4 to 0.5 ms). The analytical data quality was controlled by standard addition methods.

Estimation of Amino Acids by HPLC

Lyophilised powdered plant sample was extracted three times with 80% ethanol by incubating at 60° C. for 30 minutes in a thermomixer. The supernatant obtained after centrifugation at 13000 g for 10 minutes at 4° C. was evaporated to dryness using a centrifuge vacuum evaporator. The dried material was then re-dissolved in the deionized water and vortexed thoroughly. Contents were then filtered (Ultrafree-MC Membranes; Millipore) and the filtrate obtained was used for estimation of amino acids. The reagents and solutions required for sample derivatization were available in the kit provided by Waters (AQC dry powder, acetonitrile for dissolving the reagent and borate buffer). Derivatization was carried out according to the instructions provided in the manual, AccQ-Tag method (Meyer et al., 2008). Briefly, AQC reagent powder was dissolved in 1 mL of acetonitrile which was approximately 3.0 mg/mL, vortexed thoroughly and incubated at 50° C. for 10 min. A mixture of standard amino acids except asparagine and glutamine was available from Sigma (0.5 mM in 0.01 M HCl). A working solution of 50 pmol/μL of each amino acid was made using 0.01 M HCl after adding asparagine and glutamine separately. About 10 μL of the fluorescent dye reagent was added to a small eppendorf (0.5 mL) containing 10 μL of sample and 80 μL of borate buffer (0.2M, pH 8.8). The contents were thoroughly mixed immediately and incubated at 50° C. for 10 min. and analyzed by HPLC. Similarly, standard was prepared by derivatizing with different volumes of the working standard solution. Unused reagent could be stored at −20° C. for several weeks. Before the chromatographic analysis, the system was equilibrated with 100% eluent A (140 mM sodium acetate and 7 mM triethanolamine) and the column temperature was set to 37° C. Fluorescence detector was set at 248 nm wavelength for excitation and 395 nm for absorbance. Chromatography was carried out using a Dionex HPLC system (Summit) consisting of a gradient pump (P680), a degasser module, an autosampler (ASI-100) and a fluorescent detector (RF 2000). Data acquisition and processing was accomplished with Dionex Chromeleon 6.70 software. The gradient was accomplished with eluent A, B and C representing buffer, acetonitrile and water, respectively. Analytes were separated on a reversed-phase analytical column (AccQ Tag) coupled to a guard column (Nova-Pak C18). The column temperature was maintained at 37° C. throughout the measurement and the flow rate to 1 mL/min. The gradient was produced by the following concentration changes, t=0, 100% A; t=0.5 min, 99% A and 1% B; t=27.5 min, 95% A, and 5% B; t=28.5 min, 91% A and 9% B; t=44.5 min, 82% A, 18% B; t=47.5 min, 60% B and 40% C; t=50.5 min, 100% A and t=60 min, 100% A. During the whole run, the gradient curve was always maintained at 6. Free proline content was also assayed using the ninhydrin assay (Bates et al., 1973)

Carbon and Nitrogen Analysis.

Carbon and nitrogen analysis was carried out using the elemental analyzer (vario EL III). Instrument was switched on about 3-5 hours before actual analysis and the measurement was carried out in CN mode. About 3 to 4 mg of oven dried sample was weighed in aluminum capsule, folded and placed in the autosampler. During measurement, the capsule enclosing the sample falls into a combustion chamber with excess oxygen kept at 900° C., where it is mineralized with the help of some catalysts. Various gases formed (CO2, H₂O and NOx) then passes through a silica tube packed with copper granules held at about 500° C. (reduction tube) where the remaining oxygen is bound and nitric/nitrous oxides are reduced to N2. The leaving gas stream includes analytically important CO2, H2O, N2 and SO2. All gases are removed at appropriate traps leaving the analytically important CO2 and N2 which are subsequently detected with a thermal conductivity detector. High purity helium (Quality 5.0) is used both as a carrier and reference gas. Blank values are obtained from empty aluminum capsules and calibration is done by elemental analysis of standard substances supplied by the manufacturer.

Quantitative PCR.

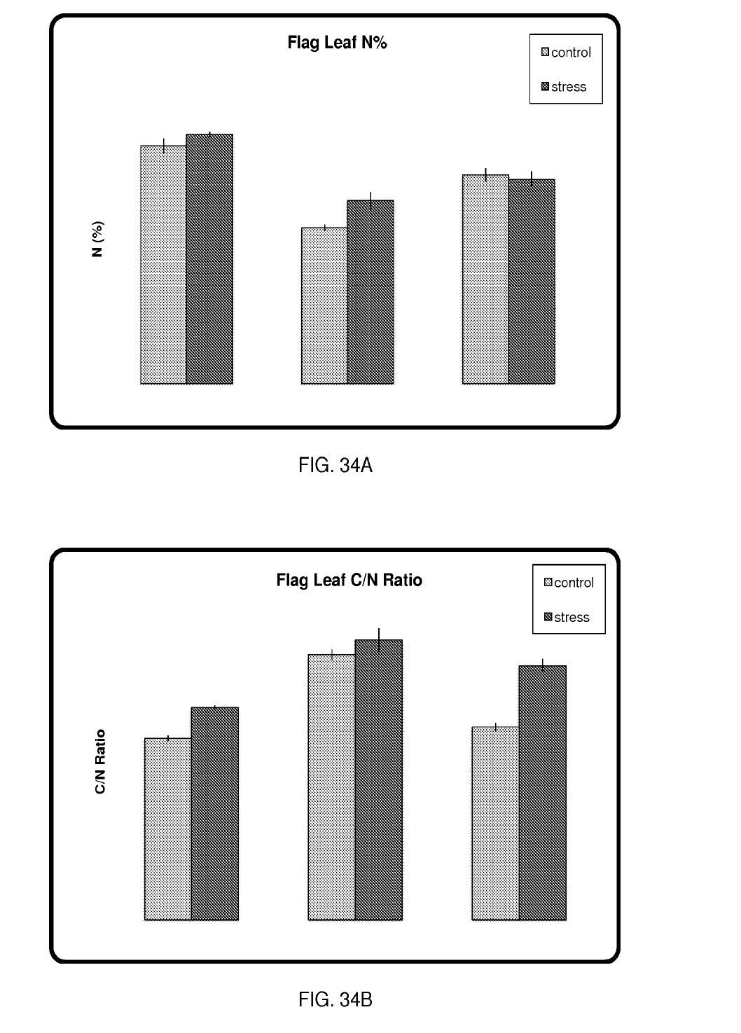

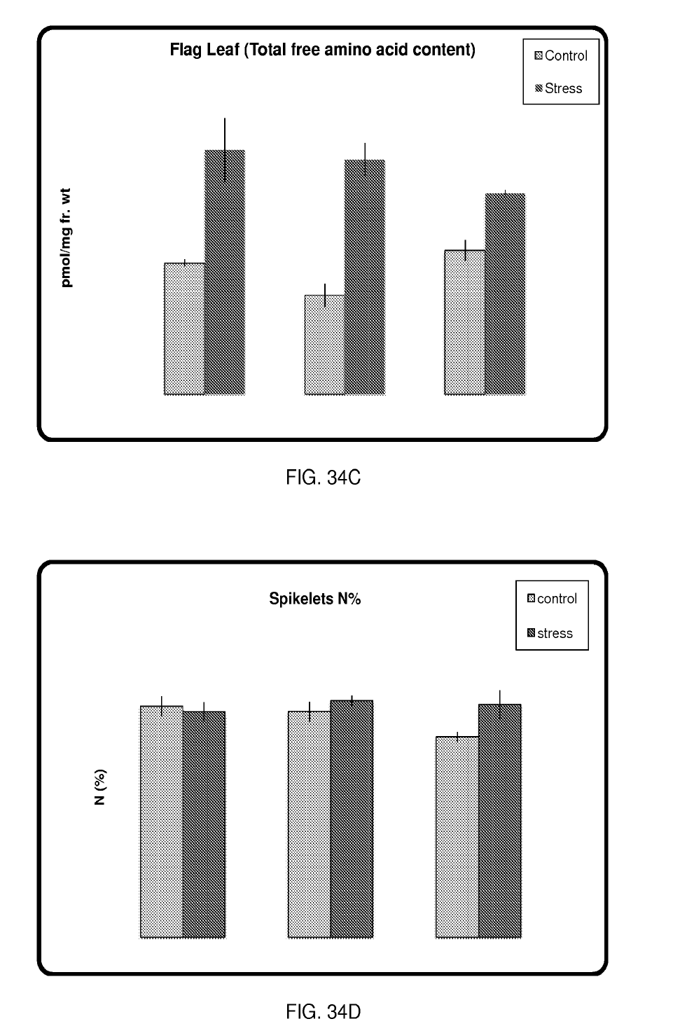

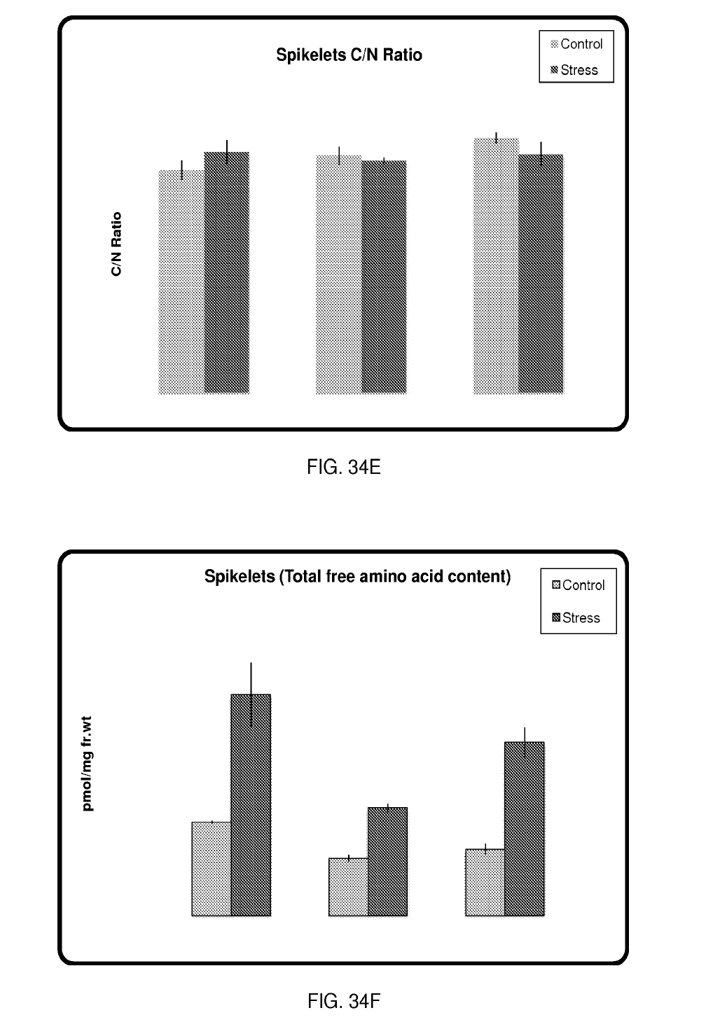

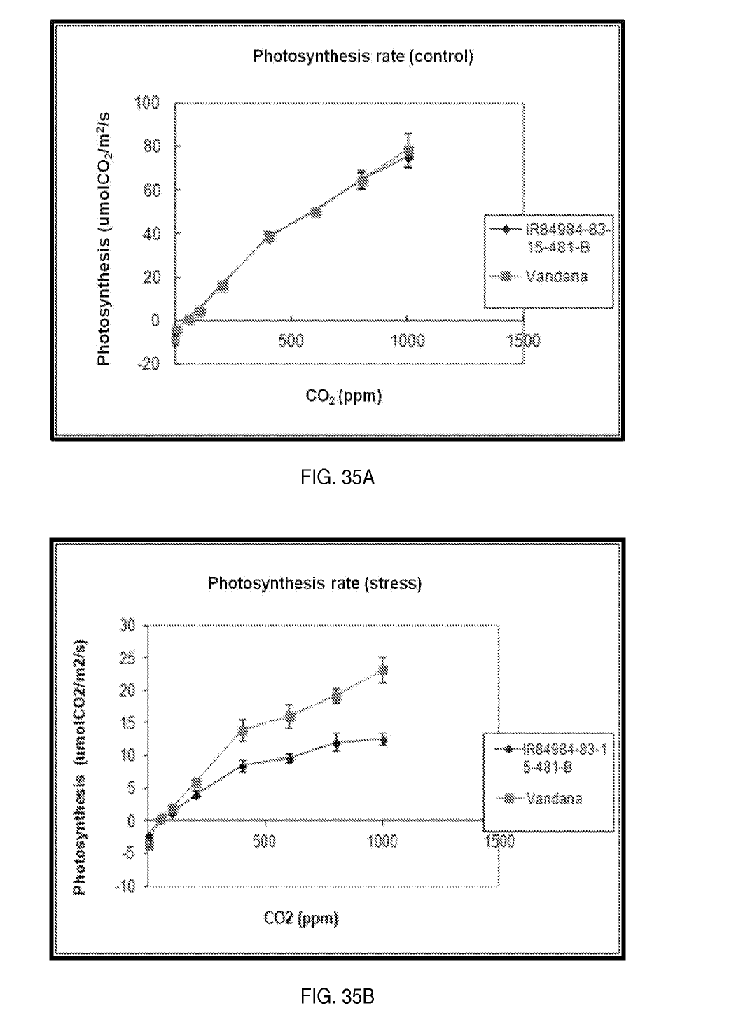

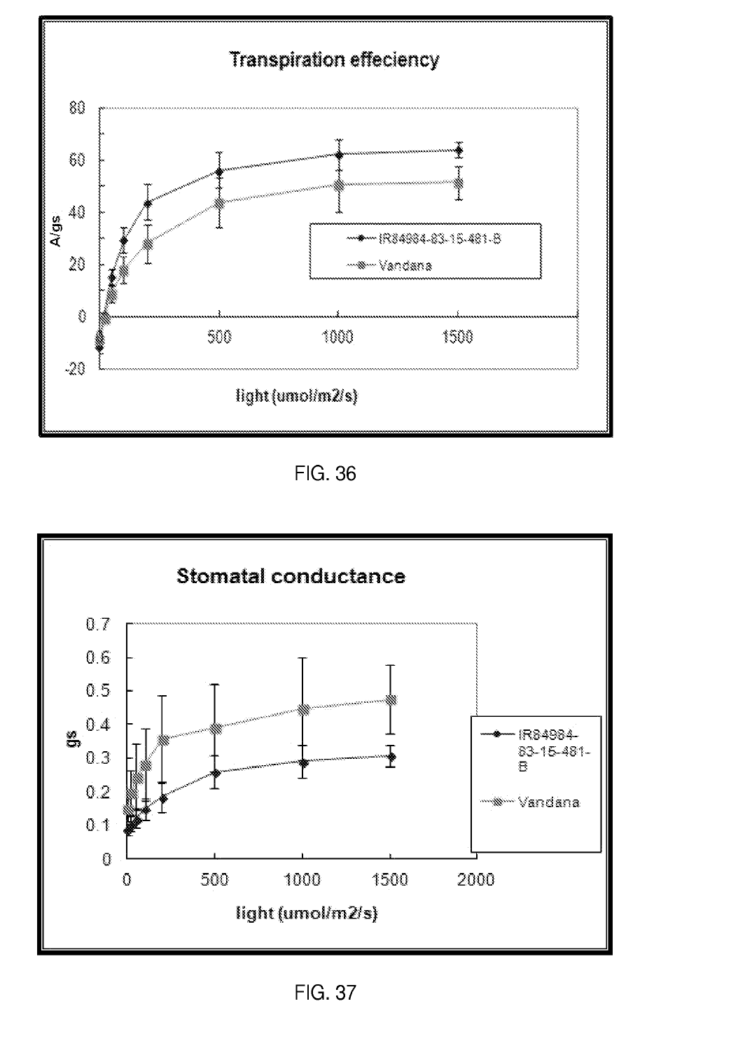

Quantitative RT-PCR analysis of few important selected proteins was performed. The primers were designed using Primer 3 (FIG. 34). All the three tissues harvest for the proteomic and the metabolomic measurements were rapidly placed in liquid nitrogen and grinded into powder, and immediately transferred to TRIzol Reagent (Invitrogen, Life Technologies). The total RNA was then extracted according to the manufacturer's recommendations. The cDNA was synthesized immediately after the RNA was extracted (ImProm-II™ Reverse Transcription System, Promega, USA). The quantitative PCR was performed with a Applied Biosystem 7500 Fast system (Applied Biosystem, 1 USA) using the SYBR® Select Real-time PCR Master Mix (Applied Biosystem, USA). Each gene was detected in quadruple simultaneously with the actin gene as an internal control.

Results

TMT DATA Analysis.

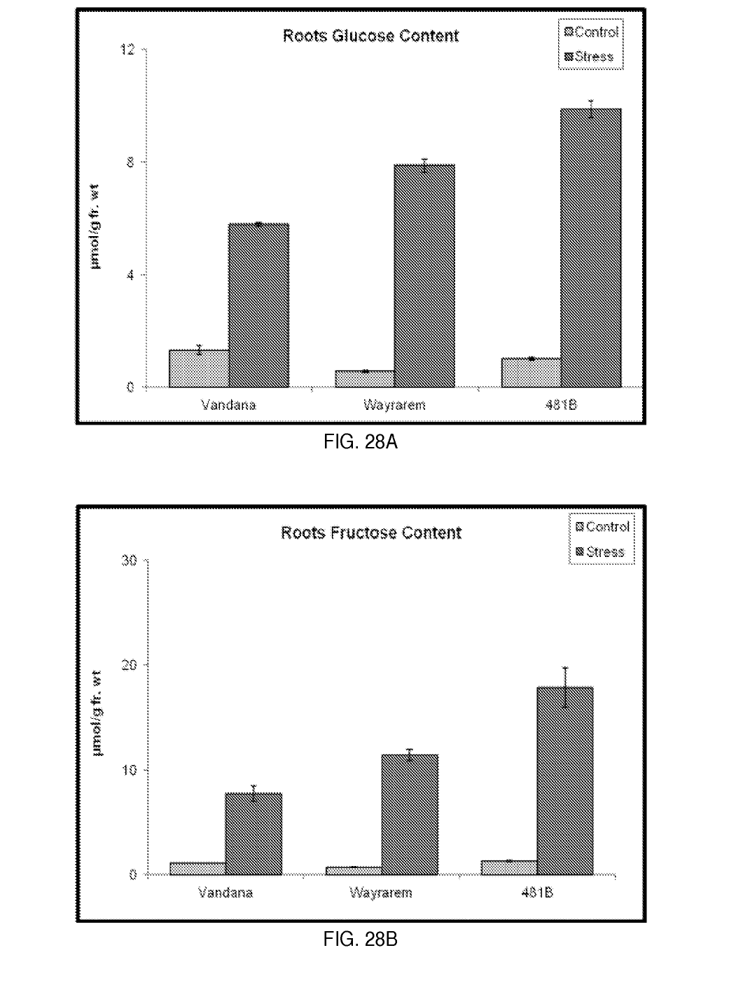

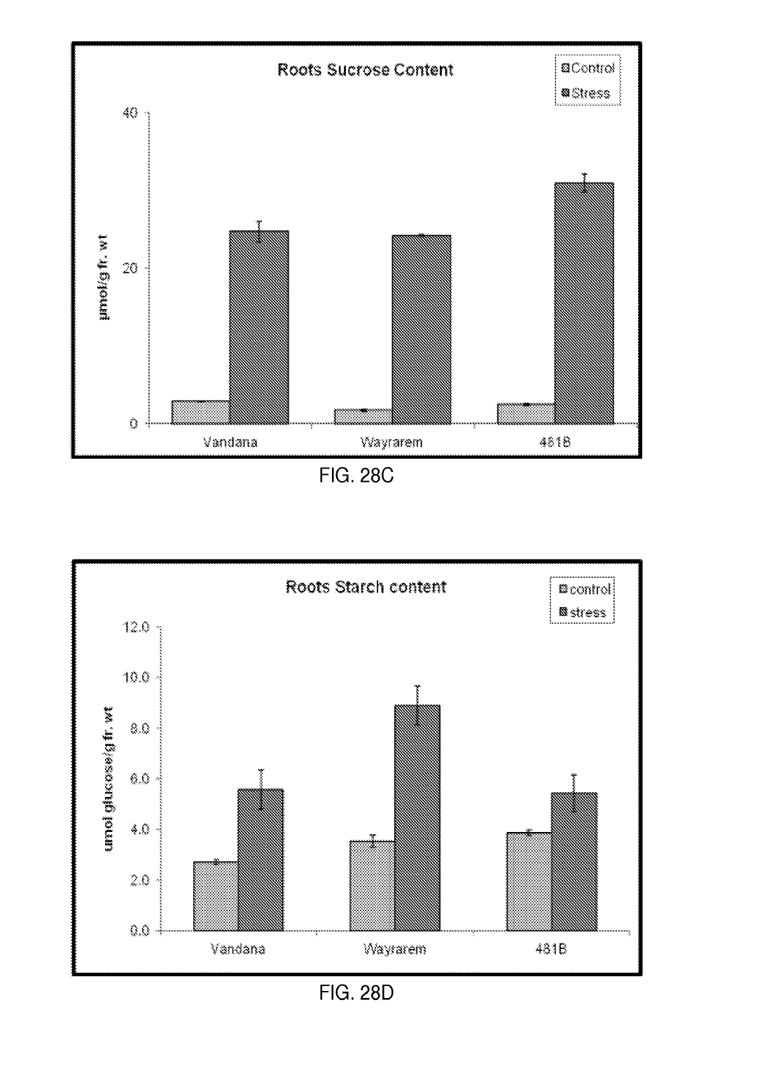

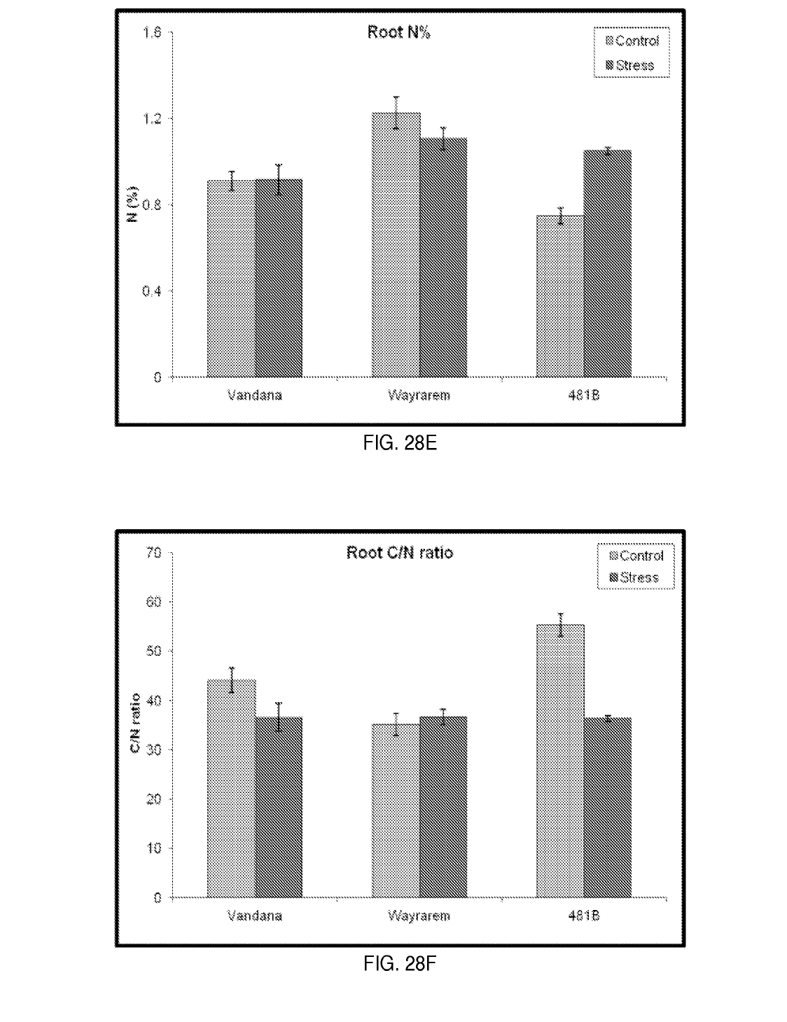

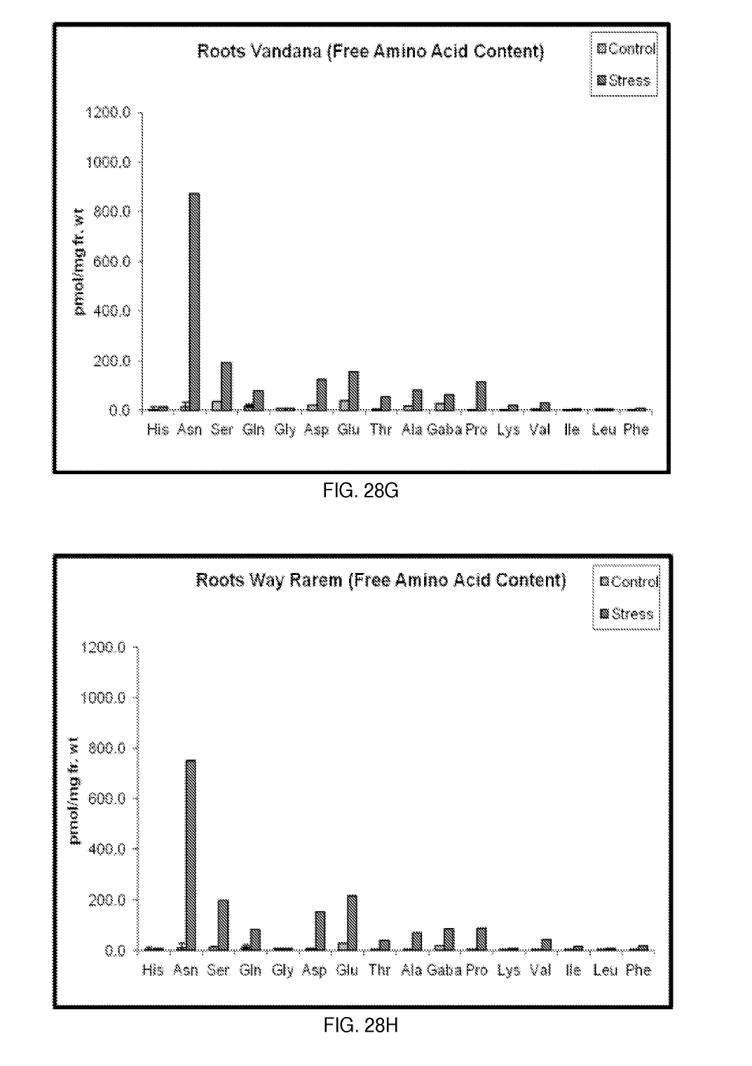

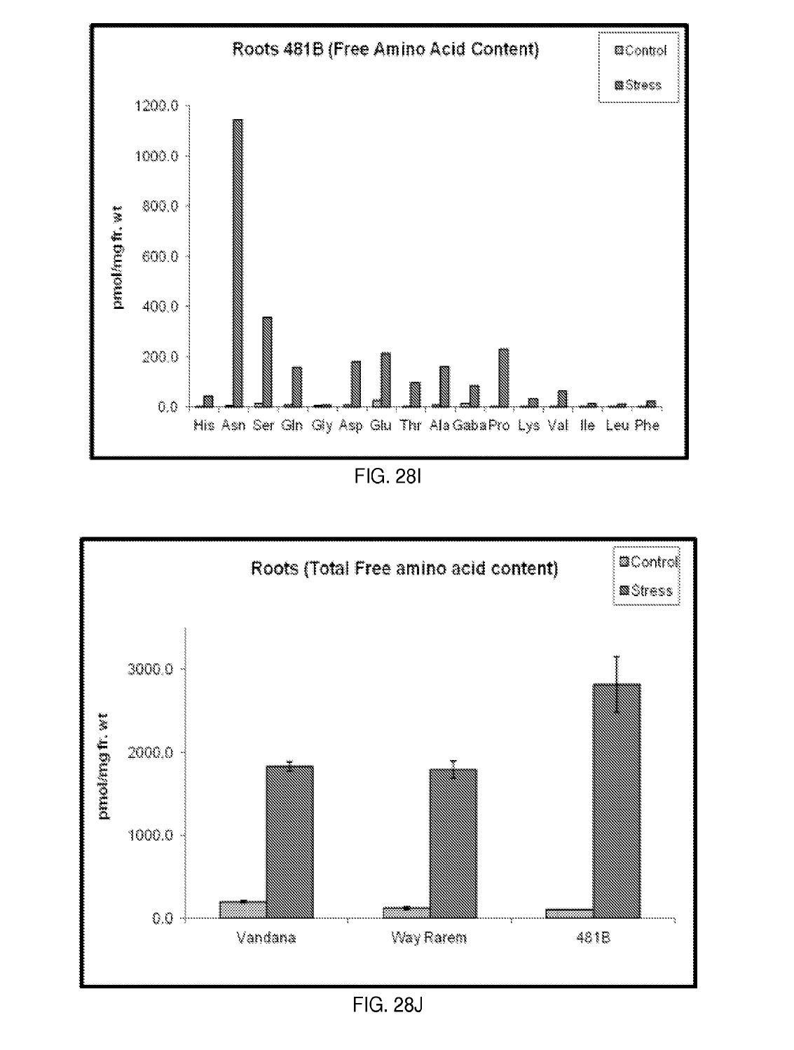

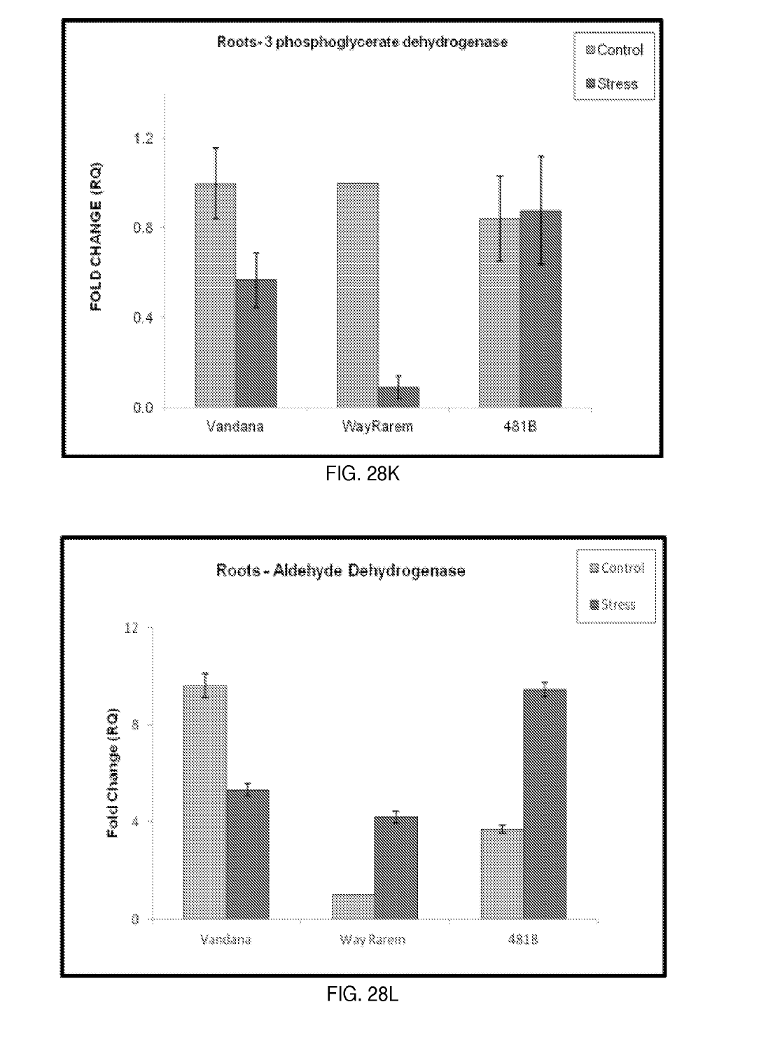

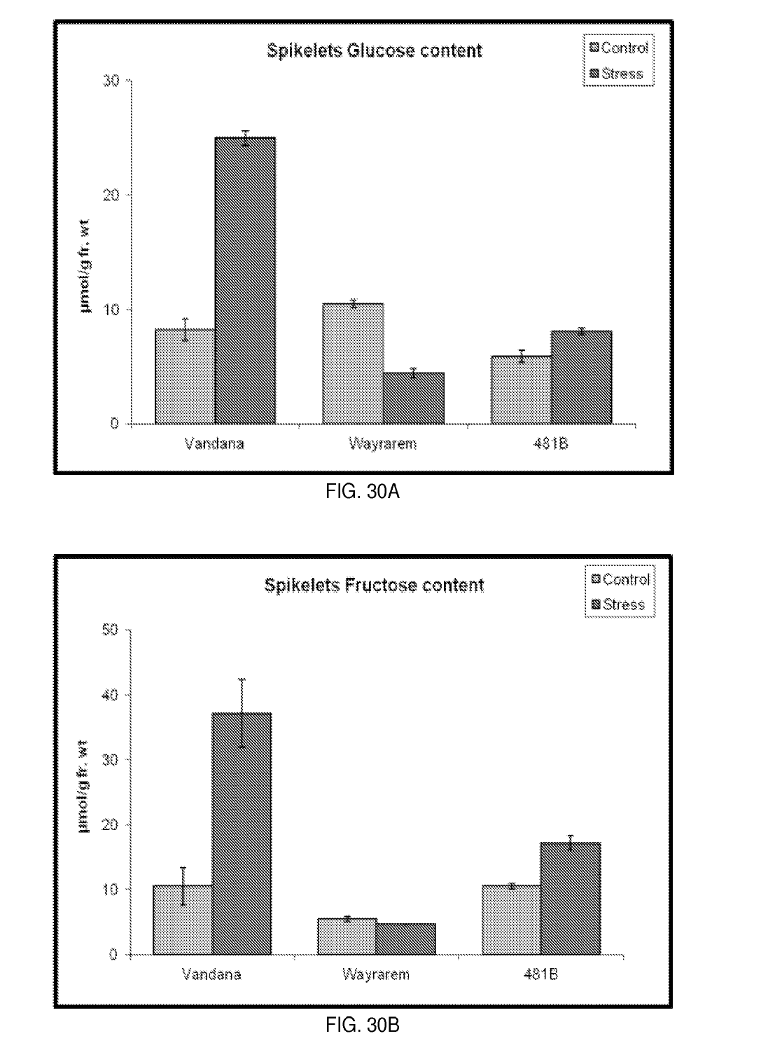

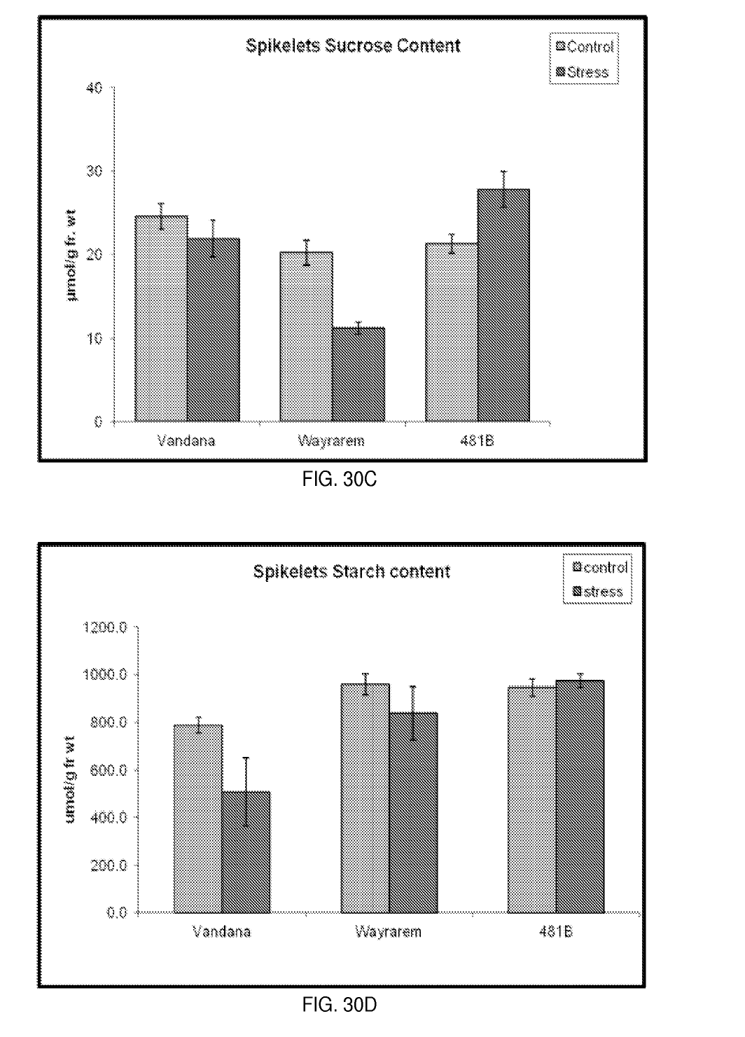

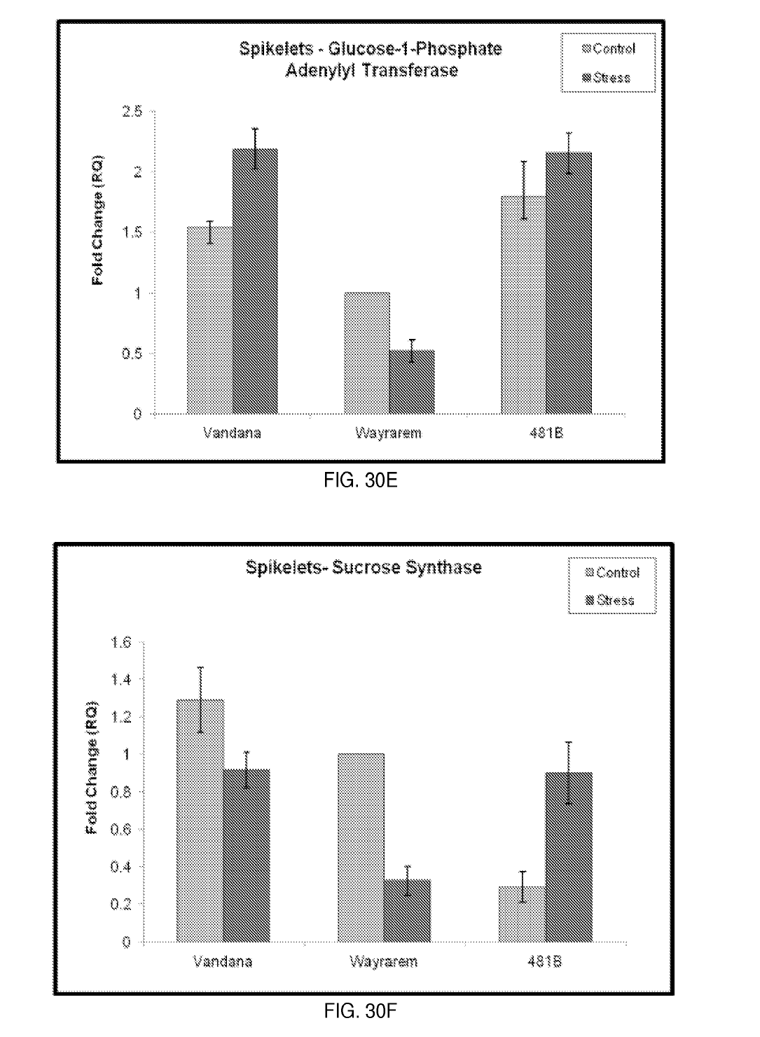

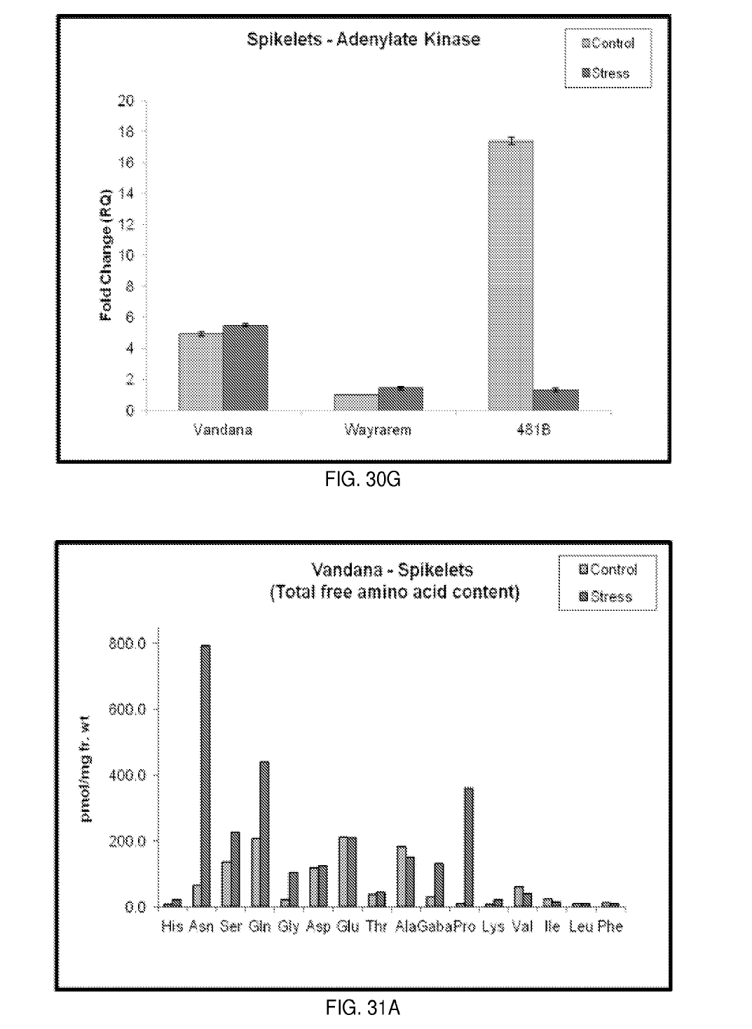

In the TMT data, the identified proteins represented comparative abundance in the 481-B with respect to that in Vandana. Proteins represented by a single peptide and with a MASCOT score of <40 were eliminated from consideration. A total of 332, 430 and 229 proteins (991 in all) were identified respectively from flag leaf, spikelets and roots as differentially expressed between Vandana and 481-B (Tables 5-7). Maximum number of proteins unique to a tissue was identified in the spikelets (167) followed by roots (106) and flag leaf (91; FIG. 28A). Flag leaf and spikelets shared maximum number of common proteins (206) between themselves. Proteins on the final list were represented as TIGR Locus identifiers and mapped onto different functional categories using the MAPMAN tool. A dynamic range of proteins was successfully identified as indicated by the coverage of proteins from a broad range of isoelectric point (4.27-11.47) and molecular weight (7 kDa-285 kDa). These were mapped onto the rice-mapping file in MAPMAN, and assigned to respective BINS (FIG. 30B). Following the MapMan ontology (Thimm et al., 2004), proteins were sorted into 26 functional categories (Tables 5-7). Considering the three tissues together, 100 proteins accounting for nearly 10% of the total identified, belonged to the group ‘protein metabolism’ (Tables 5-7). Other functional categories containing high number of proteins were redox proteins (9.31%), photosynthesis (8.55%), and stress proteins (6.37%).

Protein and Metabolite Factors Responsible for the Drought-Induced Lateral Root Growth Phenotype of qDTY12.1.

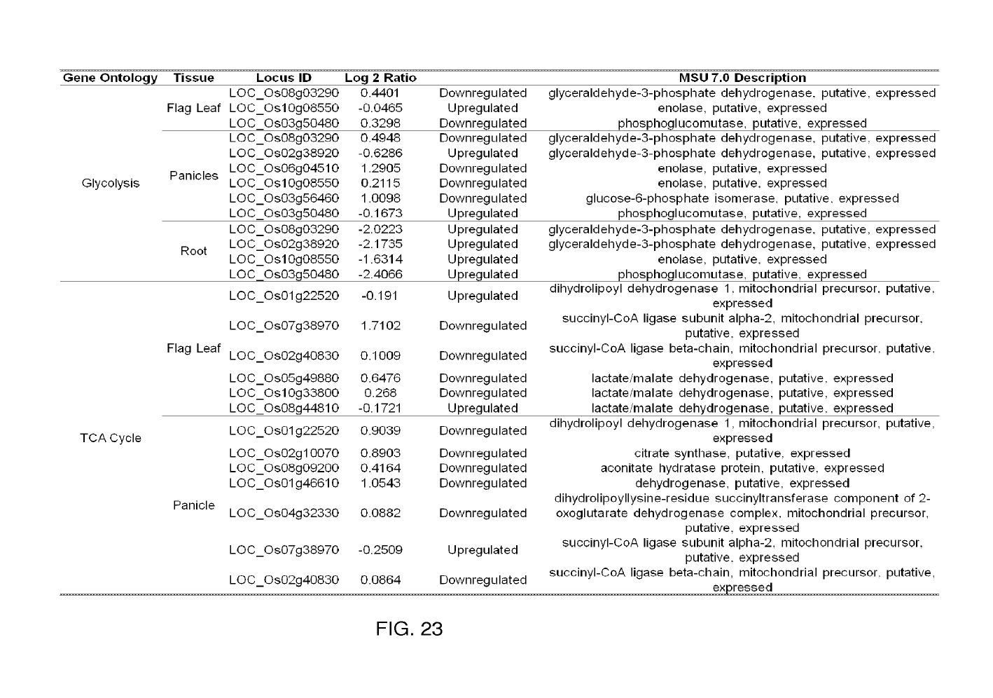

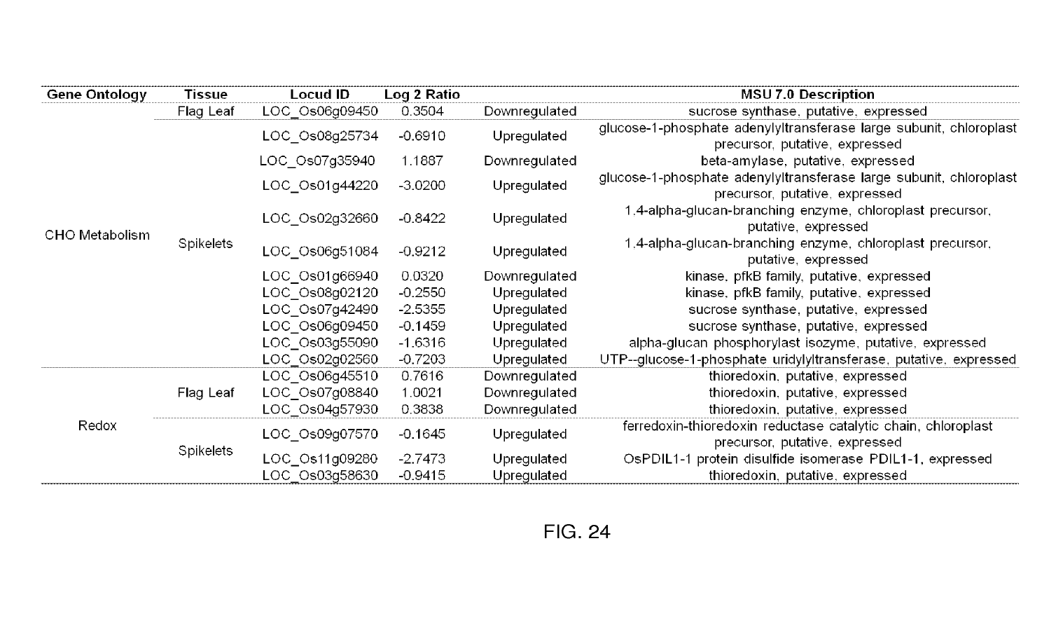

Increased lateral root growth and branching was observed in the qDTY₁₂ 481-B in comparison to Vandana under drought stress (Example 1). Roots help in continued acquisition of water and nutrients and increased lateral root and root hair proliferation has been implicated in plant sustenance under drought. Proteins implicated in the increased lateral root growth of the 481-B under drought were identified. For example, actin, tubulins and expansins were upregulated in the 481-B compared to Vandana, while actin-depolymerizing factor (ADF) was down regulated in the 481-B (FIG. 23). Actin is a ubiquitous cytoskeletal protein that is essential for a variety of purposes including cell division, and maintenance of cell integrity. Tubulins, another class of cytoskeletal proteins are functional in cell division and expansins aid in loosening the cell wall, allowing growth or expansion of the cell especially under low water potential when expansin expression is induced to aid in the cell wall plasticity. Inhibition of ADF is known to be beneficial for cell expansion and organ growth, thus ADF reduction in the 481-B facilitates lateral root growth. In conjunction with cytoskeletal and structural proteins being favorably expressed, Glyceraldehyde-3-phosphate dehydrogenase, enolase and glucose-6-phosphate isomerase belonging to the glycolytic pathway were more abundant in the roots of the 481-B (FIG. 24) indicating a favorable energy supply situation in the roots of the 481-B during stress.

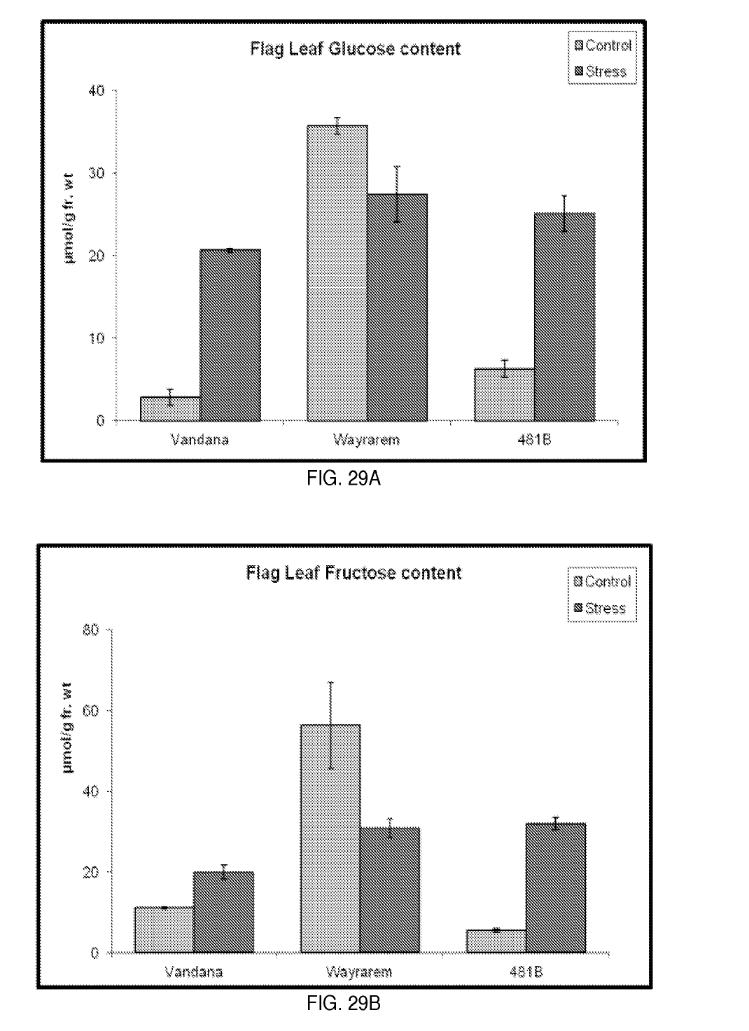

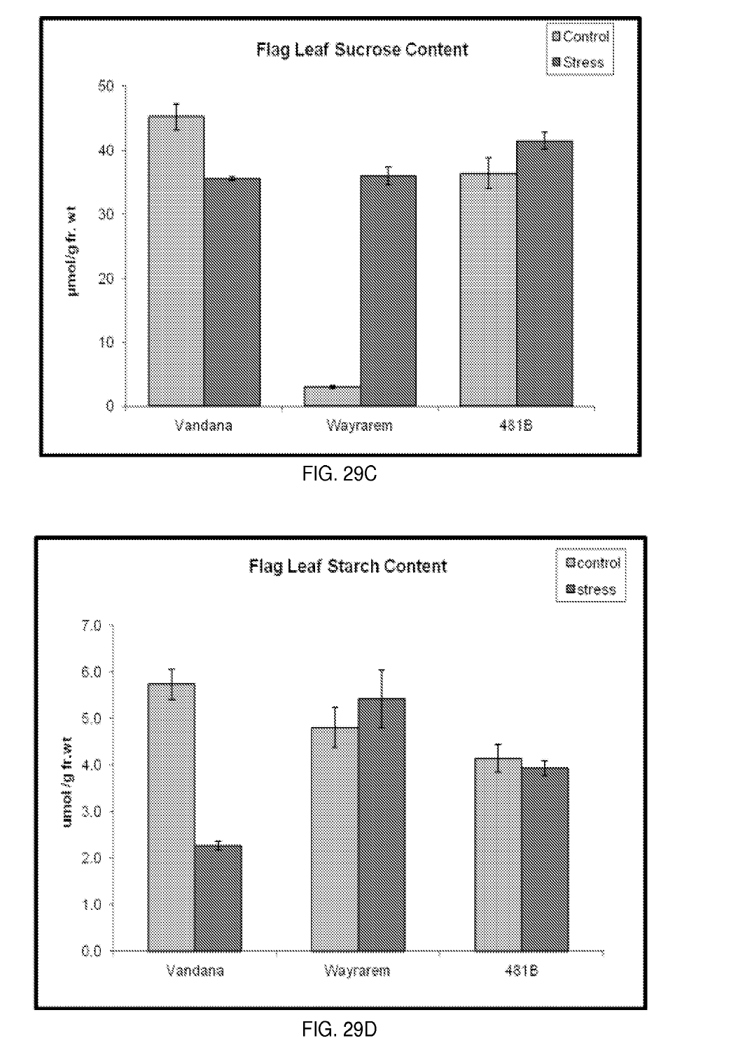

Comparatively more sucrose, fructose and glucose and less starch existed in the roots of the 481-B compared to Vandana (FIGS. 29A-29D). Freixes et al., (2002) demonstrated a high correlation between localized hexose concentration and fast growing, highly branched roots. Increase in the hexose concentration in the root increased respiration rates in wheat (Bingham and Stevenson, 1993). Thus, more sugars and less starch in the 481-B roots show the availability of the sugars more towards the energy needs for lateral root growth. Sugars are also used towards synthesizing cellulose for root growth. A cellulose synthase (OsCesA_{12.1}: LOC_Os12g29300) is one of the candidate genes in qDTY_{12.1} and it is comparatively more upregulated under drought in the 481-B roots, while an activation tag T20 DNA insertion mutant overexpressing OsCesA_{12.1} exhibited increased lateral roots under water deficit (Example 1). Therefore, the sugars in the roots of the 481-B are utilized to provide the energetic and structural component to drive lateral root growth, while their conversion into higher amounts of starch in Vandana may not be helpful. Starch accumulation in roots under drought stress occurred in a variety of plants and was correlated with impaired growth (Galvez et al., 2011), as in Vandana.

Increased accumulation of serine was also observed in the roots of the NILs compared to the parents during stress (FIGS. 29G-291). There is evidence that serine is involved in plant responses to various environmental stresses (Ho & Saito, 2001). Waditee et al., (2007) designed a strategy to increase the serine content of Arabidopsis to enhance plant tolerance to different abiotic stress, by transferring the 3-phosphoglycerate dehydrogenase gene. This led to induction of the betaine synthesis pathway and thus increased stress tolerance. An increased expression of 3-phosphoglycerate dehydrogenase and its content in the roots of the 481-B (FIGS. 23, 29K) was observed. These results demonstrate the involvement of this gene in the production of serine. Serine produced is involved directly or indirectly in generation of osmolytes like betaine, thus increasing 481-B capacity to counter stress. Serine also acts as a precursor of other limited essential amino acids in crop plants, such as methionine and cysteine.

TABLE 5
The complete set of proteins identified in flag leaf of Vandana and the 481B NIL (in triplicates),
represented as fold increase in the NIL compared to Vandana during drought stress.

| | | | Log 2 | | |
|---|---|---|---|---|---|
| Gene Ontology | MapMan Bin Description | Locus Id's | Ratio | | MSU v7.0 description |
| Photosystems | PS.lightreaction.photosystem | LOC_Os07g04840 | 0.6617 | Downregulated | PsbP, putative, expressed |
| | II.PSII polypeptide subunits | | | | |
| | PS.lightreaction.photosystem | LOC_Os01g43070 | 1.2892 | Downregulated | psbP-related thylakoid lumenal protein |
| | II.PSII polypeptide subunits | | | | 4, chloroplast precursor, putative, |
| | | | | | expressed |
| | PS.lightreaction.photosystem | LOC_Os01g31690 | −0.4935 | Upregulated | oxygen-evolving enhancer protein 1, |
| | II.PSII polypeptide subunits | | | | chloroplast precursor, putative, |
| | | | | | expressed |
| e-Carrier | PS.lightreaction.other electron | LOC_Os06g01210 | 0.5169 | Downregulated | plastocyanin, chloroplast precursor, |
| | carrier (ox/red).plastocyanin | | | | putative, expressed |
| | PS.lightreaction.other electron | LOC_Os08g01380 | −1.1488 | Upregulated | 2Fe-2S iron-sulfur cluster binding |
| | carrier (ox/red).ferredoxin | | | | domain containing protein, expressed |
| | PS.lightreaction.other electron | LOC_Os06g01850 | 1.5308 | Downregulated | ferredoxin--NADP reductase, |
| | carrier (ox/red).ferredoxin | | | | chloroplast precursor, putative, |
| | reductase | | | | expressed |
| | PS.lightreaction.other electron | LOC_Os02g01340 | −0.9887 | Upregulated | ferredoxin--NADP reductase, |
| | carrier (ox/red).ferredoxin | | | | chloroplast precursor, putative, |
| | reductase | | | | expressed |
| | PS.lightreaction.other electron | LOC_Os02g22260 | 0.0792 | Downregulated | fruit protein PKIWI502, putative, |
| | carrier (ox/red).ferredoxin | | | | expressed |
| | oxireductase | | | | |
| Photorespiration | PS.photorespiration.glycolate | LOC_Os04g53210 | 0.6346 | Downregulated | hydroxyacid oxidase 1, putative, |
| | oxydase | | | | expressed |
| | PS.photorespiration.glycolate | LOC_Os07g05820 | −0.1254 | Upregulated | hydroxyacid oxidase 1, putative, |
| | oxydase | | | | expressed |
| | PS.photorespiration.aminotrans | LOC_Os08g39300 | −0.0553 | Upregulated | aminotransferase, putative, expressed |
| | ferases peroxisomal | | | | |
| | PS.photorespiration.glycine | LOC_Os06g40940 | −0.0997 | Upregulated | glycine dehydrogenase, putative, |
| | cleavage.P subunit | | | | expressed |
| | PS.photorespiration.glycine | LOC_Os10g37180 | −0.8185 | Upregulated | glycine cleavage system H protein, |
| | cleavage.H protein | | | | putative, expressed |
| | PS.photorespiration.serine | LOC_Os03g52840 | −0.1907 | Upregulated | serine hydroxymethyltransferase, |
| | hydroxymethyltransferase | | | | mitochondrial precursor, putative, |
| | | | | | expressed |
| | PS.photorespiration. | LOC_Os02g01150 | −0.4586 | Upregulated | erythronate-4-phosphate |
| | hydroxypyruvate reductase | | | | dehydrogenase domain containing |
| | | | | | protein, expressed |
| Calvin Cycle | PS.calvin cycle | LOC_Os01g19740 | −1.4999 | Upregulated | calvin cycle protein CP12, putative, |
| | | | | | expressed |
| | PS.calvin | LOC_Os06g45710 | 0.5105 | Downregulated | phosphoglycerate kinase protein, |
| | cycle.phosphoglycerate kinase | | | | putative, expressed |
| | PS.calvin | LOC_Os05g41640 | −0.4569 | Upregulated | phosphoglycerate kinase protein, |
| | cycle.phosphoglycerate kinase | | | | putative, expressed |
| | PS.calvin | LOC_Os02g07260 | 0.8231 | Downregulated | phosphoglycerate kinase protein, |
| | cycle.phosphoglycerate kinase | | | | putative, expressed |
| | PS.calvin cycle.GAP | LOC_Os03g03720 | 0.2524 | Downregulated | glyceraldehyde-3-phosphate |
| | | | | | dehydrogenase, putative, expressed |
| | PS.calvin cycle.GAP | LOC_Os04g38600 | −0.1382 | Upregulated | glyceraldehyde-3-phosphate |
| | | | | | dehydrogenase, putative, expressed |
| | PS.calvin cycle.GAP | LOC_Os08g34210 | −0.6305 | Upregulated | aldehyde dehydrogenase, putative, |
| | | | | | expressed |
| | PS.calvin cycle.TPI | LOC_Os01g05490 | −1.3238 | Upregulated | triosephosphate isomerase, cytosolic, |
| | | | | | putative, expressed |
| | PS.calvin cycle.TPI | LOC_Os09g36450 | 0.6892 | Downregulated | triosephosphate isomerase, chloroplast |
| | | | | | precursor, putative, expressed |
| | PS.calvin cycle.aldolase | LOC_Os11g07020 | −0.9809 | Upregulated | fructose-bisphospate aldolase isozyme, |
| | | | | | putative, expressed |
| | PS.calvin cycle.aldolase | LOC_Os01g67860 | 0.6298 | Downregulated | fructose-bisphospate aldolase isozyme, |
| | | | | | putative, expressed |
| | PS.calvin cycle.aldolase | LOC_Os05g33380 | 0.1392 | Downregulated | fructose-bisphospate aldolase isozyme, |
| | | | | | putative, expressed |
| | PS.calvin cycle.aldolase | LOC_Os06g40640 | 0.3000 | Downregulated | fructose-bisphospate aldolase isozyme, |
| | | | | | putative, expressed |
| | PS.calvin cycle.FBPase | LOC_Os03g16050 | −0.0685 | Upregulated | fructose-1,6-bisphosphatase, putative, |
| | | | | | expressed |
| | PS.calvin cycle.FBPase | LOC_Os01g64660 | 0.3439 | Downregulated | fructose-1,6-bisphosphatase, putative, |
| | | | | | expressed |
| | PS.calvin cycle.transketolase | LOC_Os06g04270 | −0.1232 | Upregulated | transketolase, chloroplast precursor, |
| | | | | | putative, expressed |
| | PS.calvin cycle.seduheptulose | LOC_Os04g16680 | −0.7328 | Upregulated | fructose-1,6-bisphosphatase, putative, |
| | bisphosphatase | | | | expressed |
| | PS.calvin cycle.RPE | LOC_Os03g07300 | −1.8252 | Upregulated | ribulose-phosphate 3-epimerase, |
| | | | | | chloroplast precursor, putative, |
| | | | | | expressed |
| | PS.calvin cycle.PRK | LOC_Os02g47020 | −0.4390 | Upregulated | phosphoribulokinase/Uridine kinase |
| | | | | | family protein, expressed |
| | PS.calvin cycle.rubisco | LOC_Os11g47970 | 0.1846 | Downregulated | AAA-type ATPase family protein, |
| | interacting | | | | putative, expressed |
| Thylakoid | not assigned.unknown | LOC_Os01g05080 | 0.8638 | Downregulated | thylakoid lumenal protein, putative, |
| Proteins | | | | | expressed |
| | not assigned.unknown | LOC_Os10g35810 | 0.4761 | Downregulated | thylakoid lumenal protein, putative, |
| | | | | | expressed |
| | not assigned.unknown | LOC_Os07g37250 | −1.4741 | Upregulated | THYLAKOID FORMATION1, |
| | | | | | chloroplast precursor, putative, |
| | | | | | expressed |
| CHO | major CHO | LOC_Os06g09450 | 0.3504 | Downregulated | sucrose synthase, putative, expressed |
| Metabolism | metabolism.degradation.sucrose. | | | | |
| | Susy | | | | |
| | minor CHO metabolism. | LOC_Os02g07350 | −0.1104 | Upregulated | inositol-1-monophosphatase, putative, |
| | myoinositol.inositol phosphatase | | | | expressed |
| | minor CHO metabolism.others | LOC_Os03g41510 | −0.0057 | Upregulated | oxidoreductase, aldo/keto reductase |
| | | | | | family protein, putative, expressed |
| | minor CHO metabolism.misc | LOC_Os04g41340 | −0.5467 | Upregulated | 4-nitrophenylphosphatase, putative, |
| | | | | | expressed |
| | C1-metabolism | LOC_Os06g40940 | −0.0997 | Upregulated | glycine dehydrogenase, putative, |
| | | | | | expressed |
| | not assigned.unknown | LOC_Os08g27840 | 0.3099 | Downregulated | phosphoenolpyruvate carboxylase, |
| | | | | | putative, expressed |
| | not assigned.unknown | LOC_Os02g02560 | −0.6838 | Upregulated | UTP--glucose-1-phosphate |
| | | | | | uridylyltransferase, putative, expressed |
| | not assigned.unknown | LOC_Os01g60190 | 1.3407 | Downregulated | 2,3-bisphosphoglycerate-independent |
| | | | | | phosphoglycerate mutase, putative, |
| | | | | | expressed |
| Glycolysis | glycolysis.cytosolic | LOC_Os08g03290 | 0.4401 | Downregulated | glyceraldehyde-3-phosphate |
| | branch.glyceraldehyde 3- | | | | dehydrogenase, putative, expressed |
| | phosphate dehydrogenase | | | | |
| | (GAP-DH) | | | | |
| | glycolysis.cytosolic | LOC_Os10g08550 | −0.0465 | Upregulated | enolase, putative, expressed |
| | branch.enolase | | | | |
| | not assigned.unknown | LOC_Os03g50480 | 0.3298 | Downregulated | phosphoglucomutase, putative, |
| | | | | | expressed |
| Fermentation | fermentation.aldehyde | LOC_Os06g15990 | −0.1343 | Upregulated | aldehyde dehydrogenase, putative, |
| | dehydrogenase | | | | expressed |
| Oxidative | OPP.oxidative PP.6- | LOC_Os02g35500 | 0.2040 | Downregulated | NAD binding domain of 6- |
| Pentose | phosphogluconate | | | | phosphogluconate dehydrogenase |
| Phosphate | dehydrogenase | | | | containing protein, expressed |
| Pathway | OPP.non-reductive | LOC_Os01g70170 | 0.7529 | Downregulated | transaldolase, putative, expressed |
| | PP.transaldolase | | | | |
| | OPP.non-reductive PP.ribose | LOC_Os07g08030 | −2.2251 | Upregulated | ribose-5-phosphate isomerase A, |
| | 5-phosphate isomerase | | | | putative, expressed |
| TCA Cycle | TCA/org. | LOC_Os01g22520 | −0.1910 | Upregulated | dihydrolipoyl dehydrogenase 1, |
| | transformation.TCA.pyruvate | | | | mitochondrial precursor, putative, |
| | DH.E3 | | | | expressed |
| | TCA/org. | LOC_Os07g38970 | 1.7102 | Downregulated | succinyl-CoA ligase subunit alpha-2, |
| | transformation.TCA.succinyl- | | | | mitochondrial precursor, putative, |
| | CoA ligase | | | | expressed |
| | TCA/org. | LOC_Os02g40830 | 0.1009 | Downregulated | succinyl-CoA ligase beta-chain, |
| | transformation.TCA.succinyl- | | | | mitochondrial precursor, putative, |
| | CoA ligase | | | | expressed |
| | TCA/org. | LOC_Os05g49880 | 0.6476 | Downregulated | lactate/malate dehydrogenase, putative, |
| | transformation.TCA.malate | | | | expressed |
| | DH | | | | |
| | TCA/org. | LOC_Os10g33800 | 0.2680 | Downregulated | lactate/malate dehydrogenase, putative, |
| | transformation.other organic | | | | expressed |
| | acid transformaitons.cyt MDH | | | | |
| | TCA/org. | LOC_Os08g44810 | −0.1721 | Upregulated | lactate/malate dehydrogenase, putative, |
| | transformation.other organic | | | | expressed |
| | acid transformaitons.cyt MDH | | | | |
| | TCA/org. | LOC_Os01g45274 | −0.2152 | Upregulated | carbonic anhydrase, chloroplast |
| | transformation.carbonic | | | | precursor, putative, expressed |
| | anhydrases | | | | |
| Mitochondrial | mitochondrial electron | LOC_Os05g47980 | −0.2750 | Upregulated | ATP synthase, putative, expressed |
| Electron | transport/ATP synthesis.F1- | | | | |
| Transport | ATPase | | | | |
| Chain | not assigned.unknown | LOC_Os01g72430 | 0.1178 | Downregulated | NADPH quinone oxidoreductase, |
| | | | | | putative, expressed |
| Cell and Cell | cell wall.degradation.mannan- | LOC_Os04g54810 | 1.1963 | Downregulated | beta-D-xylosidase, putative, expressed |
| Wall Related | xylose-arabinose-fucose | | | | |
| Proteins | cell.organisation | LOC_Os09g04790 | −0.4957 | Upregulated | PAP fibrillin family domain containing |
| | | | | | protein, expressed |
| | cell.organisation | LOC_Os01g64630 | 0.1352 | Downregulated | actin, putative, expressed |
| | cell.division | LOC_Os07g38300 | −2.0972 | Upregulated | ribosome recycling factor, putative, |
| | | | | | expressed |
| | cell.cycle.peptidylprolyl | LOC_Os02g02890 | 0.4207 | Downregulated | peptidyl-prolyl cis-trans isomerase, |
| | isomerase | | | | putative, expressed |
| Lipid | lipid metabolism.″exotics″ | LOC_Os01g57570 | 0.2503 | Downregulated | NADPH-dependent FMN reductase |
| Metabolism | (steroids, squalene etc) | | | | domain containing protein, expressed |
| Amino Acid | amino acid | LOC_Os02g55420 | −0.4215 | Upregulated | aminotransferase, classes I and II, |
| Metabolism | metabolism.synthesis.central | | | | domain containing protein, expressed |
| | amino acid | | | | |
| | metabolism.aspartate.aspartate | | | | |
| | aminotransferase | | | | |
| | amino acid | LOC_Os07g01760 | −0.1129 | Upregulated | aminotransferase, classes I and II, |
| | metabolism.synthesis.central | | | | domain containing protein, expressed |
| | amino acid | | | | |
| | metabolism.alanine.alanine | | | | |
| | aminotransferase | | | | |
| | amino acid | LOC_Os08g39300 | −0.0553 | Upregulated | aminotransferase, putative, expressed |
| | metabolism.synthesis.central | | | | |
| | amino acid | | | | |
| | metabolism.alanine.alanine- | | | | |
| | glyoxylate aminotransferase | | | | |
| | amino acid | LOC_Os03g45320 | −0.1042 | Upregulated | dehydrogenase, putative, expressed |
| | metabolism.synthesis.branched | | | | |
| | chain group.leucine specific.3- | | | | |
| | isopropylmalate dehydrogenase | | | | |
| | amino acid | LOC_Os08g39300 | −0.0553 | Upregulated | aminotransferase, putative, expressed |
| | metabolism.synthesis.serine- | | | | |
| | glycine-cysteine | | | | |
| | group.glycine.serine glyoxylate | | | | |
| | aminotransferase | | | | |
| | amino acid | LOC_Os01g74650 | −0.1286 | Upregulated | cysteine synthase, mitochondrial |
| | metabolism.synthesis.serine- | | | | precursor, putative, expressed |
| | glycine-cysteine | | | | |
| | group.cysteine.OASTL | | | | |
| | amino acid | LOC_Os08g09250 | 0.2586 | Downregulated | glyoxalase family protein, putative, |
| | metabolism.degradation.aspartate | | | | expressed |
| | family.threonine | | | | |
| | amino acid | LOC_Os07g09060 | 0.1049 | Downregulated | aldehyde dehydrogenase, putative, |
| | metabolism.degradation. | | | | expressed |
| | branched-chain group.valine | | | | |
| | amino acid | LOC_Os04g53230 | 0.4622 | Downregulated | aminomethyltransferase, putative, |
| | metabolism.degradation.serine- | | | | expressed |
| | glycine-cysteine group.glycine | | | | |
| Secondary | secondary | LOC_Os07g36190 | 0.2424 | Downregulated | hydrolase, NUDIX family, domain |
| Metabolism | metabolism.isoprenoids. | | | | containing protein, expressed |
| | mevalonate pathway.isopentenyl | | | | |
| | pyrophosphate:dimethyllallyl | | | | |
| | pyrophosphate isomerase | | | | |
| | secondary metabolism.sulfur- | LOC_Os03g45320 | −0.1042 | Upregulated | dehydrogenase, putative, expressed |
| | containing.glucosinolates.synthesis. | | | | |
| | aliphatic.methylthioalkylmalate | | | | |
| | dehydrogenase (MAM-D) | | | | |
| Hormones | hormone | LOC_Os01g43090 | −0.9586 | Upregulated | oxidoreductase, aldo/keto reductase |
| | metabolism.auxin.induced- | | | | family protein, putative, expressed |
| | regulated-responsive-activated | | | | |
| | hormone | LOC_Os02g10120 | −0.2903 | Upregulated | lipoxygenase, putative, expressed |
| | metabolism.jasmonate.synthesis- | | | | |
| | degradation.lipoxygenase | | | | |
| Stress - Related | stress.biotic | LOC_Os10g34930 | −1.0021 | Upregulated | secretory protein, putative, expressed |
| Proteins | stress.abiotic.heat | LOC_Os09g30412 | 0.2942 | Downregulated | heat shock protein, putative, expressed |
| | stress.abiotic.heat | LOC_Os02g02410 | −0.1783 | Upregulated | DnaK family protein, putative, |
| | | | | | expressed |
| | stress.abiotic.heat | LOC_Os03g60620 | −0.0838 | Upregulated | DnaK family protein, putative, |
| | | | | | expressed |
| | stress.abiotic.heat | LOC_Os08g38086 | 0.1623 | Downregulated | heat shock protein, putative, expressed |
| | stress.abiotic.heat | LOC_Os04g01740 | 0.4459 | Downregulated | heat shock protein, putative, expressed |
| | stress.abiotic.heat | LOC_Os08g39140 | 0.2173 | Downregulated | heat shock protein, putative, expressed |
| | stress.abiotic.heat | LOC_Os02g53420 | 0.0308 | Downregulated | DnaK family protein, putative, |
| | | | | | expressed |
| | stress.abiotic.heat | LOC_Os11g47760 | 0.4390 | Downregulated | DnaK family protein, putative, |
| | | | | | expressed |
| | stress.abiotic.cold | LOC_Os02g02870 | 0.7089 | Downregulated | glycine-rich protein 2, putative, |
| | | | | | expressed |
| | stress.abiotic.cold | LOC_Os08g03520 | 2.0058 | Downregulated | retrotransposon protein, putative, Ty1- |
| | | | | | copia subclass, expressed |
| | stress.abiotic.unspecified | LOC_Os03g48770 | −1.8332 | Upregulated | Cupin domain containing protein, |
| | | | | | expressed |
| | stress.abiotic.unspecified | LOC_Os08g35760 | 1.5087 | Downregulated | Cupin domain containing protein, |
| | | | | | expressed |
| | not assigned.unknown | LOC_Os05g46480 | 0.9278 | Downregulated | late embryogenesis abundant protein, |
| | | | | | group 3, putative, expressed |
| | not assigned.unknown | LOC_Os11g06720 | 1.6806 | Downregulated | abscisic stress-ripening, putative, |
| | | | | | expressed |
| | not assigned.unknown | LOC_Os11g26750 | 1.2346 | Downregulated | dehydrin, putative, expressed |
| | not assigned.unknown | LOC_Os01g50910 | 0.3139 | Downregulated | late embryogenesis abundant protein, |
| | | | | | group 3, putative, expressed |
| Redox - Related | redox.thioredoxin | LOC_Os11g09280 | 1.0680 | Downregulated | OsPDIL1-1 protein disulfide isomerase |
| Proteins | | | | | PDIL1-1, expressed |
| | redox.thioredoxin | LOC_Os07g29410 | −0.4598 | Upregulated | thioredoxin, putative, expressed |
| | redox.thioredoxin | LOC_Os07g08840 | 1.0021 | Downregulated | thioredoxin, putative, expressed |
| | redox.ascorbate and | LOC_Os05g02530 | −0.4048 | Upregulated | glutathione S-transferase, N-terminal |
| | glutathione.ascorbate | | | | domain containing protein, expressed |
| | redox.ascorbate and | LOC_Os12g07820 | 0.6817 | Downregulated | OsAPx6 - Stromal Ascorbate |
| | glutathione.ascorbate | | | | Peroxidase encoding gene 5,8, |
| | | | | | expressed |
| | redox.ascorbate and | LOC_Os09g39380 | 0.2248 | Downregulated | monodehydroascorbate reductase, |
| | glutathione.ascorbate | | | | putative, expressed |
| Nucleotide | redox.ascorbate and | LOC_Os04g51300 | 0.3539 | Downregulated | peroxidase precursor, putative, |
| Metabolism | glutathione.ascorbate | | | | expressed |
| | redox.ascorbate and | LOC_Os07g49400 | 0.9700 | Downregulated | OsAPx2 - Cytosolic Ascorbate |
| | glutathione.ascorbate | | | | Peroxidase encoding gene 4, 5, 6, 8, |
| | | | | | expressed |
| | redox.ascorbate and | LOC_Os03g17690 | −1.3031 | Upregulated | OsAPx1 - Cytosolic Ascorbate |
| | glutathione.ascorbate | | | | Peroxidase encoding gene 1-8, |
| | | | | | expressed |
| | redox.ascorbate and | LOC_Os03g06740 | −0.8396 | Upregulated | glutathione reductase, putative, |
| | glutathione.glutathione | | | | expressed |
| | redox.ascorbate and | LOC_Os02g56850 | −0.3201 | Upregulated | glutathione reductase, putative, |
| | glutathione.glutathione | | | | expressed |
| | redox.ascorbate and | LOC_Os04g46960 | 0.6462 | Downregulated | glutathione peroxidase domain |
| | glutathione.glutathione | | | | containing protein, expressed |
| | redox.glutaredoxins | LOC_Os08g45140 | 0.1419 | Downregulated | OsGrx_S12 - glutaredoxin subgroup I, |
| | | | | | expressed |
| | redox.peroxiredoxin | LOC_Os06g09610 | −0.4154 | Upregulated | peroxiredoxin, putative, expressed |
| | redox.peroxiredoxin | LOC_Os02g09940 | −0.6841 | Upregulated | peroxiredoxin, putative, expressed |
| | redox.peroxiredoxin.BAS1 | LOC_Os02g33450 | −0.3210 | Upregulated | peroxiredoxin, putative, expressed |
| | redox.dismutases and catalases | LOC_Os05g25850 | 0.2342 | Downregulated | superoxide dismutase, mitochondrial |
| | | | | | precursor, putative, expressed |
| | redox.dismutases and catalases | LOC_Os07g46990 | 0.3759 | Downregulated | copper/zinc superoxide dismutase, |
| | | | | | putative, expressed |
| | redox.dismutases and catalases | LOC_Os08g44770 | −0.0177 | Upregulated | copper/zinc superoxide dismutase, |
| | | | | | putative, expressed |
| | misc.glutathione S transferases | LOC_Os01g55830 | 0.2875 | Downregulated | glutathione S-transferase, putative, |
| | | | | | expressed |
| | misc.glutathione S transferases | LOC_Os10g39740 | 0.1535 | Downregulated | glutathione S-transferase, putative, |
| | | | | | expressed |
| | misc.glutathione S transferases | LOC_Os09g29200 | −2.1616 | Upregulated | glutathione S-transferase, putative, |
| | | | | | expressed |
| | misc.glutathione S transferases | LOC_Os03g04240 | 1.9698 | Downregulated | glutathione S-transferase, putative, |
| | | | | | expressed |
| | misc.glutathione S transferases | LOC_Os10g38700 | −3.4913 | Upregulated | glutathione S-transferase, putative, |
| | | | | | expressed |
| | misc.glutathione S transferases | LOC_Os03g04220 | 1.0953 | Downregulated | glutathione S-transferase, putative, |
| | | | | | expressed |
| | misc.peroxidases | LOC_Os01g22230 | −0.2554 | Upregulated | peroxidase precursor, putative, |
| | | | | | expressed |
| | misc.peroxidases | LOC_Os07g48020 | −0.4086 | Upregulated | peroxidase precursor, putative, |
| | | | | | expressed |
| | nucleotide | LOC_Os05g51700 | −0.3549 | Upregulated | nucleoside diphosphate kinase, |
| | metabolism.phosphotransfer | | | | putative, expressed |
| | and | | | | |
| | pyrophosphatases.nucleoside | | | | |
| | diphosphate kinase | | | | |
| | nucleotide | LOC_Os10g41410 | 0.4579 | Downregulated | nucleoside diphosphate kinase, |
| | metabolism.phosphotransfer | | | | putative, expressed |
| | and | | | | |
| | pyrophosphatases.nucleoside | | | | |
| | diphosphate kinase | | | | |
| | nucleotide | LOC_Os07g30970 | 1.1458 | Downregulated | nucleoside diphosphate kinase, |
| | metabolism.phosphotransfer | | | | putative, expressed |
| | and | | | | |
| | pyrophosphatases.nucleoside | | | | |
| | diphosphate kinase | | | | |
| | nucleotide | LOC_Os02g52940 | 0.9380 | Downregulated | soluble inorganic pyrophosphatase, |
| | metabolism.phosphotransfer | | | | putative, expressed |
| | and pyrophosphatases.misc | | | | |
| | DNA.synthesis/chromatin | LOC_Os10g32348 | −0.0669 | Upregulated | PsbP, putative, expressed |
| | structure | | | | |
| | not assigned.unknown | LOC_Os01g23680 | −0.0259 | Upregulated | rossmann fold nucleotide-binding |
| | | | | | protein involved in DNA uptake, |
| | | | | | putative, expressed |
| Miscellaneous | Biodegradation of Xenobiotics | LOC_Os08g09250 | 0.2586 | Downregulated | glyoxalase family protein, putative, |
| Enzymes | | | | | expressed |
| | Biodegradation of Xenobiotics | LOC_Os09g28630 | −0.4135 | Upregulated | gibberellin receptor, putative, |
| | | | | | expressed |
| | Biodegradation of | LOC_Os02g17920 | −0.0099 | Upregulated | glyoxalase family protein, putative, |
| | Xenobiotics.lactoylglutathione | | | | expressed |
| | lyase | | | | |
| | misc.misc2 | LOC_Os01g34700 | −1.5970 | Upregulated | dienelactone hydrolase family protein, |
| | | | | | expressed |
| | misc.oxidases - copper, flavone | LOC_Os08g29170 | −1.0250 | Upregulated | dehydrogenase, putative, expressed |
| | etc. | | | | |
| | misc.alcohol dehydrogenases | LOC_Os08g43190 | −0.1080 | Upregulated | dehydrogenase, putative, expressed |
| RNA | RNA.processing.ribonucleases | LOC_Os07g33240 | 0.0570 | Downregulated | endoribonuclease, putative, expressed |
| Processing | | | | | NAD dependent |
| | RNA.regulation of | LOC_Os07g11110 | −1.2597 | Upregulated | epimerase/dehydratase family protein, |
| | transcription.unclassified | | | | putative, expressed |
| | RNA.regulation of | LOC_Os04g31700 | −0.0375 | Upregulated | methylisocitrate lyase 2, putative, |
| | transcription.unclassified | | | | expressed |
| | RNA.RNA binding | LOC_Os09g10760 | 0.5613 | Downregulated | RNA recognition motif containing |
| | | | | | protein, putative, expressed |
| | RNA.RNA binding | LOC_Os07g43810 | 1.6657 | Downregulated | RNA recognition motif containing |
| | | | | | protein, putative, expressed |
| | RNA.RNA binding | LOC_Os09g39180 | 0.3136 | Downregulated | RNA recognition motif containing |
| | | | | | protein, putative, expressed |
| Protein | N-metabolism.ammonia | LOC_Os07g46460 | −0.9547 | Upregulated | ferredoxin-dependent glutamate |
| Metabolism, | metabolism.glutamate synthase | | | | synthase, chloroplast precursor, |
| Synthesis and | | | | | putative, expressed |
| Degradation | N-metabolism.ammonia | LOC_Os02g50240 | 0.5269 | Downregulated | glutamine synthetase, catalytic domain |
| | metabolism.glutamine synthase | | | | containing protein, expressed |
| | N-metabolism.ammonia | LOC_Os04g56400 | −0.2369 | Upregulated | glutamine synthetase, catalytic domain |
| | metabolism.glutamine synthase | | | | containing protein, expressed |
| | protein.synthesis.ribosomal | LOC_Os03g62630 | −0.9647 | Upregulated | ribosomal protein S6, putative, |
| | protein.prokaryotic.chloroplast. | | | | expressed |
| | 30S subunit.S6 | | | | |
| | protein.synthesis.ribosomal | LOC_Os01g47330 | −2.3320 | Upregulated | ribosomal protein L7/L12 C-terminal |
| | protein.prokaryotic.chloroplast. | | | | domain containing protein, expressed |
| | 50S subunit.L12 | | | | |
| | protein.synthesis.ribosomal | LOC_Os01g54540 | 0.3782 | Downregulated | ribosomal protein L13, putative, |
| | protein.prokaryotic.chloroplast. | | | | expressed |
| | 50S subunit.L13 | | | | |
| | protein.synthesis.ribosomal | LOC_Os01g57956 | −0.8244 | Upregulated | chloroplast 50S ribosomal protein L14, |
| | protein.prokaryotic.chloroplast. | | | | putative, expressed |
| | 50S subunit.L14 | | | | |
| | protein.synthesis.ribosomal | LOC_Os04g16828 | −0.8244 | Upregulated | chloroplast 50S ribosomal protein L14, |
| | protein.prokaryotic.chloroplast. | | | | putative, expressed |
| | 50S subunit.L14 | | | | |
| | protein.synthesis.ribosomal | LOC_Os10g21342 | −0.8244 | Upregulated | chloroplast 50S ribosomal protein L14, |
| | protein.prokaryotic.chloroplast. | | | | putative, expressed |
| | 50S subunit.L14 | | | | |
| | protein.synthesis.ribosomal | LOC_Os08g15276 | −0.8244 | Upregulated | chloroplast 50S ribosomal protein L14, |
| | protein.prokaryotic.chloroplast. | | | | putative, expressed |
| | 50S subunit.L14 | | | | |
| | protein.synthesis.ribosomal | LOC_Os05g22722 | −0.8244 | Upregulated | chloroplast 50S ribosomal protein L14, |
| | protein.prokaryotic.chloroplast. | | | | putative, expressed |
| | 50S subunit.L14 | | | | |
| | protein.synthesis.ribosomal | LOC_Os02g06700 | 1.3440 | Downregulated | ribosomal protein, putative, expressed |
| | protein.eukaryotic.40S | | | | |
| | subunit.S14 | | | | |
| | protein.synthesis.elongation | LOC_Os02g38210 | 0.4323 | Downregulated | elongation factor Tu, putative, |
| | | | | | expressed |
| | protein.postranslational | LOC_Os04g40600 | 0.1861 | Downregulated | peptide methionine sulfoxide |
| | modification | | | | reductase, putative, expressed |
| | protein.postranslational | LOC_Os10g41400 | −0.8327 | Upregulated | peptide methionine sulfoxide |
| | modification | | | | reductase, putative, expressed |
| | protein.degradation | LOC_Os03g60740 | 0.6427 | Downregulated | expressed protein |
| | protein.degradation | LOC_Os02g58340 | 0.1390 | Downregulated | oligopeptidase, putative, expressed |
| | protein.degradation | LOC_Os08g44860 | −0.3903 | Upregulated | aminopeptidase, putative, expressed |
| | protein.degradation | LOC_Os02g55140 | 0.0521 | Downregulated | leucine aminopeptide, chloroplast |
| | | | | | precursor, putative, expressed |
| | protein.degradation.cysteine | LOC_Os04g55650 | 0.0631 | Downregulated | oryzain alpha chain precursor, |
| | protease | | | | putative, expressed |
| | protein.degradation.serine | LOC_Os04g32560 | 0.1069 | Downregulated | ATP-dependent Clp protease ATP- |
| | protease | | | | binding subunit clpA homolog |
| | | | | | CD4B, chloroplast precursor, putative, |
| | | | | | expressed |
| | protein.degradation.serine | LOC_Os02g42290 | −0.3302 | Upregulated | OsClp3 - Putative Clp protease |
| | protease | | | | homologue, expressed |
| | protein.degradation.ubiquitin. | LOC_Os05g09490 | −2.0062 | Upregulated | peptidase, T1 family, putative, |
| | proteasom | | | | expressed |
| | protein.degradation.ubiquitin. | LOC_Os06g06030 | −0.0115 | Upregulated | peptidase, T1 family, putative, |
| | proteasom | | | | expressed |
| | protein.degradation.ubiquitin. | LOC_Os02g04100 | 0.1947 | Downregulated | peptidase, T1 family, putative, |
| | proteasom | | | | expressed |
| | protein.degradation.ubiquitin. | LOC_Os08g43540 | 0.1595 | Downregulated | peptidase, T1 family, putative, |
| | proteasom | | | | expressed |
| | protein.folding | LOC_Os09g26730 | −1.9708 | Upregulated | chaperonin, putative, expressed |
| | protein.folding | LOC_Os03g64210 | 0.2617 | Downregulated | T-complex protein, putative, expressed |
| | protein.folding | LOC_Os06g02380 | 0.2695 | Downregulated | T-complex protein, putative, expressed |
| | | | | | peptidyl-prolyl cis-trans isomerase, |
| | protein.folding | LOC_Os06g45340 | −1.5229 | Upregulated | FKBP-type, putative, expressed |
| | protein.folding | LOC_Os02g39870 | −1.3684 | Upregulated | co-chaperone GrpE protein, putative, |
| | | | | | expressed |
| | protein.folding | LOC_Os07g44740 | −0.2338 | Upregulated | chaperonin, putative, expressed |
| | protein.folding | LOC_Os06g09688 | −1.4489 | Upregulated | chaperonin, putative, expressed |
| | protein.folding | LOC_Os10g32550 | 1.2254 | Downregulated | T-complex protein, putative, expressed |
| | protein.folding | LOC_Os10g41710 | −0.4063 | Upregulated | chaperonin, putative, expressed |
| | not assigned.unknown | LOC_Os05g32820 | 0.5295 | Downregulated | peptide-N4-asparagine amidase A, |
| | | | | | putative, expressed |
| Signaling | signalling.receptor | LOC_Os06g41560 | 0.1266 | Downregulated | receptor-like protein kinase, putative, |
| Proteins | kinases misc | | | | expressed |
| | signalling.calcium | LOC_Os03g29770 | −0.2462 | Upregulated | EF hand family protein, expressed |
| | signalling.14-3-3 proteins | LOC_Os08g33370 | 0.7724 | Downregulated | 14-3-3 protein, putative, expressed |
| | signalling.14-3-3 proteins | LOC_Os08g37490 | 0.4033 | Downregulated | 14-3-3 protein, putative, expressed |
| | signalling.14-3-3 proteins | LOC_Os03g50290 | 0.5988 | Downregulated | 14-3-3 protein, putative, expressed |
| | signalling.14-3-3 proteins | LOC_Os02g36974 | 0.1494 | Downregulated | 14-3-3 protein, putative, expressed |
| Development - | development.storage proteins | LOC_Os05g02520 | 0.4121 | Downregulated | cupin domain containing protein, |
| Related | | | | | expressed |
| Proteins | development.unspecified | LOC_Os04g57590 | −0.7016 | Upregulated | DJ-1 family protein, putative, |
| | | | | | expressed |
| Transport | transport.calcium | LOC_Os03g63420 | −0.4632 | Upregulated | OsGrx_S14 - glutaredoxin subgroup II, |
| | | | | | expressed |
| Unassigned | gluconeogenesis.Malate DH | LOC_Os03g56280 | −0.9337 | Upregulated | lactate/malate dehydrogenase, putative, |
| Classes | | | | | expressed |
| | metal handling | LOC_Os01g68770 | 0.9296 | Downregulated | selenium-binding protein, putative, |
| | | | | | expressed |
| | Co-factor and vitamine | LOC_Os10g01080 | −0.4295 | Upregulated | SOR/SNZ family protein, putative, |
| | metabolism | | | | expressed |
| | Co-factor and vitamine | LOC_Os07g34570 | 0.0057 | Downregulated | FAD dependent oxidoreductase |
| | metabolism.thiamine | | | | domain containing protein, expressed |
| | not assigned.unknown | LOC_Os03g60509 | 1.0562 | Downregulated | expressed protein |
| | not assigned.unknown | LOC_Os06g51330 | −0.8727 | Upregulated | expressed protein |

During stress, an increased content and up-regulation of an aldehyde dehydrogenase (methyl malonate-semialdehyde dehydrogenase, MMSDH) in the roots of the 481-B was observed compared to the parents (FIGS. 23, 29L). Aldehydes are intermediates in several fundamental metabolism pathways such as those involved with amino acid and carbohydrates. They are produced in response to environmental stress. Aldehyde dehydrogenases catalyze the irreversible oxidation of reactive aldehydes to their corresponding carboxylic acids and also act as efficient scavengers of reactive oxygen species (Perozich et al., 1999; Kirch et al., 2005). MMSDH specifically catalyzes the irreversible oxidative decarboxylation of malonate-semialdehydes and methylmalonate semialdehydes to acetyl-CoA which leads to more energy generation through TCA cycle (Oguchi et al., 2004). They play an important role in detoxifying the aldehydes as well as for energy generation in the roots of the NILs and thus help in maintaining better homeostasis required for better plant sustenance.

Protein and Metabolite Factors Involved in Drought-Induced Nitrogen Content Variations.

During drought stress, nitrogen (N) balance is of high importance to the plant. In rice under drought, growth and sustenance was better in the plants with N supply than in those without (Suralta, 2010). Increased lateral roots in the 481-B make for increased capacity to extract nutrients from the soil. N content under drought was more in the roots of the 481-B (FIG. 29E). Use of N by plants involves the processes of N uptake, assimilation, translocation and remobilization, wherein amino acids play a crucial role. Free amino acid content in the roots of the 481-B was more compared to the parents (FIGS. 29G-29J), showing that plants with more lateral roots were be the ones with better N status under drought. Proteome analysis also showed this. Aminotransferases, which are involved in N utilization through several amino acids (Robredo et al., 2011; Forde and Lea, 2007), were more abundant in the 481-B roots than in Vandana roots (FIG. 23). Glutamate synthetase was also more in the 481-B roots (FIG. 23) indicating increased glutamate amounts, which was observed to be in larger amounts in the 481-B than in Vandana 1 (FIGS. 29G-29J). Glutamate is one of the transportable amino acids, as well as a precursor for the synthesis of other amino acids such as arginine and proline (Ramanjulu et al., 1997).

Protein and Metabolite Factors for Better Plant Sustenance Under Drought.

Sugars and starch form the major reactants and products of carbohydrate metabolism, which is adjusted in response to environmental and developmental cues. Soluble sugars accumulate under stress and function as metabolic resources, structural constituents and signaling molecules in processes associated with plant growth and development (Jang & Sheen, 1997; Tran et al., 2007; Ho et al., 2001). Majority of the CO2 assimilated is converted into sucrose (Koch, 2004), which is the primary mobile sugar in the phloem translocated to the grains (Liu et al., 2012).

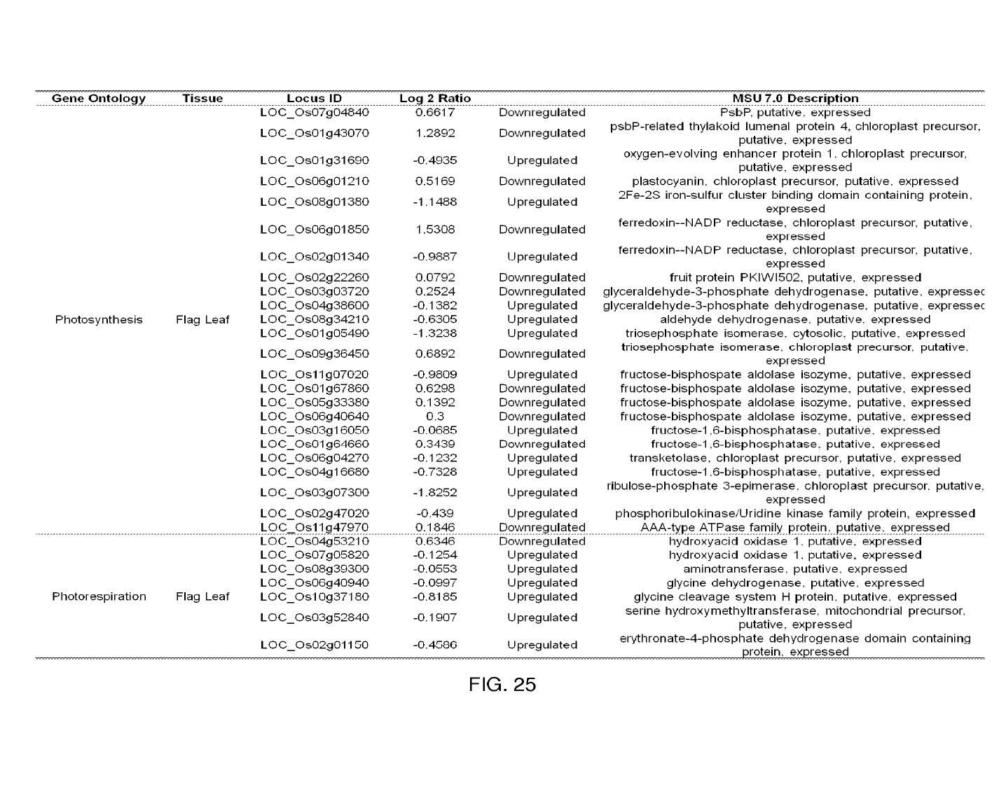

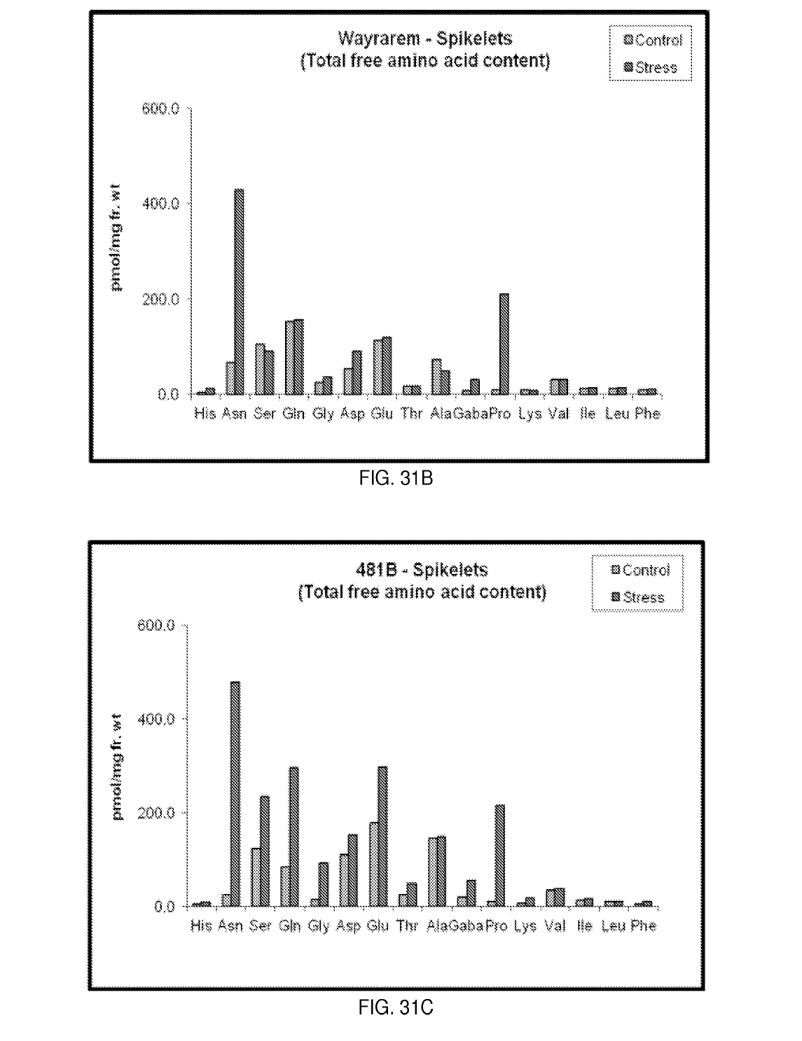

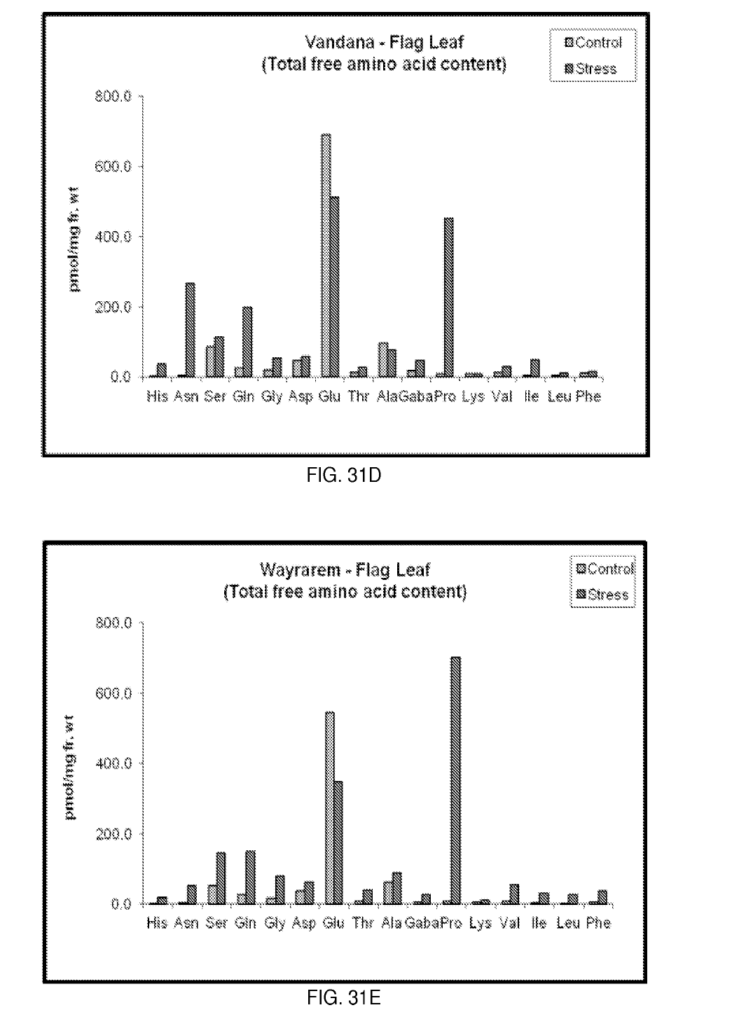

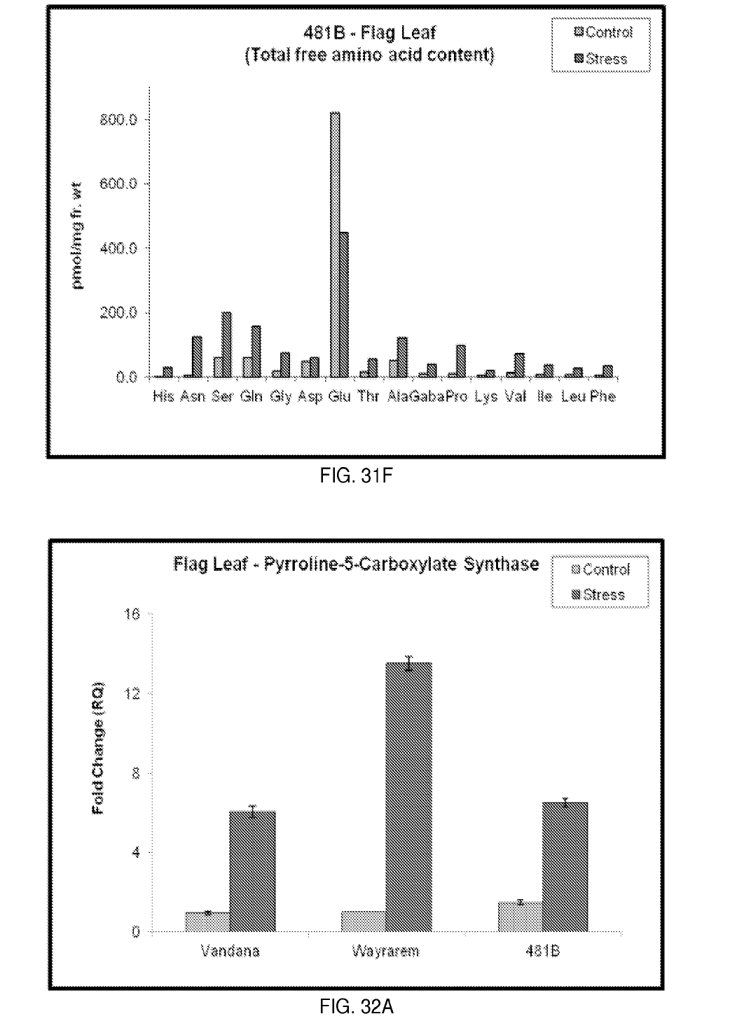

Sucrose has a role in stabilizing the membranes and proteins under water deficit (Gupta & Kaur, 2005). In the flag leaf of 481-B, a slight increase in the sucrose content was observed under stress compared to the control condition (FIG. 30C). However sucrose synthase was seen to be down-regulated in the 481-B compared to Vandana (FIG. 25). Down-regulation of sucrose synthase was an indication of feedback inhibition due to higher content of sucrose in the flag leaf. Higher accumulation of sucrose in the flag leaf of the 481-B perhaps indicated a better mechanism to be operative in the 481-B to maintain sufficient amounts of sucrose during stress as compared to the control conditions which can then be made available for remobilization to the spikelets during grain filling for starch synthesis. This was corroborated by increased sucrose and starch content in the 481-B spikelets under drought as compared to Vandana (FIGS. 31C-31D). As opposed to significant changes in the sucrose content in Vandana and WayRarem flag leaf under stress, the 481-B flag leaf exhibited limited change in sucrose content, which was also true for its starch content, while starch content in Vandana flag leaf and spikelets changed significantly under stress (FIGS. 30C, 31C-31D).

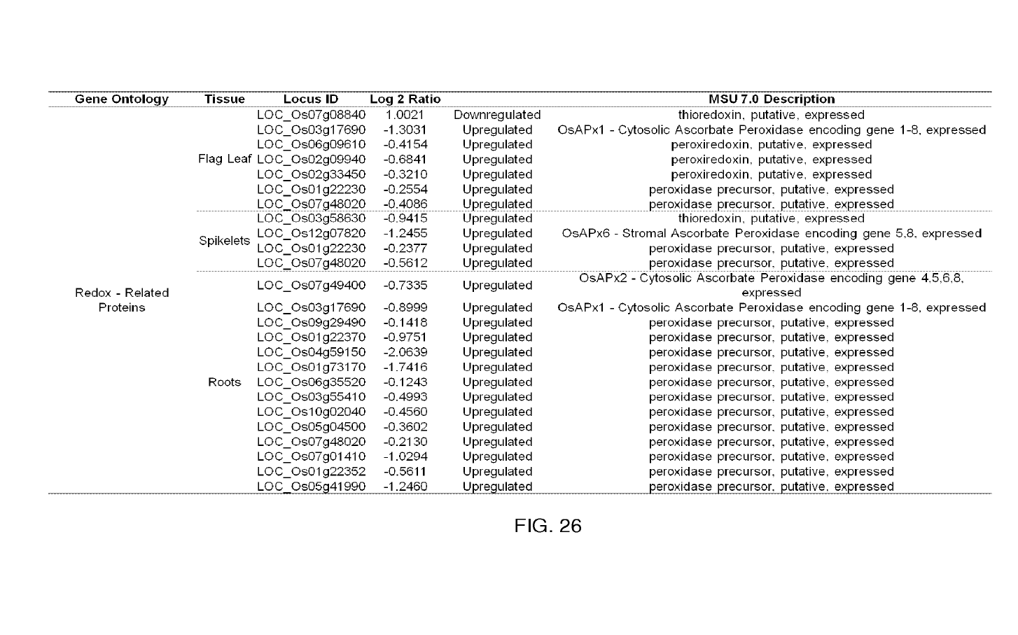

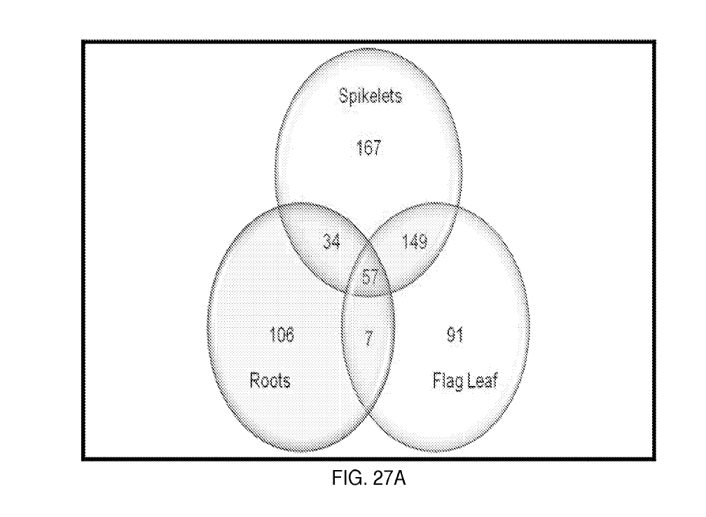

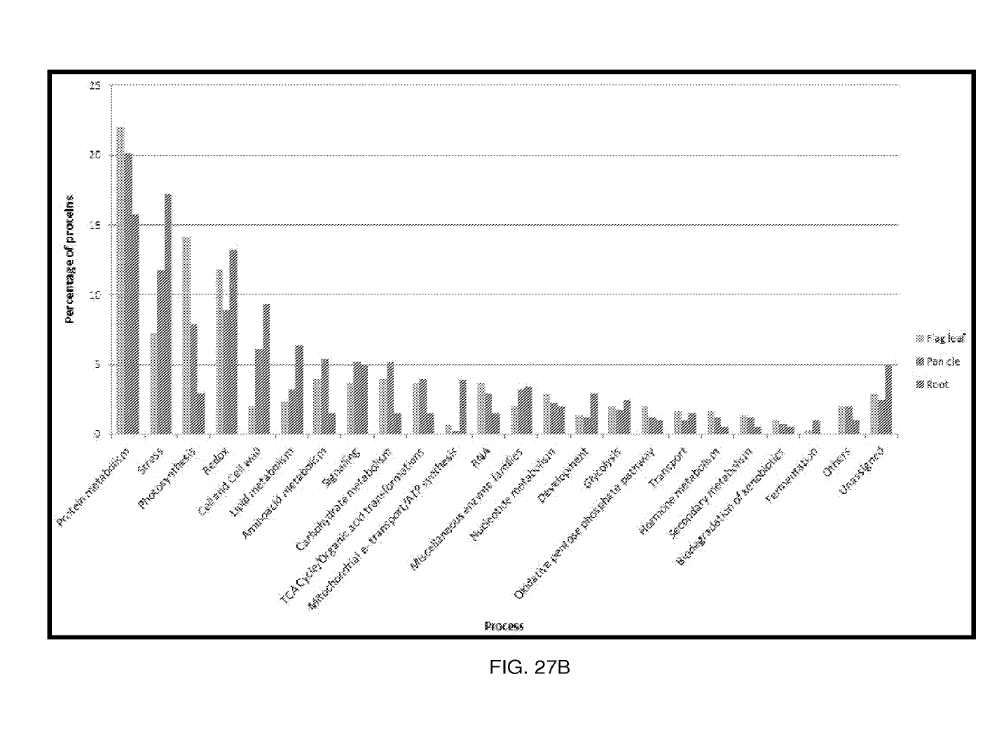

Under drought, photosynthesis is down-regulated to conserve energy and water (Pinheiro et al., 2011) and cell growth is adversely affected (Chaves et al., 2009). Sucrose and glucose are used in respiration to meet cellular energy needs. They are also substrates for osmolyte synthesis towards maintaining homeostasis by protecting membranes, enzymes and other structures against damage and denaturation (Gupta & Kaur, 2005). Fructose however can be involved in secondary metabolite synthesis, such as erythrose-1 4-P, which is a substrate for lignin and phenolic compounds synthesis (Hilal et al., 2004). In the case of the 481-B however, the reason for decreased photosynthesis is related to the sugars. For example, glucose and fructose increased under stress in the flag leaves of Vandana and 481-B but the combined amount of the three sugars (sucrose, glucose and fructose) was more in the flag leaf of the 481-B than in Vandana (FIGS. 30A-30C). Higher sugar content and decreased starch synthesis lead to feedback inhibition of photosynthesis (Paul and Foyer, 2001). Proteomics data indicated lower abundance, in the 481-B under drought, for proteins such as the PsbP and oxygen-evolving enhancer protein involved in the light-dependent photosystem reactions (Table 5). This indicates repression of CO+ fixation. Indeed, a similar photosynthetic rate was observed between 481-B and Vandana under normal conditions changed to a lower photosynthetic rate for 481-B than Vandana under drought (Table 5). However, stomatal closure during drought also prevents CO2 fixation while it is necessary to prevent transpirational water loss. Transpiration efficiency of the 481-B was higher than Vandana under drought (FIG. 40) because the stomatal conductance in the 481-B was substantially reduced under drought (FIG. 41). Calvin cycle enzymes involved in the regeneration of RuBP (Ribulose-1, 5-bisphosphate) such as triose phosphate isomerase, fructose bis-phospate aldolase, fructose-1,6-bisphosphatase and transketolase were more abundant in the 481-B (FIG. 28). Regeneration of RuBP produces CO₂ and NADPH. CO₂ is also limited by partial stomatal closure, while NADPH can feed into various biosynthetic reactions and help in redox buffering in the cell (Verslues and Sharma, 2000). Enzymes involved in the photorespiratory pathway, particularly glycolate oxidase, glycine dehydrogenase and serine hydroxymethyltransferase, were more abundant in the flag leaf of the 481-B (FIG. 26). The photorespiratory pathway aids in reducing O₂ that is involved in reactive oxygen species (ROS) generation. Yet, an inevitable consequence of drought stress is the production of ROS, which causes oxidative damage to the plant (de Carvalho, 2008). Increased amounts of antioxidant enzymes were observed in all three tissues of the 481-B, particularly ascorbate peroxidase and glutathione peroxidase (FIG. 27). These two enzymes are important in detoxification of H2O2 produced during stress (Zhang et al., 2012). Glutathione peroxidase in particular is important in scavenging ROS produced from photosynthesis (Milla et al., 2003). Differential expression of other redox proteins such as glutathione reductase, peroxiredoxin, superoxide dismutase and thioredoxins (Table 5) support the ROS detoxification and signaling processes to maintain cellular homeostasis. Thus, through subtle and coordinated changes in the photosynthetic rate, stomatal conductance, photorespiration and redox status coupled with the regeneration of RuBP, the 481-B exhibits a strategy for better plant sustenance under drought.

TABLE 6
The complete set of proteins identified in the spikelets of Vandana and the 481B NIL (in triplicates),
represented as fold increase in the 481B NIL compared to Vandana during drought stress.

| | | | Log 2 | | |
|---|---|---|---|---|---|
| Gene Ontology | MapMan Bin Description | Locus Id's | Ratio | | MSU v7.0 description |
| Photosystems | PS.lightreaction.photosystem II.PSII | LOC_Os07g36080 | 1.5637 | Downregulated | oxygen evolving enhancer |
| | polypeptide subunits | | | | protein 3 domain containing |
| | | | | | protein, expressed |
| | PS.lightreaction.photo system II.PSII | LOC_Os07g04840 | −0.0722 | Upregulated | PsbP, putative, expressed |
| | polypeptide subunits | | | | |
| | PS.lightreaction.photo system ILPSII | LOC_Os01g31690 | −0.2842 | Upregulated | oxygen-evolving enhancer |
| | polypeptide subunits | | | | protein 1, chloroplast precursor, |
| | | | | | putative, expressed |
| e-Carriers | PS.lightreaction.other electron carrier | LOC_Os06g01210 | 1.9030 | Downregulated | plastocyanin, chloroplast |
| | (ox/red).plastocyanin | | | | precursor, putative, expressed |
| | PS.lightreaction.other electron carrier | LOC_Os08g01380 | −1.3163 | Upregulated | 2Fe-2S iron-sulfur cluster |
| | (ox/red).ferredoxin | | | | binding domain containing |
| | | | | | protein, expressed |
| | PS.lightreaction.other electron carrier | LOC_Os02g01340 | −0.2630 | Upregulated | ferredoxin--NADP reductase, |
| | (ox/red).ferredoxin reductase | | | | chloroplast precursor, putative, |
| | | | | | expressed |
| | PS.lightreaction.other electron carrier | LOC_Os02g22260 | 0.0712 | Downregulated | fruit protein PKIWI502, |
| | (ox/red).ferredoxin oxireductase | | | | putative, expressed |
| Photorespiration | PS.photorespiration.glycolate oxydase | LOC_Os07g05820 | −0.2168 | Upregulated | hydroxyacid oxidase 1, |
| | | | | | putative, expressed |
| | PS.photorespiration.aminotransferases | LOC_Os08g39300 | −0.1161 | Upregulated | aminotransferase, putative, |
| | peroxisomal | | | | expressed |
| | PS.photorespiration.glycine cleavage.H | LOC_Os10g37180 | 0.6189 | Downregulated | glycine cleavage system H |
| | protein | | | | protein, putative, expressed |
| | PS.photorespiration.serine | LOC_Os03g52840 | −0.9404 | Upregulated | serine |
| | hydroxymethyltransferase | | | | hydroxymethyltransferase, |
| | | | | | mitochondrial precursor, |
| | | | | | putative, expressed |
| | PS.photorespiration.hydroxypyruvate | LOC_Os02g01150 | −0.3109 | Upregulated | erythronate-4-phosphate |
| | reductase | | | | dehydrogenase domain |
| | | | | | containing protein, expressed |
| Calvin Cycle | PS.calvin cycle.phosphoglycerate kinase | LOC_Os06g45710 | −0.2390 | Upregulated | phosphoglycerate kinase |
| | | | | | protein, putative, expressed |
| | PS.calvin cycle.phosphoglycerate kinase | LOC_Os05g41640 | 2.0834 | Downregulated | phosphoglycerate kinase |
| | | | | | protein, putative, expressed |
| | PS.calvin cycle.phosphoglycerate kinase | LOC_Os02g07260 | −0.7764 | Upregulated | phosphoglycerate kinase |
| | | | | | protein, putative, expressed |
| | PS.calvin cycle.GAP | LOC_Os03g03720 | 0.4913 | Downregulated | glyceraldehyde-3-phosphate |
| | | | | | dehydrogenase, putative, |
| | | | | | expressed |
| | PS.calvin cycle.GAP | LOC_Os04g38600 | 0.3847 | Downregulated | glyceraldehyde-3-phosphate |
| | | | | | dehydrogenase, putative, |
| | | | | | expressed |
| | PS.calvin cycle.TPI | LOC_Os01g62420 | −1.7069 | Upregulated | triosephosphate isomerase, |
| | | | | | cytosolic, putative, expressed |
| | PS.calvin cycle.TPI | LOC_Os01g05490 | −0.2935 | Upregulated | triosephosphate isomerase, |
| | | | | | cytosolic, putative, expressed |
| | PS.calvin cycle.TPI | LOC_Os09g36450 | 0.1339 | Downregulated | triosephosphate isomerase, |
| | | | | | chloroplast precursor, putative, |
| | | | | | expressed |
| | PS.calvin cycle.aldolase | LOC_Os11g07020 | 0.0211 | Downregulated | fructose-bisphospate aldolase |
| | | | | | isozyme, putative, expressed |
| | PS.calvin cycle.aldolase | LOC_Os01g67860 | −0.4051 | Upregulated | fructose-bisphospate aldolase |
| | | | | | isozyme, putative, expressed |
| | PS.calvin cycle.aldolase | LOC_Os05g33380 | −0.7749 | Upregulated | fructose-bisphospate aldolase |
| | | | | | isozyme, putative, expressed |
| | PS.calvin cycle.aldolase | LOC_Os06g40640 | −0.2202 | Upregulated | fructose-bisphospate aldolase |
| | | | | | isozyme, putative, expressed |
| | PS.calvin cycle.transketolase | LOC_Os06g04270 | 0.4282 | Downregulated | transketolase, chloroplast |
| | | | | | precursor, putative, expressed |
| | PS.calvin cycle.seduheptulose | LOC_Os04g16680 | −0.0726 | Upregulated | fructose-1,6-bisphosphatase, |
| | bisphosphatase | | | | putative, expressed |
| | PS.calvin cycle.PRK | LOC_Os02g47020 | −0.3656 | Upregulated | phosphoribulokinase/Uridine |
| | | | | | kinase family protein, |
| | | | | | expressed |
| | PS.calvin cycle.rubisco interacting | LOC_Os11g47970 | 1.0692 | Downregulated | AAA-type ATPase family |
| | | | | | protein, putative, expressed |
| Thylakoid | not assigned.unknown | LOC_Os01g05080 | 0.1848 | Downregulated | thylakoid lumenal protein, |
| Proteins | | | | | putative, expressed |
| CHO | major CHO | LOC_Os08g25734 | −0.6910 | Upregulated | glucose-1-phosphate |
| Metabolism | metabolism.synthesis.starch.AGPase | | | | adenylyltransferase large |
| | | | | | subunit, chloroplast precursor, |
| | | | | | putative, expressed |
| | | | | | glucose-1-phosphate |
| | major CHO | LOC_Os01g44220 | −3.0200 | Upregulated | adenylyltransferase large |
| | metabolism.synthesis.starch.AGPase | | | | subunit, chloroplast precursor, |
| | | | | | putative, expressed |
| | major CHO | LOC_Os02g32660 | −0.8422 | Upregulated | 1,4-alpha-glucan-branching |
| | metabolism.synthesis.starch.starch | | | | enzyme, chloroplast precursor, |
| | branching | | | | putative, expressed |
| | major CHO | LOC_Os06g51084 | −0.9212 | Upregulated | 1,4-alpha-glucan-branching |
| | metabolism.synthesis.starch.starch | | | | enzyme, chloroplast precursor, |
| | branching | | | | putative, expressed |
| | major CHO | LOC_Os01g66940 | 0.0320 | Downregulated | kinase, pfkB family, putative, |
| | metabolism.degradation.sucrose. | | | | expressed |
| | fructokinase | | | | |
| | major CHO | LOC_Os08g02120 | −0.2550 | Upregulated | kinase, pfkB family, putative, |
| | metabolism.degradation.sucrose. | | | | expressed |
| | fructokinase | | | | |
| | major CHO | LOC_Os07g42490 | −2.5355 | Upregulated | sucrose synthase, putative, |
| | metabolism.degradation.sucrose.Susy | | | | expressed |
| | major CHO | LOC_Os06g09450 | −0.1459 | Upregulated | sucrose synthase, putative, |
| | metabolism.degradation.sucrose.Susy | | | | expressed |
| | major CHO | LOC_Os03g55090 | −1.6316 | Upregulated | alpha-glucan phosphorylast |
| | metabolism.degradation.starch.starch | | | | isozyme, putative, expressed |
| | phosphorylase | | | | |
| | C1-metabolism | LOC_Os06g29180 | −0.5043 | Upregulated | erythronate-4-phosphate |
| | | | | | dehydrogenase domain |
| | | | | | containing protein, expressed |
| | not assigned.unknown | LOC_Os08g27840 | 0.8034 | Downregulated | phosphoenolpyruvate |
| | | | | | carboxylase, putative, |
| | | | | | expressed |
| | not assigned.unknown | LOC_Os02g02560 | −0.7203 | Upregulated | UTP--glucose-1-phosphate |
| | | | | | uridylyltransferase, putative, |
| | | | | | expressed |
| | not assigned.unknown | LOC_Os07g47290 | 1.0816 | Downregulated | xylose isomerase, putative, |
| | | | | | expressed |
| | not assigned.unknown | LOC_Os01g60190 | −0.0957 | Upregulated | 2,3-bisphosphoglycerate- |
| | | | | | independent phosphoglycerate |
| | | | | | mutase, putative, expressed |
| | not assigned.unknown | LOC_Os02g14770 | 0.4843 | Downregulated | phosphoenolpyruvate |
| | | | | | carboxylase, putative, |
| | | | | | expressed |
| Glycolysis | glycolysis.cytosolic | LOC_Os08g03290 | 0.4948 | Downregulated | glyceraldehyde-3-phosphate |
| | branch.glyceraldehyde 3-phosphate | | | | dehydrogenase, putative, |
| | dehydrogenase (GAP-DH) | | | | expressed |
| | glycolysis.cytosolic | LOC_Os02g38920 | −0.6286 | Upregulated | glyceraldehyde-3-phosphate |
| | branch.glyceraldehyde 3-phosphate | | | | dehydrogenase, putative, |
| | dehydrogenase (GAP-DH) | | | | expressed |
| | glycolysis.cytosolic branch.enolase | LOC_Os06g04510 | 1.2905 | Downregulated | enolase, putative, expressed |
| | glycolysis.cytosolic branch.enolase | LOC_Os10g08550 | 0.2115 | Downregulated | enolase, putative, expressed |
| | glycolysis.plastid branch.glucose-6- | LOC_Os03g56460 | 1.0098 | Downregulated | glucose-6-phosphate isomerase, |
| | phosphate isomerase | | | | putative, expressed |
| | not assigned.unknown | LOC_Os03g50480 | −0.1673 | Upregulated | phosphoglucomutase, putative, |
| | | | | | expressed |
| Fermentation | fermentation.aldehyde dehydrogenase | LOC_Os08g32870 | 0.9221 | Downregulated | aldehyde dehydrogenase, |
| | | | | | putative, expressed |
| | fermentation.aldehyde dehydrogenase | LOC_Os06g15990 | 0.0983 | Downregulated | aldehyde dehydrogenase, |
| | | | | | putative, expressed |
| Oxidative | OPP.oxidative PP.6-phosphogluconate | LOC_Os06g02144 | 1.0788 | Downregulated | 6-phosphogluconate |
| Pentose | dehydrogenase | | | | dehydrogenase, |
| Phosphate | | | | | decarboxylating, putative, |
| Pathway | | | | | expressed |
| | OPP.non-reductive PP.transaldolase | LOC_Os01g70170 | −0.2570 | Upregulated | transaldolase, putative, |
| | | | | | expressed |
| | OPP.non-reductive PP.ribose 5- | LOC_Os07g08030 | −0.1695 | Upregulated | ribose-5-phosphate isomerase |
| | phosphate isomerase | | | | A, putative, expressed |
| TCA Cycle | TCA/org. transformation.TCA.pyruvate | LOC_Os01g22520 | 0.9039 | Downregulated | dihydrolipoyl dehydrogenase 1, |
| | DH.E3 | | | | mitochondrial precursor, |
| | | | | | putative, expressed |
| | TCA/org. transformation.TCA.CS | LOC_Os02g10070 | 0.8903 | Downregulated | citrate synthase, putative, |
| | | | | | expressed |
| | TCA/org. transformation.TCA.aconitase | LOC_Os08g09200 | 0.4164 | Downregulated | aconitate hydratase protein, |
| | | | | | putative, expressed |
| | TCA/org. transformation.TCA.IDH | LOC_Os01g46610 | 1.0543 | Downregulated | dehydrogenase, putative, |
| | | | | | expressed |
| | TCA/org. transformation.TCA.2- | LOC_Os04g32330 | 0.0882 | Downregulated | dihydrolipoyllysine-residue |
| | oxoglutarate dehydrogenase | | | | succinyltransferase component |
| | | | | | of 2-oxoglutarate |
| | | | | | dehydrogenase complex, |
| | | | | | mitochondrial precursor, |
| | | | | | putative, expressed |
| | | | | | succinyl-CoA ligase subunit |
| | TCA/org. transformation.TCA.succinyl- | LOC_Os07g38970 | −0.2509 | Upregulated | alpha-2, mitochondrial |
| | CoA ligase | | | | precursor, putative, expressed |
| | TCA/org. transformation.TCA.succinyl- | LOC_Os02g40830 | 0.0864 | Downregulated | succinyl-CoA ligase beta-chain, |
| | CoA ligase | | | | mitochondrial precursor, |
| | | | | | putative, expressed |
| | TCA/org. transformation.TCA.malate | LOC_Os05g49880 | −0.2718 | Upregulated | lactate/malate dehydrogenase, |
| | DH | | | | putative, expressed |
| | TCA/org. transformation.other organic | LOC_Os10g33800 | −1.0246 | Upregulated | lactate/malate dehydrogenase, |
| | acid transformaitons.cyt MDH | | | | putative, expressed |
| | TCA/org. transformation.other organic | LOC_Os08g44810 | −0.4044 | Upregulated | lactate/malate dehydrogenase, |
| | acid transformaitons.cyt MDH | | | | putative, expressed |
| | TCA/org. transformation.other organic | LOC_Os01g52500 | 0.6078 | Downregulated | NADP-dependent malic |
| | acid transformaitons.malic | | | | enzyme, putative, expressed |
| | TCA/org. transformation.carbonic | LOC_Os01g45274 | 0.4191 | Downregulated | carbonic anhydrase, chloroplast |
| | anhydrases | | | | precursor, putative, expressed |
| Mitochondrial | mitochondrial electron transport/ATP | LOC_Os05g47980 | −0.4295 | Upregulated | ATP synthase, putative, |
| Electron | synthesis.F1-ATPase | | | | expressed |
| Transport Chain | | | | | |
| Cell and Cell | cell wall.cell wall proteins.RGP | LOC_Os03g40270 | −0.1231 | Upregulated | alpha-1,4-glucan-protein |
| Wall Related | | | | | synthase, putative, expressed |
| Proteins | cell wall.modification | LOC_Os03g01610 | 2.3651 | Downregulated | expansin precursor, putative, |
| | | | | | expressed |
| | cell wall.modification | LOC_Os06g45150 | 2.2180 | Downregulated | pollen allergen putative, |
| | | | | | expressed |
| | cell wall. modification | LOC_Os06g44470 | 2.9553 | Downregulated | pollen allergen putative, |
| | | | | | expressed |
| | cell wall.modification | LOC_Os03g01650 | 2.3651 | Downregulated | expansin precursor, putative, |
| | | | | | expressed |
| | cell.organisation | LOC_Os03g50885 | −0.2047 | Upregulated | actin, putative, expressed |
| | cell.organisation | LOC_Os02g51750 | 0.1693 | Downregulated | annexin, putative, expressed |
| | cell.organisation | LOC_Os06g05880 | 0.2065 | Downregulated | profilin domain containing |
| | | | | | protein, expressed |
| | cell.organisation | LOC_Os07g38730 | −1.3460 | Upregulated | tubulin/FtsZ domain containing |
| | | | | | protein, putative, expressed |
| | cell.organisation | LOC_Os10g17680 | 3.0523 | Downregulated | profilin domain containing |
| | | | | | protein, expressed |
| | cell.organisation | LOC_Os10g17660 | 3.0523 | Downregulated | profilin domain containing |
| | | | | | protein, expressed |
| | cell.organisation | LOC_Os03g60580 | 0.4426 | Downregulated | actin-depolymerizing factor, |
| | | | | | putative, expressed |
| | cell.organisation | LOC_Os03g61970 | −0.0750 | Upregulated | actin, putative, expressed |
| | cell.organisation | LOC_Os11g14220 | 0.3604 | Downregulated | tubulin/FtsZ domain containing |
| | | | | | protein, putative, expressed |
| | cell.division | LOC_Os04g35570 | 0.6979 | Downregulated | Regulator of chromosome |
| | | | | | condensation domain |
| | | | | | containing protein, expressed |
| | cell.cycle.peptidylprolyl isomerase | LOC_Os02g02890 | −0.0097 | Upregulated | peptidyl-prolyl cis-trans |
| | | | | | isomerase, putative, expressed |
| | cell.cycle.peptidylprolyl isomerase | LOC_Os05g01270 | 0.5344 | Downregulated | peptidyl-prolyl cis-trans |
| | | | | | isomerase, putative, expressed |
| | cell.cycle.peptidylprolyl isomerase | LOC_Os08g41390 | −0.0005 | Upregulated | peptidyl-prolyl isomerase, |
| | | | | | putative, expressed |
| | cell.cycle.peptidylprolyl isomerase | LOC_Os09g39780 | 0.1088 | Downregulated | peptidyl-prolyl cis-trans |
| | | | | | isomerase, putative, expressed |
| | not assigned.unknown | LOC_Os08g04650 | 2.8489 | Downregulated | pectinesterase inhibitor domain |
| | | | | | containing protein, expressed |
| Lipid | lipid metabolism.FA synthesis and FA | LOC_Os09g10600 | −0.5556 | Upregulated | enoyl-acyl-carrier-protein |
| Metabolism | elongation.enoyl ACP reductase | | | | reductase NADH, chloroplast |
| | | | | | precursor, expressed |
| | lipid metabolism.FA synthesis and FA | LOC_Os08g06550 | 1.0487 | Downregulated | acyl CoA binding protein, |
| | elongation.acyl-CoA binding protein | | | | putative, expressed |
| | lipid metabolism.lipid transfer proteins | LOC_Os11g24070 | 0.5022 | Downregulated | LTPL10 - Protease |
| | etc | | | | inhibitor/seed storage/LTP |
| | | | | | family protein precursor, |
| | | | | | expressed |
| | lipid metabolism.″exotics″ (steroids, | LOC_Os08g04460 | −0.4169 | Upregulated | NADPH-dependent FMN |
| | squalene etc) | | | | reductase domain containing |
| | | | | | protein, expressed |
| | not assigned.unknown | LOC_Os07g11630 | −3.7085 | Upregulated | LTPL163 - Protease |
| | | | | | inhibitor/seed storage/LTP |
| | | | | | family protein precursor, |
| | | | | | expressed |
| | not assigned.unknown | LOC_Os01g60740 | 0.4782 | Downregulated | LTPL16 - Protease |
| | | | | | inhibitor/seed storage/LTP |
| | | | | | family protein precursor, |
| | | | | | expressed |
| | not assigned.unknown | LOC_Os10g36170 | 0.6033 | Downregulated | LTPL160 - Protease |
| | | | | | inhibitor/seed storage/LTP |
| | | | | | family protein precursor, |
| | | | | | expressed |
| Amino Acid | amino acid metabolism.synthesis.central | LOC_Os08g36320 | 1.1556 | Downregulated | decarboxylase, putative, |
| Metabolism | amino acid | | | | expressed |
| | metabolism.GABA.Glutamate | | | | |
| | decarboxylase | | | | |
| | amino acid metabolism.synthesis.central | LOC_Os02g55420 | 0.0680 | Downregulated | aminotransferase, classes I and |
| | amino acid | | | | II, domain containing protein, |
| | metabolism.aspartate.aspartate | | | | expressed |
| | aminotransferase | | | | |
| | amino acid metabolism.synthesis.central | LOC_Os01g55540 | −1.4791 | Upregulated | aminotransferase, classes I and |
| | amino acid | | | | II, domain containing protein, |
| | metabolism.aspartate.aspartate | | | | expressed |
| | aminotransferase | | | | |
| | amino acid metabolism.synthesis.central | LOC_Os10g25130 | −2.3369 | Upregulated | aminotransferase, classes I and |
| | amino acid metabolism.alanine.alanine | | | | II, domain containing protein, |
| | aminotransferase | | | | expressed |
| | amino acid metabolism.synthesis.central | LOC_Os07g42600 | 0.4995 | Downregulated | aminotransferase, classes I and |
| | amino acid metabolism.alanine.alanine | | | | II, domain containing protein, |
| | aminotransferase | | | | expressed |
| | amino acid metabolism.synthesis.central | LOC_Os08g39300 | −0.1161 | Upregulated | aminotransferase, putative, |
| | amino acid metabolism.alanine.alanine- | | | | expressed |
| | glyoxylate aminotransferase | | | | |
| | amino acid | LOC_Os02g47590 | −0.2841 | Upregulated | ornithine carbamoyltransferase, |
| | metabolism. synthesis.glutamate | | | | putative, expressed |
| | family.arginine.ornithine | | | | |
| | carbamoyltransferase | | | | |
| | amino acid | LOC_Os01g22010 | −0.2171 | Upregulated | S-adenosylmethionine |
| | metabolism.synthesis.aspartate | | | | synthetase, putative, expressed |
| | family.methionine | | | | |
| | amino acid | LOC_Os01g46380 | 0.9291 | Downregulated | ketol-acid reductoisomerase, |
| | metabolism.synthesis.branched chain | | | | chloroplast precursor, putative, |
| | group.common | | | | expressed |
| | amino acid metabolism.synthesis.serine- | LOC_Os04g55720 | 0.2357 | Downregulated | D-3-phosphoglycerate |
| | glycine-cysteine | | | | dehydrogenase, chloroplast |
| | group.serine.phosphoglycerate | | | | precursor, putative, expressed |
| | dehydrogenase | | | | |
| | amino acid metabolism.synthesis.serine- | LOC_Os08g39300 | −0.1161 | Upregulated | aminotransferase, putative, |
| | glycine-cysteine group.glycine.serine | | | | expressed |
| | glyoxylate aminotransferase | | | | |
| | amino acid metabolism.synthesis.serine- | LOC_Os12g42980 | 0.0888 | Downregulated | cysteine synthase, putative, |
| | glycine-cysteine group.cysteine.OASTL | | | | expressed |
| | amino acid metabolism.synthesis.serine- | LOC_Os01g74650 | 0.0546 | Downregulated | cysteine synthase, |
| | glycine-cysteine group.cysteine.OASTL | | | | mitochondrial precursor, |
| | | | | | putative, expressed |
| | amino acid | LOC_Os09g36800 | −0.6866 | Upregulated | 3-dehydroquinate synthase, |
| | metabolism.synthesis.aromatic | | | | putative, expressed |
| | aa.chorismate.3-dehydroquinate synthase | | | | |
| | amino acid | LOC_Os03g04169 | −0.1991 | Upregulated | ATP phosphoribosyltransferase, |
| | metabolism.synthesis.histidine.ATP | | | | putative, expressed |
| | phosphoribosyl transferase | | | | |
| | amino acid | LOC_Os08g09250 | −0.0324 | Upregulated | glyoxalase family protein, |
| | metabolism.degradation.aspartate | | | | putative, expressed |
| | family.threonine | | | | |
| | amino acid | LOC_Os07g09060 | 0.8606 | Downregulated | aldehyde dehydrogenase, |
| | metabolism.degradation.branched-chain | | | | putative, expressed |
| | group.valine | | | | |
| | amino acid | LOC_Os01g51410 | −0.0856 | Upregulated | glycine dehydrogenase, |
| | metabolism.degradation.serine-glycine- | | | | putative, expressed |
| | cysteine group.glycine | | | | |
| | amino acid | LOC_Os04g53230 | 0.7379 | Downregulated | aminomethyltransferase, |
| | metabolism.degradation.serine-glycine- | | | | putative, expressed |
| | cysteine group.glycine | | | | |
| | amino acid | LOC_Os02g10310 | 1.5383 | Downregulated | fumarylacetoacetase, putative, |
| | metabolism.degradation.aromatic | | | | expressed |
| | aa.tyrosine | | | | |
| | amino acid | LOC_Os06g39344 | −0.3047 | Upregulated | enoyl-CoA hydratase/isomerase |
| | metabolism.degradation.aromatic | | | | family protein, putative, |
| | aa.tryptophan | | | | expressed |
| Secondary | secondary | LOC_Os08g38900 | −0.9877 | Upregulated | caffeoyl-CoA O- |
| Metabolism | metabolism.phenylpropanoids.lignin | | | | methyltransferase, putative, |
| | biosynthesis.CCoAOMT | | | | expressed |
| | secondary | LOC_Os08g38910 | −0.4764 | Upregulated | caffeoyl-CoA O- |
| | metabolism.phenylpropanoidslignin | | | | methyltransferase, putative, |
| | biosynthesis.CCoAOMT | | | | expressed |
| Hormones | hormone metabolism.abscisic | LOC_Os03g57690 | 0.3420 | Downregulated | aldehyde oxidase, putative, |
| | acid.synthesis-degradation | | | | expressed |
| | hormone metabolism.auxin.induced- | LOC_Os01g43090 | −0.4052 | Upregulated | oxidoreductase, aldo/keto |
| | regulated-responsive-activated | | | | reductase family protein, |
| | | | | | putative, expressed |
| | hormone metabolism.auxin.induced- | LOC_Os03g58170 | −0.1093 | Upregulated | stem-specific protein TSJT1, |
| | regulated-responsive-activated | | | | putative, expressed |
| Stress - Related | stress | LOC_Os02g43020 | −0.1599 | Upregulated | heat shock protein STI, |
| Proteins | | | | | putative, expressed |
| | stress.biotic | LOC_Os10g39680 | 0.1957 | Downregulated | CHIT14 - Chitinase family |
| | | | | | protein precursor, expressed |
| | stress.biotic | LOC_Os04g41620 | 0.2360 | Downregulated | CHIT2 - Chitinase family |
| | | | | | protein precursor, expressed |
| | stress.biotic | LOC_Os07g11510 | −2.2587 | Upregulated | RAL6 - Seed allergenic protein |
| | | | | | RA5/RA14/RA17 precursor, |
| | | | | | expressed |
| | stress.biotic | LOC_Os07g11330 | −3.6452 | Upregulated | RAL2 - Seed allergenic protein |
| | | | | | RA5/RA14/RA17 precursor, |
| | | | | | expressed |
| | stress.biotic | LOC_Os07g11380 | −3.7525 | Upregulated | RAL4 - Seed allergenic protein |
| | | | | | RA5/RA14/RA17 precursor, |
| | | | | | expressed |
| | stress.biotic | LOC_Os03g46070 | −0.3440 | Upregulated | thaumatin, putative, expressed |
| | stress.biotic | LOC_Os07g11410 | −2.0776 | Upregulated | RALS - Seed allergenic protein |
| | | | | | RA5/RA14/RA17 precursor, |
| | | | | | expressed |
| | stress.biotic | LOC_Os10g34930 | 0.0366 | Downregulated | secretory protein putative, |
| | | | | | expressed |
| | stress.biotic.PR-proteins.proteinase | LOC_Os04g44470 | −2.6935 | Upregulated | KUN1 - Kunitz-type trypsin |
| | inhibitors.trypsin inhibitor | | | | inhibitor precursor, expressed |
| | stress.abiotic.heat | LOC_Os09g30412 | 0.2260 | Downregulated | heat shock protein, putative, |
| | | | | | expressed |
| | stress.abiotic.heat | LOC_Os02g02410 | −1.2578 | Upregulated | DnaK family protein, putative, |
| | | | | | expressed |
| | stress.abiotic.heat | LOC_Os02g48110 | −1.2334 | Upregulated | DnaK family protein, putative, |
| | | | | | expressed |
| | stress.abiotic.heat | LOC_Os01g62290 | 0.8692 | Downregulated | DnaK family protein, putative, |
| | | | | | expressed |
| | stress.abiotic.heat | LOC_Os03g02260 | 0.5838 | Downregulated | DnaK family protein, putative, |
| | | | | | expressed |
| | stress.abiotic.heat | LOC_Os03g60620 | −1.0429 | Upregulated | DnaK family protein, putative, |
| | | | | | expressed |
| | stress.abiotic.heat | LOC_Os08g38086 | −0.2952 | Upregulated | heat shock protein, putative, |
| | | | | | expressed |
| | stress.abiotic.heat | LOC_Os04g01740 | 0.5565 | Downregulated | heat shock protein, putative, |
| | | | | | expressed |
| | stress.abiotic.heat | LOC_Os03g31300 | −0.2978 | Upregulated | chaperone protein clpB 1, |
| | | | | | putative, expressed |
| | stress.abiotic.heat | LOC_Os03g16040 | −0.2767 | Upregulated | hsp20/alpha crystallin family |
| | | | | | protein, putative, expressed |
| | stress.abiotic.heat | LOC_Os03g15960 | 1.5066 | Downregulated | hsp20/alpha crystallin family |
| | | | | | potent, puutuve, expressed |
| Redox - Related | stress.abiotic.heat | LOC_Os01g04370 | 1.5200 | Downregulated | hsp20/alpha crystallin family |
| Proteins | | | | | protein, putative, expressed |
| | stress.abiotic.heat | LOC_Os05g44340 | −1.4170 | Upregulated | heat shock protein 101, |
| | | | | | putative, expressed |
| | stress.abiotic.heat | LOC_Os01g08560 | −0.1604 | Upregulated | DnaK family protein, putative, |
| | | | | | expressed |
| | stress.abiotic.heat | LOC_Os05g38530 | 0.9983 | Downregulated | DnaK family protein, putative, |
| | | | | | expressed |
| | stress.abiotic.heat | LOC_Os02g53420 | 0.1441 | Downregulated | DnaK family protein, putative, |
| | | | | | expressed |
| | stress.abiotic.heat | LOC_Os11g47760 | −0.6065 | Upregulated | DnaK family protein, putative, |
| | | | | | expressed |
| | stress.abiotic.heat | LOC_Os05g35400 | −0.9377 | Upregulated | DnaK family protein, putative, |
| | | | | | expressed |
| | stress.abiotic.heat | LOC_Os06g50300 | 0.1202 | Downregulated | heat shock protein, putative, |
| | | | | | expressed |
| | stress.abiotic.cold | LOC_Os08g03520 | 0.5390 | Downregulated | retrotransposon protein, |
| | | | | | putative, Ty1-copia subclass, |
| | | | | | expressed |
| | stress.abiotic.unspecified | LOC_Os09g39950 | 1.5346 | Downregulated | POEI23 - Pollen Ole e I |
| | | | | | allergen and extensin family |
| | | | | | protein precursor, expressed |
| | stress.abiotic.unspecified | LOC_Os08g35760 | 0.2065 | Downregulated | Cupin domain containing |
| | | | | | protein, expressed |
| | stress.abiotic.unspecified | LOC_Os01g14670 | 2.7465 | Downregulated | Cupin domain containing |
| | | | | | protein, expressed |
| | | | | | pathogenesis-related Bet v I |
| | not assigned.unknown | LOC_Os08g28670 | −1.1951 | Upregulated | family protein, putative, |
| | | | | | expressed |
| | not assigned.unlmown | LOC_Os07g41810 | −2.2856 | Upregulated | stress responsive A/B Barrel |
| | | | | | domain containing protein, |
| | | | | | expressed |
| | not assigned.unknown | LOC_Os03g21040 | −0.5425 | Upregulated | stress responsive protein, |
| | | | | | putative, expressed |
| | not assigned.unknown | LOC_Os05g46480 | 0.4281 | Downregulated | late embryogenesis abundant |
| | | | | | protein, group 3, putative, |
| | | | | | expressed |
| | redox.thioredoxin | LOC_Os11g09280 | −2.7473 | Upregulated | OsPDIL1-1 protein disulfide |
| | | | | | isomerase PDIL1-1, expressed |
| | redox.thioredoxin | LOC_Os01g68480 | 0.0593 | Downregulated | thioredoxin, putative, expressed |
| | redox.thioredoxin | LOC_Os07g08840 | 0.1347 | Downregulated | thioredoxin, putative, expressed |
| | redox.thioredoxin | LOC_Os02g01010 | −1.9793 | Upregulated | OsPDIL1-4 protein disulfide |
| | | | | | isomerase PDIL1-4, expressed |
| | redox.ascorbate and glutathione.ascorbate | LOC_Os05g02530 | 0.7293 | Downregulated | glutathione S-transferase, N- |
| | | | | | terminal domain containing |
| | | | | | protein, expressed |
| | redox.ascorbate and glutathione.ascorbate | LOC_Os08g44340 | 1.4738 | Downregulated | monodehydroascorbate |
| | | | | | reductase, putative, expressed |
| | redox.ascorbate and glutathione.ascorbate | LOC_Os12g07820 | −1.2455 | Upregulated | OsAPx6 - Stromal Ascorbate |
| | | | | | Peroxidase encoding gene 5,8, |
| | | | | | expressed |
| | redox.ascorbate and glutathione.ascorbate | LOC_Os09g39380 | −0.1827 | Upregulated | monodehydroascorbate |
| | | | | | reductase, putative, expressed |
| | redox.ascorbate and glutathione.ascorbate | LOC_Os07g49400 | 0.0966 | Downregulated | OsAPx2 - Cytosolic Ascorbate |
| | | | | | Peroxidase encoding gene |
| | | | | | 4,5,6,8, expressed |
| | redox.ascorbate and glutathione.ascorbate | LOC_Os03g17690 | 0.3218 | Downregulated | OsAPx1 - Cytosolic Ascorbate |
| | | | | | Peroxidase encoding gene 1-8, |
| | | | | | expressed |
| | redox.ascorbate and | LOC_Os02g56850 | 0.7565 | Downregulated | glutathione reductase, putative, |
| | glutathione.glutathione | | | | expressed |
| | redox.glutaredoxins | LOC_Os04g42930 | 0.9443 | Downregulated | OsGrx_C2.2 - glutaredoxin |
| | | | | | subgroup I, expressed |
| | redox.peroxiredoxin | LOC_Os06g09610 | 1.0303 | Downregulated | peroxiredoxin, putative, |
| | | | | | expressed |
| | redox.peroxiredoxin | LOC_Os01g48420 | 0.3974 | Downregulated | peroxiredoxin, putative, |
| | | | | | expressed |
| | redox.peroxiredoxin | LOC_Os02g09940 | 1.0529 | Downregulated | peroxiredoxin, putative, |
| | | | | | expressed |
| | redox.peroxiredoxin.BAS1 | LOC_Os02g33450 | 0.1564 | Downregulated | peroxiredoxin, putative, |
| | | | | | expressed |
| | redox.dismutases and catalases | LOC_Os05g25850 | −0.0756 | Upregulated | superoxide dismutase, |
| | | | | | mitochondrial precursor, |
| | | | | | putative, expressed |
| | redox.dismutases and catalases | LOC_Os07g46990 | 1.2151 | Downregulated | copper/zinc superoxide |
| | | | | | dismutase, putative, expressed |
| | redox.dismutases and catalases | LOC_Os02g02400 | 0.5274 | Downregulated | catalase isozyme A, putative, |
| | | | | | expressed |
| | redox.dismutases and catalases | LOC_Os08g44770 | 0.8000 | Downregulated | copper/zinc superoxide |
| | | | | | dismutase, putative, expressed |
| | misc.glutathione S transferases | LOC_Os01g55830 | 0.5385 | Downregulated | glutathione S-transferase, |
| | | | | | putative, expressed |
| | misc.glutathione S transferases | LOC_Os01g27360 | −0.0515 | Upregulated | glutathione S-transferase, |
| | | | | | putative, expressed |
| | misc.glutathione S transferases | LOC_Os10g38700 | 0.0612 | Downregulated | glutathione S-transferase, |
| | | | | | putative, expressed |
| | misc.glutathione S transferases | LOC_Os03g04220 | 0.5483 | Downregulated | glutathione S-transferase, |
| | | | | | putative, expressed |
| | misc.peroxidases | LOC_Os09g29490 | 0.6445 | Downregulated | peroxidase precursor, putative, |
| | | | | | expressed |
| | misc.peroxidases | LOC_Os01g22230 | −0.2377 | Upregulated | peroxidase precursor, putative, |
| | | | | | expressed |
| | misc.peroxidases | LOC_Os07g48020 | −0.5612 | Upregulated | peroxidase precursor, putative, |
| | | | | | expressed |
| Nucleotide | nucleotide | LOC_Os02g41590 | −0.0175 | Upregulated | kinase, pfkB family, putative, |
| Metabolism | metabolism.salvage.nucleoside | | | | expressed |
| | kinases.adenosine kinase | | | | |
| | nucleotide metabolism.phosphotransfer | LOC_Os05g51700 | −0.0434 | Upregulated | nucleoside diphosphate kinase, |
| | and pyrophosphatases.nucleoside | | | | putative, expressed |
| | diphosphate kinase | | | | |
| | nucleotide metabolism.phosphotransfer | LOC_Os10g41410 | −1.2549 | Upregulated | nucleoside diphosphate kinase, |
| | and pyrophosphatases.nucleoside | | | | putative, expressed |
| | diphosphate kinase | | | | |
| | nucleotide metabolism.phosphotransfer | LOC_Os07g30970 | 0.0015 | Downregulated | nucleoside diphosphate kinase, |
| | and pyrophosphatases.nucleoside | | | | putative, expressed |
| | diphosphate kinase | | | | |
| | nucleotide metabolism.phosphotransfer | LOC_Os04g59040 | 0.0943 | Downregulated | soluble inorganic |
| | and pyrophosphatases.misc | | | | pyrophosphatase, putative, |
| | | | | | expressed |
| | DNA.synthesis/chromatin structure | LOC_Os02g05330 | 0.3958 | Downregulated | DEAD-box ATP-dependent |
| | | | | | RNA helicase, putative, |
| | | | | | expressed (″DEAD″ disclosed |
| | | | | | as SEQ ID NO: 181) |
| | DNA.repair | LOC_Os01g36090 | 0.3489 | Downregulated | DNA-damage-repair/toleration |
| | | | | | protein DRT102, putative, |
| | | | | | expressed |
| | DNA.unspecified | LOC_Os01g13700 | −0.0045 | Upregulated | DNA-binding protein-related, |
| | | | | | putative, expressed |
| Miscellaneous | Biodegradation of Xenobiotics | LOC_Os08g09250 | −0.0324 | Upregulated | glyoxalase family protein, |
| Enzymes | | | | | putative, expressed |
| | misc.misc2 | LOC_Os01g34700. | 0.2412 | Downregulated | dienelactone hydrolase family |
| | | | | | protein, expressed |
| | misc.misc2 | LOC_Os05g33100 | 0.3476 | Downregulated | endo-1,3;1,4-beta-D-glucanase |
| | | | | | precursor, putative, expressed |
| | misc.O-methyl transferases | LOC_Os08g06100 | −0.6499 | Upregulated | O-methyltransferase, putative, |
| | | | | | expressed |
| | misc.oxidases - copper, flavone etc. | LOC_Os08g29170 | −0.0531 | Upregulated | dehydrogenase, putative, |
| | | | | | expressed |
| | misc.nitrilases, *nitrile lyases, berberine | LOC_Os06g35590 | 1.5422 | Downregulated | reticuline oxidase-like protein |
| | bridge enzymes, reticuline oxidases, | | | | precursor, putative, expressed |
| | troponine reductases | | | | |
| | misc.alcohol dehydrogenases | LOC_Os08g43190 | −1.3744 | Upregulated | dehydrogenase, putative, |
| | | | | | expressed |
| | misc.myrosinases-lectin-jacalin | LOC_Os01g24710 | 2.4570 | Downregulated | jacalin-like lectin domain |
| | | | | | containing protein, expressed |
| | misc.short chain | LOC_Os04g44950 | −0.3304 | Upregulated | short-chain |
| | dehydrogenase/reductase (SDR) | | | | dehydrogenase/reductase, |
| | | | | | putative, expressed |
| RNA | RNA.processing.ribonucleases | LOC_Os08g33710 | 0.6885 | Downregulated | ribonuclease T2 family domain |
| Processing | | | | | containing protein, expressed |
| | RNA.processing.ribonucleases | LOC_Os07g33240 | −0.1039 | Upregulated | endoribonuclease, putative, |
| | | | | | expressed |
| | RNA.regulation of transcription.Zn- | LOC_Os02g32350 | −0.9490 | Upregulated | TUDOR protein with multiple |
| | finger(CCHC) | | | | SNc domains, putative, |
| | | | | | expressed |
| | RNA.regulation of | LOC_Os04g31700 | −0.4509 | Upregulated | methylisocitrate lyase 2, |
| | transcription.unclassified | | | | putative, expressed |
| | RNA.regulation of | LOC_Os01g13700 | −0.0045 | Upregulated | DNA-binding protein-related, |
| | transcription.unclassified | | | | putative, expressed |
| | RNA.RNA binding | LOC_Os09g10760 | −0.0249 | Upregulated | RNA recognition motif |
| | | | | | containing protein, putative, |
| | | | | | expressed |
| | RNA.RNA binding | LOC_Os09g39180 | −0.0152 | Upregulated | RNA recognition motif |
| | | | | | containing protein, putative, |
| | | | | | expressed |
| Protein | N-metabolism.ammonia | LOC_Os07g46460 | −1.2889 | Upregulated | ferredoxin-dependent glutamate |
| Metabolism, | metabolism.glutamate synthase | | | | synthase, chloroplast precursor, |
| Synthesis and | | | | | putative, expressed |
| Degradation | N-metabolism.ammonia | LOC_Os02g50240 | −0.1724 | Upregulated | glutamine synthetase, catalytic |
| | metabolism.glutamine synthase | | | | domain containing protein, |
| | | | | | expressed |
| | N-metabolism.ammonia | LOC_Os04g56400 | 0.5629 | Downregulated | glutamine synthetase, catalytic |
| | metabolism.glutamine synthase | | | | domain containing protein, |
| | | | | | expressed |
| | protein.synthesis.ribosomal | LOC_Os01g47330 | 1.8344 | Downregulated | ribosomal protein L7/L12 C- |
| | protein.prokaryotic.chloroplast.50S | | | | terminal domain containing |
| | subunit.L12 | | | | protein, expressed |
| | protein.synthesis.ribosomal | LOC_Os03g38000 | −1.1640 | Upregulated | 40S ribosomal protein S3-1, |
| | protein.eukaryotic.40S subunit.S3 | | | | putative, expressed |
| | protein.synthesis.ribosomal | LOC_Os03g08440 | −0.2717 | Upregulated | ribosomal protein S2, putative, |
| | Protein.eukaryotic.40S subunit.SA | | | | expressed |
| | protein.synthesis.ribosomal | LOC_Os07g10720 | −0.7793 | Upregulated | 40S ribosomal protein S15a, |
| | protein.eukaryotic.40S subunit.S15A | | | | putative, expressed |
| | protein.synthesis.ribosomal | LOC_Os01g67134 | −0.4599 | Upregulated | ribosomal L18p/L5e family |
| | protein.eukaryotic.60S subunit.L5 | | | | protein, putative, expressed |
| | protein.synthesis.ribosomal | LOC_Os08g13690 | −0.5173 | Upregulated | 60S ribosomal protein L7, |
| | protein.eukaryotic.60S subunit.L7 | | | | putative, expressed |
| | protein.synthesis.ribosomal | LOC_Os02g18380 | −0.5112 | Upregulated | 60S ribosomal protein L27-3, |
| | protein.eukaryotic.60S subunit.L27 | | | | putative, expressed |
| | protein.synthesis.ribosomal | LOC_Os08g03640 | −0.6702 | Upregulated | 60S acidic ribosomal protein |
| | protein.eukaryotic.60S subunit.P0 | | | | P0, putative, expressed |
| | protein.synthesis.ribosomal | LOC_Os05g37330 | 1.1622 | Downregulated | 60S acidic ribosomal protein, |
| | protein.eukaryotic.60S subunit.P2 | | | | putative, expressed |
| | protein.synthesis.ribosomal | LOC_Os01g13080 | −0.4186 | Upregulated | 60S acidic ribosomal protein, |
| | protein.eukaryotic.60S subunit.P3 | | | | putative, expressed |
| | protein.synthesis.initiation | LOC_Os03g55150 | 0.2479 | Downregulated | eukaryotic translation initiation |
| | | | | | factor 5A, putative, expressed |
| | protein.synthesis.initiation | LOC_Os07g40580 | −0.3394 | Upregulated | eukaryotic translation initiation |
| | | | | | factor 5A, putative, expressed |
| | protein.synthesis.elongation | LOC_Os02g12800 | −0.3715 | Upregulated | elongation factor 1-gamma, |
| | | | | | putative, expressed |
| | protein.synthesis.elongation | LOC_Os07g42300 | −0.4568 | Upregulated | elongation factor protein, |
| | | | | | putative, expressed |
| | protein.synthesis.elongation | LOC_Os03g08020 | 0.1874 | Downregulated | elongation factor Tu, putative, |
| | | | | | expressed |
| | protein.synthesis.elongation | LOC_Os03g08050 | 0.1874 | Downregulated | elongation factor Tu, putative, |
| | | | | | expressed |
| | protein.synthesis.elongation | LOC_Os02g32030. | 0.2202 | Downregulated | elongation factor, putative, |
| | | | | | expressed |
| | protein.synthesis.elongation | LOC_Os03g08010 | 0.1874 | Downregulated | elongation factor Tu, putative, |
| | | | | | expressed |
| | protein.targeting.secretory | LOC_Os01g59790 | 0.0847 | Downregulated | ADP-ribosylation factor, |
| | pathway.unspecified | | | | putative, expressed |
| | protein.postranslational modification | LOC_Os04g40600. | −0.4652 | Upregulated | peptide methionine sulfoxide |
| | | | | | reductase, putative, expressed |
| | protein.degradation | LOC_Os02g55140 | −0.1154 | Upregulated | leucine aminopeptidase, |
| | | | | | chloroplast precursor, putative, |
| | | | | | expressed |
| | protein.degradation.cysteine protease | | −0.2603 | Upregulated | oryzain alpha chain precursor, |
| | | | | | putative, expressed |
| | protein.degradation.serine protease | LOC_Os10g01134 | −0.1560 | Upregulated | OsSCP46 - Putative Serine |
| | | | | | Carboxypeptidase homologue, |
| | | | | | expressed |
| | protein.degradation.serine protease | LOC_Os04g32560 | 0.5961 | Downregulated | ATP-dependent Clp protease |
| | | | | | ATP-binding subunit clpA |
| | | | | | homolog CD4B,chloroplast |
| | | | | | precursor, putative, expressed |
| | protein.degradation.ubiquitin.ubiquitin | LOC_Os03g13170 | 0.0678 | Downregulated | ubiquitin fusion protein, |
| | | | | | putative, expressed |
| | protein.degradation.ubiquitin.E2 | LOC_Os01g48280 | −0.3306 | Upregulated | ubiquitin-conjugating enzyme, |
| | | | | | putative, expressed |
| | protein.degradation.ubiquitin.proteasom | LOC_Os05g09490 | −0.1796 | Upregulated | peptidase, T1 family, putative, |
| | | | | | expressed |
| | protein.degradation.ubiquitin.proteasom | LOC_Os01g59600 | −0.1712 | Upregulated | peptidase, T1 family, putative, |
| | | | | | expressed |
| | protein.degradation.ubiquitin.proteasom | LOC_Os02g53060 | 0.3061 | Downregulated | peptidase, T1 family, putative, |
| | | | | | expressed |
| | protein.degradation.ubiquitin.proteasom | LOC_Os02g56000 | −0.5454 | Upregulated | 26S protease regulatory subunit |
| | | | | | 6A, putative, expressed |
| | protein.degradation.ubiquitin.proteasom | LOC_Os06g06030 | 0.0959 | Downregulated | peptidase, T1 family, putative, |
| | | | | | expressed |
| | protein.degradation.ubiquitin.proteasom | LOC_Os04g36700 | 0.6341 | Downregulated | proteasome subunit putative, |
| | | | | | expressed |
| | protein.degradation.ubiquitin.proteasom | LOC_Os02g04100 | −0.2910 | Upregulated | peptidase, T1 family, putative, |
| | | | | | expressed |
| | protein.degradation.ubiquitin.proteasom | LOC_Os03g08280 | −0.5019 | Upregulated | peptidase, T1 family, putative, |
| | | | | | expressed |
| | protein.degradation.ubiquitin.proteasom | LOC_Os03g48930 | −0.1722 | Upregulated | peptidase, T1 family, putative, |
| | | | | | expressed |
| | protein.folding | LOC_Os09g26730 | 0.6859 | Downregulated | chaperonin, putative, expressed |
| | protein.folding | LOC_Os06g02380 | −0.4236 | Upregulated | T-complex protein, putative, |
| | | | | | expressed |
| | protein.folding | LOC_Os02g39870 | −0.1779 | Upregulated | co-chaperone GrpE protein, |
| | | | | | putative, expressed |
| | protein.folding | LOC_Os07g44740 | 0.2354 | Downregulated | chaperonin, putative, expressed |
| | protein.folding | LOC_Os06g09688 | −0.3274 | Upregulated | chaperonin, putative, expressed |
| | protein.folding | LOC_Os10g32550 | 0.1087 | Downregulated | T-complex protein, putative, |
| | | | | | expressed |
| Signaling | signalling.receptor kinases.misc | LOC_Os06g41560 | 0.1485 | Downregulated | receptor-like protein kinase, |
| Proteins | | | | | putative, expressed |
| | signalling.calcium | LOC_Os08g44660 | 3.1484 | Downregulated | EF hand family protein, |
| | | | | | putative, expressed |
| | signalling.calcium | LOC_Os03g29770 | 1.0111 | Downregulated | EF hand family protein, |
| | | | | | expressed |
| | signalling.calcium | LOC_Os07g48780 | 2.5072 | Downregulated | OsCam1-2 - Calmodulin |
| | | | | | expressed |
| | signalling.calcium | LOC_Os07g14270 | −1.0132 | Upregulated | calreticulin precursor protein, |
| | | | | | putative, expressed |
| | signalling.calcium | LOC_Os03g20370 | 2.5072 | Downregulated | OsCam1-1 - Calmodulin |
| | | | | | expressed |
| | signalling.G-proteins | LOC_Os07g31370 | −0.5342 | Upregulated | ms-related protein, putative, |
| | | | | | expressed |
| | signalling.G-proteins | LOC_Os01g37800 | −0.3751 | Upregulated | ras-related protein, putative, |
| | | | | | expressed |
| | signalling.G-proteins | LOC_Os05g49890 | −0.9823 | Upregulated | ras-related protein, putative, |
| | | | | | expressed |
| | signalling.G-proteins | LOC_Os01g15010 | 0.0147 | Downregulated | miro, putative, expressed |
| | signalling.14-3-3 proteins | LOC_Os08g33370 | −0.7621 | Upregulated | 14-3-3 protein putative, |
| | | | | | expressed |
| | signalling.14-3-3 proteins | LOC_Os08g37490 | −0.6166 | Upregulated | 14-3-3 protein putative, |
| | | | | | expressed |
| | signalling.14-3-3 proteins | LOC_Os03g50290 | −0.0962 | Upregulated | 14-3-3 protein putative, |
| | | | | | expressed |
| | signalling.14-3-3 proteins | LOC_Os02g36974 | −1.0262 | Upregulated | 14-3-3 protein putative, |
| | | | | | expressed |
| Development - | development.storage proteins | LOC_Os03g57960 | −3.1262 | Upregulated | cupin domain containing |
| Related Proteins | | | | | protein, expressed |
| | development.storage proteins | LOC_Os05g02520 | 0.0517 | Downregulated | cupin domain containing |
| | | | | | protein, expressed |
| | development.unspecified | LOC_Os01g49290 | −1.4699 | Upregulated | WD repeat-containing protein, |
| | | | | | putative, expressed |
| | not assigned.unknown | LOC_Os05g41970 | −4.1859 | Upregulated | SSA1 - 2S albumin seed |
| | | | | | storage family protein |
| | | | | | precursor, expressed |
| Transport | transport.- and v-ATPases.H+- | LOC_Os06g45120 | 0.1731 | Downregulated | ATP synthase, putative, |
| Proteins | transporting two-sector ATPase | | | | expressed |
| | transport.p- and v-ATPases.H+- | LOC_Os06g37180 | −0.3423 | Upregulated | ATP synthase, putative, |
| | transporting two-sector ATPase | | | | expressed |
| | transport.misc | LOC_Os05g35460 | 0.9191 | Downregulated | patellin protein, putative, |
| | | | | | expressed |
| Unassigned | gluconeogenesis.Malate DH | LOC_Os03g56280 | −0.0558 | Upregulated | lactate/malate dehydrogenase, |
| Classes | | | | | putative, expressed |
| | gluconeogenese/glyoxylate | LOC_Os05g33570 | −1.9356 | Upregulated | pyruvate, phosphate dikinase, |
| | cycle.pyruvate dikinase | | | | chloroplast precursor, putative, |
| | | | | | expressed |
| | metal handling | LOC_Os01g68770 | −0.0763 | Upregulated | selenium-binding protein, |
| | | | | | putative, expressed |
| | Co-factor and vitamine metabolism.folate | LOC_Os09g36800 | −0.6866 | Upregulated | 3-dehydroquinate synthase, |
| | & vitamine K | | | | putative, expressed |
| | not assigned.unknown | LOC_Os01g71300 | −0.1383 | Upregulated | S-formylglutathione hydrolase, |
| | | | | | putative, expressed |
| | not assigned.unknown | LOC_Os03g22460 | −0.1045 | Upregulated | expressed protein |
| | not assigned.unknown | LOC_Os02g55810 | 1.8521 | Downregulated | CXXXC9 - Cysteine-rich |
| | | | | | protein with paired CXXXC |
| | | | | | motifs precursor, expressed |
| | not assigned.unknown | LOC_Os09g17660 | 0.4829 | Downregulated | expressed protein |
| | not assigned.unknown | LOC_Os11g06570 | −0.5549 | Upregulated | transposon protein, putative, |
| | | | | | CACTA, En/Spm sub-class, |
| | | | | | expressed |
| | not assigned.unknown | LOC_Os03g60509 | −0.1358 | Upregulated | expressed protein |

Protein and Metabolite Factors for Better Source and Sink Capacity for Yield Under Drought.

Compared to Vandana, higher sucrose and starch content in the flag leaf as well as in the spikelets of the 481-B during stress, indicated better source and sink capacity in the 481-B (FIGS. 30, 31). This was further supported by the observation that although the activity of two starch biosynthesis enzymes, Glucose-1-phosphate adenylyltransferase and sucrose synthase is known to be acutely impaired under drought (Ahmadi and Baker, 2001; Smith et al., 1997), the flag leaf and the spikelets of the 481-B contained larger amounts of these enzymes than the Vandana flag leaf and spikelets (FIGS. 25, 31E-31F). The other key proteins identified for carbohydrate metabolism were starch branching enzyme (1,4-α-glucan branching enzyme), β-amylase and adenylate kinase. β-amylases degrade starch to maltose in a series of reactions to supply energy and carbon skeletons for metabolism during dark period (Smith et al., 2005; He et al., 2011). In the spikelets of the 481-B, β-amylase was down-regulated (FIG. 25). This showed reduced starch degradation consistent with the presence of higher starch in the spikelets of the 481-B (FIG. 31D). A higher amount of starch indicates reduced adenylate kinase (Oliver et al., 2008) and the spikelets of the 481-B exhibited adenylate kinase down-regulation (FIG. 31G). A higher amount of starch in the spikelets of the 481-B also explains the observation of upregulated sucrose synthase and 1,4-α-glucan branching enzyme (FIGS. 25, 31F). Additionally, thioredoxins were upregulated in spikelets of the 481-B (FIG. 25) where they may be involved in the post-translational redox-activation of ADP23 glucose pyrophosphorylase (Meyer et al., 1999; Tiessen et al., 2002; Oliver et al., 2008) towards starch synthesis. Down-regulation of thioredoxins in flag leaf (FIG. 25) showed that despite the upregulated AGPase, starch synthesis is inhibited as observed (FIG. 30D) and simple sugars accumulated for translocation towards spikelets.

Proteomic data also showed down-regulation of the glycolysis related proteins in flag leaf of the 481-B compared to Vandana. The respiratory pathways are generally accelerated during drought stress (Haupt-Herting et al., 2001). However, Glyceraldehyde 3-phosphate dehydrogenase, phosphoglucomutase, glucose 6-phosphate isomerase and enolase were down-regulated in the flag leaf and spikelets of 481-B (FIG. 24) indicating down-regulation of glycolysis and more sugars available in the flag leaf and the spikelets (FIGS. 30, 31) to serve as osmoticum and as a source for starch synthesis respectively. Taken together the proteome data for carbohydrate metabolism and the metabolite data 1 for sugars and starch indicate that better yield in the 481-B resides in better assimilate source and sink capacity and better translocation/remobilization capacity of the 481-B.

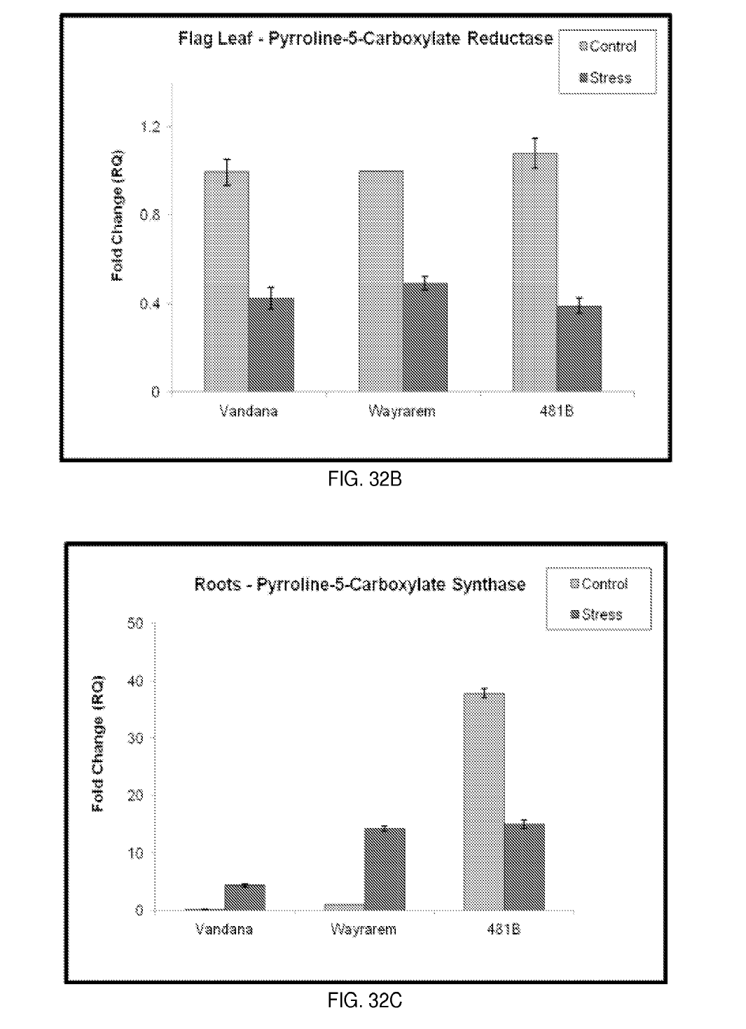

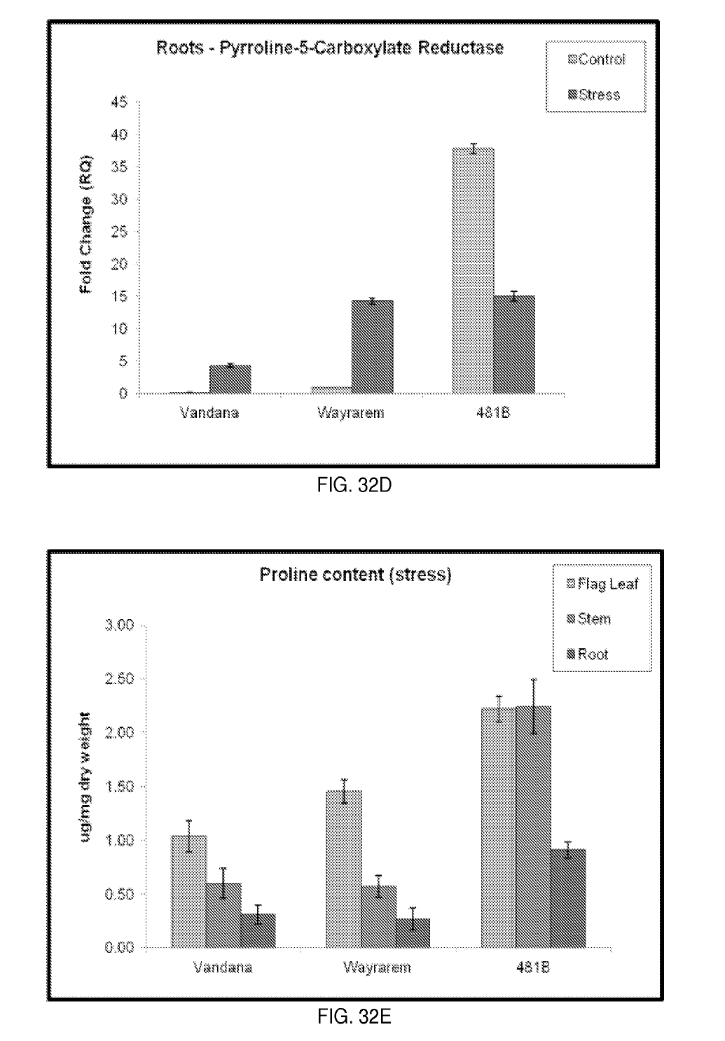

Additionally, amino acids play a key role in cellular processes during grain filling. They serve as precursors feeding into other anabolic pathways; play a major anaplerotic role to feed the intermediates of the TCA cycle (Ashraf and Foolad, 2007; Sweetlove et al., 2010); may act as osmolytes under drought (Verslues and Sharma, 2010) and define the overall N status. Newly acquired N is insufficient for the high demands during grain filling and N remobilization and partitioning are crucial for grain filling. Reduction of glutamate noticed in the flag leaf under drought occurs due to its translocation, to the spikelets, which show higher glutamate in the spikelets of the 481-B only (FIGS. 32A-32C), indicating better remobilization capacity of the 481-B. Another transportable amino acid aspartate is also present in larger amounts in the spikelets of the 481-B (FIG. 32C). Overall, better N remobilization from flag leaf to spikelets in the 481-B compared to the parents was observed (FIG. 38).

TABLE 7
The complete set of proteins identified in roots of Vandana and the 481B NIL (in triplicates),
represented as fold increase in the 481B NIL compared to Vandana during drought stress.

| | | | Log 2 | | |
|---|---|---|---|---|---|
| Gene Ontology | MapMan Bin Description | Locus Id's | Ratio | | MSU v7.0 description |
| e-Carrier | PS.lightreaction.other | LOC_Os03g61960 | 1.6104 | Downregulated | 2Fe-2S iron-sulfur cluster binding domain |
| | electron carrier | | | | containing protein, expressed |
| | (ox/red).ferredoxin | | | | |
| Calvin | PS.calvin | LOC_Os06g45710 | −2.0766 | Upregulated | phosphoglycerate kinase protein, putative, |
| Cycle | cycle.phosphoglycerate | | | | expressed |
| | kinase | | | | |
| | PS.calvin | LOC_Os02g07260 | −2.3320 | Upregulated | phosphoglycerate kinase protein, putative, |
| | cycle.phosphoglycerate | | | | expressed |
| | kinase | | | | |
| | PS.calvin cycle.TPI | LOC_Os01g05490 | −1.6061 | Upregulated | triosephosphate isomerase, cytosolic, |
| | | | | | putative, expressed |
| | PS.calvin cycle.aldolase | LOC_Os01g67860 | −2.4698 | Upregulated | fructose-bisphospate aldolase isozyme, |
| | | | | | putative, expressed |
| CHO | major CHO | LOC_Os06g09450 | −2.8827 | Upregulated | sucrose synthase, putative, expressed |
| Metabolism | metabolism.degradation.sucrose. | | | | |
| | Susy | | | | |
| Glycolysis | glycolysis.cytosolic | LOC_Os08g03290 | −2.0223 | Upregulated | glyceraldehyde-3-phosphate |
| | branch.glyceraldehyde 3- | | | | dehydrogenase, putative, expressed |
| | phosphate dehydrogenase | | | | |
| | (GAP-DH) | | | | |
| | glycolysis.cytosolic | LOC_Os02g38920 | −2.1735 | Upregulated | glyceraldehyde-3-phosphate |
| | branch.glyceraldehyde 3- | | | | dehydrogenase, putative, expressed |
| | phosphate dehydrogenase | | | | |
| | (GAP-DH) | | | | |
| | glycolysis.cytosolic | LOC_Os10g08550 | −1.6314 | Upregulated | enolase, putative, expressed |
| | branch.enolase | | | | |
| | not assigned.unknown | LOC_Os03g50480 | −2.4066 | Upregulated | phosphoglucomutase, putative, expressed |
| Oxidadtive | OPP.non-reductive | LOC_Os01g70170 | −3.6772 | Upregulated | transaldolase, putative, expressed |
| Pentose | PP.transaldolase | | | | |
| Phosphate | | | | | |
| Pathway | | | | | |
| TCA Cycle | TCA/org. | LOC_Os01g22520 | 0.5806 | Downregulated | dihydrolipoyl dehydrogenase 1, |
| | transformation.TCA.pyruvate | | | | mitochondrial precursor, putative, |
| | DH.E3 | | | | expressed |
| | TCA/org. | LOC_Os05g49880 | −1.9303 | Upregulated | lactate/malate dehydrogenase, putative, |
| | transformation.TCA.malate | | | | expressed |
| | DH | | | | |
| | TCA/org. | LOC_Os10g33800 | −2.2644 | Upregulated | lactate/malate dehydrogenase, putative, |
| | transformation.other organic | | | | expressed |
| | acid transformaitons.cyt | | | | |
| | MDH | | | | |
| Mitochondrial | mitochondrial electron | LOC_Os03g27290 | 1.8064 | Downregulated | cytochrome c oxidase subunit, putative, |
| Electron | transport/ATP | | | | expressed |
| Transport | synthesis.cytochrome c | | | | |
| Chain | oxidase | | | | |
| | mitochondrial electron | LOC_Os07g42910 | 0.1653 | Downregulated | cytochrome c oxidase subunit, putative, |
| | transport/ATP | | | | expressed |
| | synthesis.cytochrome c | | | | |
| | oxidase | | | | |
| | mitochondrial electron | LOC_Os08g37320 | 1.8375 | Downregulated | expressed protein |
| | transport/ATP synthesis.F1- | | | | |
| | ATPase | | | | |
| | mitochondrial electron | LOC_Os07g31300 | 1.8367 | Downregulated | ATP synthase delta chain, mitochondrial |
| | transport/ATP synthesis.F1- | | | | precursor, putative, expressed |
| | ATPase | | | | |
| | mitochondrial electron | LOC_Os05g47980 | −1.3455 | Upregulated | ATP synthase, putative, expressed |
| | transport/ATP synthesis.F1- | | | | |
| | ATPase | | | | |
| Cell and | cell wall.cell wall | LOC_Os01g47780 | 0.7048 | Downregulated | fasciclin domain containing protein, |
| Cell Wall - | proteins.AGPs.AGP | | | | expressed |
| Related | cell wall.cell wall | LOC_Os05g48890 | 0.5626 | Downregulated | fasciclin domain containing protein, |
| Proteins | proteins.AGPs.AGP | | | | expressed |
| | cell wall.cell wall | LOC_Os01g06580 | 0.1585 | Downregulated | fasciclin domain containing protein, |
| | proteins.AGPs.AGP | | | | expressed |
| | cell.organisation | LOC_Os09g33810 | 0.8809 | Downregulated | ankyrin repeat domain containing protein, |
| | | | | | putative, expressed |
| | cell.organisation | LOC_Os07g38730 | −3.1326 | Upregulated | tubulin/FtsZ domain containing protein, |
| | | | | | putative, expressed |
| | cell.organisation | LOC_Os01g64630 | −2.7243 | Upregulated | actin, putative, expressed |
| | cell.organisation | LOC_Os03g60580 | 2.1547 | Downregulated | actin-depolymerizing factor, putative, |
| | | | | | expressed |
| | cell.organisation | LOC_Os03g48540 | −0.0225 | Upregulated | expressed protein |
| | cell.organisation | LOC_Os06g46000 | −2.6175 | Upregulated | tubulin/FtsZ domain containing protein, |
| | | | | | putative, expressed |
| | cell.organisation | LOC_Os11g14220 | −3.4083 | Upregulated | tubulin/FtsZ domain containing protein, |
| | | | | | putative, expressed |
| | not assigned.unknown | LOC_Os05g39250 | 1.9046 | Downregulated | phosphatidylethanolamine-binding |
| | | | | | protein, putative, expressed |
| | not assigned.unknown | LOC_Os03g04110 | −0.9124 | Upregulated | lysM domain-containing GPI-anchored |
| | | | | | protein precursor, putative, expressed |
| Lipid | lipid metabolism.FA | LOC_Os08g06550 | 2.4541 | Downregulated | acyl CoA binding protein, putative, |
| Metabolism | synthesis and FA | | | | expressed |
| | elongation.acyl-CoA binding | | | | |
| | protein | | | | |
| | lipid metabolism.″exotics″ | LOC_Os08g04460 | −1.0480 | Upregulated | NADPH-dependent FMN reductase |
| | (steroids, squalene etc) | | | | domain containing protein, expressed |
| | not assigned.unknown | LOC_Os05g40010 | 1.1150 | Downregulated | LTPL17 - Protease inhibitor/seed |
| | | | | | storage/LTP family protein precursor, |
| | | | | | expressed |
| | not assigned.unknown | LOC_Os07g43290 | 0.2648 | Downregulated | LTPL56 - Protease inhibitor/seed |
| | | | | | storage/LTP family protein precursor, |
| | | | | | expressed |
| | not assigned.unknown | LOC_Os03g26820 | −0.8372 | Upregulated | LTPL52 - Protease inhibitor/seed |
| | | | | | storage/LTP family protein precursor, |
| | | | | | expressed |
| | misc.protease inhibitor/seed | LOC_Os04g33920 | 1.2606 | Downregulated | LTPL102 - Protease inhibitor/seed |
| | storage/lipid transfer protein | | | | storage/LTP family protein precursor, |
| | (LTP) family protein | | | | expressed |
| | misc.protease inhibitor/seed | LOC_Os07g09970 | 1.4528 | Downregulated | LTPL84 - Protease inhibitor/seed |
| | storage/lipid transfer protein | | | | storage/LTP family protein precursor, |
| | (LTP) family protein | | | | expressed |
| | misc.protease inhibitor/seed | LOC_Os05g06780 | 2.5306 | Downregulated | LTPL104 - Protease inhibitor/seed |
| | storage/lipid transfer protein | | | | storage/LTP family protein precursor, |
| | (LTP) family protein | | | | expressed |
| Amino | amino acid | LOC_Os08g09250 | −2.2837 | Upregulated | glyoxalase family protein, putative, |
| Acid | metabolism.degradation.aspartate | | | | expressed |
| Metabolism | family.threonine | | | | |
| | amino acid | LOC_Os07g09060 | −1.5902 | Upregulated | aldehyde dehydrogenase, putative, |
| | metabolism.degradation. | | | | expressed |
| | branched-chain group.valine | | | | |
| Secondary | secondary | LOC_Os08g38900 | −1.8384 | Upregulated | caffeoyl-CoA O-methyltransferase, |
| Metabolism | metabolism.phenylpropanoids.lignin | | | | putative, expressed |
| | biosynthesis.CCoAOMT | | | | |
| Hormones | hormone | LOC_Os08g41290 | 0.7677 | Downregulated | AIR12, putative, expressed |
| | metabolism.auxin.induced- | | | | |
| | regulated-responsive- | | | | |
| | activated | | | | |
| Stress - | stress.biotic | LOC_Os05g33140 | 0.7055 | Downregulated | CHITS - Chitinase family protein |
| Related | | | | | precursor, expressed |
| Proteins | stress.biotic | LOC_Os07g03710 | 1.0875 | Downregulated | SCP-like extracellular protein, expressed |
| | stress.biotic | LOC_Os09g21210 | −1.7680 | Upregulated | beta-glucan-binding protein 4, putative, |
| | | | | | expressed |
| | stress.biotic | LOC_Os10g11500 | 0.6809 | Downregulated | SCP-like extracellular protein, expressed |
| | stress.biotic | LOC_Os03g46060 | 0.0245 | Downregulated | thaumatin family domain containing |
| | | | | | protein, expressed |
| | stress.biotic | LOC_Os05g15770 | −1.5297 | Upregulated | glycosyl hydrolase, putative, expressed |
| | stress.biotic | LOC_Os05g33130 | −3.4115 | Upregulated | CHIT17 - Chitinase family protein |
| | | | | | precursor, expressed |
| | stress.biotic | LOC_Os03g46070 | 1.1931 | Downregulated | thaumatin, putative, expressed |
| | stress.biotic | LOC_Os10g18820 | 0.4468 | Downregulated | dirigent, putative, expressed |
| | stress.biotic | LOC_Os10g34920 | 0.9595 | Downregulated | secretory protein, putative, expressed |
| | stress.biotic.PR-proteins | LOC_Os07g01660 | 0.9938 | Downregulated | dirigent, putative, expressed |
| | stress.abiotic.heat | LOC_Os04g36750 | 1.6417 | Downregulated | hsp20/alpha crystallin family protein, |
| | | | | | putative, expressed |
| | stress.abiotic.heat | LOC_Os01g62290 | 1.0118 | Downregulated | DnaK family protein, putative, expressed |
| | stress.abiotic.heat | LOC_Os03g02260 | −0.9892 | Upregulated | DnaK family protein, putative, expressed |
| | stress.abiotic.heat | LOC_Os04g01740 | −2.7931 | Upregulated | heat shock protein, putative, expressed |
| | stress.abiotic.heat | LOC_Os03g15960 | 1.5378 | Downregulated | hsp20/alpha crystallin family protein, |
| | | | | | putative, expressed |
| | stress.abiotic.heat | LOC_Os01g04370 | 1.2218 | Downregulated | hsp20/alpha crystallin family protein, |
| | | | | | putative, expressed |
| | stress.abiotic.heat | LOC_Os08g39140 | −2.3938 | Upregulated | heat shock protein, putative, expressed |
| | stress.abiotic.heat | LOC_Os05g38530 | −2.9993 | Upregulated | DnaK family protein, putative, expressed |
| | stress.abiotic.heat | LOC_Os06g50300 | −2.8550 | Upregulated | heat shock protein, putative, expressed |
| | stress.abiotic.cold | LOC_Os08g03520 | 1.6074 | Downregulated | retrotransposon protein putative, Ty1- |
| | | | | | copia subclass, expressed |
| | stress.abiotic.drought/salt | LOC_Os01g13210 | 1.9693 | Downregulated | salt stress root protein RS1, putative, |
| | | | | | expressed |
| | stress.abiotic.unspecified | LOC_Os03g48770 | 1.3813 | Downregulated | Cupin domain containing protein, |
| | | | | | expressed |
| | stress.abiotic.unspecified | LOC_Os01g18170 | 0.1738 | Downregulated | Cupin domain containing protein, |
| | | | | | expressed |
| | stress.abiotic.unspecified | LOC_Os01g14670 | −0.2366 | Upregulated | Cupin domain containing protein, |
| | | | | | expressed |
| | stress.abiotic.unspecified | LOC_Os08g08960 | −0.1897 | Upregulated | Cupin domain containing protein, |
| | | | | | expressed |
| | not assigned.unknown | LOC_Os01g50910 | −0.1233 | Upregulated | late embryogenesis abundant protein, |
| | | | | | group 3, putative, expressed |
| | not assigned.unknown | LOC_Os05g47470 | 1.0238 | Downregulated | VIP1 protein, putative, expressed |
| | not assigned.unknown | LOC_Os05g46480 | 1.0024 | Downregulated | late embryogenesis abundant protein, |
| | | | | | group 3, putative, expressed |
| | not assigned.unknown | LOC_Os11g06720 | 1.7088 | Downregulated | abscisic stress-ripening, putative, |
| | | | | | expressed |
| | not assigned.unknown | LOC_Os11g26750 | 0.2255 | Downregulated | dehydrin, putative, expressed |
| Redox - | redox.thioredoxin | LOC_Os11g09280 | −0.4074 | Upregulated | OsPDIL1-1 protein disulfide isomerase |
| Related | | | | | PDIL1-1, expressed |
| Protein | redox.thioredoxin | LOC_Os07g08840 | 2.2353 | Downregulated | thioredoxin, putative, expressed |
| | redox.ascorbate and | LOC_Os05g02530 | −0.2316 | Upregulated | glutathione S-transferase, N-terminal |
| | glutathione.ascorbate | | | | domain containing protein, expressed |
| | redox.ascorbate and | LOC_Os07g49400 | −0.7335 | Upregulated | OsAPx2 - Cytosolic Ascorbate Peroxidase |
| | glutathione.ascorbate | | | | encoding gene 4,5,6,8, expressed |
| | redox.ascorbate and | LOC_Os03g17690 | −0.8999 | Upregulated | OsAPx1 - Cytosolic Ascorbate Peroxidase |
| | glutathione.ascorbate | | | | encoding gene 1-8, expressed |
| | redox.glutaredoxins | LOC_Os04g42930. | 1.6128 | Downregulated | OsGrx C2.2 - glutaredoxin subgroup I, |
| | | | | | expressed |
| | redox.dismutases and | LOC_Os07g46990 | 2.5119 | Downregulated | copper/zinc superoxide dismutase, |
| | catalases | | | | putative, expressed |
| | redox.dismutases and | LOC_Os08g44770 | 0.3297 | Downregulated | copper/zinc superoxide dismutase, |
| | catalases | | | | putative, expressed |
| | misc.glutathione S | LOC_Os01g55830 | −2.3557 | Upregulated | glutathione S-transferase, putative, |
| | transferases | | | | expressed |
| | misc.peroxidases | LOC_Os09g29490 | −0.1418 | Upregulated | peroxidase precursor, putative, expressed |
| | misc.peroxidases | LOC_Os01g22370 | −0.9751 | Upregulated | peroxidase precursor, putative, expressed |
| | misc.peroxidases | LOC_Os02g14160 | 0.5096 | Downregulated | peroxidase precursor, putative, expressed |
| | misc.peroxidases | LOC_Os04g59150 | −2.0639 | Upregulated | peroxidase precursor, putative, expressed |
| | misc.peroxidases | LOC_Os01g73170 | −1.7416 | Upregulated | peroxidase precursor, putative, expressed |
| | misc.peroxidases | LOC_Os06g35520 | −0.1243 | Upregulated | peroxidase precursor, putative, expressed |
| | misc.peroxidases | LOC_Os03g55410 | −0.4993 | Upregulated | peroxidase precursor, putative, expressed |
| | misc.peroxidases | LOC_Os10g02040 | −0.4560 | Upregulated | peroxidase precursor, putative, expressed |
| | misc.peroxidases | LOC_Os05g04500 | −0.3602 | Upregulated | peroxidase precursor, putative, expressed |
| | misc.peroxidases | LOC_Os07g48020 | −0.2130 | Upregulated | peroxidase precursor, putative, expressed |
| | misc.peroxidases | LOC_Os07g01410 | −1.0294 | Upregulated | peroxidase precursor, putative, expressed |
| | misc.peroxidases | LOC_Os01g22352 | −0.5611 | Upregulated | peroxidase precursor, putative, expressed |
| | misc.peroxidases | LOC_Os07g48030 | 0.2461 | Downregulated | peroxidase precursor, putative, expressed |
| | misc.peroxidases | LOC_Os05g41990 | −1.2460 | Upregulated | peroxidase precursor, putative, expressed |
| Nucleotide | DNA.synthesis/chromatin | LOC_Os06g48750 | −2.7569 | Upregulated | DEAD-box ATP-dependent RNA |
| Metabolism | structure | | | | helicase, putative, expressed (″DEAD″ |
| | | | | | disclosed as SEQ ID NO: 181) |
| | DNA.synthesis/chromatin | LOC_Os05g49860 | 1.5039 | Downregulated | Core histone H2A/H2B/H3/H4 domain |
| | structure.histone | | | | containing protein, putative, expressed |
| Miscellaneous | Biodegradation of | LOC_Os08g09250 | −2.2837 | Upregulated | glyoxalase family protein, putative, |
| Enzymes | Xenobiotics | | | | expressed |
| | misc.beta 1,3 glucan | LOC_Os07g35480 | 1.1377 | Downregulated | glucan endo-1,3-beta-glucosidase |
| | hydrolases.glucan endo-1,3- | | | | precursor, putative, expressed |
| | beta-glucosidase | | | | |
| | misc.O-methyl transferases | LOC_Os11g20160 | −1.8477 | Upregulated | O-methyltransferase, putative, expressed |
| | misc.myrosinases-lectin- | LOC_Os01g24710 | −0.2644 | Upregulated | jacalin-like lectin domain containing |
| | jacalin | | | | protein, expressed |
| | misc.plastocyanin-like | LOC_Os06g46740 | −0.7111 | Upregulated | early nodulin 20 precursor, putative, |
| | | | | | expressed |
| RNA | RNA.processing.ribonucleases | LOC_Os07g33240 | 1.2769 | Downregulated | endoribonuclease, putative, expressed |
| Processing | RNA.regulation of | LOC_Os09g33810 | 0.8809 | Downregulated | ankyrin repeat domain containing protein, |
| | transcription.AtSR | | | | putative, expressed |
| | Transcription Factor family | | | | |
| | RNA.regulation of | LOC_Os10g39300 | −0.3913 | Upregulated | aspartic proteinase nepenthesin, putative, |
| | transcription.unclassified | | | | expressed |
| Protein | protein.synthesis.ribosomal | LOC_Os01g67134 | −2.3733 | Upregulated | ribosomal L18p/L5e family protein, |
| Metabolism, | protein.eukaryotic.60S | | | | putative, expressed |
| Synthesis | subunit.L5 | | | | |
| and | protein.synthesis.ribosomal | LOC_Os02g47140 | −0.7944 | Upregulated | L11 domain containing ribosomal protein, |
| Degradation | protein.eukaryotic.60S | | | | putative, expressed |
| | subunit.L12 | | | | |
| | protein.synthesis.ribosomal | LOC_Os06g48780 | 1.6960 | Downregulated | 60S acidic ribosomal protein, putative, |
| | protein.eukaryotic.60S | | | | expressed |
| | subunit.P3 | | | | |
| | protein.synthesis.initiation | LOC_Os03g55150 | 1.2295 | Downregulated | eukaryotic translation initiation factor SA, |
| | | | | | putative, expressed |
| | protein.synthesis.elongation | LOC_Os03g08020 | −2.1295 | Upregulated | elongation factor Tu, putative, expressed |
| | protein.synthesis.elongation | LOC_Os03g08050 | −2.1295 | Upregulated | elongation factor Tu, putative, expressed |
| | protein.synthesis.elongation | LOC_Os03g08010 | −2.1295 | Upregulated | elongation factor Tu, putative, expressed |
| | protein.degradation.cysteine | LOC_Os05g41460 | 0.2691 | Downregulated | cysteine proteinase inhibitor precursor |
| | protease | | | | protein, putative, expressed |
| | protein.degradation.cysteine | LOC_Os04g57440 | 0.2596 | Downregulated | oryzain beta chain precursor, putative, |
| | protease | | | | expressed |
| | protein.degradation.cysteine | LOC_Os04g55650 | −0.0267 | Upregulated | oryzain alpha chain precursor, putative, |
| | protease | | | | expressed |
| | protein.degradation.aspartate | LOC_Os10g39300 | −0.3913 | Upregulated | aspartic proteinase nepenthesin, putative, |
| | protease | | | | expressed |
| | protein.degradation.serine | LOC_Os03g41419 | −0.1811 | Upregulated | seipin domain containing protein, |
| | protease | | | | putative, expressed |
| | protein.degradation.ubiquitin | LOC_Os09g25320 | −0.2443 | Upregulated | ubiquitin family protein, putative, |
| | ubiquitin | | | | expressed |
| | protein.degradation.ubiquitin | LOC_Os09g39500 | −0.3355 | Upregulated | ubiquitin fusion protein, putative, |
| | ubiquitin | | | | expressed |
| | protein.folding | LOC_Os08g25090 | −0.0902 | Upregulated | co-chaperone GrpE protein, putative, |
| | | | | | expressed |
| | protein.folding | LOC_Os07g44740 | 1.7354 | Downregulated | chaperonin, putative, expressed |
| | protein.folding | LOC_Os06g09688 | 0.8771 | Downregulated | chaperonin, putative, expressed |
| | protein.folding | LOC_Os10g32550 | −1.7110 | Upregulated | T-complex protein, putative, expressed |
| | not assigned.unknown | LOC_Os01g11010 | 0.3982 | Downregulated | peptide-N4-asparagine amidase A, |
| | | | | | putative, expressed |
| | not assigned.unknown | LOC_Os01g03360 | 1.9320 | Downregulated | BBTI5 - Bowman-Birk type bran trypsin |
| | | | | | inhibitor precursor, expressed |
| Signaling | signalling.receptor | LOC_Os04g56430 | 0.7112 | Downregulated | cysteine-rich receptor-like protein kinase, |
| Proteins | kinases.misc | | | | putative, expressed |
| | signalling.calcium | LOC_Os01g04330 | 1.4649 | Downregulated | OsCML16 - Calmodulin-related calcium |
| | | | | | sensor protein, expressed |
| | signalling.calcium | LOC_Os03g20370 | 2.4802 | Downregulated | OsCam1-1 - Calmodulin, expressed |
| | signalling.MAP kinases | LOC_Os08g01310 | −0.9161 | Upregulated | phospholipase C, putative, expressed |
| | signalling.14-3-3 proteins | LOC_Os08g33370 | −1.7188 | Upregulated | 14-3-3 protein, putative, expressed |
| | signalling.14-3-3 proteins | LOC_Os04g38870 | −2.5741 | Upregulated | 14-3-3 protein, putative, expressed |
| | not assigned.unknown | LOC_Os04g25650 | 0.6821 | Downregulated | cysteine-rich receptor-like protein kinase, |
| | | | | | putative, expressed |
| Developmental - | development.late | LOC_Os01g12580 | 0.7772 | Downregulated | late embryogenesis abundant protein, |
| Related | embryogenesis abundant | | | | putative, expressed |
| Proteins | development.late | LOC_Os06g23350 | 0.6569 | Downregulated | late embryogenesis abundant protein D- |
| | embryogenesis abundant | | | | 34, putative, expressed |
| | development.unspecified | LOC_Os06g13030 | −1.6951 | Upregulated | OsLIM - LIM domain protein, putative |
| | | | | | actin-binding protein and transcription |
| | | | | | factor, expressed |
| | not assigned.unknown | LOC_Os05g28210 | 1.0307 | Downregulated | small hydrophilic plant seed protein, |
| | | | | | putative, expressed |
| | not assigned.unknown | LOC_Os03g07180 | −1.3908 | Upregulated | embryonic protein DC-8, putative, |
| | | | | | expressed |
| Transport | transport and v-ATPases | LOC_Os04g51270 | 1.5409 | Downregulated | vacuolar ATPase G subunit, putative, |
| Proteins | | | | | expressed |
| | transport.misc | LOC_Os05g35460 | 1.3513 | Downregulated | patellin protein, putative, expressed |
| Unassigned | metal handling.binding, | LOC_Os08g10480 | 1.2253 | Downregulated | heavy metal-associated domain containing |
| Classes | chelation and storage | | | | protein, expressed |
| | not assigned.unknown | LOC_Os03g15920 | −0.6574 | Upregulated | expressed protein |
| | not assigned.unknown | LOC_Os03g15910 | −0.6574 | Upregulated | membrane protein, putative, expressed |
| | not assigned.unknown | LOC_Os10g14050 | 0.1468 | Downregulated | expressed protein |
| | not assigned.unknown | LOC_Os06g12580 | −0.0268 | Upregulated | pro-resilin precursor, putative, expressed |
| | not assigned.unknown | LOC_Os08g01370 | 0.3251 | Downregulated | expressed protein |
| | not assigned.unknown | LOC_Os10g36180 | 0.0225 | Downregulated | expressed protein |

Proline Metabolism: A New Paradigm in Drought Tolerant Rice Crops.

Proline is a highly water soluble amino acid, which exists as a zwitterion and is known to accumulate to increase cellular osmolarity in plants experiencing dehydration stress and to play an important role in redox buffering and in transferring energy as per cellular demands (Verslues and Sharma, 2010). Such properties of proline depend not only on proline itself but on its metabolic cycle as well. Glutamate is the source of proline synthesis through pyrroline 5-carboxylate synthetase (P5CS) and pyrroline-5-carboxylate reductase (P5CR) and is also the product of proline degradation through proline dehydrogenase and pyrroline-5-carboxylate dehydrogenase. Mutant and transgenic approaches to functionally validate the function of these genes under salt and cold stress have been undertaken, however there is limited understanding of the role of proline in crops under drought. “The more the better” is the popular strategy but higher accumulation of proline has never shown any direct correlation to yield under stress. Other functions for proline depend on spatial and temporal control of proline metabolism to act as redox buffer and meet the plants' needs for energy (Szabados and Savouré, 2010; Verslues and Sharma, 2010).

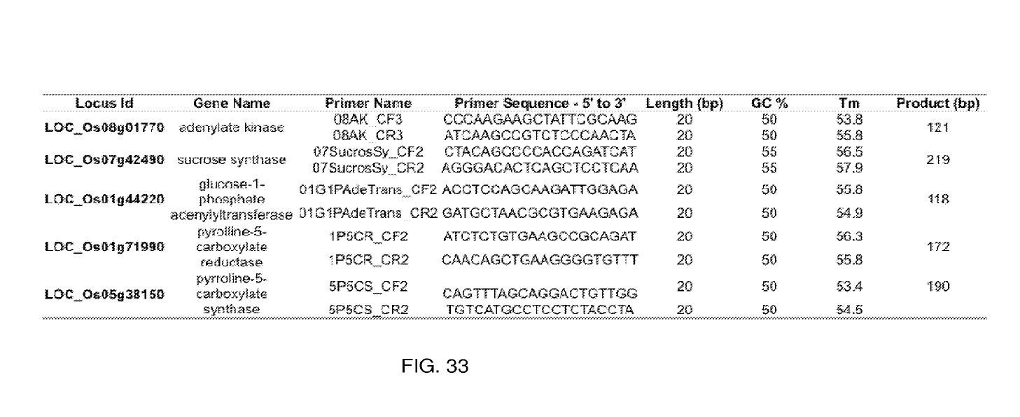

Under drought, both parents had significantly higher proline content in the flag leaf than the 481-B (FIGS. 32D-32F). The proline synthesis genes were more up-regulated in the flag leaf of the parents than in that of the 481-B (FIGS. 33A-33B). The roots on the other hand had much higher proline content in the 481-B than in the parents (FIG. 33E). However, the roots of the NILs showed a significant down-regulation of the proline synthesis genes, whereas they were up-regulated in the parents (FIGS. 33C-D). These results indicated that during stress proline is translocated from the leaf to the roots in the 481-B. Colorimetric measurement of the proline content during stress revealed much higher levels of proline in the stem of the 481-B than in that of the parental plants (FIG. 33E). Proline supply from the shoot and its catabolism in the root was shown to be essential for continued growth at low water potential in Arabidopsis (Sharma et al., 2011). Earlier, maize root tips were shown to accumulate high levels of proline during stress due to proline translocation and not due to de novo synthesis (Verslues and Sharp, 1999). Such observations indicate that stress-induced tissue-specific differences in proline metabolism and accumulation are more important than previously thought. The roots of the 481-B thus seem better equipped to counter stress and perform its critical functions towards drought tolerance.

Whether or not proline accumulation is a stress indicator or part of an adaptive response has long been debated. Earlier work correlated proline to stress and thus higher proline accumulations indicated more stress (Stewart and Hanson, 1980). However, recently higher proline content has been associated with better drought and salt tolerance (Ben Hassine et al., 2008; Evers et al., 2010; Kant et al., 2006). Reverse genetics and other molecular approaches have established that stress induced proline accumulation is useful; however they have also provided further insights into different functions of proline with respect to maintenance of homeostasis (Kishor and Sreenivasulu, 2013). Its role in osmotic adjustment have been shown in the drought stress, where decrease in soil water potential lead to higher levels of proline in unvacuolated cells of the root tip, mainly in the chloroplast stroma and the cytoplasm (Verslues and Sharp, 1999; Bussis and Heineke, 1998). Proline is proposed to stabilize cellular structures and membranes through hydrophilic interactions and hydrogen bonding and to maintain turgor pressure and water content (Verslues and Sharma, 2010). In the case of the 481-B drought-induced lateral root growth depends on the combined capacity of proline to act as a redox buffer as a source of energy and as an osmoticum to maintain due cellular homeostasis and tissue functionality.

While the invention has been described with reference to various and preferred embodiments, it should be understood by those skilled in the art that various changes may be made and equivalents may be substituted for elements thereof without departing from the essential scope of the invention. In addition, many modifications may be made to adapt a particular situation or material to the teachings of the invention without departing from the essential scope thereof. For example in addition to being applicable to rice, the materials and methods disclosed herein may also be applied to other cereal grass, including but not limited to corn, wheat, barley, sorghum, millet, oats, and rye

Therefore, it is intended that the invention not be limited to the particular embodiment disclosed herein contemplated for carrying out this invention, but that the invention will include all embodiments falling within the scope of the claims.

What is claimed is:

1. A method of improving lateral root growth and water uptake in a cereal grass comprising:
a) crossing a crossing plant of one variety of cereal grass having chromosomal DNA that comprises a nucleic acid comprising qDTY_{12.1}, or a yield-improving part thereof, with a recipient plant of a distinct variety of cereal grass having chromosomal DNA that does not include a nucleic acid comprising qDTY_{12.1}, or a yield-improving part thereof; and
b) selecting one or more progeny plants having chromosomal DNA that comprises a nucleic acid comprising qDTY_{12.1}, or a yield-improving part thereof,
wherein qDTY_{12.1}, or a yield-improving part thereof, is detected in the crossing plant, recipient plant, or one or more progeny plants by analyzing genomic DNA from the crossing plant, the recipient plant, or one or more progeny plant, or germplasm, pollen, or seed thereof, for the presence of at least one molecular marker linked to qDTY_{12.1}, or a yield-improving part thereof, wherein the at least one molecular marker is selected from the group consisting of: RM28048; RM28076; RM28089; RM28099; RM28130; RM511; RM1261; RM28166; RM28199; and Indel-8, and wherein a selected one or more progeny plant having DNA that comprises a nucleic acid comprising qDTY_{12.1}, or a yield-improving part thereof, has improved lateral root growth and water uptake.

2. The method of claim 1, further comprising the steps:
a) backcrossing the one or more selected progeny plants to produce backcross progeny plants; and
b) selecting one or more backcross progeny plants having chromosomal DNA that comprises a nucleic acid comprising qDTY_{12.1}, or a yield-improving part thereof, wherein qDTY_{12.1}, or a yield-improving part thereof, is detected in the one or more backcross progeny plants by analyzing genomic DNA from the one or more backcross progeny plants, or germplasm, pollen, or seed thereof, for the presence of at least one molecular marker linked to qDTY_{12.1}, or a yield-improving part thereof, wherein the at least one molecular marker is selected from the group consisting of: RM28048; RM28076; RM28089; RM28099; RM28130; RM511; RM1261; RM28166; RM28199; and Indel-8.

3. The method of claim 2, wherein steps a) and b) are repeated one or more times to produce third or higher backcross progeny plants having chromosomal DNA that comprises a nucleic acid comprising qDTY_{12.1}, or a yield-improving part thereof, wherein qDTY_{12.1}, or a yield-improving part thereof, is detected in the one or more backcross progeny plants by analyzing genomic DNA from the one or more backcross progeny plants, or germplasm, pollen, or seed thereof, for the presence of at least one molecular marker linked to qDTY_{12.1}, or a yield-improving part thereof, wherein the at least one molecular marker is selected from the group consisting of: RM28048; RM28076; RM28089; RM28099; RM28130; RM511; RM1261; RM28166; RM28199; and Indel-8.

4. The method of claim 3, wherein physiological and morphological characteristics of the recipient plant, other than those of lateral root growth and water uptake, are retained.

5. The method of claim 1, wherein at least one of the crossing plant and the recipient plant has chromosomal DNA comprising a nucleic acid having at least 70% sequence identity to Ulp1.

6. The method of claim 1, wherein the selected one or more progeny plants is further selected for having increased lateral root growth relative to a control plant.

7. The method of claim 1, wherein the selected one or more progeny plants is further selected for having increased lateral root growth relative to a control plant in both well watered and drought conditions.

8. The method of claim 1, wherein the selected one or more progeny plants is further selected for having improved yield under drought conditions relative to a control plant.

9. The method of claim 1, wherein the selected one or more progeny plants is further selected for having at least one trait associated with improved yield under drought conditions selected from the group consisting of: increased sucrose content in flag leaf relative to a control plant; increased sucrose content in spikelets relative to a control plant; increased starch content in spikelets relative to a control plant; and increased carbon reserves in roots relative to a control plant.

10. The method of claim 1, wherein the cereal grass is selected from the group consisting of: rice; corn;
wheat; barley; sorghum; millet; oats; and rye.

11. The method of claim 1, wherein the cereal grass is rice.

12. The method of claim 1, wherein the cereal grass is corn.

13. The method of claim 1, wherein the crossing plant is a rice plant selected from the group consisting of: WayRarem; IR79971-B-102-B; and IR74371-46-1-1.

14. The method of claim 1, wherein the recipient plant is a rice plant selected from the group consisting of: Vandana; Kalinga 3; Anjali; IR64; Swarna; Sambha Mahsuri; MTU1010, Lalat; Naveen; Sabitri; BR11; BR29; BR28; TDK1; TDK 9; and Chirang.

15. The method of claim 1, wherein the yield improving part of qDTY_{12.1} comprises one or more nucleic acids sharing at least 70% identity with one or more nucleic acids of the group of nucleic acids consisting of: SEQ ID NO: 2 (OsNAM_{12.1}); SEQ ID NO: 3 (OsGPDP_{12.1}); SEQ ID NO: 4 (OsGPDP_{12.1}); SEQ ID NO: 5 (OsGPDP_{12.1}); SEQ ID NO: 6 (OsSTPK_{12.1}); SEQ ID NO: 7 (OsSTPK_{12.1}); SEQ ID NO: 8 (OsSTPK_{12.1}); SEQ ID NO: 9 (OsPOle_{12.1}); SEQ ID NO: 10 (OsMtN3_{12.1}); SEQ ID NO: 11 (OsWAK_{12.1}); SEQ ID NO: 12 (OsCesA_{12.1}); SEQ ID NO: 13 (OsGDP_{12.1}); SEQ ID NO: 14 (OsARF_{12.1}); SEQ ID NO: 15 (OsARF_{12.1}); SEQ ID NO: 16 (OsARF_{12.1}); SEQ ID NO: 17 (OsARF_{12.1}); SEQ ID NO: 18 (OsARF_{12.1}); SEQ ID NO: 20 (OsAmh_{12.1}); and SEQ ID NO: 21 (OsAmh_{12.1}).

16. The method of claim 1, wherein the yield improving part of qDTY_{12.1} comprises a nucleic acid sharing an identity with SEQ ID NO: 2 (OsNAM_{12.1}) selected from the group consisting of: at least 70% identity; at least 75% identity; at least 80% identity; at least 85% identity; at least 90% identity; at least 95% identity; at least 96% identity; at least 97% identity; at least 98% identity; at least 99% identity; and 100% identity.

17. The method of claim 1, wherein the crossing plant, in addition to having chromosomal DNA that comprises a nucleic acid comprising qDTY12.1, or a yield-improving part thereof, also comprises a nucleic acid comprising qDTY_{2.3}.

18. The method of claim 1, wherein the recipient plant has chromosomal DNA that comprises a nucleic acid comprising qDTY_{2.3}.

19. A method of improving lateral root growth and water uptake in a cereal grass comprising:
a) crossing a crossing plant of one variety of cereal grass having chromosomal DNA that comprises a nucleic acid sharing an identity with SEQ ID NO: 2 (OsNAM_{12.1}) selected from the group consisting of: at least 70% identity; at least 75% identity; at least 80% identity; at least 85% identity; at least 90% identity; at least 95% identity; at least 96% identity; at least 97% identity; at least 98% identity; at least 99% identity; and 100% identity, with a recipient plant of a distinct variety of cereal grass having chromosomal DNA that does not include a nucleic acid sharing at least 70% identity with SEQ ID NO: 2 (OsNAM_{12.1}); and
b) selecting one or more progeny plants having chromosomal DNA that comprises a nucleic acid sharing an identity with SEQ ID NO: 2 (OsNAM_{12.}) selected from the group consisting of: at least 70% identity; at least 75% identity; at least 80% identity; at least 85% identity; at least 90% identity; at least 95% identity; at least 96% identity; at least 97% identity; at least 98% identity; at least 99% identity; and 100% identity.

20. The method of claim 19, wherein the nucleic acid sharing an identity with SEQ ID NO: 2 (OsNAM_{12.}) selected from the group consisting of: at least 70% identity; at least 75% identity; at least 80% identity; at least 85% identity; at least 90% identity; at least 95% identity; at least 96% identity; at least 97% identity; at least 98% identity; at least 99% identity; and 100% identity is detected by RT-PCR.

21. The method of claim 19, further comprising the steps:
c) backcrossing the one or more selected progeny plants produce backcross progeny plants; and
d) selecting one or more backcross progeny plants having chromosomal DNA that comprises a nucleic acid sharing an identity with SEQ ID NO: 2 (OsNAM_{12.}) selected from the group consisting of: at least 70% identity; at least 75% identity; at least 80% identity; at least 85% identity; at least 90% identity; at least 95% identity; at least 96% identity; at least 97% identity; at least 98% identity; at least 99% identity; and 100% identity.

22. The method of claim 21, wherein steps c) and d) are repeated one or more times to produce third or higher backcross progeny plants having chromosomal DNA that comprises a nucleic acid sharing an identity with SEQ ID NO: 2 (OsNAM_{12.}) selected from the group consisting of: at least 70% identity; at least 75% identity; at least 80% identity; at least 85% identity; at least 90% identity; at least 95% identity; at least 96% identity; at least 97% identity; at least 98% identity; at least 99% identity; and 100% identity.

23. The method of claim 19, wherein at least one of the crossing plant and the recipient plant has chromosomal DNA comprising a nucleic acid having at least 70% sequence identity to Ulp1, wherein Ulp1 encodes a functional deSUMOylating protease capable of deSUMOylating a polypeptide encoded by the nucleic acid sharing an identity with SEQ ID NO: 2 (OsNAM_{12.}) selected from the group consisting of: at least 70% identity; at least 75% identity; at least 80% identity; at least 85% identity; at least 90% identity; at least 95% identity; at least 96% identity; at least 97% identity; at least 98% identity; at least 99% identity; and 100% identity.

24. The method of claim 19, wherein the selected one or more progeny plants is further selected for having increased lateral root growth relative to a control plant.

25. The method of claim 19, wherein the selected one or more progeny plants is further selected for having increased lateral root growth relative to a control plant in both well watered and drought conditions.

26. The method of claim 19, wherein the selected one or more progeny plants is further selected for having improved yield under drought conditions relative to a control plant.

27. The method of claim 19, wherein the selected one or more progeny plants is further selected for having at least one trait associated with improved yield under drought conditions selected from the group consisting of: increased sucrose content in flag leaf relative to a control plant; increased sucrose content in spikelets relative to a control plant; increased starch content in spikelets relative to a control plant; and increased carbon reserves in roots relative to a control plant

28. The method of claim 19, wherein the cereal grass is selected from the group consisting of: rice; corn; wheat; barley; sorghum; millet; oats; and rye.

29. The method of claim 19, wherein the cereal grass is rice.

30. The method of claim 19, wherein the cereal grass is corn.

31. The method of claim 19, wherein the crossing plant is a rice plant selected from the group consisting of: WayRarem; IR79971-B-102-B; and IR74371-46-1-1.

32. The method of claim 19, wherein the recipient plant is a rice plant selected from the group consisting of: Vandana; Kalinga 3; Anjali; IR64; Swarna; Sambha Mahsuri; MTU1010, Lalat; Naveen; Sabitri; BR11; BR29; BR28; TDK1; TDK 9; and Chirang.

33. The method of claim 19, wherein the crossing plant has chromosomal DNA that comprises one or more nucleic acids sharing an identity selected from the group consisting of: at least 70% identity; at least 75% identity; at least 80% identity; at least 85% identity; at least 90% identity; at least 95% identity; at least 96% identity; at least 97% identity; at least 98% identity; at least 99% identity; and 100% identity, with one or more nucleic acids of the group of nucleic acids consisting of: SEQ ID NO: 3 (OsGPDP_{12.1}); SEQ ID NO: 4 (OsGPDP_{12.1}); SEQ ID NO: 5 (OsGPDP_{12.1}); SEQ ID NO: 6 (OsSTPK_{12.1}); SEQ ID NO: 7 (OsSTPK_{12.1}); SEQ ID NO: 8 (OsSTPK_{12.1}); SEQ ID NO: 9 (OsPOle_{12.1}); SEQ ID NO: 10 (OsMtN3_{12.1}); SEQ ID NO: 11 (OsWAK_{12.1}); SEQ ID NO: 12 (OsCesA_{12.1}); SEQ ID NO: 13 (OsGDP_{12.1}); SEQ ID NO: 14 (OsARF_{12.1}); SEQ ID NO: 15 (OsARF_{12.1}); SEQ ID NO: 16 (OsARF_{12.1}); SEQ ID NO: 17 (OsARF_{12.1}); SEQ ID NO: 18 (OsARF_{12.1}); SEQ ID NO: 20 (OsAmh_{12.1}); and SEQ ID NO: 21 (OsAmh_{12.1}).

34. The method of claim 19, wherein the crossing plant has chromosomal DNA that comprises a nucleic acid comprising qDTY_{2.3} and the nucleic acid comprising qDTY_{2.3} is selected for in the recipient plant.

35. The method of claim 19, wherein the recipient plant has chromosomal DNA that comprises a nucleic acid comprising qDTY_{2.3}.

36. A method for selecting a cereal grass plant having improved lateral root growth and water uptake relative to a control cereal grass plant, comprising:
a) inducing expression or increasing expression in a cereal grass plant a nucleic acid comprising qDTY_{12.1}, or a yield-improving part thereof, wherein the induced or increased expression of the nucleic acid comprising qDTY_{12.1}, or a yield-improving part thereof, is obtained by transforming and expressing in the cereal grass plant the nucleic acid comprising qDTY_{12.1}, or a yield-improving part thereof; and
b) selecting a cereal grass plant having improved lateral root growth and water uptake relative to a control cereal grass plant, wherein the cereal grass plant having improved lateral root growth and water uptake relative to a control cereal grass plant is selected by analyzing genomic DNA from the cereal grass plant, or germplasm, pollen, or seed thereof, and detecting therein at least one molecular marker linked to qDTY12.1, or a yield-improving part thereof, wherein the at least one molecular marker is selected from the group consisting of: RM28048; RM28076; RM28089; RM28099; RM28130; RM511; RM1261; RM28166; RM28199; and Indel-8.

37. The method of claim 36, wherein the cereal grass plant has chromosomal DNAcomprising a nucleic acid having at least 70% sequence identity to Ulp1.

38. The method of claim 36, wherein the selected cereal grass plant is further selected for having improved yield under drought conditions compared to a control cereal grass plant.

39. The method of claim 36, wherein the induced or increased expression of the nucleic acid comprising qDTY12.1, or a yield-improving part thereof, is a result of introducing and expressing the nucleic acid comprising qDTY_{12.1}, or a yield-improving part thereof, in the cereal grass plant under control of at least one promoter functional in plants.

40. The method of claim 36, wherein the at least one promoter and the nucleic acid comprising qDTY_{12.1}, or yield improving part thereof, are operably linked.

41. The method of claim 36, wherein the cereal grass plant is selected from the group consisting of: rice; corn; wheat; barley; sorghum; millet; oats; and rye.

42. The method of claim 36, wherein the yield-improving part of qDTY_{12.1} comprises one or more nucleic acids sharing an identity selected from the group consisting of: at least 70% identity; at least 75% identity; at least 80% identity; at least 85% identity; at least 90% identity; at least 95% identity; at least 96% identity; at least 97% identity; at least 98% identity; at least 99% identity; and 100% identity, with one or more nucleic acids of the group of nucleic acids consisting of: SEQ ID NO: 2 (OsNAM_{12.1}); SEQ ID NO: 3 (OsGPDP_{12.1}); SEQ ID NO: 4 (OsGPDP_{12.1}); SEQ ID NO: 5 (OsGPDP_{12.1}); SEQ ID NO: 6 (OsSTPK_{12.1}); SEQ ID NO: 7 (OsSTPK_{12.1}); SEQ ID NO: 8 (OsSTPK_{12.1}); SEQ ID NO: 9 (OsPOle_{12.1}); SEQ ID NO: 10 (OsMtN3_{12.1}); SEQ ID NO: 11 (OsWAK_{12.1}); SEQ ID NO: 12 (OsCesA_{12.1}); SEQ ID NO: 13 (OsGDP_{12.1}); SEQ ID NO: 14 (OsARF_{12.1}); SEQ ID NO: 15 (OsARF_{12.1}); SEQ ID NO: 16 (OsARF_{12.1}); SEQ ID NO: 17 (OsARF_{12.1}); SEQ ID NO: 18 (OsARF_{12.1}); SEQ ID NO: 20 (OsAmh₁₂); and SEQ ID NO: 21 (OsAmh_{12.1}).

43. The method of claim 36, wherein the yield improving part of qDTY_{12.1} comprises a nucleic acid sharing an identity with SEQ ID NO: 2 (OsNAM_{12.1}) selected from the group consisting of: at least 70% identity; at least 75% identity; at least 80% identity; at least 85% identity; at least 90% identity; at least 95% identity; at least 96% identity; at least 97% identity; at least 98% identity; at least 99% identity; and 100% identity.

44. A method for generating a cereal grass plant having improved lateral root growth and water uptake relative to a control cereal grass plant comprising:
a) transforming a cereal grass plant cell, cereal grass plant, or part thereof with a construct comprising:
(1) a nucleic acid encoding a polypeptide having a sequence identity selected from the group consisting of: at least 70% identity; at least 75% identity; at least 80% identity; at least 85% identity; at least 90% identity; at least 95% identity; at least 96% identity; at least 97% identity; at least 98% identity; at least 99% identity; and 100% identity to nucleic acid sequence SEQ ID NO: 2 (OsNAM_{12.1});
(2) a promoter operably linked to the nucleic acid; and
(3) a transcription termination sequence; and
b) expressing the construct in a cereal grass plant cell, cereal grass plant, or part thereof, thereby generating a cereal grass plant having improved lateral root growth and water uptake relative to a control cereal grass plant.

45. The method of claim 44, wherein the construct further comprises one or more nucleic acids sharing at an identity selected from the group consisting of: at least 70% identity; at least 75% identity; at least 80% identity; at least 85% identity; at least 90% identity; at least 95% identity; at least 96% identity; at least 97% identity; at least 98% identity; at least 99% identity; and 100% identity, with one or more nucleic acids of the group of nucleic acids consisting of: SEQ ID NO: 3 (OsGPDP_{12.1}); SEQ ID NO: 4 (OsGPDP_{12.1}); SEQ ID NO: 5 (OsGPDP_{12.1}); SEQ ID NO: 6 (OsSTPK_{12.1}); SEQ ID NO: 7 (OsSTPK_{12.1}); SEQ ID NO: 8 (OsSTPK_{12.1}); SEQ ID NO: 9 (OsPOle_{12.1}); SEQ ID NO: 10 (OsMtN3_{12.1}); SEQ ID NO: 11 (OsWAK_{12.1}); SEQ ID NO: 12 (OsCesA_{12.1}); SEQ ID NO: 13 (OsGDP_{12.1}); SEQ ID NO: 14 (OsARF_{12.1}); SEQ ID NO: 15 (OsARF_{12.1}); SEQ ID NO: 16 (OsARF_{12.1}); SEQ ID NO: 17 (OsARF_{12.1}); SEQ ID NO: 18 (OsARF_{12.1}); SEQ ID NO: 20 (OsAmh_{12.1}); and SEQ ID NO: 21 (OsAmh_{12.1}).

46. The method of the claim 44, wherein the construct further comprises a nucleic acid having at least 70% sequence identity to Ulp1, wherein the nucleic acid encoding a deSUMOylating protease encodes a functional deSUMOylating protease capable of deSUMOylating a polypeptide encoded by the nucleic acid sharing an identity with SEQ ID NO: 2 (OsNAM_{12.}) selected from the group consisting of: at least 70% identity; at least 75% identity; at least 80% identity; at least 85% identity; at least 90% identity; at least 95% identity; at least 96% identity; at least 97% identity; at least 98% identity; at least 99% identity; and 100% identity.

47. The method of the claim 44, further comprising a step of selecting for a cereal grass plant having improved lateral root growth relative to a control cereal grass plant.

48. The method of claim 44, further comprising a step of selecting for a cereal grass plant having improved yield under drought conditions relative to a control cereal grass plant.

49. The method of claim 44, further comprising a step of selecting for a cereal grass plant having a phenotype comprising one or more characteristics selected from the group consisting of: increased sucrose content in flag leaf relative to a control plant; increased sucrose content in spikelets relative to a control plant; increased starch content in spikelets relative to a control plant; and increased carbon reserves in roots relative to a control plant.

50. The method of claim 44, wherein the cereal grass plant cell, cereal grass plant, or part thereof is selected from the group consisting of: rice; corn; wheat; barley; sorghum; millet; oats; and rye.

51. A method for the production of a transgenic cereal grass plant having improved lateral root growth and water uptake relative to a control cereal grass plant comprising:
a) transforming and expressing in a cereal grass plant cell at least one nucleic acid having at a sequence identity selected from the group consisting of: at least 70% identity; at least 75% identity; at least 80% identity; at least 85% identity; at least 90% identity; at least 95% identity; at least 96% identity; at least 97% identity; at least 98% identity; at least 99% identity; and 100% identity with one or more nucleic acids of the group of nucleic acids consisting of: SEQ ID NO: 2 (OsNAM_{12.1}); SEQ ID NO: 3 (OsGPDP_{12.1}); SEQ ID NO: 4 (OsGPDP_{12.1}); SEQ ID NO: 5 (OsGPDP_{12.1}); SEQ ID NO: 6 (OsSTPK_{12.1}); SEQ ID NO: 7 (OsSTPK_{12.1}); SEQ ID NO: 8 (OsSTPK_{12.1}); SEQ ID NO: 9 (OsPOle_{12.1}); SEQ ID NO: 10 (OsMtN3_{12.1}); SEQ ID NO: 11 (OsWAK_{12.1}); SEQ ID NO: 12 (OsCesA_{12.1}); SEQ ID NO: 13 (OsGDP_{12.1}); SEQ ID NO: 14 (OsARF_{12.1}); SEQ ID NO: 15 (OsARF_{12.1}); SEQ ID NO: 16 (OsARF_{12.1}); SEQ ID NO: 17 (OsARF_{12.1}); SEQ ID NO: 18 (OsARF_{12.1}); SEQ ID NO: 20 (OsAmh_{12.1}); and SEQ ID NO: 21 (OsAmh_{12.1}); and
b) cultivating the cereal grass plant cell under conditions promoting plant growth and development, and obtaining transformed plants expressing one or more of OsNAM_{12.1}, OsGPDP_{12.1}, OsSTPK_{12.1}, OsPOle_{12.1}, OsMtN3_{12.1}, OsWAK_{12.1}, OsCesA_{12.1}, OsGDP_{12.1}, OsARF_{12.1}, and OsAmh_{12.1}.

52. The method of claim 51, further comprising transforming and expressing in the cereal grass plant cell a nucleic acid having at least 70% sequence identity to Ulp1, wherein Ulp1 encodes a functional deSUMOylating protease capable of deSUMOylating a polypeptide encoded by the nucleic acid having at least 70% sequence identity with SEQ ID NO: 2 (OsNAM_{12.1}).

53. The method of claim 51, further comprising a step of selecting for a cereal grass plant having improved lateral root growth relative to a control cereal grass plant.

54. The method of claim 51, further comprising a step of selecting for a cereal grass plant having improved yield under drought conditions relative to a control cereal grass plant.

55. The method of claim 51, further comprising a step of selecting for a cereal grass plant having a phenotype comprising one or more characteristics selected from the group consisting of: increased sucrose content in flag leaf relative to a control plant; increased sucrose content in spikelets relative to a control plant; increased starch content in spikelets relative to a control plant; and increased carbon reserves in roots relative to a control plant.

56. A transgenic plant cell comprising:
a) at least one promoter that is functional in plants; and
b) at least one nucleic acid having a sequence identity selected from the group consisting of: at least 70% identity; at least 75% identity; at least 80% identity; at least 85% identity; at least 90% identity; at least 95% identity; at least 96% identity; at least 97% identity; at least 98% identity; at least 99% identity; and 100% identity with one or more nucleic acids of the group of nucleic acids consisting of: SEQ ID NO: 2 (OsNAM_{12.1}); SEQ ID NO: 3 (OsGPDP_{12.1}); SEQ ID NO: 4 (OsGPDP_{12.1}); SEQ ID NO: 5 (OsGPDP_{12.1}); SEQ ID NO: 6 (OsSTPK_{12.1}); SEQ ID NO: 7 (OsSTPK_{12.1}); SEQ ID NO: 8 (OsSTPK_{12.1}); SEQ ID NO: 9 (OsPOle_{12.1}); SEQ ID NO: 10 (OsMtN3_{12.1}); SEQ ID NO: 11 (OsWAK_{12.1}); SEQ ID NO: 12 (OsCesA_{12.1}); SEQ ID NO: 13 (OsGDP_{12.1}); SEQ ID NO: 14 (OsARF_{12.1}); SEQ ID NO: 15 (OsARF_{12.1}); SEQ ID NO: 16 (OsARF_{12.1}); SEQ ID NO: 17 (OsARF_{12.1}); SEQ ID NO: 18 (OsARF_{12.1}); SEQ ID NO: 20 (OsAmh_{12.1}); and SEQ ID NO: 21 (OsAmh_{12.1}), wherein the promoter and polynucleotide are operably linked and incorporated into the plant cell chromosomal DNA.

57. The transgenic plant cell of claim 56, further comprising a nucleic acid having at least 70% sequence identity to Ulp1, wherein Ulp1 encodes a functional deSUMOylating proteas capable of deSUMOylating a polypeptide encoded by the nucleic acid having at least 70% sequence identity with SEQ ID NO: 2 (OsNAM_{12.1}).

58. The transgenic plant cell of claim 56, wherein the type of plant cell is selected from the group consisting of: rice plant cell; corn plant cell; wheat plant cell; barley plant cell; sorghum plant cell; millet plant cell; oats plant cell; and rye plant cell.

59. The transgenic plant cell of claim 56, wherein the plant cell is homozygous for the at least one nucleic acids.

60. A transgenic plant comprising a plurality of transgenic plant cells of claim 56.

61. A transgenic plant comprising:
a) at least one promoter that is functional in plants; and
b) at least one nucleic acid having a sequence identity selected from the group consisting of: at least 70% identity; at least 75% identity; at least 80% identity; at least 85% identity; at least 90% identity; at least 95% identity; at least 96% identity; at least 97% identity; at least 98% identity; at least 99% identity; and 100% identity with one or more nucleic acids of the group of nucleic acids consisting of: SEQ ID NO: 2 (OsNAM_{12.1}); SEQ ID NO: 3 (OsGPDP_{12.1}); SEQ ID NO: 4 (OsGPDP_{12.1}); SEQ ID NO: 5 (OsGPDP_{12.1}); SEQ ID NO: 6 (OsSTPK_{12.1}); SEQ ID NO: 7 (OsSTPK_{12.1}); SEQ ID NO: 8 (OsSTPK_{12.1}); SEQ ID NO: 9 (OsPOle_{12.1}); SEQ ID NO: 10 (OsMtN3_{12.1}); SEQ ID NO: 11 (OsWAK_{12.1}); SEQ ID NO: 12 (OsCesA_{12.1}); SEQ ID NO: 13 (OsGDP_{12.1}); SEQ ID NO: 14 (OsARF_{12.1}); SEQ ID NO: 15 (OsARF_{12.1}); SEQ ID NO: 16 (OsARF_{12.1}); SEQ ID NO: 17 (OsARF_{12.1}); SEQ ID NO: 18 (OsARF_{12.1}); SEQ ID NO: 20 (OsAmh_{12.1}); and SEQ ID NO: 21 (OsAmh_{12.1}),
wherein the promoter and polynucleotide are operably linked and incorporated into the plant cell chromosomal DNA.

62. The transgenic plant of claim 61, wherein the plant is selected from the group consisting of: rice; corn; wheat; barley; sorghum; millet; oats; and rye.

63. The transgenic plant of claim 61, wherein the transgenic plant is homozygous for the at least one nucleic acid.

64. A seed of a plant of claim 61.

65. A plant part of a plant of claim 61.

66. A method for selecting transgenic plants having improved lateral root growth and water uptake relative to a control plant, comprising:
a) screening a population of plants for increased lateral root growth and water uptake, wherein plants in the population comprise a transgenic plant cell having recombinant DNA incorporated into its chromosomal DNA, wherein the recombinant DNA comprises a promoter that is functional in a plant cell and that is functionally linked to an open reading frame of a nucleic acid having a sequence identity selected from the group consisting of: at least 70% identity; at least 75% identity; at least 80% identity; at least 85% identity; at least 90% identity; at least 95% identity; at least 96% identity; at least 97% identity; at least 98% identity; at least 99% identity; and 100% identity with one or more nucleic acids of the group of nucleic acids consisting of: SEQ ID NO: 2 (OsNAM_{12.1}); SEQ ID NO: 3 (OsGPDP_{12.1}); SEQ ID NO: 4 (OsGPDP_{12.1}); SEQ ID NO: 5 (OsGPDP_{12.1}); SEQ ID NO: 6 (OsSTPK_{12.1}); SEQ ID NO: 7 (OsSTPK_{12.1}); SEQ ID NO: 8 (OsSTPK_{12.1}); SEQ ID NO: 9 (OsPOle_{12.1}); SEQ ID NO: 10 (OsMtN3_{12.1}); SEQ ID NO: 11 (OsWAK_{12.1}); SEQ ID NO: 12 (OsCesA_{12.1}); SEQ ID NO: 13 (OsGDP_{12.1}); SEQ ID NO: 14 (OsARF_{12.1}); SEQ ID NO: 15 (OsARF_{12.1}); SEQ ID NO: 16 (OsARF_{12.1}); SEQ ID NO: 17 (OsARF_{12.1}); SEQ ID NO: 18 (OsARF_{12.1}); SEQ ID NO: 20 (OsAmh_{12.1}); and SEQ ID NO: 21 (OsAmh_{12.1}), wherein individual plants in said population that comprise the transgenic plant cell exhibit increased yield under drought conditions relative to control plants which do not comprise the transgenic plant cell; and
b) selecting from said population one or more plants that exhibit lateral root growth and water uptake greater than the lateral root growth and water uptake in control plants which do not comprise the transgenic plant cell.

67. The method of claim 66, further comprising selecting one or more plants that exhibit increased yield under drought conditions at a level greater than the yield under drought conditions in control plants that do not comprise the transgenic plant cell.

68. The method of claim 66, further comprising a step of collecting seeds from the one or more plants selected in step b).

69. The method of claim 66, wherein the plant is selected from the group consisting of: rice; corn; wheat; barley; sorghum; millet; oat; and rye.

70. The method of claim 66, wherein the plant is rice.

71. The method of claim 66, wherein the plant is corn.

72. The plant, plant cell, or any one of the methods herein, wherein the % identity is selected from the group consisting of: 70%; 75%; 80%; 85%; 90%; 95%; 96%; 97%; 98%; 99%; and 100%.

73. A method of improving lateral root growth and water uptake in a cereal grass plant comprising modifying a nucleic acid encoding no-apical meristem (NAM) transcription factor in a cereal grass so that the nucleic acid encoding the NAM transcription factor shares an identity with SEQ ID NO: 2 (OsNAM_{12.}) selected from the group consisting of: at least 90% identity; at least 95% identity; at least 96% identity; at least 97% identity; at least 98% identity; at least 99% identity; and 100% identity.

74. The method of claim 73, further comprising modifying one or more nucleic acids encoding one or more genes selected from the group consisting of GPDP; STPK; POle; MtN3; WAK, CesA; GDP; ARF; and Amh so that the one or more nucleic acids share an identity selected from the group consisting of: at least 95% identity; at least 96% identity; at least 97% identity; at least 98% identity; at least 99% identity; and 100% identity with one or more nucleic acids of the group of nucleic acids consisting of: SEQ ID NO: 3 (OsGPDP_{12.1}); SEQ ID NO: 4 (OsGPDP_{12.1}); SEQ ID NO: 5 (OsGPDP_{12.1}); SEQ ID NO: 6 (OsSTPK_{12.1}); SEQ ID NO: 7 (OsSTPK_{12.1}); SEQ ID NO: 8 (OsSTPK_{12.1}); SEQ ID NO: 9 (OsPOle_{12.1}); SEQ ID NO: 10 (OsMtN3_{12.1}); SEQ ID NO: 11 (OsWAK_{12.1}); SEQ ID NO: 12 (OsCesA_{12.1}); SEQ ID NO: 13 (OsGDP_{12.1}); SEQ ID NO: 14 (OsARF_{12.1}); SEQ ID NO: 15 (OsARF_{12.1}); SEQ ID NO: 16 (OsARF_{12.1}); SEQ ID NO: 17 (OsARF_{12.1}); SEQ ID NO: 18 (OsARF_{12.1}); SEQ ID NO: 20 (OsAmh_{12.1}); and SEQ ID NO: 21 (OsAmh_{12.1}).

75. The method of claim 73, wherein the cereal grass comprises a nucleic acid comprising qDTY_{2.3}.

76. The method of claim 73, wherein the cereal grass further comprises a nucleic acid having at least 70% sequence identity to Ulp1, wherein Ulp1 encodes a functional deSUMOylating protease capable of deSUMOylating a polypeptide encoded by the nucleic acid sharing an identity with SEQ ID NO: 2 (OsNAM_{12.}) selected from the group consisting of: at least 95% identity; at least 96% identity; at least 97% identity; at least 98% identity; at least 99% identity; and 100% identity.

77. The method of claim 73, wherein modifying the nucleic acid is performed using a technique selected from the group consisting of: transgenic method; crossing; backcrossing; protoplast fusion; doubled haploid technique; embryo rescue; zinc-finger nucleases; transcription activator-like effector nucleases; and clustered regularly interspaced short palindromic repeat (CRISPR)/Cas-based RNA-guided DNA endonucleases.

78. The method of claim 73, wherein the wherein the cereal grass is selected from the group consisting of: rice; corn; wheat; barley; sorghum; millet; oats; and rye.

79. The method of claim 73, wherein the cereal grass is rice.

80. The method of claim 73, wherein the cereal grass is corn.
